(19) United States
(12) Patent Application Publication
(10) Pub. No.: US 2008/0125587 A1
(43) Pub. Date: May 29, 2008

(54) Synthesis of triazole compounds that modulate HSP90 activity

(76) Inventors: Dinesh U. Chimmanamada, Arlington, MA (US); Chi-Wan Lee, Grafton, MA (US); David James, Cambridge, MA (US); Shijie Zhang, Nashua, NH (US); Weiwen Ying, Ayer, MA (US); Junghyun Chae, Youngdengpo-gu (KR); Teresa Przewloka, Tewksbury, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/807,333

(22) Filed: May 25, 2007

Related U.S. Application Data
(60) Provisional application No. 60/808,342, filed on May 25, 2006.
(60) Provisional application No. 60/808,376, filed on May 25, 2006.
(60) Provisional application No. 60/808,375, filed on May 25, 2006.
(60) Provisional application No. 60/902,031, filed on Feb. 16, 2007.

(51) Int. Cl. C07D 249/12; C07D 407/02; C07D 403/02; C07D 401/02; C07D 401/04; C07D 403/10; C07D 417/02; C07D 487/04; C07D 413/12; C07D 413/02

(52) U.S. Cl. 544/105; 548/263.2; 546/167; 546/272.4; 546/143; 548/253; 548/178; 544/277; 544/132; 548/217

(57) ABSTRACT

The present invention provides novel methods of preparing triazole compounds which inhibit the activity of Hsp90. One embodiment of the invention is directed to methods for preparing a triazole compound represented by the following Structural Formula:

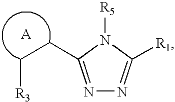

or a tautomer, a pharmaceutically acceptable salt, solvate, or clathrate, or a prodrug thereof, comprising the steps of: a) reacting an amide represented by the following Structural Formula:

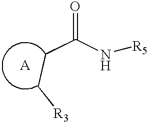

with a thionation reagent to form a thioamide; b) reacting the thioamide of step a) with hydrazine to form a hydrazonamide; c) reacting the hydrazonamide of step b) with a carbonylation or a thiocarbonylation reagent.

In one embodiment, the present invention is a method of synthesis of a compound of formula (IA)

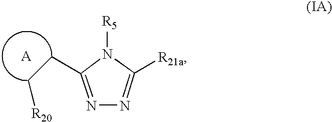

or a tautomer, a pharmaceutically acceptable salt, solvate, or clathrate, or a prodrug thereof, comprising reacting a compound of formula (IIA)

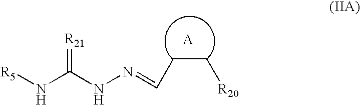

with an oxidizing agent, thereby producing a compound of formula (IA).

The present invention is also directed to a method of preparing a compound or a tautomer thereof represented by the following Structural Formula:

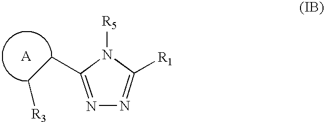

or a tautomer, a pharmaceutically acceptable salt, solvate, or clathrate, or a prodrug thereof. The method comprises the step of reacting a first starting compound represented by the following Structural Formula:

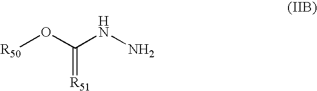

in the presence of a mercuric salt, with a second starting compound represented by the following Structural Formula:

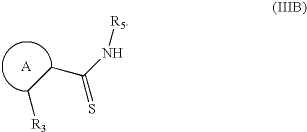

### RELATED APPLICATIONS

This application claims the benefit of U.S. Provisional Application No. 60/808,342, filed on May 25, 2006, U.S. Provisional Application No. 60/808,376, filed on May 25, 2006, U.S. Provisional Application No. 60/808,375, filed on May 25, 2006, and U.S. Provisional Application No. 60/902,031, filed on Feb. 16, 2007. The entire teachings of the above applications are incorporated herein by reference.

### BACKGROUND OF THE INVENTION

Certain triazole-based hsp90 inhibitors, such as the compounds described in U.S. Publication No. 20060167070, incorporated herein by reference in its entirety, show promise in the treatment of proliferative disorders, such as cancer. However, the molecules described in the referenced patent application contain a triazolone ring system, the construction of which is difficult. Synthetic processes currently available for preparing these compounds are unsuitable for commercial scale synthesis. Therefore, the need exists for improved synthese of these compounds.

### SUMMARY OF THE INVENTION

The present invention is directed to novel synthetic methods for preparing certain [1,2,4]-trizole compounds, which are suitable for industrial-scale synthesis with minimal purification required.

One embodiment of the invention is directed to a method (method I) of preparing a triazole compound represented by Structural Formula (I):

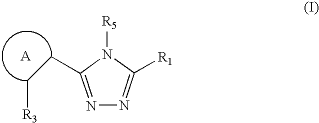

or a tautomer, a pharmaceutically acceptable salt, solvate, clathrate, or a prodrug thereof.

The method of preparing a [1,2,4]triazole compound comprises the steps of:
a) reacting an amide represented by Structural Formula (II):

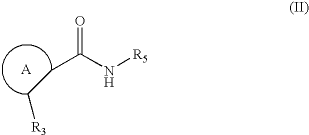

with a thionation reagent to form a thioamide represented by Structural Formula (III):

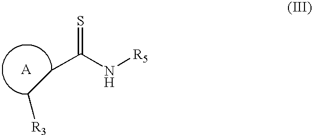

b) reacting the thioamide of Step a) with hydrazine to form a hydrazonamide represented by the Structural Formula (IV):

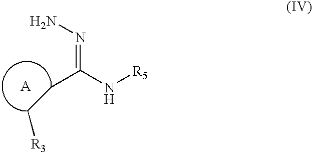

c) reacting the hydrazonamide of step b) with a carbonylation, a thiocarbonylation reagent or a compound of structural formula R₇N═C(X)₂ to form the [1,2,4]triazole compound. Any protecting groups on the product formed in step c) are removed.

In Structural Formulas (I)-(IV), variables are defined as the following:
ring A is an aryl or a heteroaryl optionally further substituted with one or more substituents in addition to R₃;
R₃ is —OR₂₆, —SR₂₆, —O(CH₂)ₘOR_{A}, O(CH₂)ₘSR_{B}, —O(CH₂)ₘNR₇R_{C}, —S(CH₂)ₘOR_{A}, —S(CH₂)ₘSR_{B}, —S(CH₂)ₘNR₇R_{C}, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —SS(O)ₚOR₇, —NR₇S(O)ₚOR₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂, OR_{A}, SR_{B}, NR₇R_{C}, NR₂₆R_{C}, or N(R_{C})₂, wherein R_{A} is a hydroxyl protecting group; R_{B} is a thiol protecting group, R_{C}, for each occurrence, is H or an amine protecting group, provided at least one R_{C} is an amine protecting group;
R₅ is an optionally substituted heteroaryl, an optionally substituted aryl, an optionally substituted cycloaliphatic, or an optionally substituted alkyl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₂₆ is a C1-C6 alkyl;
p, for each occurrence, is, independently, 0, 1 or 2;
m, for each occurrence, is independently, 1, 2, 3, or 4;
in Structural Formula (I), R₁ is —OH, —SH or —NHR₇; and X is a leaving group.

More specifically, the present invention is directed to a method of preparing a triazole compound represented by the Structural Formula (V):

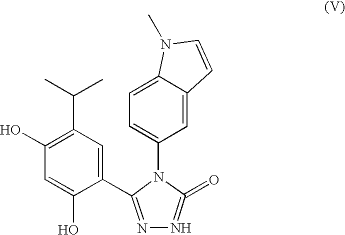

or a tautomer, a pharmaceutically acceptable salt, solvate, clathrate, or a prodrug thereof. The method of preparing the [1,2,4]triazole compound comprises the steps of:
a) reacting an amide represented by Structural Formula (VI):

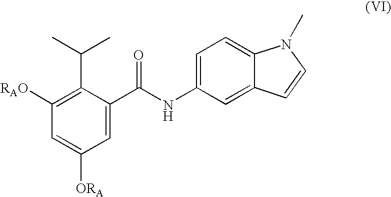

with a thionation reagent to form a thioamide represented by Structural Formula (VII):

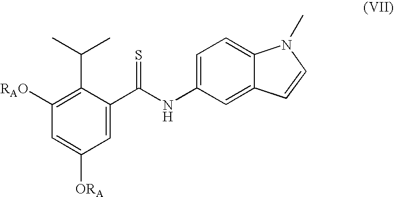

b) reacting the thioamide of step a) with hydrazine to form a hydrazonamide represented by Structural Formula (VIII):

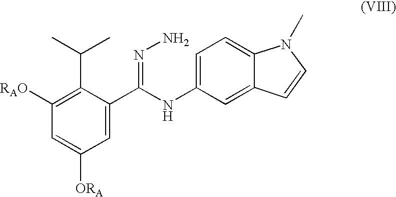

c) reacting the hydrazonamide of step b) with a carbonylation reagent to form a protected triazole compound; and
d) deprotecting the protected triazole compound formed in step c) to form the triazole compound;
wherein R_{A} is a hydroxyl protecting group.

Another embodiment of the invention is directed to a method of preparing the thioamide represented by Structural Formula (III) by reacting the amide represented by Structural Formula (II) with thionation reagent.

The present invention is also directed to a method of preparing the hydrazonamide represented by Structural Formula (IV) by reacting the thioamide of Structural Formula (III) with hydrazine.

Another embodiment of the invention is directed to a method of preparing the [1,2,4]triazole compound by reacting the hydrazonamide of Structural Formula (IV) with a carbonylation reagent, a thiocarbonylation reagent or an isocyanide.

Other embodiments of the present invention are synthetic intermediates in the preparation of the [1,2,4]triazole compound represented by Structural Formula (III) and Structural Formula (IV) by the methods disclosed herein.

In one embodiment, the present invention is a method (method II) of synthesis of a compound of Structural Formula (IA)

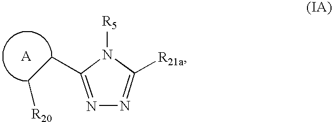

a tautomer, a pharmaceutically acceptable salt, solvate, clathrate, or a prodrug thereof, comprising reacting a compound of Structural Formula (IIA)

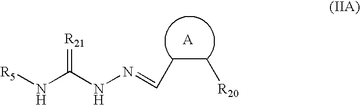

with an oxidizing agent, thereby producing a compound of Structural Formula (IA). In Structural Formulas (IA) and (IIA):

ring A is an aryl or a heteroaryl, wherein the aryl or the heteroaryl are optionally further substituted with one or more substituents in addition to R₂₀;

R₅ is an optionally substituted cycloalkyl, an optionally substituted cycloalkenyl, a substituted alkyl, a substituted phenyl, an optionally substituted heteroaryl or an optionally substituted 8 to 14 membered aryl;

R₂₀ is —ORₚ₁, —NHRₚ₃ or —N(Rₚ₃)₂, wherein Rₚ₁, for each occurrence, is independently selected from groups suitable for protecting hydroxyl, and Rₚ₃, for each occurrence, is independently selected from groups suitable for protecting an amino group;

R₂₁ is O, NH, or NR₂₆, and R₂₁ₐ is OH, NH₂ or NHR₂₆; and

R₂₆ is a C1-C6 alkyl.

In another embodiment, the present invention is a method of synthesis of a compound of Structural Formula (IIA),

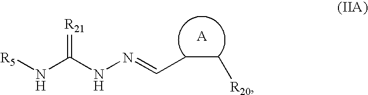

comprising reacting a compound of Structural Formula (IIIA)

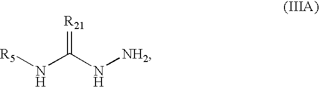

with a compound of Structural Formula (IVA)

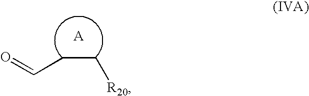

in the presence of an acid, thereby producing a compound of Structural Formula (IIA). The values of the substituents in Structural Formulas (IIIA) and (IVA) are as defined with reference to Structural Formulas (IIA) and (IA).

In another embodiment, the present invention is a method of synthesis of a compound of Structural Formula (XXXIA)

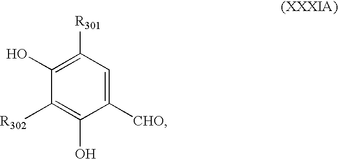

comprising the step of reacting the compound of for Structural Formula (XXXA)

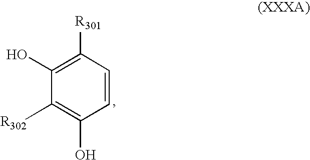

with POCl₃ in dimethyl formamide (DMF). Substituents R₃₀₁ and R₃₀₂ are each independently —H, an alkyl, an aryl, a heteroaryl, an aralkyl, a heteraralkyl, each optionally substituted by one or more of an alkyl, alkoxy, haloalkyl, halogen nitro, cyano or alkyl alkanoate groups.

In another embodiment, the present invention is a method of synthesis of the compound of Structural Formula (XXA)

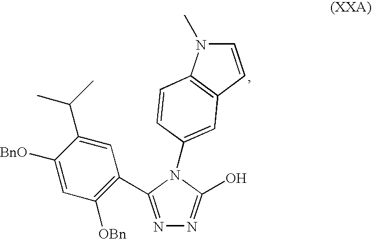

comprising reacting a compound of Structural Formula (XXIA)

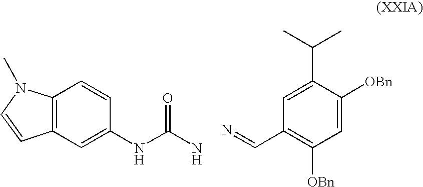

with an oxidizing agent, thereby producing a compound of formula (XXA).

In another embodiment, the present invention is a compound of Structural Formula (IIA):

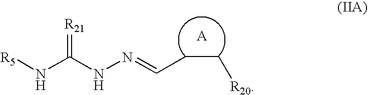

The values and the preferred values of the substituents in Structural Formula (IIA) are as defined above.

Another embodiment of the present invention is directed to a method (method III) of preparing a compound thereof represented by the following Structural Formula:

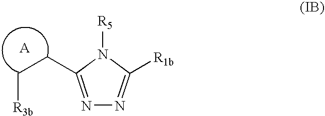

or a tautomer, a pharmaceutically acceptable salt, solvate, clathrate, or a prodrug thereof. The method comprises the step of reacting a first starting compound represented by the following Structural Formula:

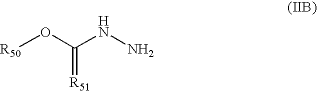

in the presence of a mercuric salt, with a second starting compound represented by the following Structural Formula:

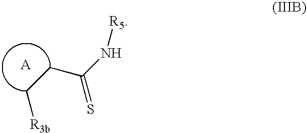

R_{1b} is —OH, —SH or —NHR₆₀; preferably, R_{1b} is —OH or —SH.

R₆₀ is H, an optionally substituted alkyl group, or an optionally substituted cycloalkyl group.

Ring A is an aryl or a heteroaryl, wherein the aryl group and the heteroaryl group represented by ring A is optionally further substituted with one or more substituents in addition to R_{3b}.

R_{3b} is —OR₁₀₀, —SR₁₀₁, —N(R₁₀₂)₂, —NR₇R₁₀₂, —OR₂₆, —SR₂₆, —NR₂₆R₁₀₂, —O(CH₂)ₘOR₁₀₀, O(CH₂)ₘSR₁₀₁, —O(CH₂)ₘNR₇R₁₀₂, —S(CH₂)ₘOR₁₀₀, S(CH₂)ₘSR₁₀₁, —S(CH₂)ₘNR₇R₁₀₂, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂. Preferably, R_{3b} is —OR₁₀₀, —SR₁₀₁, —N(R₁₀₂)₂, —NR₇R₁₀₂, —OR₂₆, —SR₂₆, —NR₂₆R₁₀₂, —O(CH₂)ₘOR₁₀₀, —O(CH₂)ₘSR₁₀₁, —O(CH₂)ₘNR₇R₁₀₂, S(CH₂)ₘOR₁₀₀, —S(CH₂)ₘSR₁₀₁, and —S(CH₂)ₘNR₇R₁₀₂.

Each R₁₀₀, independently, is a hydroxyl protecting group.

Each R₁₀₁, independently, is a thiol protecting group.

Each R₁₀₂, independently, is —H or an amino protecting group, provided that at least one group represented by R₁₀₂ is a protecting group.

R₅ is an optionally substituted aryl group, an optionally substituted heteroaryl group, an optionally substituted cycloalkyl group, an optionally substituted cycloakenyl group, or a substituted alkyl group, wherein each of the aryl group, heteroaryl group, cycloaryl group, cycloalkyl group, cycloalkenyl group, and alkyl group is substituted with one or more substituents independently selected from the group consisting of an optionally substituted alkyl group, an optionally substituted alkynyl, an optionally substituted cycloalkyl group, an optionally substituted cycloalkenyl group, an optionally substituted heteroaryl group, an optionally substituted aralyalkyl group, or an optionally substituted heteraralkyl group.

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, or R₇, taken together with the oxygen atom to which it is bonded, forms an optionally substituted heterocyclyl or an optionally substituted heteroaryl.

R₁₀ and R₁₁, for each occurrence, are, independently, amine protecting group, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl.

R₂₆ is a lower alkyl group.

R₅₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl.

R₅₁ is ═O, ═S or ═NR₆₀.

p, for each occurrence, is, independently, 0, 1 or 2.

m, for each occurrence, is, independently, 1, 2, 3, or 4.

In another embodiment, the present invention is directed to a method of preparing a compound thereof represented by the following Structural Formula:

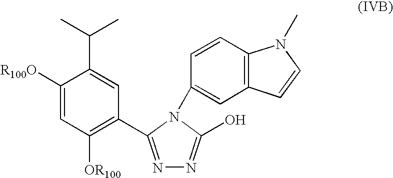

or a tautomer, a pharmaceutically acceptable salt, solvate, clathrate, or a prodrug thereof. The method comprises the step of reacting a first starting compound represented by the following Structural Formula:

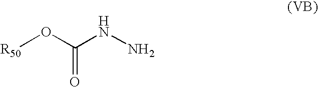

in the presence of a mercuric salt, with a second starting compound represented by the following structural formula

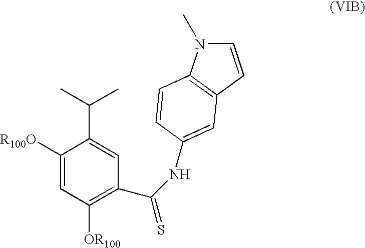

Each R₁₀₀, independently, is a hydroxyl protecting group; and R₅₀ is an alkyl.

In alternative embodiment, the present invention is directed to a method of preparing a compound thereof represented by the following Structural Formula:

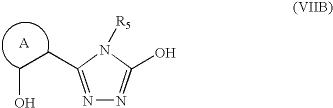

or a tautomer, a pharmaceutically acceptable salt, solvate, clathrate, or a prodrug thereof. The method comprises the steps of:
1) reacting a thionation reagent with a compound represented by the following Structural Formula:

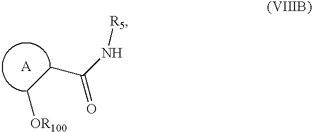

thereby forming a first product represented by the following Structural Formula:

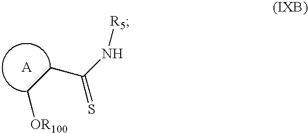

2) in the presence of a mercuric salt, reacting the first product with

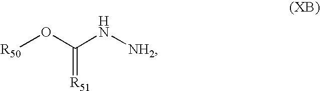

thereby forming a second product represented by the following Structural Formula:

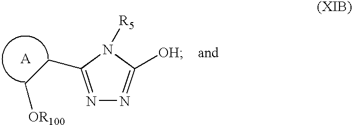

3) deprotecting the second product, thereby forming the compound represented by Structural Formula (XIB).

Values for ring A, R_{3b}, R₅, R₇, R₈, R₁₀, R₁₁, R₂₆, R₅₀, R₅₁, R₁₀₀, R₁₀₁, R₁₀₂, p, and m are as described above in Structural Formulas (IB)-(IIIB).

Preferably, R_{3b} is —OR₁₀₀, —SR₁₀₁, —N(R₁₀₂)₂, —NR₇R₁₀₂, —OR₂₆, —SR₂₆, —NR₂₆R₁₀₂, —O(CH₂)ₘOR₁₀₀, —O(CH₂)ₘSR₁₀₁, —O(CH₂)ₘNR₇R₁₀₂, —S(CH₂)ₘOR₁₀₀, —S(CH₂)ₘSR₁₀₁, or —S(CH₂)ₘNR₇R₁₀₂.

The methods of the present invention described above overcomes the problem of poor selectivity and eliminates the need of high temperature heating in the prior methods. Instead, the methods provides compounds in high yield and with clean crystallization that is obtained under moderate temperature.

### BRIEF DESCRIPTION OF THE DRAWINGS

FIG. 1 shows a synthetic scheme for preparing [1,2,4]triazole compound represented by Structural Formula (I).

### DETAILED DESCRIPTION OF THE INVENTION

The present invention is directed to novel synthetic methods for synthesizing certain [1,2,4]-triazole compounds, which inhibit the activity of Hsp90 and are useful in the treatment of proliferative disorders, such as cancer.

Unless otherwise specified, the terms used herein are defined as follows:

As used herein, the term “alkyl” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. The term “(C₁-C₆)alkyl” means a saturated straight chain or branched non-cyclic hydrocarbon having from 1 to 6 carbon atoms. Representative (C₁-C₆)alkyl groups are those shown above having from 1 to 6 carbon atoms. Alkyl groups included in compounds of this invention may be optionally substituted with one or more substituents.

As used herein, the term “alkenyl” means a saturated straight chain or branched non-cyclic hydrocarbon having from 2 to 10 carbon atoms and having at least one carbon-carbon double bond. Representative straight chain and branched (C₂-C₁₀)alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. Alkenyl groups may be optionally substituted with one or more substituents.

As used herein, the term “alkynyl” means a saturated straight chain or branched non-cyclic hydrocarbon having from 2 to 10 carbon atoms and having at lease one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkynyl groups may be optionally substituted with one or more substituents.

As used herein, the term “cycloalkyl” means a saturated, mono- or polycyclic alkyl radical having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, -cyclodecyl, octahydro-pentalenyl, and the like. Cycloalkyl groups may be optionally substituted with one or more substituents.

As used herein, the term “cycloalkenyl” means a mono- or poly-cyclic non-aromatic alkyl radical having at least one carbon-carbon double bond in the cyclic system and from 3 to 20 carbon atoms. Representative cycloalkenyls include cyclopentenyl, cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl, 1,2,3,4,5,8-hexahydronaphthalenyl and the like. Cycloalkenyl groups may be optionally substituted with one or more substituents.

As used herein, the term “haloalkyl” means and alkyl group in which one or more (including all) the hydrogen radicals are replaced by a halo group, wherein each halo group is independently selected from —F, —Cl, —Br, and —I. The term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker.

As used herein, an “haloalkoxy” is an haloalkyl group which is attached to another moiety via an oxygen linker.

As used herein, the term an “aromatic ring” or “aryl” means a hydrocarbon monocyclic or polycyclic radical in which at least one ring is aromatic. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Aryl groups may be optionally substituted with one or more substituents. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups may be optionally substituted with one or more substituents.

As used herein, the term “heterocyclyl” means a monocyclic (typically having 3- to 10-members) or a polycyclic (typically having 7- to 20-members) heterocyclic ring system which is either a saturated ring or a unsaturated non-aromatic ring. A 3- to 10-membered heterocycle can contain up to 5 heteroatoms; and a 7- to 20-membered heterocycle can contain up to 7 heteroatoms. Typically, a heterocycle has at least on carbon atom ring member. Each heteroatom is independently selected from nitrogen, which can be oxidized (e.g., N(O)) or quaternized; oxygen; and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, the hydrogen on a nitrogen may be substituted with a tert-butoxycarbonyl group. Furthermore, the heterocyclyl may be optionally substituted with one or more substituents. Only stable isomers of such substituted heterocyclic groups are contemplated in this definition.

As used herein, the term “heteroaromatic”, “heteroaryl” or like terms means a monocyclic or polycyclic heteroaromatic ring comprising carbon atom ring members and one or more heteroatom ring members. Each heteroatom is independently selected from nitrogen, which can be oxidized (e.g., N(O)) or quaternized; oxygen; and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, a isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings. Heteroaryl groups may be optionally substituted with one or more substituents.

As used herein, the term “(C₅)heteroaryl” means an aromatic heterocyclic ring of 5 members, wherein at least one carbon atom of the ring is replaced with a heteroatom such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like.

As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl and the like. Heteroaralkyl groups may be optionally substituted with one or more substituents.

As used herein, the term “halogen” or “halo” means —F, —Cl, —Br or —I.

Suitable substituents for an alkyl, alkylene, alkenyl, alkynyl, cycloalkyl, cycloalkenyl, heterocyclyl, aryl, aralkyl, heteroaryl, and heteroaralkyl groups include any substituent which will form a stable compound of the invention. Examples of substituents for an alkyl, alkylene, alkenyl, alkynyl, cycloalkyl, cycloalkenyl, heterocyclyl, aryl, aralkyl, heteroaryl, and heteroarylalkyl include R²⁸ a haloalkyl, —C(O)NR₂₈R₂₉, —C(S)NR₂₈R₂₉, —C(NR₃₂)NR₂₈R₂₉, —NR₃₀C(O)R₃₁, —NR₃₀C(S)R₃₁, —NR₃₀C(NR₃₂)R₃₁, halo, —OR₃₀, cyano, nitro, haloalkoxy, —C(O)R₃₀, —C(S)R₃₀, —C(NR₃₂)R₃₀, —NR₂₈R₂₉, —C(O)OR₃₀, —C(S)OR₃₀, —C(NR₃₂)OR₃₀, —OC(O)R₃₀, —OC(S)R₃₀, —OC(NR₃₂)R₃₀, —NR₃₀C(O)NR₂₈R₂₉, —NR₃₀C(S)NR₂₈R₂₉, —NR₃₀C(NR₃₂)NR₂₈R₂₉, —OC(O)NR₂₈R₂₉, —OC(S)NR₂₈R₂₉, —OC(NR₃₂)NR₂₈R₂₉, —NR₃₀C(O)OR₃₁, —NR₃₀C(S)OR₃₁, —NR₃₀C(NR₃₂)OR₃₁, —S(O)ₕR₃₀, —OS(O)ₚR₃₀, —NR₃OS(O)ₚR₃₀, —S(O)ₚNR₂₈R₂₉, OS(O)ₚNR₂₈R₂₉, or —NR₃OS(O)ₚNR₂₈R₂₉, wherein R₂₈ and R₂₉, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₂₈ and R₂₉ taken together with the nitrogen to which they are attached is optionally substituted heterocyclyl or optionally substituted heteroaryl. Preferably R₂₈ and R₂₉, for each occurrence are, independently, H, alkyl, alkenyl, alkynyl, an cycloalkyl, cycloalkenyl, heterocyclyl, aryl, heteroaryl, aralkyl, or heteraralkyl; or R₂₈ and R₂₉ taken together with the nitrogen to which they are attached is optionally substituted heterocyclyl or optionally substituted heteroaryl. In certain embodiments, the substituents are not —C(O)NR₂₈R₂₉, —NR₃₀C(O)R₃₁, —C(O)OR₃₀, —NR₃₀C(O)NR₂₈R₂₉, —OC(O)NR₂₈R₂₉, —NR₃₀C(O)OR₃₁.

R₃₀ and R₃₁ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; and

R₃₂, for each occurrence is, independently, H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, —C(O)R₃₀, —C(O)NR₂₈R₂₉, —S(O)ₚR₃₀, or —S(O)ₚNR₂₈R₂₉;

p, for each occurrence, is independently, 1 or 2; and

h is 0, 1 or 2.

In addition, alkyl, cycloalkyl, alkylene, a heterocyclyl, and any saturated portion of a alkenyl, cycloalkenyl, alkynyl, aralkyl, and heteroaralkyl groups, may also be substituted with ═O, ═S, ═N—R₃₂.

When a heterocyclyl, heteroaryl, or heteroaralkyl group contains a nitrogen atom, it may be substituted or unsubstituted. When a nitrogen atom in the aromatic ring of a heteroaryl group has a substituent the nitrogen may be a quaternary nitrogen.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl and a “lower alkenyl” or “lower alkynyl” refers to an alkenyl or alkynyl radical having from 2 to 4 carbon atoms, respectively.

Unless indicated otherwise, the compounds of the invention containing reactive functional groups (such as (without limitation) carboxy, hydroxy, thiol, and amino moieties) also include protected derivatives thereof, such as those found in T. W. Greene, Protecting Group in Organic Synthesis, Wiley & Sons, Inc. 1999 (hereinafter “Greene”), the entire teachings of which are incorporated by reference. “Protected derivatives” are those compounds in which a reactive site or sites are blocked with one or more protecting groups.

Examples of suitable protecting groups for hydroxyl groups include ethers (e.g., methoxymethyl, methylthiomethyl, (phenyldimethylsilyl)methoxymethyl, benzyloxymethyl, p-methoxbenzyloxymethyl, p-nitrobenzyloxymethyl, o-nitrobenzyloxymethyl, (4-methoxyphenoxy)methyl, guaiacolmethyl, t-butoxymethyl, 4-pentenyloxymethyl, siloxymethyl, 2-methoxyethoxymethyl, 2,2,2-trichloroethoxymethyl, bis(2-chloroethoxy)methyl, 2-(trimethylsilyl)ethoxymethyl, menthoxymethyl, tetrahydropyranyls, 1-ethoxyethyl, 1-(2-chloroethoxy)ethyl, 1-[2-(trimethylsilyl)ethoxy]ethyl, 1-methyl-1-methoxyethyl, methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2,6-difluorobenzyl, p-acylaminobenzyl), silyl ethers (e.g., trimethylsilyl, triethylsilyl, triisopropylsilyl, dimethylisopropylsilyl, diethylisopropylsilyl, dimethylhexylsilyl, t-butyldimethylsilyl, t-butyldiphenylsilyl, tribenzylsilyl, tri-p-xylysilyl, triphenylsilyl, diphenylmethylsilyl, di-t-butylmethylsilyl, tris(trimethylsilyl)silyl:sisyl, (2-hydroxystyryl)dimethylsilyl, and (2-hydroxystyryl)diisopropylsilyl), esters (e.g., benzoylformate, acetates, chloroacetate, dichloroacetate, trichloroacetate, trifluoroacetate, methoxyacetate, triphenylmethoxyacetate, phenoxyacetate, p-chlorophenoxyacetate, phenylacetate, p-P-phenylacetate, and diphenylacetate, nicotinate and the like), and 3-phenylpropionate), carbonates (e.g., methoxylmethyl, 9-fluorenylmethyl, 2,2,2-trichloroethyl, 1,1-dimethyl-2,2,2-trichloroethyl, 2-(trimethylsilyl)ethyl, 2-(phenylsulfonyl)ethyl, 2-(triphenylphosphino)ethyl, isobutyl, vinyl, allyl, p-nitrophenyl, benzyl, p-methoxybenzyl, 3,4-dimethoxybenzyl, o-nitrobenzyl, and p-nitrobenzyl) and other suitable hydroxyl protecting groups recited in Greene.

Examples of suitable protecting groups for phenols groups include ethers (e.g. methyls (e.g. methoxymethyl, benzyloxymethyl, methoxyethoxymethyl, 2-(trimethylsilyl)ethoxymethyl, methylthiomethyl, phenylthiomethyl, azidomethyl, cyanomethyl, 2,2-dichloro-1,1-difluoroethyl, 2-chloroethyl, and 2-bromoethyl) tetrahydropyranyl, and 1-ethoxyethyl), silyl ethers (e.g. trimethylsilyl, t-butyldimethylsilyl, t-butyldiphenylsilyl, triisopropylsilyl and the like) esters (e.g. formate, acetate, levulinate, pivaloate, benzoate, 9-fluorenecarboxylate, xanthenecarboxylate and the like), carbonates (e.g. methyl, 1-adamantyl, t-butyl, 4-methylsulfinylbenzyl, 2,4-dimethylpent-3-yl, 2,2,2-trichloroethyl, vinyl, benzyl, aryl carbamates and the like).

Examples of suitable protecting groups for thiol groups include thioethers (e.g., S-alkyl, S-benzyl, S-p-methoxybenzyl, S-o- or p-hydroxy- or acetoxybenzyl, S-p-nitrobenzyl, S-2,4,6-trimethylbenzyl, S-2,4,6-trimethoxybenzyl, S-4-picolyl, S-2-quinolinylmethyl, S-2-picolyl N-oxido, S-9-anthrylmethyl, S-phenyl, S-2,4-dinitrophenyl, S-t-butyl, S-methoxymethyl, S-isobutoxymethyl, and S-benzyloxymethyl), thioesters (e.g., S-acetyl, S-benzoyl, S-trifluoroacetyl, S—N-[[(p-biphenylyl)isopropoxy]carbonyl]-n-methyl-Y-aminothiobutyrate, S—N-(t-butoxycarbonyl)-n-methyl-γ-aminothiobutyrate, and the like), thiocarbonated derivatives (e.g., S-2,2,2-trichloroethoxycarbonyl, S-t-butoxycarbonyl, S-benzyloxycarbonyl, S-p-methoxybenzyloxycarbonyl and the like), thiocarbamate derivatives (e.g., S—(N-ethyl), S—(N-methoxymethyl)).

Examples of suitable protecting groups for amino groups include carbamates (e.g, methyl, ethyl, 9-fluorenylmethyl, 9-(2-sulfo)fluorenylmethyl, 9-(2,7-dibromo)fluorenylmethyl, 17-tetrabenzo[a,c,g,i]fluoemylthmethyl, 2-chloro-3-indenylmethyl, benz[f]inden-3-ylmethyl-2,7-di-t-butyl-[9-(10,10-dioxo-10,10,10,10-tetrahydrothioxanthyl)]methyl, 1,1-dioxobenzo[b]thiophene-2-ylmethyl, 2,2,2-tricholoroethyl, 2-trimethylsilylethyl, 2-phenylethyl, 1-(1-adamantyl)-1-methylethyl, 2-chloroethyl, 1,1-dimethyl-2-haloethyl, 1,1-dimethyl-2-dibromethyl, 1,1-dimethyl-2,2,2-trichloroethyl, 1-methyl-1-(4-biphenylyl)ethyl, 1-(3,5-di-t-butylphenyl)-1-methylethyl, t-butyl, 1-adamantyl, 2-adamantyl, vinyl, allyl, 1-isopropylallyl, cinnamyl, 4-nitrocinnamyl, benzyl, p-methoxybenzyl, p-nitrobenzyl, p-bromobenzyl, p-chlorobenzyl, 2,4-dichlorobenzyl, m-nitrophenyl, 3,5-dimethoxybenzyl, 1-methyl-1-(3,5-dimethoxyphenyl)ethyl, α-methylnitropiperonyl, o-nitrobenzyl, 3,4-dimethoxy-6-nitrobenzyl, and phenyl(o-nitrophenyl)methyl), amides (e.g., n-formyl, n-acetyl, n-chloroacetyl, n-trichloroacetyl, n-trifluoroacetyl, n-phenylacetyl, and n-3-phenylpropionyl), N-alkyl and N-aryl amines (e.g., n-methyl, n-t-butyl, n-allyl, n-benzyl, n-4-methoxybenzyl, n-2,4-dimethoxybenzyl, and n-2-hydroxybenzyl).

Other suitable protecting groups are well known to those of ordinary skill in the art and include those found in T. W. Greene, Protecting Groups in Organic Synthesis, John Wiley & Sons, Inc. 1981, the entire teachings of which are incorporated herein by reference.

As used herein, the term “compound(s) of Structural Formulas (I)-(VIII), (IA), (IA′), (IIA)-(IVA), (XXA), (XXIA), (XXXA), (XXXIA) or (IB)-(XIB)” and similar terms refers to a compound of Structural Formulas (I)-(VIII), (IA), (IA′), (IIA)-(IVA), (XXA), (XXIA), (XXXA), (XXXIA) or (IB)-(XIB), or Tables 1 and 2, or a tautomer, pharmaceutically acceptable salt, solvate, clathrate, hydrate, polymorph or prodrug thereof, and also include protected derivatives thereof.

The compounds synthesized by the methods of the present invention may contain one or more chiral centers and/or double bonds and, therefore, exist as stereoisomers, such as double-bond isomers (i.e., geometric isomers), enantiomers, or diastereomers. According to this invention, the chemical structures depicted herein, including the compounds of this invention, encompass all of the corresponding compounds' enantiomers, diastereomers and geometric isomers, that is, both the stereochemically pure form (e.g., geometrically pure, enantiomerically pure, or diastereomerically pure) and isomeric mixtures (e.g., enantiomeric, diastereomeric and geometric isomeric mixtures). In some cases, one enantiomer, diastereomer or geometric isomer will possess superior activity or an improved toxicity or kinetic profile compared to other isomers. In those cases, such enantiomers, diastereomers and geometric isomers of compounds of this invention are preferred.

The compounds synthesized by the methods of the present invention can be obtained in a form of polymorphs, salts, including a pharmaceutically acceptable salt, solvates or clathrates.

As used herein, the term “polymorph” means solid crystalline forms of a compound of the present invention or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a compound of the present invention or a salt thereof, that further includes a stoichiometric or non-stoichiometric amount of water bound by non-covalent intermolecular forces.

As used herein, the term “clathrate” means a compound of the present invention or a salt thereof in the form of a crystal lattice that contains spaces (e.g., channels) that have a guest molecule (e.g., a solvent or water) trapped within.

As used herein and unless otherwise indicated, the term “prodrug” means a derivative of a compound that can hydrolyze, oxidize, or otherwise react under biological conditions (in vitro or in vivo) to provide a compound of this invention. Prodrugs may become active upon such reaction under biological conditions, or they may have activity in their unreacted forms. Examples of prodrugs contemplated in this invention include, but are not limited to, analogs or derivatives of compounds of Structural Formulas (I), (V), (IA), (IA′), (XXA), (IB), (IVB), (VIIB), (XIB), or Tables 1 and 2 that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of Structural Formulas (I), (V), (IA), (IA′), (XXA), (IB), (IVB), (VIIB), (XIB) or Tables 1 and 2 that comprise —NO, —NO₂, —ONO, or —ONO₂ moieties. Prodrugs can typically be prepared using well-known methods, such as those described by 1 BURGER'S MEDICINAL CHEMISTRY AND DRUG DISCOVERY (1995) 172-178, 949-982 (Manfred E. Wolff ed., 5^{th} ed).

As used herein and unless otherwise indicated, the terms “biohydrolyzable amide”, “biohydrolyzable ester”, “biohydrolyzable carbamate”, “biohydrolyzable carbonate”, “biohydrolyzable ureide” and “biohydrolyzable phosphate analogue” mean an amide, ester, carbamate, carbonate, ureide, or phosphate analogue, respectively, that either: 1) does not destroy the biological activity of the compound and confers upon that compound advantageous properties in vivo, such as improved water solubility, improved circulating half-life in the blood (e.g., because of reduced metabolism of the prodrug), improved uptake, improved duration of action, or improved onset of action; or 2) is itself biologically inactive but is converted in vivo to a biologically active compound. Examples of biohydrolyzable amides include, but are not limited to, lower alkyl amides, α-amino acid amides, alkoxyacyl amides, and alkylaminoalkylcarbonyl amides. Examples of biohydrolyzable esters include, but are not limited to, lower alkyl esters, alkoxyacyloxy esters, alkyl acylamino alkyl esters, and choline esters. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, the term “pharmaceutically acceptable salt,” is a salt formed from, for example, an acid and a basic group of one of the compounds of Structural Formulas (I), (V), (IA), (IA′), (XXA), (IB), (IVB), (VIIB), (XIB), or Tables 1 and 2. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The term “pharmaceutically acceptable salt” also refers to a salt prepared from a compound of Structural Formulas (I), (V), (IA), (IA′), (XXA), (IB), (IVB), (VIIB), (XIB), or Tables 1 and 2 having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of Structural Formulas (I), (V), (IA), (IA′), (XXA), (IB), (IVB), (VIIB), (XIB), or Tables 1 and 2 having a basic functional group, such as an amine functional group, and a pharmaceutically acceptable inorganic or organic acid. Suitable acids include, but are not limited to, hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, and p-toluenesulfonic acid.

As used herein, the term “pharmaceutically acceptable solvate,” is a solvate formed from the association of one or more pharmaceutically acceptable solvent molecules to one of the compounds of Structural Formulas (I), (V), (IA), (IA′), (XXA), (IB), (IVB), (VIIB), (XIB), or Tables 1 and 2. The term solvate includes hydrates (e.g., hemihydrate, monohydrate, dihydrate, trihydrate, tetrahydrate, and the like).

A pharmaceutically acceptable carrier may contain inert ingredients which do not unduly inhibit the biological activity of the compounds. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

The compounds synthesized by the methods of the present invention are defined herein by their chemical structures and/or chemical names. Where a compound is referred to by both a chemical structure and a chemical name, and the chemical structure and chemical name conflict, the chemical structure is determinative of the compound's identity.

Only those choices and combinations of substituents that result in a stable structure are contemplated. Such choices and combinations will be apparent to those of ordinary skill in the art and may be determined without undue experimentation.

As used herein, a composition that “substantially” comprises a compound means that the composition contains more than about 80% by weight, more preferably more than about 90% by weight, even more preferably more than about 95% by weight, and most preferably more than about 97% by weight of the compound.

As used herein, a reaction that is “substantially complete” means that the reaction contains more than about 80% by weight of the desired product, more preferably more than about 90% by weight of the desired product, even more preferably more than about 95% by weight of the desired product, and most preferably more than about 97% by weight of the desired product.

As used herein, a racemic mixture means about 50% of one enantiomer and about 50% of is corresponding enantiomer relative to a chiral center in the molecule. The invention encompasses all enantiomerically-pure, enantiomerically-enriched, diastereomerically pure, diastereomerically enriched, and racemic mixtures of the compounds of the invention.

Enantiomeric and diastereomeric mixtures can be resolved into their component enantiomers or diastereomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

In certain instances, tautomeric forms of the compounds disclosed herein exist, such as the tautomeric structures shown below:

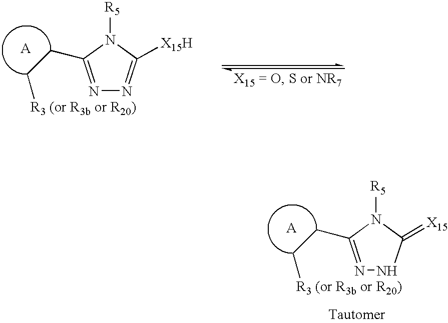

It is to be understood that when a compound is represented by a structural formula herein, all other tautomeric forms which may exist for the compound are encompassed by the structural formula.

The invention can be understood more fully by reference to the following detailed description and illustrative examples, which are intended to exemplify non-limiting embodiments of the invention.

The present invention provides novel synthetic methods (methods I, II and III) suitable for manufacturing [1,2,4]-triazole compounds on an industrial scale.

For method I, the synthesis begins with an amidation reaction of the starting carboxylic acid represented by the following structural formula:

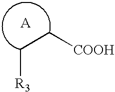

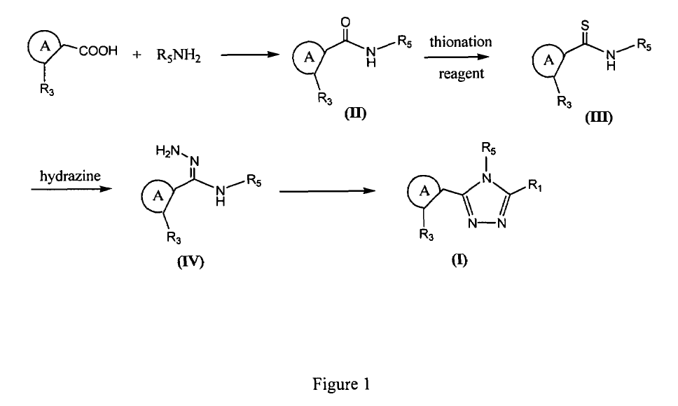

with an amine R₅NH₂ to form an amide represented by Structural Formula (II). The amide is then thionated to form a thioamide represented by Structural Formula (III). The thioamide is reacted with hydrazine to form a hydrazonamide compound represented by Structural Formula (IV), which is cyclized with a carbonylation reagent, thoicarbonylation reagent or an isocyanide to form the [1,2,4]trizole compound represented by Structural Formula (I). This synthesis is shown schematically in FIG. 1. A detailed description of each reaction in the synthesis is provided below.

The starting carboxylic acid is first converted to the amide intermediate represented by Structural Formula (II) by reacting the starting carboxylic acid of Structural Formula (VI) with the amine R₅NH₂. Methods for converting a carboxylic acid to an amide are well known in the art.

Typically, the carboxylic acid is first converted into a more reactive derivative with a leaving group that is more readily displaced by an amine group than —OH. A “leaving group” is a group which can readily be displaced by a nucleophile. For example, a carboxylic acid can be converted to a more reactive acyl halide, typically acyl chloride. Suitable reagents and conditions for converting a carboxylic acid to an acyl halide are well known in the art and are described, for example, in March, “Advanced organic Chemistry—Reactions, Mechanisms and Structure”, 5th Edition, John Wiley & Sons, 2001, pages 523-524, and references cited therein. Examples of suitable reagents include thionyl chloride, oxalyl chloride, phosphorus trichloride and phosphorous pentachloride. Typically, each carboxylic acid group is reacted with about one equivalent or a slight excess of thionyl chloride, oxalyl chloride, phosphorus trichloride and phosphorous pentachloride in an inert solvent such as an ethereal solvent (e.g., diethyl ether, tetrahydrofuran or 1,4-dioxane), a halogenated solvent (e.g. methylene chloride or 1,2-dichloroethane) or aromatic solvent (e.g., benzene or toluene). When the carboxylic acid is amidated following an initial conversion of carboxylic acid to acyl halide, stoichiometric amount of the carboxylic acid and amine can be used. Alternatively, excess of either the carboxylic acid or amine can be used. When oxalyl chloride is used, a tertiary amine is often added to accelerate the reaction in quantities ranging from a catalytic amount to about one equivalent relative to oxalyl chloride. Typically tertiary amine can be used is triethylamine. The reaction is generally carried in inert, aprotic solvents, for example, halogenated solvents such as methylene chloride, dichloroethane and dimethylformamide. Suitable reaction temperature generally range from between about 0° C. to 100° C., preferably between about 0° C. to about ambient temperature.

Alternatively, the carboxylic acid is first converted into an “activated ester”. An ester —COOR is said to be “activated” when —OR is readily displaced by an amine or hydrazine. —OR is more easily displaced as R becomes more electron withdrawing. Some activated esters are sufficiently stable that they can be isolated, e.g., esters wherein R is phenyl or substituted phenyl. For example, diphenylmalonate can be prepared from malonyl chloride and phenol, both commercially available from Aldrich Chemical Co., Milwaukee, Wis., by procedures described above Other activated esters are more reactive and are generally prepared and used in situ.

Formation of an activated ester in situ requires a “coupling agent”, also referred to as a “carboxylic acid activating agent”, which is a reagent that replaces the hydroxyl group of a carboxyl acid with a group which is susceptible to nucleophilic displacement. Examples of coupling agents include 1,1′-carbonyldiimidazole (CDI), isobutyl chloroformate, dimethylaminopropylethyl-carbodiimide (EDC), dicyclohexyl carbodiimide (DCC). When amidating a carboxylic acid by in situ activation using a coupling reagent, stoichiometric amount of the carboxylic acid and amine can be used. Alternatively, excess of either the carboxylic acid or the amine can be used. The reaction is generally carried in inert, aprotic solvents, for example, halogenated solvents such as methylene chloride, dichloroethane and dimethylformamide.

The amide of Structural Formula (II) is then reacted with a thionation reagents to form a thioamide. “Thionation reagent” is a reagent which, under suitable conditions, can convert a ketone, ester, or amide into a thioketone, thioester or thioamide, respectively. There are many thionation reagents known to one of ordinary skill in the art. Examples include Lawesson's Reagent, tetraphosphorous pentasulfide, Scheeren's reagent (P₄S₁₀—Na₂S), P₄S₁₀—N(ethyl)₃, Davy's Reagent and Heimgarner's reagent. Also known are conditions suitable for carrying out these conversions with thionation reagents. For example, such conditions are disclosed in Fieser and Fieser, “Reagents for Organic Synthesis”, Volume 1, John Wiley & Sons (1975) page 870-871, Fieser and Fieser, “Reagents for Organic Synthesis”, Volume 5, John Wiley & Sons, (1975) page 653 and publication cited therein. Suitable conditions are also described in March, “Advanced Organic Chemistry—Reactions, Mechanisms and Structure”, Fifth Edition, John Wiley & Sons, 2001, pages 496, 509, 1184-1185, 1331; Bull. Soc. Chim. Belg. 87:223, 229, 525 (1978), Synthesis 1979: 941 (1979), Tetrahedron 35: 2433 (1979) Tetrahedron 21: 4061 (1980); Tetrahedron, (1985), 41, 2567; Org. Synth. VII, 372; and Tetrahedron Lett., (1986), 27, 3445; and references cited therein. (All of these references are incorporated herein). There are many thionation reagents known to one of ordinary skill in the art. Descriptions of these reagents can also be found in Metzner and Thuillier “Sulfur Reagents in Organic Synthesis”, Academic Press, 1994. The relevant portions of these publications are incorporated herein by reference.

To thionate the amide of Structural Formula (II), it may be desirable to use a slight excess of the amide, for example up to about 5 equivalents, preferably no more than about 1.5 equivalents. It may also be desirable to use excess thionation reagent. In some cases, it may be desirable to use equal equivalents of the amide and the thionation reagent. Suitable inert solvents include ethereal solvents (e.g., diethyl ether, tetrahydrofuran, glyme and 1,4-dioxane), aromatic solvents (e.g., benzene and toluene) or chlorinated solvents (e.g., methylene chloride and 1,2-dichloroethane). The reaction is carried out at temperatures ranging from about room temperature to about 150° C., preferably from about 75° C. to about 125° C. In a preferred embodiment, the thionation reagent is Lawesson's reagent. Representative conditions for carrying out thionation reaction are found in Examples 1 and 2.

Preferably, the reaction mixture of the amide and thionation reagent is treated with a water soluble amine after completion of the reaction. As used herein, a “water soluble amine” may include any amines (e.g., methylamine), ammonium hydroxide, and hydrazines. In a more specific embodiment, the water soluble amine is aqueous ammonium hydroxide. In another more specific embodiment, the water soluble amine is hydrazine. Typically, excess ammonium hydroxide solution is used, for example up to 10 equivalents, preferably up to 5 equivalents, even more preferably up to 2 equivalents. Detailed description of a representative procedure is found in Example 1.

The thioamide of Structural Formula (II) is then converted to hydrazonamide of Structural Formula (III) by reacting the thioamide with hydrazine in an inert solvent. Preferably, excess of hydrazine is used, for example up to 100 equivalents, up to 50 equivalents, up to 10 equivalents. In some cases, it may be desirable to use excess of thioamide or equal equivalents of thioamide and hydrazine. Suitable inert solvents include ethereal solvents (e.g., diethyl ether, tetrahydrofuran, glyme and 1,4-dioxane), aromatic solvents (e.g., benzene and toluene) or chlorinated solvents (e.g., methylene chloride and 1,2-dichloroethane). The reaction is carried out at temperatures ranging from about room temperature to about 150° C., preferably from about 80° C. to about 100° C. Representative conditions for carrying out these reactions are found in Examples 1 and 2.

The thioamide of Structural Formula (III) is then cyclized with a carbonylation reagent, a thiocarbonylation reagent or a compound of structural formula R₇N═C(X)₂, wherein X is a leaving group, to form the [1,2,4]triazole compound of Structural Formula (I).

As used herein, a “carbonylation reagent” is a compound represented by a structural formula of X—C(═O)—X, where X a readily displaced leaving group to facilitate the cyclization reaction with the hydrazonamide of Structural Formula (IV) to form the triazole compound of Structural Formula (I), wherein R₁ is —OH. As used herein, a “leaving group” is a group that can be displaced by a nucleophile. For example, X can be a imidazoyl group, a halide, more specifically, a chloride. Examples of carbonylation reagent may be used include phosgene, carbonyldiimidazole, diphenyl carbonate, bis(4-nitrophenyl)carbonate, bis(pentafluorophenyl)carbonate, bis(trichloromethyl)carbonate, 4-nitrophenyl chloroformate, phenyl chloroformate, trichloromethyl chloroformate. In a specific embodiment, the carbonylation reagent is carbonyldiimidazole. The hydrazonamide of Structural Formula (IV) is converted to the triazole compound of Structural Formula (I) or a tautomer, pharmaceutically acceptable salt, solvate, clathrate, or a prodrug thereof, by reacting the hydrazonamide with a carbonylation reagent in an inert solvent. Suitable inert solvents include ethereal solvents (e.g., diethyl ether, tetrahydrofuran, glyme and 1,4-dioxane), aromatic solvents (e.g., benzene and toluene), chlorinated solvents (e.g., methylene chloride and 1,2-dichloroethane) or ethyl acetate. The reaction is carried out at temperatures ranging from about room temperature to about 150° C., preferably from about room temperature to about 100° C., more preferably from about room temperature to about 40° C. Typically, excess of the carbonylation reagent is used, for example, up to 10 equivalent, more preferably, up to 5 equivalent, even more preferably, up to 1.5 equivalent. In some case, it may be desirable to use excess of the hydrazonamide, or equal equivalents of the hydrazonamide and the carbonylation reagent.

As used herein, a “thiocarbonylation reagent” is a compound represented by a structural formula of X—S(═O)—X, where X a readily displaced leaving group to facilitate the cyclization reaction with the hydrazonamide of Structural Formula (IV) to form the triazole compound of Structural Formula (I), wherein R₁ is —SH. For example, X can be a imidazoyl group, a halide, more specifically, a chloride. Examples of thiocarbonylation reagent may be used include thiocarbonyldiimidazole and thiophosgene. In a specific embodiment, the thiocarbonylation reagent is thiocarbonyldiimidazole. The hydrazonamide of Structural Formula (IV) is converted to the triazole compound of Structural Formula (I) or a tautomer, pharmaceutically acceptable salt, solvate, clathrate, or a prodrug thereof, by reacting the hydrazonamide with a thiocarbonylation reagent in an inert solvent. Suitable inert solvents include ethereal solvents (e.g., diethyl ether, tetrahydrofuran, glyme and 1,4-dioxane), aromatic solvents (e.g., benzene and toluene), chlorinated solvents (e.g., methylene chloride and 1,2-dichloroethane) or ethyl acetate. The reaction is carried out at temperatures ranging from about room temperature to about 150° C., preferably from about room temperature to about 100° C., more preferably from about room temperature to about 40° C. Typically, excess of the carbonylation reagent is used, for example, up to 10 equivalent, more preferably, up to 5 equivalent, even more preferably, up to 1.5 equivalent. In some case, it may be desirable to use excess of the hydrazonamide, or equal equivalents of the hydrazonamide and the thiocarbonylation reagent.

In accordance with the present invention, the hydrazonamide of Structural Formula (IV) can react with a compound of structural formula R₇N═C(X)₂ to form the triazole compound of Structural Formula (I), wherein R₁ is —NHR₇. X is a readily displaced leaving group that facilitates the cyclization reaction of R₇N═C(X)₂ with the hydrazonamide of Structural Formula (IV). For example, X can be a imidazolyl group, a halide, more specifically a chloride. In a specific embodiment, X is —Cl. The hydrazomide of Structural Formula (IV) is converted to the triazole compound of Structural Formula (I) or a tautomer, pharmaceutically acceptable salt, solvate, clathrate, or a prodrug thereof, by reacting the hydrazonamide with R₇N═C(X)₂ in an inert solvent. Suitable inert solvents include ethereal solvents (e.g., diethyl ether, tetrahydrofuran, glyme and 1,4-dioxane), aromatic solvents (e.g., benzene and toluene), chlorinated solvents (e.g., methylene chloride and 1,2-dichloroethane) or ethyl acetate. The reaction is carried out at temperatures ranging from about room temperature to about 150° C., preferably from about room temperature to about 100° C., more preferably from about room temperature to about 40° C., even more preferably, the reaction is carried out at room temperature. Typically, excess of the carbonylation reagent is used, for example, up to 5 equivalent, more preferably, up to 5 equivalent, even more preferably, up to 1.5 equivalent. In some case, it may be desirable to use excess of the hydrazonamide, or equal equivalents of the hydrazonamide and the thiocarbonylation reagent.

In a specific embodiment, the compound of Structural Formula (V) is prepared by the disclosed methods. The synthesis of the compound of Structural Formula (V) comprises an initial step of thionating the amide of Structural Formula (VI) with a thionation reagent to form a thioaminde of Structural Formula (VII). The thioamide is then reacted with hydrazine to form a hydrazonamide of Structural Formula (VIII). The hydrazonamide is reacted with a carbonylation reagent to form the compound of Structural Formal (V). More specifically, the thionation reagent is Lawesson's reagent and the thionation reagent is carbonyldiimidazole. Any remaining protecting groups can be removed by standard methods following formation of the hydrazonamide.

Method II of the present invention provides a method of synthesizing a compound of Structural Formula (IA):

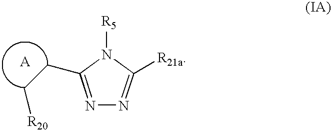

The method comprises reacting a compound of Structural Formula (IIA)

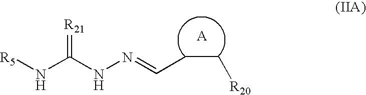

with an oxidizing agent, thereby producing a compound of Structural Formula (IA).

Specifically, by reacting a compound of Structural Formula (IIIA)

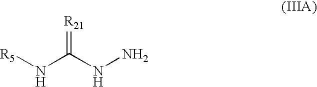

with a compound of Structural Formula (IVA),

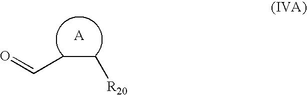

in the presence of an acid, a compound of Structural Formula (IIA)

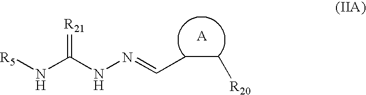

is prepared.

By reacting a compound of Structural Formula (IIA):

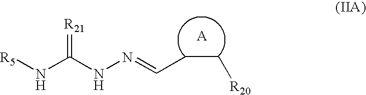

with an oxidizing agent, a compound of Structural Formula (IA)

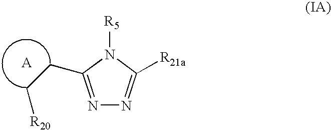

is prepared.

By removing any protecting groups present in the compound of Structural Formula (IA) (i.e. “deprotecting the compound”),

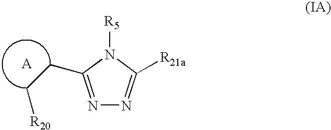

a compound of Structural Formula (IA′)

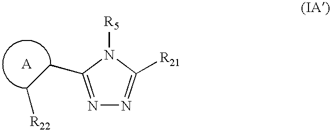

can be prepared.

The list of values and preferred values in formulas (IA), (IA′), (IIA), (IIIA) and (IVA) are defined above. Additionally, in formula (IA′), R₂₂ is —OH, or —NH₂. The conditions for the above reactions will be described below.

Preferably, the oxidizing agent is K₃Fe(CN)₆, MnO₂, Br₂, N-bromosuccinimide or N-chlorosuccinimide. More preferably, the oxidizing agent is K₃Fe(CN)₆. One skilled in the art will appreciate that some oxidizing agents (e.g., K₃Fe(CN)₆, MnO₂) are commonly used in combination with a base. Where a base is used, any organic or inorganic base can be used, such as a hydroxide base (e.g., NaOH, KOH, LiOH), amine bases (e.g., ammonia, allylamide, dialkylamine) or 1,1,1,3,3,3-hexamethyl-disilazane (HMDS). Preferably, the base is non-nucleophilic. The molar ratio of the base to the oxidizing agent can be about 5:1, 4:1, 3:1, 2:1; 1:1, 1:2, 1:3, 1:4 or 1:5. Preferably, equimolar ratio of the oxidizing agent and the base is used.

The oxidizing cyclization is generally carried out in polar solvent. The polar solvent can be a polar protic solvent, such as water or an alcohol; a polar aprotic aromatic solvent such as nitrobenzene; or a polar aprotic solvent such as nitromethane, dimethyl acetamide (DMA), N,N-dimethyl formamide (DMF), dimethyl sulfoxide (DMSO), hexamethyl phosphoramide (HMPA), or N-methylpyrrolidone (NMP).

The molar ratio of the compound of Structural Formula (IIA) to the oxidizing agent can vary greatly. Although equimolar amount can be used, the compound of formula (II) is typically used in excess. Generally, the molar ratio of the compound of formula (II) to an oxidizing agent can be 1000:1, 900:1, 800:1, 700:1, 600:1, 500:1, 400:1, 300:1, 200:1, 100:1, 50:1, 20:1, 10:1 For example, when KFe(CN)₆₁NaOH is used, the molar ratio of the compound (IIA) to KFe(CN)₆ is from about 500:1 to about 200:1, preferably from 350:1 to 300:1; and the molar ration of the compound (IIA) to NaOH is from about 600:1 to 400:1, preferably from 550:1 to 450:1.

Generally, the reaction temperature can be from about 50° C. to about 150° C., preferably, from about 70° C. to about 120° C., more preferably, from about 90° C. to about 110° C.

Specific examples of a cyclization reaction that converts a compound of formula (IIA) into a compound of Structural Formula (IA′) are described in the Exemplification section.

In another embodiment, the compound of Structural Formula (IIA) is prepared by reacting a compound of Structural Formula (IIIA)

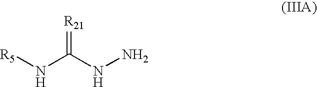

with a compound of Structural Formula (IVA),

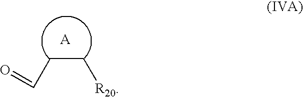

in the presence of an acid. Typically, a catalytic amount of acid is used. “Catalytic amount” typically means a molar ratio from about 0.1 to about 0.001 of the acid catalyst to the reagents. In one embodiment, catalytic amount is 0.01 equivalents. Any acid catalyst can be used, such as organic acids (e.g., formic acid, acetic acid, trifluoroacetic acid), sulfonic acids (e.g., methanesulfonic acid, benzenesulfonic acid and the like), and mineral acids (sulfuric acid, hydrochloric acid, and the like).

General conditions for such a reaction are known in the art and are described, for example, in March, “Advanced Organic Chemistry—Reactions, Mechanisms and Structure”, Third Edition, John Wiley & Sons, (1985). While excess of one reagent over the other can be used, typically, equimolar amounts of the compounds of formulas (IIIA) and (IVA) are employed.

Any suitable solvent in which reagents are soluble and with which reagents do not react can be used. The reaction is most commonly carried out in an alcoholic solvent such as methanol or ethanol with water as co-solvent (e.g., between 0% and about 50% volume/volume (v/v), preferably between about 5% and about 15% v/v).

The reaction is allowed to proceed at a temperature from about 30° C. to about 150° C., preferably from about 40° C. to about 130° C., more preferably, from about 50° C. to about 120° C., even more preferably, from about 60° C. to about 100° C.

Specific examples of a reaction of a compound of Structural Formula (IIIA) and a compound of Structural Formula (IVA) are described in the Exemplification section.

The compound of Structural Formula (IA) can further be deprotected, thereby producing a compound of Structural Formula (IA′)

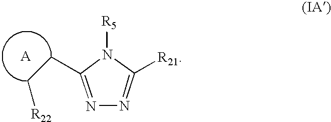

### In Structural Formula (IA′), R₂₂ is —OH, or —NH₂.

In another embodiment, the present invention is a method of synthesis of a compound of Structural Formula (IIA),

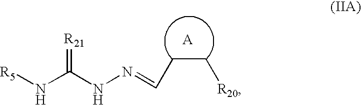

comprising reacting a compound of Structural Formula (IIIA)

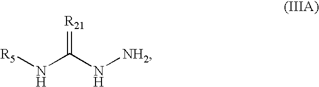

with a compound of Structural Formula (IVA)

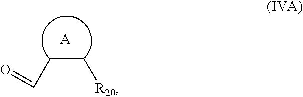

in the presence of acid catalyst. The conditions for this reaction are described above. Methods of preparing the compound of formula (IIIA) are illustrated in the Exemplification section and can be used generally by selecting appropriate starting materials.

In a specific embodiment, in formulas (IA), (IIA), (IIIA) and (IVA), R₂₀ is —ORₚ₁, Rₚ₁ is a benzyl group and the step of deprotecting the compound of formula (IA) comprises reacting a compound of formula (IA) with hydrogen in the presence of palladium-on-charcoal catalyst.

In another specific embodiment, formulas (IA), (IIA), (IIIA) and (IVA), R₂₀ is —ORₚ₁, Rₚ₁ is a benzyl group and the step of deprotecting the compound of formula (IA) comprises reacting a compound of formula (IA) with ammonium formate in the presence of a hydrogen catalyst.

Method III of the present invention begins with an amidation reaction of the starting carboxylic acid represented by the following Structural Formula:

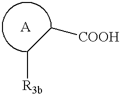

with an amine R₅NH₂ to form an amide represented by Structural Formula (XIIB). The amide is then thionated to form a thioamide represented by Structural Formula (IXB).

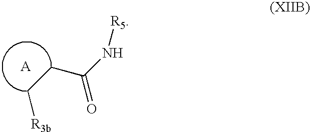

Values for R_{3b}, R₅ and ring A in Structural Formula (XIIB) are as described in Structural Formulas (IB)-(IIIB).

The thioamide is reacted with hydrazino carboxylate in the presence of a mercuric salt to form the [1,2,4]-trizole compound represented by Structural Formula (IB). This synthesis is shown schematically in Scheme 1 of Example 5. A detailed description of each reaction in the synthesis is provided below.

The starting carboxylic acid is first converted to the amide intermediate represented by Structural Formula (XIIB) by reacting the starting carboxylic acid with the amine R₅NH₂. Methods for converting a carboxylic acid to an amide are well known in the art and as described above for method I.

The amide of Structural Formula (XIIB) is then reacted with a thionation reagents to form a thioamide. “Thionation reagent” is as described above for method I.

To thionate the amide of Structural Formula (XIIB), it may be desirable to use a slight excess of the amide, for example up to about 5 equivalents, preferably no more than about 1.5 equivalents. It may also be desirable to use excess thionation reagent. In some cases, it may be desirable to use equal equivalents of the amide and the thionation reagent. Suitable inert solvents include ethereal solvents (e.g., diethyl ether, tetrahydrofuran, glyme and 1,4-dioxane), aromatic solvents (e.g., benzene and toluene) or chlorinated solvents (e.g., methylene chloride and 1,2-dichloroethane). The reaction is carried out at temperatures ranging from about room temperature to about 150° C., preferably from about 75° C. to about 125° C. In a preferred embodiment, the thionation reagent is Lawesson's reagent. Representative conditions for carrying out thionation reaction are found in Exemplification.

The thioamide is then reacted with a hydrazino carboxylate in the presence of a mercuric salt. Although equal molar amounts of hydrazino carboxylate, thioamide and mercuric salt can be used, typically, an excess amount of the hydrazino carboxylate and mercuric salt (e.g., from 1-10 equivalents, 1-5 equivalents or 1-2.5 equivalents) relative to the thioamide is employed for this synthesis. More typically, at least about two molar equivalents of the hydrazino carboxylate and mercuric salt relative to the thioamide, or preferably from 2.0 to about 2.5 equivalents. Optionally, an excess of the thioamide can be used.

Suitable solvent can be any inert organic solvent which is able to dissolve the hydrazino carboxylate, the thioamide and the mercuric salt when mixed. The organic solvent can generally be selected from a C1-C4 aliphatic alcohol (e.g., methanol, ethanol, 1-propanol, 2-propanol, or the like), a C1-C4 aliphatic ketone (e.g., acetone, methyl ethyl ketone, 2-butanone, or the like), a C2-C8 aliphatic ether (e.g., diethyl ether, THF, dioxane, dipropyl ether, diisopropyl ether, or the like), a glycol (e.g., ethylene glycol, propylene glycol, tetramethylene glycol, or the like), an alkyl glycol ether (e.g., ethylene glycol dimethyl ether, or the like), an aromatic solvent (e.g., benzene, toluene, or the like) and acetonitrile. Preferably, the organic solvent can be selected from tetrahydrofuran or dioxane, and more preferably, dioxane.

Suitable reaction temperature ranges between about 50° C. and about 150° C., preferably between about 90° C. and about 120° C.

Suitable mercuric salts include mercuric halides (HgF₂, HgCl₂ and HgBr₂), mercury acetate and HgO, preferably, mercuric halides, and more preferably, HgCl₂.

Optionally, a base such as an amine base (e.g. ammonia, alkyl amines, dialkyl amines, trialkyl amines, optionally substituted amines, optionally substituted cycloalkylamines, N-alkylphthalimide, pyridine, aminopyridines, pyrrolidine, p-toluidine, aniline, p-nitroaniline, azetidine, morpholine, piperidine or the like) can be added to the mixture of the hydrazino carboxylate, thioamide and mercuric salt.

Typically, concentration of the reagents is between 0.005 M and 1.0 M, or preferably, between 0.010 M and 0.500 M.

The synthesis of the triazole compound further includes the step of deprotecting the compound represented by Structural Formula (IB). The products of this deprotecting reaction are triazole-based hsp90 inhibitors.

Variables in Structural Formulas (I)-(IV), (IA)-(IVA), (IB)-(IIIB) are as described above.

In a first specific embodiment, R₅ in Structural Formulas (I)-(IV), (IA), (IA′), (IIA), (IIIA), (IB), (IIIB), (VIIB), (VIIIB), (IXB) and (XIB) is optionally substituted heteroaryl or an optionally substituted 8 to 14 membered aryl. The remainder of the variables are as described in Structural Formulas (I)-(IV), (IA), (IA′), (IIA), (IIIA), (IB), (IIIB), (VIIB), (VIIIB), (IXB) and (XIB).

In a second specific embodiment, R₅ in Structural Formulas (I)-(IV), (IA), (IA′), (IIA), (IIIA), (IB), (IIIB), (VIIB), (VIIIB), (IXB) and (XIB) is a substituted phenyl. The remainder of the variables are as described in Structural Formulas (I)-(IV), (IA), (IA′), (IIA), (IIIA), (IB), (IIIB), (VIIB), (VIIIB), (IXB) and (XIB).

In a third specific embodiment, R₅ in Structural Formulas (I)-(IV), (IA), (IA′), (IIA), (IIIA), (IB), (IIIB), (VIIB), (VIIIB), (IXB) and (XIB) is an optionally substituted cycloalkyl, an optionally substituted cycloalkenyl, or a substituted alkyl.

In a fourth specific embodiment, R₅ in Structural Formulas (I)-(IV), (IA), (IA′), (IIA), (IIIA), (IB), (IIIB), (VIIB), (VIIIB), (IXB) and (XIB) is an optionally substituted naphthyl. The remainder of the variables are as described in Structural Formulas (I)-(IV), (IA), (IA′), (IIA), (IIIA), (IB), (IIIB), (VIIB), (VIIIB), (IXB) and (XIB).

In a fifth specific embodiment, R₅ in Structural Formulas (I)-(IV), (IA), (IA′), (IIA), (IIIA), (IB), (IIIB), (VIIB), (VIIB), (IXB) and (XIB) is represented by the following Structural Formula:

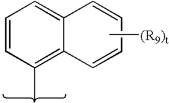

wherein:

for Structural Formulas (I)-(IV), R₉, for each occurrence, is independently a substituent selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, halo, cyano, nitro, guanadino, a haloalkyl, a heteroalkyl, —NR₁₀R₁₁ (provided R₁₀ and R₁₁ are not —H), —OR₇ (provided R₇ is not —H), —SR₇ (provided R₇ is not H), —S(O)ₚR₇, —OS(O)ₚR₇, —NR₈S(O)ₚR₇, —S(O)ₚNR₁₀R₁₁, —OP(O)(OR₇)₂, or —SP(O)(OR₇)₂, —OR_{A}, —SR_{B}, —N(R_{c})₂ or two R₉ groups taken together with the carbon atoms to which they are attached form a fused ring;

for Structural Formulas (IA), (IA′), (IIA), and (IIIA), R₉, for each occurrence, is independently a substituent selected from: —ORₚ₁, —NHRₚ₃, —N(Rₚ₃)₂, —O(CH₂)ₘORₚ₁, or —(CH₂)ₘORₚ₁; an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl; halo, cyano, or nitro; —NR₁₀R₁₁, or —OR₇; —O(CH₂)ₘNR₇Rₚ₃; —C(O)R₇, —C(O)OR₇; —C(O)NR₁₀R₁₁; —OC(O)R₇, —OC(O)OR₇, —OC(O)NR₁₀R₁₁; —NR₈C(O)R₇, or —NR₇C(O)NR₁₀R₁₁; —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁; —NR₈S(O)ₚR₇, —NR₇S(O)ₚNR₁₀R₁₁, —NR₇S(O)ₚOR₇. Alternatively, two R₉ groups taken together with the carbon atoms to which they are attached form a fused ring;

R₇ and R₉, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

or R₁₀ and R₁₁, taken together with the nitrogen to which they are attached, form an optionally substituted heterocyclyl or an optionally substituted heteroaryl; and

p, for each occurrence, is, independently, 0, 1 or 2; and

m, for each occurrence, is independently, 1, 2, 3, or 4;

for Structural Formulas (IB), (IIIB), (VIIB), (VIIIB), (IXB) and (XIB), R₉, for each occurrence, is independently a substituent selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, protected hydroxyalkyl, alkoxyalkyl, halo, cyano, nitro, guanadino, a haloalkyl, a heteroalkyl, —NR₁₀R₁₁, —OR₁₀₀ and —C(O)R₇; or two R₉ groups taken together with the carbon atoms to which they are attached form a fused ring; and

m for Structural Formulas (I)-(IV), (IA), (IA′), (IIA), (IIIA), (IB), (IIIB), (VIIB), (VIIB), (IXB) and (XIB) is zero or an integer from 1 to 7.

The remainder of the variables are as described in Structural Formulas (I)-(IV), (IA), (IA′), (IIA), (IIIA), (IB), (IIIB), (VIIB), (VIIB), (IXB) and (XIB).

In a sixth specific embodiment, R₅ in Structural Formulas (I)-(IV), (IA), (IA′), (IIA), (IIIA), (IB), (IIIB), (VIIB), (VIIB), (IXB) and (XIB) is represented by the following Structural Formula:

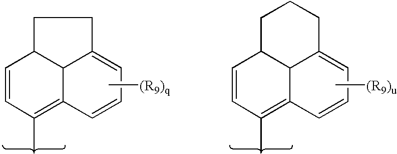

wherein q is zero or an integer from 1 to 7; and

u is zero or an integer from 1 to 8. The remainder of the variables are as described in the fifth specific embodiment.

In a seventh specific embodiment, R₅ in Structural Formulas (I)-(IV), (IA), (IA′), (IIA), (IIIA), (IB), (IIIB), (VIIB), (VIIB), (IXB) and (XIB) is an optionally substituted indolyl. Preferably, R₅ is an indolyl represented by the following Structural Formula:

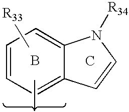

wherein:

for Structural Formulas (I)-(IV), R₃₃ is a halo, a lower alkyl, a lower alkoxy, a lower haloalkyl, a lower haloalkoxy, or a lower alkyl sulfanyl;

R₃₄ is H, or a lower alkyl;

Ring B and Ring C are optionally substituted with one or more substituents in addition to R₃₃ and R₃₄. The remainder of the variables are as described in Structural Formulas (I)-(IV);

for Structural Formulas (IA), (IA′), (IIA), and (IIIA), R₃₃ is H; —ORₚ₁, —NHRₚ₃ or —N(Rₚ₃)₂, a halo, a lower alkyl, a lower alkoxy, a lower haloalkyl, or a lower haloalkoxy; R₃₄ is H, —ORₚ₁, —NHRₚ₃ or —N(Rₚ₃)₂, a C1-C6 alkyl, or a lower alkylcarbonyl; and ring B and ring C are optionally substituted with one or more substituents in addition to R₃₃ and R₃₄. The remainder of the variables are as described in Structural Formulas (IA), (IA′), (IIA), and (IIIA);

for Structural Formulas (IB), (IIIB), (VIIB), (VIIIB), (IXB) and (XIB), R₃₃ is a halo, a lower alkyl, a lower alkoxy, a lower haloalkyl, and a lower haloalkoxy, and a lower alkyl sulfanyl; R₃₄ is —H, a lower alkyl, or a lower acyl; Rings B and Ring C are optionally substituted with one or more substituents in addition to R₃₃ and R₃₄; and the remainder of the variables are as described in Structural Formulas (IB), (IIIB), (VIIB), (VIIIB), (IXB) and (XIB).

In a eighth specific embodiment, R₅ in Structural Formulas (I)-(IV), (IA), (IA′), (IIA), (IIIA), (IB), (IIIB), (VIIB), (VIIB), (IXB) and (XIB) is a substituted phenyl.

For Structural Formulas (I)-(IV), the phenyl group is optionally substituted with:

i) one substituent selected from nitro, cyano, a haloalkoxy, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, guanadino, —NR₁₀R₁₁ (provided R₁₀ and R₁₁ are not —H)—O—R₂₀, —SR₇ (provided R₇ is not H), —S(O)ₚR₇, —OS(O)ₚR₇, —NR₈S(O)ₚR₇, —S(O)ₚNR₁₀R₁₁, —OP(O)(OR₇)₂, —SP(O)(OR₇)₂, —OR_{A}, —SR_{B}, or —N(R_{C})₂; or

ii) two to five substituents selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, —F, —Br, —I, cyano, nitro, guanadino, a haloalkyl, a heteroalkyl, —NR₁₀R₁₁ (provided R₁₀ and R₁₁ are not H)—OR₇—SR₇, —S(O)ₚR₇, —OS(O)ₚR₇, —NR₈S(O)ₚR₇, —S(O)ₚNR₁₀R₁₁, —OP(O)(OR₇)₂, or —SP(O)(OR₇)₂, —S(O)ₚOR₇, —OR_{A}, —SR_{B}, or —N(R_{C})₂,

wherein R₇, R₈, R₁₀, R₁₁, R_{A}, R_{B}, R_{C}, and p are as described above for Structural Formulas (I)-(IV).

For Structural Formulas (IA), (IA′), (IIA), and (IIIA), the substituents for the phenyl group is selected from the group consisting of —ORₚ₁, —NHRₚ₃, —N(Rₚ₃)₂, —O(CH₂)ₘORₚ₁, or —(CH₂)ₘORₚ₁; an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl; halo, cyano, or nitro; —NR₁₀R₁₁, or —OR₇; —O(CH₂)ₘNR₇Rₚ₃; —C(O)R₇, —C(O)OR₇; —C(O)NR₁₀R₁₁; —OC(O)R₇, —OC(O)OR₇, —OC(O)NR₁₀R₁₁; —NR₈C(O)R₇, or —NR₇C(O)NR₁₀R₁₁; —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁; —NR₈S(O)ₚR₇, —NR₇S(O)ₚNR₁₀R₁₁, —NR₇S(O)ₚOR₇. Values and specific values for Rₚ₁, Rₚ₃, R₇, R₈, R₁₀, R₁₁, p and m are as defined above with reference to formulas (IA), (IA′), (IIA), and (IIIA)

For Structural Formulas (IB), (IIIB), (VIIB), (VIIIB), (IXB) and (XIB), the phenyl group is substituted with:

i) the one substituent selected from nitro, cyano, a haloalkoxy, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, protected hydroxylalkyl, alkoxyalkyl, guanadino, —OR₁₀₀, —SR₁₀₁, —N(R₁₀₂)₂, —NR₁₀R₁₁, —OR₇, or —C(O)R₇; or

ii) two to five substituents selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, protected hydroxyalkyl, alkoxyalkyl, —F, —Br, —I, cyano, nitro, guanadino, a haloalkyl, a heteroalkyl, —OR₁₀₀, —SR₁₀₁, —N(R₁₀₂)₂, —NR₁₀R₁₁, —OR₁₀₀, or —C(O)R₇; and; and the values and preferred values of the remaining variables are as described in Structural Formulas (IB), (IIIB), (VIIB), (VIIIB), (IXB) and (XIB).

Preferably, for Structural Formulas (I)-(IV), (IA), (IA′), (IIA), (IIIA), (IB), (IIIB), (VIIB), (VIIIB), (IXB) and (XIB), R₅ is represented by the following structural formula:

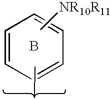

R₁₀ and R₁₁ are as described above.

In a ninth specific embodiment, R₅ in Structural Formulas (I)-(IV), (IA), (IA′), (IIA), (IIIA), (IB), (IIIB), (VIIB), (VIIB), (IXB) and (XIB) is selected from the group consisting of:

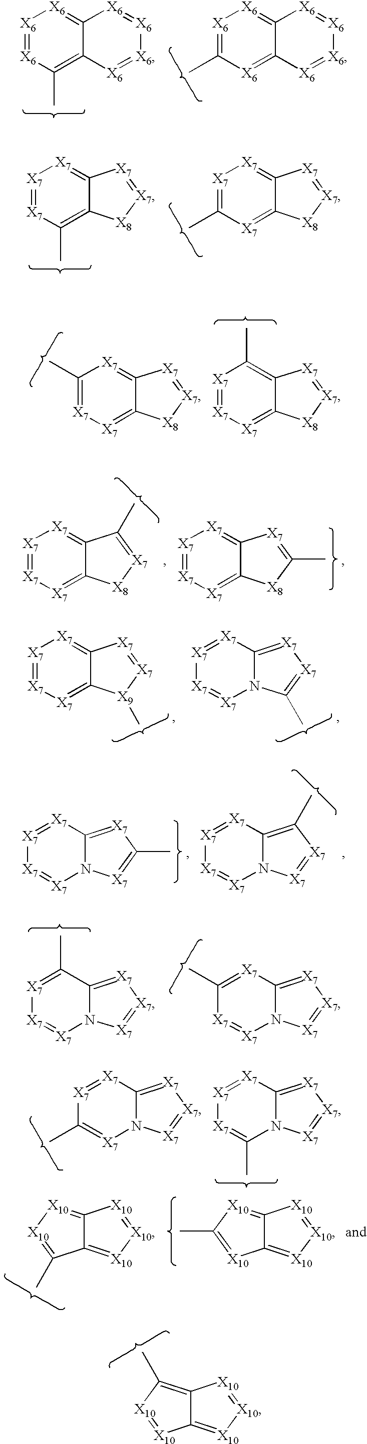

wherein:

X₆, for each occurrence, is independently CH, CR₉, N, N(O), N⁺(R₁₇), provided that at least three X₆ groups are independently selected from CH and CR₉;

X₇, for each occurrence, is independently CH, CR₉, N, N(O), N⁺(R₁₇), provided that at least three X₇ groups are independently selected from CH and CR₉;

X₈, for each occurrence, is independently CH₂, CHR₉, C(R₉)₂, S, S(O)ₚ, NR₇, or NR₁₇;

X₉, for each occurrence, is independently N or CH;

X₁₀, for each occurrence, is independently CH, CR₉, N, N(O), N⁺(R₁₇), provided that at least one X₁₀ is selected from CH and CR₉;

R₁₇, for each occurrence, is independently —H, an alkyl, an aralkyl.

For Structural Formulas (IA), (IA′), (IIA), and (IIIA), R₁₇ can also be —C(O)R₇, —C(O)OR₇ or —C(O)NR₁₀R₁₁.

For Structural Formulas (IB), (IIIB), (VIIB), (VIIIB), (IXB) and (XIB), R₁₇ can also be —C(O)R₇.

Values and specific values for the remainder of the variables are as described above in the fifth specific embodiment.

Preferably, for the ninth specific embodiment, R₅ is an optionally substituted indolyl, an optionally substituted benzoimidazolyl, an optionally substituted indazolyl, an optionally substituted 3H-indazolyl, an optionally substituted indolizinyl, an optionally substituted quinolinyl, an optionally substituted isoquinolinyl, an optionally substituted benzoxazolyl, an optionally substituted benzo[1,3]dioxolyl, an optionally substituted benzofuryl, an optionally substituted benzothiazolyl, an optionally substituted benzo[d]isoxazolyl, an optionally substituted benzo[d]isothiazolyl, an optionally substituted thiazolo[4,5-c]pyridinyl, an optionally substituted thiazolo[5,4-c]pyridinyl, an optionally substituted thiazolo[4,5-b]pyridinyl, an optionally substituted thiazolo[5,4-b]pyridinyl, an optionally substituted oxazolo[4,5-c]pyridinyl, an optionally substituted oxazolo[5,4-c]pyridinyl, an optionally substituted oxazolo[4,5-b]pyridinyl, an optionally substituted oxazolo[5,4-b]pyridinyl, an optionally substituted imidazopyridinyl, an optionally substituted benzothiadiazolyl, benzoxadiazolyl, an optionally substituted benzotriazolyl, an optionally substituted tetrahydroindolyl, an optionally substituted azaindolyl, an optionally substituted quinazolinyl, an optionally substituted purinyl, an optionally substituted imidazo[4,5-a]pyridinyl, an optionally substituted imidazo[1,2-a]pyridinyl, an optionally substituted 3H-imidazo[4,5-b]pyridinyl, an optionally substituted 1H-imidazo[4,5-b]pyridinyl, an optionally substituted 1H-imidazo[4,5-c]pyridinyl, an optionally substituted 3H-imidazo[4,5-c]pyridinyl, an optionally substituted pyridopyrdazinyl, and optionally substituted pyridopyrimidinyl, an optionally substituted pyrrolo[2,3]pyrimidyl, an optionally substituted pyrazolo[3,4]pyrimidyl an optionally substituted cyclopentaimidazolyl, an optionally substituted cyclopentatriazolyl, an optionally substituted pyrrolopyrazolyl, an optionally substituted pyrroloimidazolyl, an optionally substituted pyrrolotriazolyl, or an optionally substituted benzo[b]thienyl.

In a tenth specific embodiment, R₅ in Structural Formulas (I)-(IV), (IA), (IA′), (IIA), (IIIA), (IB), (IIIB), (VIIB), (VIIIB), (IXB) and (XIB) is selected from the group consisting of:

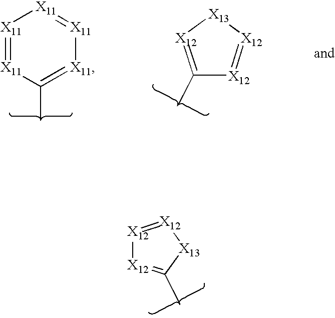

wherein:

X₁₁, for each occurrence, is independently CH, CR₉, N, N(O), or N⁺(R₁₇), provided that at least one X₁₁ is N, N(O), or N⁺(R₁₇) and at least two X₁₁ groups are independently selected from CH and CR₉;

X₁₂, for each occurrence, is independently CH, CR₉, N, N(O), N⁺(R₁₇), provided that at least one X₁₂ group is independently selected from CH and CR₉;

X₁₃, for each occurrence, is independently O, S, S(O)ₚ, NR₇, or NR₁₇.

Values and specific values for the remainder of the variables are as described in the ninth specific embodiment.

In a eleventh specific embodiment, R₅ in Structural Formulas (I)-(IV), (IA), (IA′), (IIA), (IIIA), (IB), (IIIB), (VIIB), (VIIB), (IXB) and (XIB) is an optionally substituted cycloalkyl, and optionally substituted cycloalkenyl, or a substituted alkyl, wherein the alkyl group is substituted with one or more substituents independently selected from the following groups:

for Structural Formulas (I)-(IV), the one or more substituents for the alkyl group are independently selected from the group consisting of an optionally substituted alkynyl, an optionally substituted cycloalkyl, an optionally substituted cycloalkenyl, an optionally substituted heteroaryl, an optionally substituted aralkyl, an optionally substituted heteraralkyl, halo, cyano, nitro, guanadino, a haloalkyl, —NR₁₀R₁₁ (provided R₁₀ and R₁₁ is not H), —OR₇ (provided R₇ is not H), —SR₇ (provided R₇ is not H), —S(O)ₚR₇, —OS(O)ₚR₇, —NR₈S(O)ₚR₇, —S(O)ₚNR₁₀R₁₁, —OR_{A}, —SR_{B}, or —N(R_{C})₂, wherein values and specific values for R₇, R₈, R₁₀, R₁₁, R_{A}, R_{B}, R_{C}, and p are as described for the Structural Formulas (I)-(IV);

for Structural Formulas (IA), (IA′), (IIA), and (IIIA), the one or more substituents for the alkyl or the cycloalkyl group are independently selected from the group consisting of —ORₚ₁, —NHRₚ₃, —N(Rₚ₃)₂, —O(CH₂)ₘORₚ₁, or —(CH₂)ₘORₚ₁; an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl; halo, cyano, or nitro; —NR₁₀R₁₁, or —OR₇; —O(CH₂)ₘNR₇Rₚ₃; —C(O)R₇, —C(O)OR₇; —C(O)NR₁₀R₁₁; —OC(O)R₇, —OC(O)OR₇, —OC(O)NR₁₀R₁₁; —NR₈C(O)R₇, or —NR₇C(O)NR₁₀R₁₁; —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁; —NR₈S(O)ₚR₇, —NR₇S(O)ₚNR₁₀R₁₁, —NR₇S(O)ₚOR₇. Values and preferred values for Rₚ₁, R₃, R₇, R₈, R₁₀, R₁₁, p and m are as defined above with reference to Structural Formulas (IA), (IA′), (IIA), and (IIIA).

for Structural Formulas (IB), (IIIB), (VIIB), (VIIIB), (IXB) and (XIB), the one or more substituents for the alkyl group are independently selected from the group consisting of an optionally substituted alkynyl, an optionally substituted cycloalkyl, an optionally substituted cycloalkenyl, an optionally substituted heteroaryl, an optionally substituted aralkyl, an optionally substituted heteraralkyl, halo, cyano, nitro, guanadino, a haloalkyl, —NR₁₀R₁₁, —OR₁₀₀, and —C(O)R₇.

Values and specific values for the remainder of the variables are as described above for Structural Formulas (I)-(IV), (IA), (IA′), (IIA), (IIIA), (IB), (IIIB), (VIIB), (VIIB), (IXB) and (XIB).

In a more specific embodiment, R₅ in Structural Formulas (I)-(IV), (IA), (IA′), (IIA), (IIIA), (IB), (IIIB), (VIIB), (VIIIB), (IXB) and (XIB) is an optionally substituted cycloalkyl or an optionally substituted cycloalkenyl. The remainder of the variables are as described above in the eleventh specific embodiment.

In another more specific embodiment, R₅ in Structural Formulas (I)-(IV), (IA), (IA′), (IIA), (IIIA), (IB), (IIIB), (VIIB), (VIIIB), (IXB) and (XIB), is a substituted alkyl. The remainder of the variables are as described above in the eleventh specific embodiment.

In a twelfth specific embodiment, ring A in Structural Formulas (I)-(IV), (IA), (IA′), (IIA), (IVA), (IB), (IIIB), (VIIB), (VIIIB), (IXB), and (XIB) is presented by Structural Formula (IX):

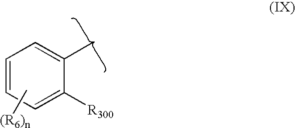

wherein:
For Structural Formulas (I)-(IV), R₃₀₀ is R₃ as described in Structural Formulas (I)-(IV). R₆, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteroaralkyl, halo, cyano, nitro, guanadino, a haloalkyl, a heteroalkyl, alkoxy, haloalkoxy, —NR₁₀R₁₁ (provided R₁₀ and R₁₁ are not —H), —OR₇ (provided R₇ is not H), —C(NR₈)OR₇, —C(NR₈)NR₁₀R₁₁, —C(NR₈)SR₇, —OC(S)OR₇, —OC(NR₈)OR₇, —SC(NR₈)OR₇—SC(S)OR₇, —OC(S)NR₁₀R₁₁, —OC(NR₈)NR₁₀R₁₁, —SC(NR₈)NR₁₀R₁₁, —SC(S)NR₁₀R₁₁, —OC(NR₈)R₇, —SC(NR₈)R₇, —NR₇C(S)OR₇, —NR₇C(NR₈)R₇, —NR₇C(NR₈)OR₇, —NR₇C(S)NR₁₀R₁₁, —NR₇C(NR₈)NR₁₀R₁₁, —SR₇ (provided R₇ is not H), —S(O)ₚR₇, —OS(O)ₚR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂, —OR_{A}, —SR_{B}, or —N(R_{C})₂;
For Structural Formulas (IA), (IA′), (IIA) and (IVA), R₃₀₀ is R₂₀ as described in Structural Formulas (IA), (IA′), (IIA) and (IVA). R₆, for each occurrence, is independently a substituent selected from: —ORₚ₁, —NHRₚ₃, —N(Rₚ₃)₂, —O(CH₂)ₘORₚ₁, or —(CH₂)ₘORₚ₁; an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl; halo, cyano, or nitro; —NR₁₀R₁₁, or —OR₇; —O(CH₂)ₘNR₇Rₚ₃; —C(O)R₇, —C(O)OR₇; —C(O)NR₁₀R₁₁; —OC(O)R₇, —OC(O)OR₇, —OC(O)NR₁₀R₁₁; —NR₈C(O)R₇, or —NR₇C(O)NR₁₀R₁₁; —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁; —NR₈S(O)ₚR₇, —NR₇S(O)ₚNR₁₀R₁₁, —NR₇S(O)ₚOR₇;
For Structural Formulas (IB), (IIIB), (VIIB), (VIIIB), (IXB), and (XIB), R₃₀₀ is R_{3b} as described above in Structural Formulas (IB), (IIIB), (VIIB), (VIIIB), (IXB), and (XIB). R₆, for each occurrence, is independently an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteroaralkyl, halo, cyano, nitro, guanadino, a haloalkyl, a heteroalkyl, alkoxy, a protected hydroxyalkyl, haloalkoxy, —NR₁₀R₁₁, —OR₁₀₀, —C(O)R₇, or —SR₁₀₁; or two R₆ groups, taken together with the carbon atoms to which they are attached, form a fused ring;
For Structural Formulas (I)-(IV), (IA), (IA′), (IIA), (IVA), (IB), (IIIB), (VIIB), (VIIIB), (IXB), and (XIB), n is zero or an integer from 1 to 4. The remainder of the variables are as described above in Structural Formulas (I)-(IV), (IA), (IA′), (IIA), (IVA), (IVA), (IB), (IIIB), (VIIB), (VIIIB), (IXB), and (XIB).

In a more specific embodiment, R₅ in compounds of Structural Formulas (I)-(IV), (IA), (IA′), (IIA), (IB), (IIIB), (VIIB), (VIIIB), (IXB), and (XIB) is an optionally substituted phenyl, wherein the phenyl group is substituted with substituents as described in the eighth specific embodiment. Values and specific values for the remainder of the variables are as described in the twelfth specific embodiment. Preferably, for Structural Formulas (I)-(IV), R₃ is —OR_{A}, SR_{B}, N(R_{C})₂.

In another more specific embodiment, R₅ in compounds of Structural Formulas (I)-(IV), (IA), (IA′), (IIA), (IB), (IIIB), (VIIB), (VIIIB), (IXB), and (XIB), is an optionally substituted cycloalkyl, and optionally substituted cycloalkenyl, or a substituted alkyl, wherein the alkyl group is substituted with one or more substituents independently selected from the group described above in the eleventh specific embodiment. Values and specific values for the remainder of the variables are as described in the twelfth specific embodiment. In a even more specific embodiment, R₅ is an optionally substituted cycloalkyl or an optionally substituted cycloalkenyl. In another even more specific embodiment, R₅ is an optionally substituted alkyl.

In another more specific embodiment, R₅ in the compounds of Structural Formulas (I)-(IV), (IA), (IA′), (IIA), (IB), (IIIB), (VIIB), (VIIIB), (IXB), and (XIB) is represented by the following structural formula:

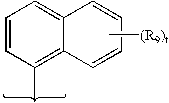

wherein R₉ and m are as described in the fifth specific embodiment. Values and specific values for the remainder of the variables are as described the twelfth specific embodiment.

In another more specific embodiment, R₅ in compounds of Structural Formulas (I)-(IV), (IA), (IA′), (IIA), (IB), (IIIB), (VIIB), (VIIIB), (IXB), and (XIB), is selected from the group consisting of the following structural formulas:

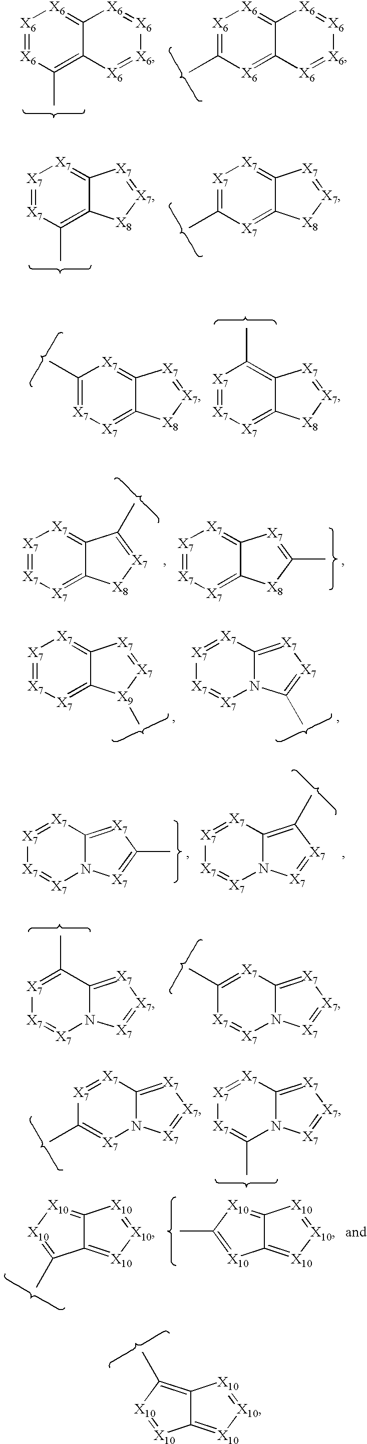

wherein X₆, X₇, X₈, X₉, X₁₀ and R₁₇ are as described in the ninth specific embodiment. Values and specific values for the remainder of the variables are as described in the twelfth specific embodiment. Preferably, R₅ is an optionally substituted indolyl, an optionally substituted benzoimidazolyl, an optionally substituted indazolyl, an optionally substituted 3H-indazolyl, an optionally substituted indolizinyl, an optionally substituted quinolinyl, an optionally substituted isoquinolinyl, an optionally substituted benzoxazolyl, an optionally substituted benzo[1,3]dioxolyl, an optionally substituted benzofuryl, an optionally substituted benzothiazolyl, an optionally substituted benzo[d]isoxazolyl, an optionally substituted benzo[d]isothiazolyl, an optionally substituted thiazolo[4,5-c]pyridinyl, an optionally substituted thiazolo[5,4-c]pyridinyl, an optionally substituted thiazolo[4,5-b]pyridinyl, an optionally substituted thiazolo[5,4-b]pyridinyl, an optionally substituted oxazolo[4,5-c]pyridinyl, an optionally substituted oxazolo[5,4-c]pyridinyl, an optionally substituted oxazolo[4,5-b]pyridinyl, an optionally substituted oxazolo[5,4-b]pyridinyl, an optionally substituted imidazopyridinyl, an optionally substituted benzothiadiazolyl, benzoxadiazolyl, an optionally substituted benzotriazolyl, an optionally substituted tetrahydroindolyl, an optionally substituted azaindolyl, an optionally substituted quinazolinyl, an optionally substituted purinyl, an optionally substituted imidazo[4,5-a]pyridinyl, an optionally substituted imidazo[1,2-a]pyridinyl, an optionally substituted 3H-imidazo[4,5-b]pyridinyl, an optionally substituted 1H-imidazo[4,5-b]pyridinyl, an optionally substituted 1H-imidazo[4,5-c]pyridinyl, an optionally substituted 3H-imidazo[4,5-c]pyridinyl, an optionally substituted pyridopyrdazinyl, and optionally substituted pyridopyrimidinyl, an optionally substituted pyrrolo[2,3]pyrimidyl, an optionally substituted pyrazolo[3,4]pyrimidyl an optionally substituted cyclopentaimidazolyl, an optionally substituted cyclopentatriazolyl, an optionally substituted pyrrolopyrazolyl, an optionally substituted pyrroloimidazolyl, an optionally substituted pyrrolotriazolyl, or an optionally substituted benzo[b]thienyl.

In another more specific embodiment, R₅ in the compounds of Structural Formulas (I)-(IV), (IA), (IA′), (IIA), (IB), (IIIB), (VIIB), (VIIIB), (IXB), and (XIB), is selected from the group consisting of the following structural formulas:

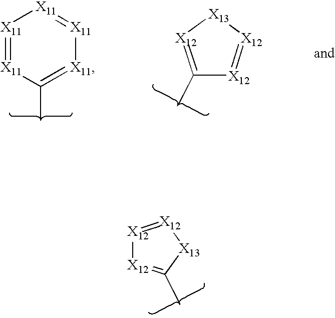

wherein X₁₁, X₁₂, X₁₃, R₉ and R₁₇ are defined as described in the tenth specific embodiment. Values and specific values for the remainder of the variables are as described in the twelfth specific embodiment.

In another more specific embodiment, R₅ in the compounds of Structural Formulas (I)-(IV), (IA), (IA′), (IIA), (IB), (IIIB), (VIIB), (VIIIB), (IXB), and (XIB) is an optionally substituted indolyl. Preferably, R₅ is an indolyl represented by the following Structural Formula:

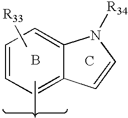

wherein R₃₃, R₃₄, ring B and ring C are as described above in the seventh specific embodiment. Values and specific values for the remainder of the variables are as described above in the twelfth specific embodiment.

In the thirteenth specific embodiment, ring A in compounds represented by Structural Formulas (I)-(IV), (IA), (IA′), (IIA), (IVA), (IB), (IIIB), (VIIB), (VIIIB), (IXB), and (XIB) is represented by the Structural Formula (X):

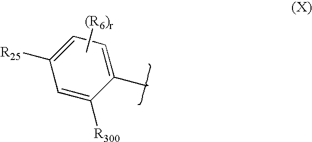

wherein:
for Structural Formulas (I)-(IV), R₂₅ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteroaralkyl, halo, cyano, nitro, guanadino, a haloalkyl, a heteroalkyl, alkoxy, haloalkoxy, or —NR₁₀R₁₁ (provided R₁₀ and R₁₁ are not H);
for Structural Formulas (IA), (IA′), (IIA), and (IVA), R₂₅ is —ORₚ₁, —NHRₚ₃, —N(Rₚ₃)₂, —O(CH₂)ₘORₚ₁, or —(CH₂)ₘORₚ₁; an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl; halo, cyano, or nitro; —NR₁₀R₁₁, or —OR₇; —O(CH₂)ₘNR₇Rₚ₃; —C(O)R₇, —C(O)OR₇; —C(O)NR₁₀R₁₁; —OC(O)R₇, —OC(O)OR₇, —OC(O)NR₁₀R₁₁; —NR₈C(O)R₇, or —NR₇C(O)NR₁₀R₁₁; —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁; —NR₈S(O)ₚR₇, —NR₇S(O)ₚNR₁₀R₁₁, or —NR₇S(O)ₚOR₇;
for Structural Formulas (IB), (IIIB), (VIIB), (VIIIB), (IXB), and (XIB), R₂₅ is a substituent selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, protected hydroxyalkyl, alkoxyalkyl, halo, cyano, nitro, guanadino, a haloalkyl, a heteroalkyl, —NR₁₀R₁₁, —OR₁₀₀, and —C(O)R₇;
r for Structural Formulas (I)-(IV), (IA), (IA′), (IIA), (IVA), (IB), (IIIB), (VIIB), (VIIIB), (IXB), and (XIB) is zero or an integer from 1 to 3.

Values and specific values for the remainder of the variables are as described above in the twelfth specific embodiment.

In a more specific embodiment, R₂₅ in Structural Formulas (I)-(IV) is —OR_{A}, —SR_{B}, —N(R_{C})₂, —OC(S)OR₇, —OC(NR₈)OR₇, —SC(NR₈)OR₇, —SC(S)OR₇, —OC(S)NR₁₀R₁₁, —SC(S)NR₁₀R₁₁, —OC(NR₈)R₇, —SC(NR₈)R₇, —OS(O)ₚR₇, —S(O)ₚOR₇, —SS(O)ₚOR₇, —SS(O)ₚR₇, —OP(O)(OR₇)₂, or —SP(O)(OR₇)₂, wherein p is 0, 1, or 2; Values and specific values for the remainder of the variables are as described in the thirteenth specific embodiment.

In another more specific embodiment, R₂₅ in Structural Formulas (IA), (IA′), (IIA), and (IVA) is a —ORₚ₁, —NHRₚ₃, —N(Rₚ₃)₂, —O(CH₂)ₘORₚ₁, or —(CH₂)ₘORₚ₁.

In another more specific embodiment, R₃₃ is H, —ORₚ₁, —NHRₚ₃, —N(Rₚ₃)₂, —O(CH₂)ₘORₚ₁, or —(CH₂)ₘORₚ₁; and R₃₄ is a C₁-C₆ alkyl.

In another more specific embodiment, R_{3b} and R₂₅ in Structural Formulas (IB), (IIIB), (VIIB), (VIIIB), (IXB), and (XIB) are —OR₁₀₀, —SR₁₀₁, or —N(R₁₀₂)₂. Even more specifically, R_{1b} is —SH or —OH; R_{3b} and R₂₅ are —OR₁₀₀; and R₅₁ is ═O or ═S. Even more specifically, R_{1b} is —SH or —OH; R_{3b} and R₂₅ are —OR₁₀₀; R₅₁ is ═O or ═S; and R₆ is an optionally substituted lower alkyl, a C3-C6 cycloalkyl, a lower alkoxy, a lower alkyl sulfanyl, or —NR₁₀R₁₁.

In another more specific embodiment, R₅ in Structural Formulas (I)-(IV), (IA), (IA′), (IIA), (IB), (IIIB), (VIIB), (VIIIB), (IXB), and (XIB) is an optionally substituted phenyl, wherein the phenyl group is substituted with substituents as described in the eighth specific embodiment. Values and specific values for the remainder of the variables are as described in the thirteenth specific embodiment. Preferably, R₃ is —OR_{A}, SR_{B}, N(R_{C})₂.

In another more specific embodiment, R₅ in compounds of Structural Formulas (I)-(IV), (IA), (IA′), (IIA), (IB), (IIIB), (VIIB), (VIIIB), (IXB), and (XIB), is an optionally substituted cycloalkyl, and optionally substituted cycloalkenyl, or a substituted alkyl, wherein the alkyl group is substituted with one or more substituents independently selected from the group as described above in the eleventh specific embodiment. The remainder of the variables are as described in the thirteenth specific embodiment. In a even more specific embodiment, R₅ is an optionally substituted cycloalkyl or an optionally substituted cycloalkenyl. In another even more specific embodiment, R₅ is an optionally substituted alkyl.

In another more specific embodiment, R₅ in the compounds of Structural Formulas (I)-(IV), (IA), (IA′), (IIA), (IB), (IIIB), (VIIB), (VIIIB), (IXB), and (XIB) is represented by the following structural formula:

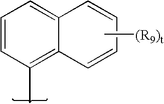

wherein R₉ and m are as described in the fifth specific embodiment. Values and specific values for the remainder of the variables are as described the thirteenth specific embodiment.

In another more specific embodiment, R₅ in compounds of Structural Formulas (I)-(IV), (IA), (IA′), (IIA), (IB), (IIIB), (VIIB), (VIIIB), (IXB), and (XIB), is selected from the group consisting of the following structural formulas:

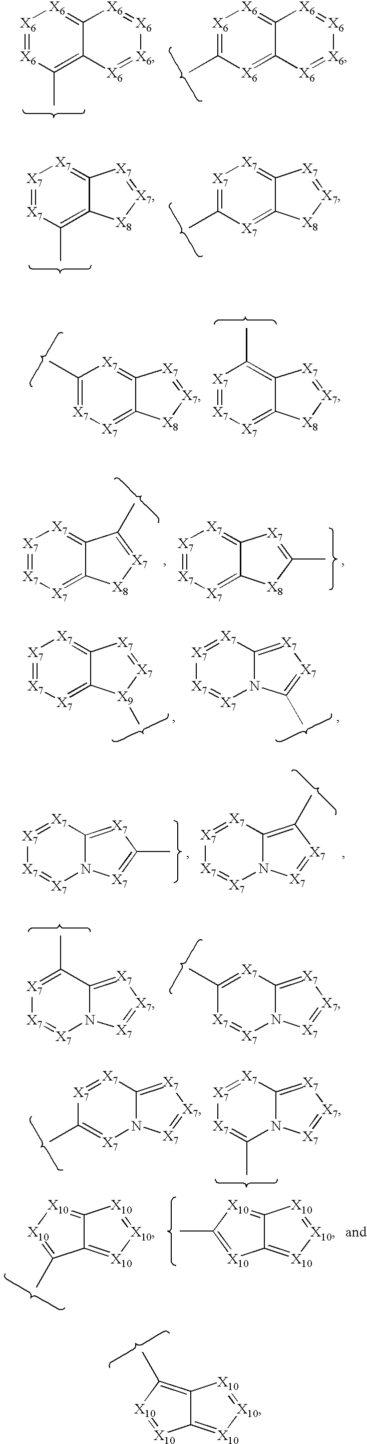

wherein X₆, X₇, X₈, X₉, X₁₀ and R₁₇ are as described in the ninth specific embodiment. The remainder of the variables are as described in the thirteenth specific embodiment. Preferably, R₅ is an optionally substituted indolyl, an optionally substituted benzoimidazolyl, an optionally substituted indazolyl, an optionally substituted 3H-indazolyl, an optionally substituted indolizinyl, an optionally substituted quinolinyl, an optionally substituted isoquinolinyl, an optionally substituted benzoxazolyl, an optionally substituted benzo[1,3]dioxolyl, an optionally substituted benzofuryl, an optionally substituted benzothiazolyl, an optionally substituted benzo[d]isoxazolyl, an optionally substituted benzo[d]isothiazolyl, an optionally substituted thiazolo[4,5-c]pyridinyl, an optionally substituted thiazolo[5,4-c]pyridinyl, an optionally substituted thiazolo[4,5-b]pyridinyl, an optionally substituted thiazolo[5,4-b]pyridinyl, an optionally substituted oxazolo[4,5-c]pyridinyl, an optionally substituted oxazolo[5,4-c]pyridinyl, an optionally substituted oxazolo[4,5-b]pyridinyl, an optionally substituted oxazolo[5,4-b]pyridinyl, an optionally substituted imidazopyridinyl, an optionally substituted benzothiadiazolyl, benzoxadiazolyl, an optionally substituted benzotriazolyl, an optionally substituted tetrahydroindolyl, an optionally substituted azaindolyl, an optionally substituted quinazolinyl, an optionally substituted purinyl, an optionally substituted imidazo[4,5-a]pyridinyl, an optionally substituted imidazo[1,2-a]pyridinyl, an optionally substituted 3H-imidazo[4,5-b]pyridinyl, an optionally substituted 1H-imidazo[4,5-b]pyridinyl, an optionally substituted 1H-imidazo[4,5-c]pyridinyl, an optionally substituted 3H-imidazo[4,5-c]pyridinyl, an optionally substituted pyridopyrdazinyl, and optionally substituted pyridopyrimidinyl, an optionally substituted pyrrolo[2,3]pyrimidyl, an optionally substituted pyrazolo[3,4]pyrimidyl an optionally substituted cyclopentaimidazolyl, an optionally substituted cyclopentatriazolyl, an optionally substituted pyrrolopyrazolyl, an optionally substituted pyrroloimidazolyl, an optionally substituted pyrrolotriazolyl, or an optionally substituted benzo[b]thienyl.

In another more specific embodiment, R₅ in Structural Formulas (I)-(IV), (IA), (IA′), (IIA), (IB), (IIIB), (VIIB), (VIIIB), (IXB), and (XIB) is an optionally substituted indolyl. Preferably, R₅ is an indolyl represented by the following Structural Formula:

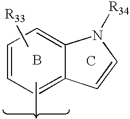

wherein R₃₃, R₃₄, ring B and ring C are as described above in the seventh specific embodiment. Values and specific values for the remainder of the variables are as described above in the thirteenth specific embodiment. In a even more specific embodiment, for Structural Formulas (IA), (IA′) and (IIA), R₂₅ is a —ORₚ₁, —NHRₚ₃, —N(Rₚ₃)₂, —O(CH₂)ₘORₚ₁, or —(CH₂)ₘORₚ₁. In another even more specific embodiment, for Structural Formulas (IA), (IA′) and (IIA), R₃₃ is H, —ORₚ₁, —NHRₚ₃, —N(Rₚ₃)₂, —O(CH₂)ₘORₚ₁, or —(CH₂)ₘORₚ₁ and R₃₄ is a C₁-C₆ alkyl. In another even more specific embodiment, for Structural Formulas (IA), (IA′) and (IIA), R₂₅ is a —ORₚ₁, —NHRₚ₃, —N(Rₚ₃)₂, —O(CH₂)ₘORₚ₁, or —(CH₂)ₘORₚ₁; R₃₃ is H, —ORₚ₁, —NHRₚ₃, —N(Rₚ₃)₂, —O(CH₂)ₘORₚ₁, or (CH₂)ₘORₚ₁ and R₃₄ is a C1-C6 alkyl. In a even more specific embodiment, for Structural Formulas (IA), (IA′) and (IIA), R₂₁ is O; R₆ is a C1-C6 alkyl, a C1-C6 haloalkyl, a C1-C6 alkoxy, a C1-C6 haloalkoxy, a C3-C6 cycloalkyl or —NR₁₀R₁₁. In another even more specific embodiment, for Structural Formulas (IA), (IA′) and (IIA), R₆ is a C1-C6 alkyl and R₃₃ is H. In another even more specific embodiment, for Structural Formulas (IA), (IA′) and (IIA), R₃₃ is —H and ring B is unsubstituted. In another even more specific embodiment, for Structural Formulas (IA), (IA′) and (IIA), R₂₀ and R₂₅ are —OH, and R₆ is a C1-C6 alkyl. In another even more specific embodiment, for Structural Formula (IA), (IA′) and (IIA), R₂₁ is O; R₆ is a C1-C6 alkyl and R₃₃ is H. In another even more specific embodiment, for Structural Formulas (IA), (IA′) and (IIA), R₂₁ is O; R₆ is a C₁-C₆ alkyl; R₃₃ is H and ring B is unsubstituted. In yet another even more embodiment, for Structural Formula (IA), (IA′) and (IIA), R₂₁ is O; R₆ is a C1-C6 alkyl; R₃₃ is H; ring B is unsubstituted; R₂₀ and R₂₅ are —OH, and R₆ is a C1-C6 alkyl.

In another more specific embodiment, R₅ in the compounds of Structural Formulas (I)-(IV), (IA), (IA′), (IIA), (IB), (IIIB), (VIIB), (VIIIB), (IXB), and (XIB), is selected from the group consisting of the following structural formulas:

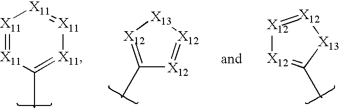

wherein X₁₁, X₁₂, X₁₃, R₉ and R₁₇ are defined as described in the tenth specific embodiment. Values and specific values for the remainder of the variables are as described in the thirteenth specific embodiment.

In a fourteenth specific embodiment, ring A of the compounds of Structural Formulas (I)-(IV), (IA), (IA′), (IIA), (IVA), (IB), (IIIB), (VIIB), (VIIIB), (IXB), and (XIB), is represented by Structural Formula (XI):

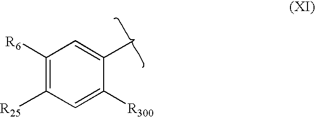

wherein R₁, R₃₀₀, R₅, R₆, and R₂₅ are defined as the thirteenth specific embodiment. More specifically, R₂₅ in Structural Formulas (I)-(IV) is —OR_{A}, —SR_{B}, —N(R_{C})₂, —OC(S)OR₇, —OC(NR₈)OR₇, —SC(NR₈)OR₇, —SC(S)OR₇, —OC(S)NR₁₀R₁₁, —SC(S)NR₁₀R₁₁, —OC(NR₈)R₇, —SC(NR₈)R₇, —OS(O)ₚR₇, —S(O)ₚOR₇, —SS(O)ₚOR₇, —SS(O)ₚR₇, —OP(O)(OR₇)₂, or —SP(O)(OR₇)₂, wherein p is 0, 1, or 2. In a more specific embodiment, for Structural Formulas (I)-(IV), R₃ and R₂₅ are —OR_{A}. Even more specifically, R₆ is a lower alkyl, C₃-C₆ cycloalkyl, lower alkoxy, a lower alkyl sulfanyl, or —NR₁₀R₁₁.

In a more specific embodiment, for Structural Formulas (IA′), (IIA) and (IVA), R₂₁ is O; R₆ is a C1-C6 alkyl, a C1-C6 haloalkyl, a C1-C6 alkoxy, a C1-C6 haloalkoxy, a C3-C6 cycloalkyl or —NR₁₀R₁₁. In another more specific embodiment, for Structural Formulas (IA′), (IIA) and (IVA), R₆ is a C1-C6 alkyl and R₃₃ is H. In another more specific embodiment, for Structural Formulas (IA′), (IIA) and (IVA), R₃₃ is —H and ring B is unsubstituted. In yet another more specific embodiment, for Structural Formulas (IA′), (IIA) and (IVA), R₂₀ and R₂₅ are —OH, and R₆ is a C1-C6 alkyl.

In another more specific embodiment, for Structural Formulas (IB), (IIIB), (VIIB), (VIIIB), (IXB), and (XIB), R_{3b} and R₂₅ are —OR₁₀₀, —SR₁₀₁, or —N(R₁₀₂)₂.

In another more specific embodiment, for Structural Formulas (IB), (IIIB), (VIIB), (VIIIB), (IXB), and (XIB), R₁ is —SH, R₃ and R₂₅ are —OR₁₀₀; and R₅₁ is ═O or ═S.

In another more specific embodiment, for Structural Formulas (IB), (IIIB), (VIIB), (VIIIB), (IXB), and (XIB), R_{1b} is —SH or —OH; R_{3b} and R₂₅ are —OR₁₀₀; R₅₁ is ═O or ═S; and R₆ is an optionally substituted lower alkyl, a C3-C6 cycloalkyl, a lower alkoxy, a lower alkyl sulfanyl, or —NR₁₀R₁₁.

In another more specific embodiment, R₅ in Structural Formulas (I)-(IV), (IA), (IA′), (IIA), (IB), (IIIB), (VIIB), (VIIIB), (IXB), and (XIB), is an optionally substituted phenyl, wherein the phenyl group is substituted with substituents as described above in the eighth specific embodiment. Values and specific values for the remainder of the variables are as described above in the fourteenth specific embodiment. Even more specifically, for Structural Formulas (I)-(IV), R₃ and R₂₅ are —OR_{A}. Even more specifically, for Structural Formulas (I)-(IV), R₃ and R₂₅ are —OR_{A}; R₆ is a lower alkyl, C3-C6 cycloalkyl, a lower alkoxy, a lower alkyl sulfanyl, or —NR₁₀R₁₁.

In another more specific embodiment, R₅ in the compounds of Structural Formulas (I)-(IV), (IA), (IA′), (IIA), (IB), (IIIB), (VIIB), (VIIIB), (IXB), and (XIB), is represented by the following structural formula:

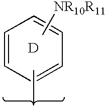

and values and specific values for the remainder of the variables are as described in the fourteenth specific embodiment. Preferably, R₁₀ and R₁₁ are each independently a hydrogen, a C1-C6 straight or branched alkyl, optionally substituted by —OR_{A}, —CN, —SR_{A}, —N(R_{C})₂, a C1-C6 alkoxy, alkylsulfanyl, dialkylamino or a cycloalkyl; or R₁₀ and R₁₁ taken together with the nitrogen to which they are attached form a substituted or unsubstituted nonaromatic, nitrogen-containing heterocyclyl. More preferably, R₁₀ and R₁₁ are each independently a hydrogen, methyl, ethyl, propyl, isopropyl, or taken together with the nitrogen to which they are attached, are:

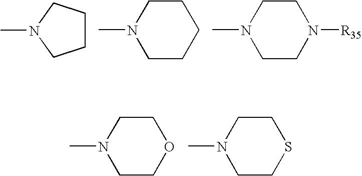

In another more specific embodiment, R₅ in compounds of Structural Formulas (I)-(IV), (IA), (IA′), (IIA), (IB), (IIIB), (VIIB), (VIIIB), (IXB), and (XIB), is an optionally substituted cycloalkyl, and optionally substituted cycloalkenyl, or a substituted alkyl, wherein the alkyl group is substituted with one or more substituents independently selected from the group described above in the eleventh specific embodiment. Values and specific values for the remainder of the variables are as described in the fourteenth specific embodiment. In a even more specific embodiment, R₅ is an optionally substituted cycloalkyl or an optionally substituted cycloalkenyl. In another even more specific embodiment, R₅ is an optionally substituted alkyl.

In another more specific embodiment, R₅ in the compounds of Structural Formulas (I)-(IV), (IA), (IA′), (IIA), (IB), (IIIB), (VIIB), (VIIIB), (IXB), and (XIB) is represented by the following structural formula:

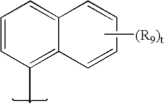

wherein R₉ and m are as described in the fifth specific embodiment. Values and specific values for the remainder of the variables are as described the fourteenth specific embodiment. In a even more specific embodiment, for Structural Formulas (I)-(IV), R₃ and R₂₅ are —OR_{A}; R₆ is a lower alkyl, a C3-C6 cycloalkyl, a lower alkoxy, a lower alkyl sulfanyl, or —NR₁₀R₁₁; and R₉ for each occurrence, is independently selected from the group consisting of —OR_{A}, —SR_{B}, halo, a lower haloalkyl, cyano, a lower alkyl, a lower alkoxy, and a lower alkyl sulfanyl.

In another more specific embodiment, R₅ in compounds of Structural Formulas (I)-(IV), (IA), (IA′), (IIA), (IB), (IIIB), (VIIB), (VIIIB), (IXB), and (XIB), is selected from the group consisting of the following structural formulas:

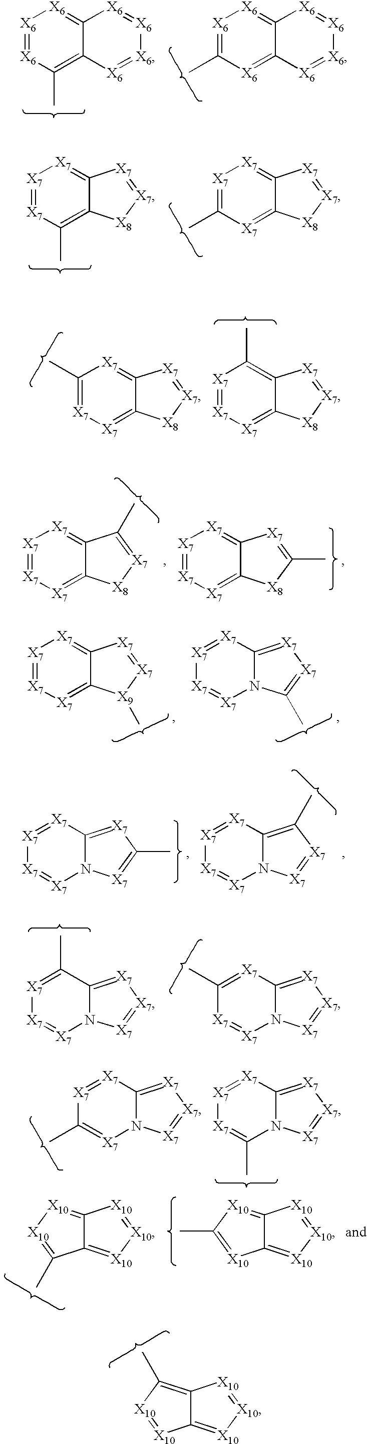

wherein X₆, X₇, X₈, X₉, X₁₀ and R₁₇ are as described in the ninth specific embodiment. The remainder of the variables are as described in the fourteenth specific embodiment. Preferably, R₅ is an optionally substituted indolyl, an optionally substituted benzoimidazolyl, an optionally substituted indazolyl, an optionally substituted 3H-indazolyl, an optionally substituted indolizinyl, an optionally substituted quinolinyl, an optionally substituted isoquinolinyl, an optionally substituted benzoxazolyl, an optionally substituted benzo[1,3]dioxolyl, an optionally substituted benzofuryl, an optionally substituted benzothiazolyl, an optionally substituted benzo[d]isoxazolyl, an optionally substituted benzo[d]isothiazolyl, an optionally substituted thiazolo[4,5-c]pyridinyl, an optionally substituted thiazolo[5,4-c]pyridinyl, an optionally substituted thiazolo[4,5-b]pyridinyl, an optionally substituted thiazolo[5,4-b]pyridinyl, an optionally substituted oxazolo[4,5-c]pyridinyl, an optionally substituted oxazolo[5,4-c]pyridinyl, an optionally substituted oxazolo[4,5-b]pyridinyl, an optionally substituted oxazolo[5,4-b]pyridinyl, an optionally substituted imidazopyridinyl, an optionally substituted benzothiadiazolyl, benzoxadiazolyl, an optionally substituted benzotriazolyl, an optionally substituted tetrahydroindolyl, an optionally substituted azaindolyl, an optionally substituted quinazolinyl, an optionally substituted purinyl, an optionally substituted imidazo[4,5-a]pyridinyl, an optionally substituted imidazo[1,2-a]pyridinyl, an optionally substituted 3H-imidazo[4,5-b]pyridinyl, an optionally substituted 1H-imidazo[4,5-b]pyridinyl, an optionally substituted 1H-imidazo[4,5-c]pyridinyl, an optionally substituted 3H-imidazo[4,5-c]pyridinyl, an optionally substituted pyridopyrdazinyl, and optionally substituted pyridopyrimidinyl, an optionally substituted pyrrolo[2,3]pyrimidyl, an optionally substituted pyrazolo[3,4]pyrimidyl an optionally substituted cyclopentaimidazolyl, an optionally substituted cyclopentatriazolyl, an optionally substituted pyrrolopyrazolyl, an optionally substituted pyrroloimidazolyl, an optionally substituted pyrrolotriazolyl, or an optionally substituted benzo[b]thienyl. In a even more specific embodiment, R₆ is a lower alkyl, a C3-C6 cycloalkyl, a lower alkoxy, a lower alkyl sulfanyl, or —NR₁₀R₁₁. In another even more specific embodiment, R₆ is a lower alkyl, a C3-C6 cycloalkyl, a lower alkoxy, a lower alkyl sulfanyl, or —NR₁₀R₁₁; and R₃ and R₂₅ are —OR_{A}.

In another more specific embodiment, R₅ is represented by the following Structural Formula:

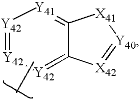

wherein values and specific values for the variables are as described below in the eighteenth specific embodiment. In a even more specific embodiment, R₆ is selected from the group consisting of —H, a lower alkyl, a lower alkoxy, a lower cycloalkyl, and a lower cycloalkoxy.

In another more specific embodiment, R₅ in the compounds of Structural Formulas (I)-(IV), (IA), (IA′), (IIA), (IB), (IIIB), (VIIB), (VIIIB), (IXB), and (XIB), is selected from the group consisting of the following structural formulas:

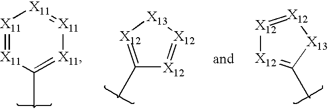

wherein X₁₁, X₁₂, X₁₃, R₉ and R₁₇ are defined as described in the tenth specific embodiment. The remainder of the variables are as described in the fourteenth specific embodiment. In a even more specific embodiment, R₆ is a lower alkyl, a C3-C6 cycloalkyl, a lower alkoxy, a lower alkyl sulfanyl, or —NR₁₀R₁₁. In another even more specific embodiment, R₆ is a lower alkyl, a C3-C6 cycloalkyl, a lower alkoxy, a lower alkyl sulfanyl, or —NR₁₀R₁₁; and R₃ and R₂₅ are —OR_{A}.

In another more specific embodiment, R₅ in Structural Formulas (I)-(IV), (IA), (IA′), (IIA), (IB), (IIIB), (VIIB), (VIIIB), (IXB), and (XIB) is an optionally substituted indolyl. Preferably, R₅ is an indolyl represented by the following Structural Formula:

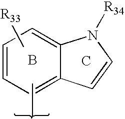

wherein R₃₃, R₃₄, ring B and ring C are as described above in the seventh specific embodiment. Values and specific values for the remainder of the variables are as described above in the thirteenth specific embodiment. In a even more specific embodiment, for Structural Formulas (IA), (IA′) and (IIA), R₂₅ is a —ORₚ₁, —NHRₚ₃, —N(Rₚ₃)₂, —O(CH₂)ₘORₚ₁, or —(CH₂)ₘORₚ₁; R₃₃ is H, —ORₚ₁, —NHRₚ₃, —N(Rₚ₃)₂, —O(CH₂)ₘORₚ₁, or —(CH₂)ₘORₚ₁; and R₃₄ is a C₁-C₆ alkyl. In another more specific embodiment, for Structural Formulas (IA), (IA′) and (IIA), R₂₁ is O; R₆ is a C1-C6 alkyl, a C1-C6 haloalkyl, a C1-C6 alkoxy, a C1-C6 haloalkoxy, a C3-C6 cycloalkyl or —NR₁₀R₁₁. In another even more specific embodiment, for Structural Formulas (IA), (IA′) and (IIA), R₆ is a C1-C6 alkyl and R₃₃ is H. In another even more specific embodiment, for Structural Formulas (IA), (IA′) and (IIA), R₃₃ is —H and ring B is unsubstituted. In another even more specific embodiment, for Structural Formulas (IA), (IA′) and (IIA), R₆ is a C1-C6 alkyl; R₃₃ is H; and ring B is unsubstituted. In yet another even more embodiment, In another even more specific embodiment, for Structural Formulas (IA), (IA′) and (IIA), R₂₀ and R₂₅ are —OH, and R₆ is a C1-C6 alkyl.

In a fifteenth specific embodiment, ring A in compounds of Structural Formulas (I)-(IV), (IA), (IA′), (IIA), (IVA), (IB), (IIIB), (VIIB), (VIIIB), (IXB), and (XIB), is represented by one of Structural Formulas (XII):

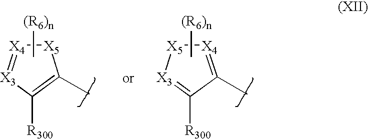

X₃ and X₄ are each, independently, N, N(O), N⁺(R₁₇), CH or CR₆; and

X₅ is O, S, NR₁₇, CH═CH, CH═CR₆, CR₆═CH, CR₆═CR₆, CH═N, CR₆═N, CH═N(O), CR₆═N(O), N═CH, N═CR₆, N(O)═CH, N(O)═CR₆, N⁺(R₁₇)═CH, N⁺(R₁₇)═CR₆, CH═N⁺(R₁₇), CR₆═N⁺(R₁₇), or N═N; wherein R₁₇ is defined as in the ninth specific embodiment. Values and specific values for the remainder of the variables is as described in the twelfth specific embodiment.

In a more specific embodiment, R₅ in the compounds of Structural Formulas (I)-(IV), (IA), (IA′), (IIA), (IB), (IIIB), (VIIB), (VIIIB), (IXB), and (XIB), is an optionally substituted phenyl, wherein the phenyl group is substituted with substituents as described in the eighth specific embodiment. The remainder of the variables are as described in the fifteenth specific embodiment. More specifically, for Structural Formulas (I)-(IV), R₃ and R₂₅ are —OR_{A}. Even more specifically, R₆ is a lower alkyl, C₃-C₆ cycloalkyl, lower alkoxy, a lower alkyl sulfanyl, or —NR₁₀R₁₁.

In another more specific embodiment, R₅ in compounds of Structural Formulas (I)-(IV), (IA), (IA′), (IIA), (IB), (IIIB), (VIIB), (VIIIB), (IXB), and (XIB), is an optionally substituted cycloalkyl, and optionally substituted cycloalkenyl, or a substituted alkyl, wherein the alkyl group is substituted with one or more substituents independently selected from the group described above in the eleventh specific embodiment. The remainder of the variables are as described in the fifteenth specific embodiment. In a more specific embodiment, R₅ is an optionally substituted cycloalkyl or an optionally substituted cycloalkenyl. In another more specific embodiment, R₅ is an optionally substituted alkyl.

In another more specific embodiment, R₅ in the compounds of Structural Formulas (I)-(IV), (IA), (IA′), (IIA), (IB), (IIIB), (VIIB), (VIIIB), (IXB), and (XIB) is represented by the following structural formula:

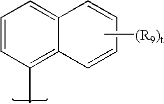

wherein R₉ and m are as described in the fifth specific embodiment. Values and specific values for the remainder of the variables are as described the fifteenth specific embodiment.

In another more specific embodiment, R₅ in compounds of Structural Formulas (I)-(IV), (IA), (IA′), (IIA), (IB), (IIIB), (VIIB), (VIIIB), (IXB), and (XIB), is selected from the group consisting of the following structural formulas:

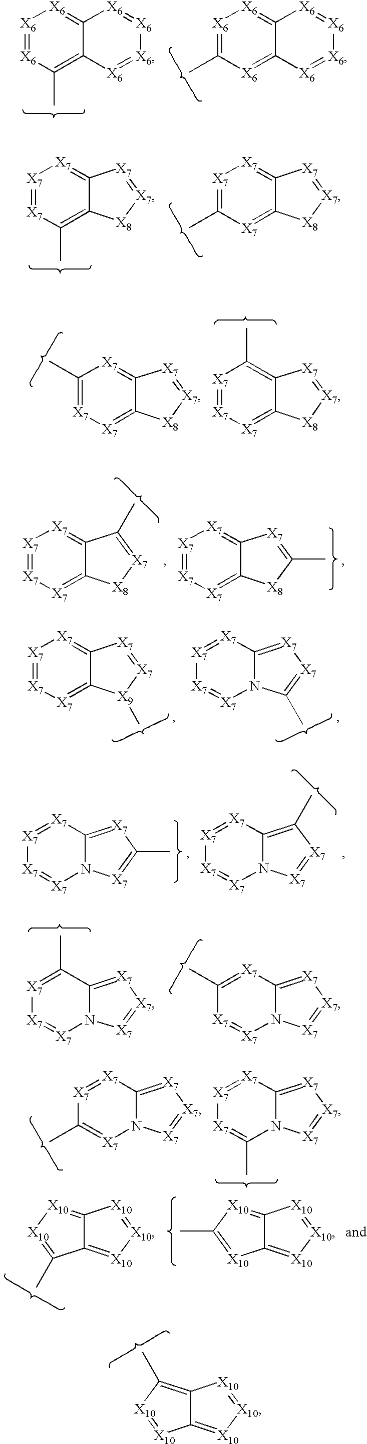

wherein X₆, X₇, X₈, X₉, X₁₀ and R₁₇ are as described in the ninth specific embodiment. Values and specific values for the remainder of the variables are as described above in the fifteenth specific embodiment. Preferably, R₅ is an optionally substituted indolyl, an optionally substituted benzoimidazolyl, an optionally substituted indazolyl, an optionally substituted 3H-indazolyl, an optionally substituted indolizinyl, an optionally substituted quinolinyl, an optionally substituted isoquinolinyl, an optionally substituted benzoxazolyl, an optionally substituted benzo[1,3]dioxolyl, an optionally substituted benzofuryl, an optionally substituted benzothiazolyl, an optionally substituted benzo[d]isoxazolyl, an optionally substituted benzo[d]isothiazolyl, an optionally substituted thiazolo[4,5-c]pyridinyl, an optionally substituted thiazolo[5,4-c]pyridinyl, an optionally substituted thiazolo[4,5-b]pyridinyl, an optionally substituted thiazolo[5,4-b]pyridinyl, an optionally substituted oxazolo[4,5-c]pyridinyl, an optionally substituted oxazolo[5,4-c]pyridinyl, an optionally substituted oxazolo[4,5-b]pyridinyl, an optionally substituted oxazolo[5,4-b]pyridinyl, an optionally substituted imidazopyridinyl, an optionally substituted benzothiadiazolyl, benzoxadiazolyl, an optionally substituted benzotriazolyl, an optionally substituted tetrahydroindolyl, an optionally substituted azaindolyl, an optionally substituted quinazolinyl, an optionally substituted purinyl, an optionally substituted imidazo[4,5-a]pyridinyl, an optionally substituted imidazo[1,2-a]pyridinyl, an optionally substituted 3H-imidazo[4,5-b]pyridinyl, an optionally substituted 1H-imidazo[4,5-b]pyridinyl, an optionally substituted 1H-imidazo[4,5-c]pyridinyl, an optionally substituted 3H-imidazo[4,5-c]pyridinyl, an optionally substituted pyridopyrdazinyl, and optionally substituted pyridopyrimidinyl, an optionally substituted pyrrolo[2,3]pyrimidyl, an optionally substituted pyrazolo[3,4]pyrimidyl an optionally substituted cyclopentaimidazolyl, an optionally substituted cyclopentatriazolyl, an optionally substituted pyrrolopyrazolyl, an optionally substituted pyrroloimidazolyl, an optionally substituted pyrrolotriazolyl, or an optionally substituted benzo[b]thienyl.

In another more specific embodiment, R₅ in the compounds of Structural Formulas (I)-(IV), (IA), (IA′), (IIA), (IB), (IIIB), (VIIB), (VIIIB), (IXB), and (XIB), is selected from the group consisting of the following structural formulas:

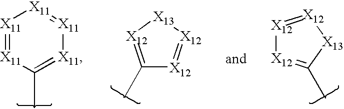

wherein X₁₁, X₁₂, X₁₃, R₉ and R₁₇ are defined as described in the tenth specific embodiment. Values and specific values for the remainder of the variables are as described in the fifteenth specific embodiment.

In another more specific embodiment, R₅ in Structural Formulas (I)-(IV), (IA), (IA′), (IIA), (IB), (IIIB), (VIIB), (VIIIB), (IXB), and (XIB) is an optionally substituted indolyl. Preferably, R₅ is an indolyl represented by the following Structural Formula:

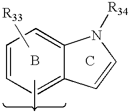

wherein R₃₃, R₃₄, ring B and ring C are as described above in the seventh specific embodiment. Values and specific values for the remainder of the variables are as described above in the thirteenth specific embodiment. In a even more specific embodiment, for Structural Formulas (XIIA), R₂₁ is O; R₆ is a C1-C6 alkyl, a C1-C6 haloalkyl, a C1-C6 alkoxy, a C1-C6 haloalkoxy, a C3-C6 cycloalkyl or —NR₁₀R₁₁. In another even more specific embodiment, for Structural Formula (XIIA), R₆ is a C1-C6 alkyl and R₃₃ is H. In another even more specific embodiment, for Structural Formula (XIIA), R₃₃ is —H and ring B is unsubstituted. In yet another even more embodiment, for Structural Formula (XIIA), R₂₀ and R₂₅ are —OH, and R₆ is a C1-C6 alkyl.

In a sixteenth specific embodiment, ring A in compounds represented by Structural Formulas (I)-(IV), (IA), (IA′), (IIA), (IVA), (IB), (IIIB), (VIIB), (VIIB), (IXB), and (XIB), is selected from Structural Formula (XIII):

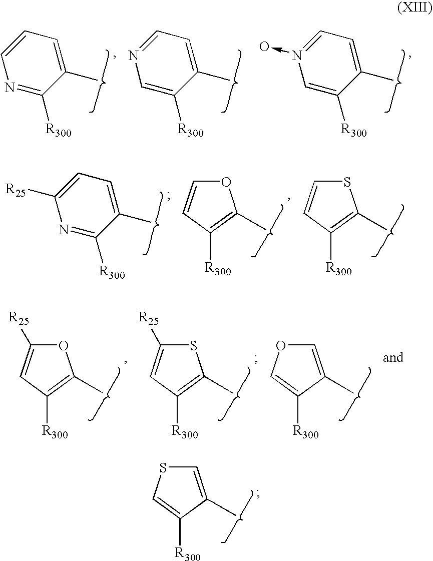

wherein R₁, R₃₀₀, R₅, and R₂₅ are as described above in the thirteenth specific embodiment. Specifically, for Structural Formulas (IB), (IIIB), (VIIB), (VIIIB), (IXB), and (XIB), R₂₅ is a halo, a haloalkyl, a haloalkoxy, a heteroalkyl, —OR₁₀₀, —SR₁₀₁, —N(R₁₀₂)₂, —NR₇R₁₀₂, —OR₂₆, —SR₂₆, —NR₂₆R₁₀₂, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OCH₂C(O)R₇, —SCH₂C(O)R₇, and —NR₇CH₂C(O)R₇. k is 1, 2, 3, or 4. The values and specific values of the remaining variables are as described above in the fifteenth specific embodiment.

In a more specific embodiment, R₅ in the compounds of Structural Formulas (I)-(IV), (IA), (IA′), (IIA), (IB), (IIIB), (VIIB), (VIIIB), (IXB), and (XIB), is an optionally substituted phenyl, wherein the phenyl group is substituted with substituents as described in the eighth specific embodiment. Values and specific values for the remainder of the variables are as described in the sixteenth specific embodiment. Even more specifically, for Structural Formulas (I)-(IV), R₃ and R₂₅ are —OR_{A}. Even more specifically, R₆ is a lower alkyl, C3-C6 cycloalkyl, lower alkoxy, a lower alkyl sulfanyl, or —NR₁₀R₁₁.

In another more specific embodiment, R₅ in compounds of Structural Formulas (I)-(IV), (IA), (IA′), (IIA), (IB), (IIIB), (VIIB), (VIIIB), (IXB), and (XIB), is an optionally substituted cycloalkyl, and optionally substituted cycloalkenyl, or a substituted alkyl, wherein the alkyl group is substituted with one or more substituents independently selected from the group described above in the eleventh specific embodiment. Values and specific values for the remainder of the variables are as described in the sixteenth specific embodiment. In a more specific embodiment, R₅ is an optionally substituted cycloalkyl or an optionally substituted cycloalkenyl. In another more specific embodiment, R₅ is an optionally substituted alkyl.

In another more specific embodiment, R₅ in the compounds of Structural Formulas (I)-(IV), (IA), (IA′), (IIA), (IB), (IIIB), (VIIB), (VIIIB), (IXB), and (XIB) is represented by the following structural formula:

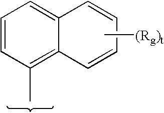

wherein R₉ and m are as described in the fifth specific embodiment. The remainder of the variables are as described the sixteenth specific embodiment.

In another more specific embodiment, R₅ in compounds of Structural Formulas (I)-(IV), (IA), (IA′), (IIA), (IB), (IIIB), (VIIB), (VIIIB), (IXB), and (XIB), is selected from the group consisting of the following structural formulas:

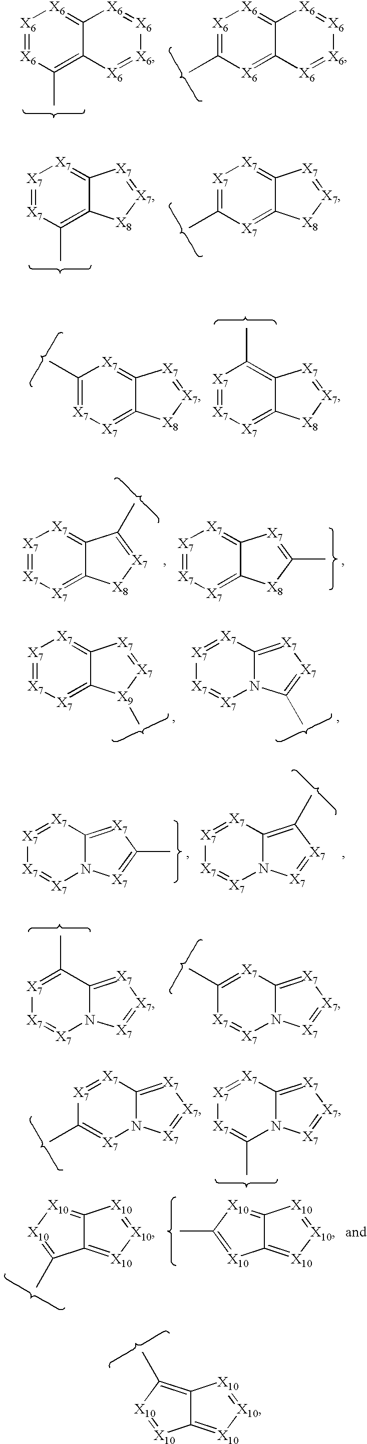

wherein X₆, X₇, X₈, X₉, X₁₀ and R₁₇ are as described in the ninth specific embodiment. Values and specific values for the remainder of the variables are as described in the sixteenth specific embodiment. Preferably, R₅ is an optionally substituted indolyl, an optionally substituted benzoimidazolyl, an optionally substituted indazolyl, an optionally substituted 3H-indazolyl, an optionally substituted indolizinyl, an optionally substituted quinolinyl, an optionally substituted isoquinolinyl, an optionally substituted benzoxazolyl, an optionally substituted benzo[1,3]dioxolyl, an optionally substituted benzofuryl, an optionally substituted benzothiazolyl, an optionally substituted benzo[d]isoxazolyl, an optionally substituted benzo[d]isothiazolyl, an optionally substituted thiazolo[4,5-c]pyridinyl, an optionally substituted thiazolo[5,4-c]pyridinyl, an optionally substituted thiazolo[4,5-b]pyridinyl, an optionally substituted thiazolo[5,4-b]pyridinyl, an optionally substituted oxazolo[4,5-c]pyridinyl, an optionally substituted oxazolo[5,4-c]pyridinyl, an optionally substituted oxazolo[4,5-b]pyridinyl, an optionally substituted oxazolo[5,4-b]pyridinyl, an optionally substituted imidazopyridinyl, an optionally substituted benzothiadiazolyl, benzoxadiazolyl, an optionally substituted benzotriazolyl, an optionally substituted tetrahydroindolyl, an optionally substituted azaindolyl, an optionally substituted quinazolinyl, an optionally substituted purinyl, an optionally substituted imidazo[4,5-a]pyridinyl, an optionally substituted imidazo[1,2-a]pyridinyl, an optionally substituted 3H-imidazo[4,5-b]pyridinyl, an optionally substituted 1H-imidazo[4,5-b]pyridinyl, an optionally substituted 1H-imidazo[4,5-c]pyridinyl, an optionally substituted 3H-imidazo[4,5-c]pyridinyl, an optionally substituted pyridopyrdazinyl, and optionally substituted pyridopyrimidinyl, an optionally substituted pyrrolo[2,3]pyrimidyl, an optionally substituted pyrazolo[3,4]pyrimidyl an optionally substituted cyclopentaimidazolyl, an optionally substituted cyclopentatriazolyl, an optionally substituted pyrrolopyrazolyl, an optionally substituted pyrroloimidazolyl, an optionally substituted pyrrolotriazolyl, or an optionally substituted benzo[b]thienyl.

In another more specific embodiment, R₅ in the compounds of Structural Formulas (I)-(IV), (IA), (IA′), (IIA), (IB), (IIIB), (VIIB), (VIIIB), (IXB), and (XIB), is selected from the group consisting of the following structural formulas:

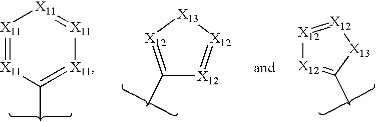

wherein X₁₁, X₁₂, X₁₃, R₉ and R₁₇ are defined as described in the tenth specific embodiment. Values and specific values for the remainder of the variables are as described in the sixteenth specific embodiment.

In another more specific embodiment, R₅ in Structural Formulas (I)-(IV), (IA), (IA′), (IIA), (IB), (IIIB), (VIIB), (VIIIB), (IXB), and (XIB) is an optionally substituted indolyl. Preferably, R₅ is an indolyl represented by the following Structural Formula:

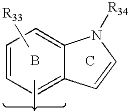

wherein R₃₃, R₃₄, ring B and ring C are as described above in the seventh specific embodiment. Values and specific values for the remainder of the variables are as described above in the thirteenth specific embodiment. In a even more specific embodiment, for Structural Formulas (XIIA), R₂₁ is O; R₆ is a C1-C6 alkyl, a C1-C6 haloalkyl, a C1-C6 alkoxy, a C1-C6 haloalkoxy, a C3-C6 cycloalkyl or —NR₁₀R₁₁. In another even more specific embodiment, for Structural Formula (XIIA), R₆ is a C1-C6 alkyl and R₃₃ is H. In another even more specific embodiment, for Structural Formula (XIIA), R₃₃ is —H and ring B is unsubstituted. In yet another even more embodiment, for Structural Formula (XIIA), R₂₀ and R₂₅ are —OH, and R₆ is a C1-C6 alkyl.

In a seventeenth specific embodiment, compounds of Structural Formulas (I)-(IV), (IA), (IA′), (IIA), (IB), (IIIB), (VIIB), (VIIIB), (IXB), and (XIB) are defined as the following:

R₅ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

ring A is represented by Structural Formulas (XIV):

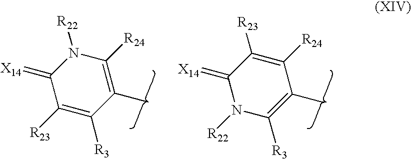

wherein,

X₁₄ is O, S, or NR₇;

for Structural Formulas (I)-(IV):

R₂₂, for each occurrence, is independently an —H or is selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, a haloalkyl, —S(O)ₚR₇, or —S(O)ₚNR₁₀R₁₁; and

R₂₃ and R₂₄, for each occurrence, are independently —H or are selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, halo, cyano, nitro, guanadino, a haloalkyl, a heteroalkyl, —NR₁₀R₁₁ (provided R₁₀ and R₁₁ are not H), —OR₇ (provided R₇ is not H), —SR₇ (provided R₇ is not H), —S(O)ₚR₇, —OS(O)ₚR₇, —NR₈S(O)ₚR₇, or —S(O)ₚNR₁₀R₁₁;

for Structural Formulas (IA), (IA′) and (IIA):

R₂₂, for each occurrence, is independently an —H or is selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, a haloalkyl, —C(O)R₇, —C(O)OR₇, —OC(O)R₇, —C(O)NR₁₀R₁₁, —NR₈C(O)R₇, —S(O)ₚR₇, —S(O)ₚOR₇ or —S(O)ₚNR₁₀R₁₁; and

R₂₃ and R₂₄, for each occurrence, are independently —H or are selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —NR₈S(O)ₚR₇, or —S(O)ₚNR₁₀R₁₁;

for Structural Formulas (IB), (IIIB), (VIIB), (VIIIB), (IXB), and (XIB):

R₂₂, for each occurrence, is independently an —H or is selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, a haloalkyl, or —C(O)R₇;

R₂₃ and R₂₄, for each occurrence, are independently —H or are selected from the group consisting of an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, halo, cyano, nitro, guanadino, a haloalkyl, a heteroalkyl, —NR₁₀R₁₁, —OR₇, or —C(O)R₇;

Values and specific values for the remainder of the variables are as described in Structural Formulas (I)-(IV), (IA), (IA′), (IIA), (IB), (IIIB), (VIIB), (VIIIB), (IXB), and (XIB).

In a more specific embodiment, R₅ is an optionally substituted alkyl, an optionally substituted cycloalkyl, an optionally substituted aryl or an optionally substituted heteroaryl. The remainder of the variables are as described in the seventeenth specific embodiment.

In another more specific embodiment, R₂₂ is —H, or an alkyl, an aralkyl. The remainder of the variables are as described in the seventeenth specific embodiment.

In another more specific embodiment, X₁₄ is O. The remainder of the variables are as described in the seventeenth specific embodiment.

In a eighteenth specific embodiment, the compounds of Structural Formulas (I)-(IV), (IA), (IA′), (IIA), (IB), (IIIB), (VIIB), (VIIIB), (IXB), and (XIB) are defined as the following or tautomers, pharmaceutically acceptable salts, solvates, clarthrates, or prodrugs thereof:

Ring A is represented by the following Structural Formula:

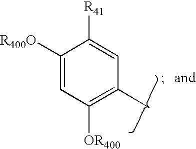

R₅ is represented by the following Structural Formula:

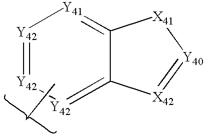

wherein:

X₄₁ is O, S, or NR₄₂;

X₄₂ is CR₄₄ or N;

Y₄₀ is N or CR₄₃;

Y₄₁ is N or CR₄₅;

Y₄₂, for each occurrence, is independently N, C or CR₄₆;

R₄₁, R₄₂, R₄₃, R₄₄, R₄₅, R₄₆, and R₄₀₀ are defined as the following:

for Structural Formulas (I)-(IV), R₄₁ is —H, —OR_{A}, —SR_{B}, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, —NR₁₀R₁₁ (provided R₁₀ and R₁₁ are not H), —OR₇ (provided R₇ is not H), —C(NR₈)OR₇, —C(NR₈)R₇, —C(NR₈)NR₁₀R₁₁, —C(NR₈)SR₇, —OC(S)OR₇, —OC(NR₈)OR₇, —SC(NR₈)OR₇, —SC(S)OR₇, —OC(S)NR₁₀R₁₁, —OC(NR₈)NR₁₀R₁₁, —SC(NR₈)NR₁₀R₁₁, —SC(S)NR₁₀R₁₁, —OC(NR₈)R₇, —SC(NR₈)R₇, —NR₇C(S)OR₇, —NR₇C(NR₈)R₇, —NR₇C(NR₉)OR₇, —NR₇C(S)NR₁₀R₁₁, —NR₇C(NR₈)NR₁₀R₁₁, —SR₇ (provided R₇ is not H), —S(O)ₚR₇, —OS(O)ₚR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;

R₄₂ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, a haloalkyl, a heteroalkyl, —S(O)ₚR₇, or —S(O)ₚNR₁₀R₁₁;

R₄₃ and R₄₄ are, independently, —H, —OR_{A}, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, halo, cyano, nitro, guanadino, a haloalkyl, a heteroalkyl, —SR₇ (provided R₇ is not H), —S(O)ₚR₇, —OS(O)ₚR₇, —NR₈S(O)ₚR₇, —S(O)ₚNR₁₀R₁₁, or R₄₃ and R₄₄ taken together with the carbon atoms to which they are attached form an optionally substituted cycloalkenyl, an optionally substituted aryl, an optionally substituted heterocyclyl, or an optionally substituted heteroaryl;

R₄₅ is —H, —OR_{A}, —SR_{B}, —N(R_{C})₂, —OR₂₆, —SR₂₆, —O(CH₂)ₘOR_{A}, —O(CH₂)ₘSR_{B}, —O(CH₂)ₘNR₇R_{C}, —S(CH₂)ₘOR_{A}, —S(CH₂)ₘSR_{B}, —S(CH₂)ₘNR₇R_{C}, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —SS(O)ₚOR₇, —NR₇S(O)ₚOR₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;

R₄₆, for each occurrence, is independently, selected from the group consisting of H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, —NR₁₀R₁₁ (provided R₁₀ and R₁₁ are not H), —OR₇ (provided R₇ is not H), —SR₇ (provided R₇ is not H), —S(O)ₚR₇, —OS(O)ₚR₇, —NR₈S(O)ₚR₇, or —S(O)ₚNR₁₀R₁₁;

R₄₀₀ is R_{A} as described for Structural Formulas (I)-(IV).

for Structural Formulas (IA), (IA′) and (IIA), R₄₁ is —H, —ORₚ₁, —NHRₚ₃, —N(Rₚ₃)₂, —O(CH₂)ₘORₚ₁, or —(CH₂)ₘORₚ₁; an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl; halo, cyano, or nitro; —NR₁₀R₁₁, or —OR₇; —O(CH₂)ₘNR₇Rₚ₃; —C(O)R₇, —C(O)OR₇; —C(O)NR₁₀R₁₁; —OC(O)R₇, —OC(O)OR₇, —OC(O)NR₁₀R₁₁; —NR₈C(O)R₇, or —NR₇C(O)NR₁₀R₁₁; —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁; —NR₈S(O)ₚR₇, —NR₇S(O)ₚNR₁₀R₁₁, —NR₇S(O)ₚOR₇;

R₄₂ is —H, —ORₚ₁, —NHRₚ₃, —N(Rₚ₃)₂, —O(CH₂)ₘORₚ₁, or —(CH₂)ₘORₚ₁; an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl; halo, cyano, or nitro; —NR₁₀R₁₁, or —OR₇; —O(CH₂)ₘNR₇Rₚ₃; —C(O)R₇, —C(O)OR₇; —C(O)NR₁₀R₁₁; —OC(O)R₇, —OC(O)OR₇, —OC(O)NR₁₀R₁₁; —NR₈C(O)R₇, or —NR₇C(O)NR₁₀R₁₁; —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁; —NR₈S(O)ₚR₇, —NR₇S(O)ₚNR₁₀R₁₁, —NR₇S(O)ₚOR₇;

R₄₃ and R₄₄ are, independently, —H, —ORₚ₁, —NHRₚ₃, —N(Rₚ₃)₂, —O(CH₂)ₘORₚ₁, or —(CH₂)ₘORₚ₁; an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl; halo, cyano, or nitro; —NR₁₀R₁₁, or —OR₇; —O(CH₂)ₘNR₇Rₚ₃; —C(O)R₇, —C(O)OR₇; —C(O)NR₁₀R₁₁; —OC(O)R₇, —OC(O)OR₇, —OC(O)NR₁₀R₁₁; —NR₈C(O)R₇, or —NR₇C(O)NR₁₀R₁₁; —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁; —NR₈S(O)ₚR₇, —NR₇S(O)ₚNR₁₀R₁₁, —NR₇S(O)ₚOR₇;

or R₄₃ and R₄₄ taken together with the carbon atoms to which they are attached form an optionally substituted cycloalkenyl, an optionally substituted aryl, an optionally substituted heterocyclyl, or an optionally substituted heteroaryl;

R₄₅ is —H, —ORₚ₁, —NHRₚ₃, —N(Rₚ₃)₂, —O(CH₂)ₘORₚ₁, or —(CH₂)ₘORₚ₁; an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl; halo, cyano, or nitro; —NR₁₀R₁₁, or —OR₇; —O(CH₂)ₘNR₇Rₚ₃; —C(O)R₇, —C(O)OR₇; —C(O)NR₁₀R₁₁; —OC(O)R₇, —OC(O)OR₇, —OC(O)NR₁₀R₁₁; —NR₈C(O)R₇, or —NR₇C(O)NR₁₀R₁₁; —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁; —NR₈S(O)ₚR₇, —NR₇S(O)ₚNR₁₀R₁₁, —NR₇S(O)ₚOR₇;

R₄₆, for each occurrence, is independently, a lower alkyl;

R₄₀₀ is Rₚ₁ as described in Structural Formulas (IA), (IA′) and (IIA);

for Structural Formulas (IB), (IIIB), (VIIB), (VIIIB), (IXB), and (XIB), R₄₂ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, protected hydroxyalkyl that is optionally substituted, an optionally substituted alkoxyalkyl, an optionally substituted haloalkyl, an optionally substituted heteroalkyl, and —C(O)R₇.

R₄₃ and R₄₄ are, independently, —H, —OR₁₀₀, —N(R₁₀₂)₂, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, protected hydroxyalkyl, alkoxyalkyl, halo, cyano, nitro, guanadino, a haloalkyl, a heteroalkyl, —C(O)R₇, or —SR₁₀₁. Or R₄₃ and R₄₄ taken together with the carbon atoms to which they are attached form an optionally substituted cycloalkenyl, an optionally substituted aryl, an optionally substituted heterocyclyl, or an optionally substituted heteroaryl;

R₄₅ is —H, —OR₁₀₀, —SR₁₀₁, —N(R₁₀₂)₂, —O(CH₂)ₘOR₁₀₀, O(CH₂)ₘSR₁₀₁, —O(CH₂)ₘN(R₁₀₂)₂, —NR₁₀R₁₁, S(CH₂)ₘOR₁₀₀, —S(CH₂)ₘSR₁₀₁, —S(CH₂)ₘN(R₁₀₂)₂, —OCH₂C(O)R₇, —SCH₂C(O)R₇, or —NR₇CH₂C(O)R₇; and

R₄₆, for each occurrence, is independently, selected from the group consisting of H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, —NR₁₀R₁₁, —OR₁₀₀, —C(O)R₇, and —SR₁₀₁. Or two R₄₆ groups taken together with the carbon atoms to which they are attached form a fused ring;

R₄₁ is —H, —OR_{A}, —SR_{B}, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, —NR₁₀R₁₁ or —C(O)R₇. More specifically, R₄ is selected from the group consisting of —H, a lower alkyl, a lower alkoxy, a lower cycloalkyl and a lower cycloalkoxy.

R₄₀₀ is R₁₀₀ as described in Structural Formulas (IB), (IIIB), (VIIB), (VIIIB), (IXB), and (XIB).

Values and specific values for the remainder of the variables are as described in Structural Formulas (I)-(IV), (IA), (IA′), (IIA), (IB), (IIIB), (VIIB), (VIIIB), (IXB) and (XIB).

In a more specific embodiment, for Structural Formulas (I)-(IV), X₄₁ is NR₄₂ and X₄₂ is CR₄₄. The remainder of the variables are as described in the eighteenth specific embodiment.

In another more specific embodiment, for Structural Formulas (I)-(IV), X₄₁ is NR₄₂ and X₄₂ is N. Values and specific values for the remainder of the variables are as described in the eighteenth specific embodiment.

In another more specific embodiment, for Structural Formulas (I)-(IV), R₄₁ is selected from the group consisting of —H, lower alkyl, lower alkoxy, lower cycloalkyl, and lower cycloalkoxy. More specifically, R₄₁ is selected from the group consisting of H, methyl, ethyl, propyl, isopropyl, cyclopropyl, methoxy, ethoxy, propoxy, and cyclopropoxy. Values and specific values for the remainder of the variables are as described in the eighteenth specific embodiment.

In another more specific embodiment, for Structural Formulas (I)-(IV), X₄₁ is NR₄₂, and R₄₂ is selected from the group consisting of —H, a lower alkyl, a lower cycloalkyl, wherein each R₂₇ is independently —H or a lower alkyl. More specifically,

R₄₂ is selected from the group consisting of —H, methyl, ethyl, n-propyl, isopropyl, cyclopropyl, n-butyl, sec-butyl, tert-butyl, n-pentyl, n-hexyl, —CH₂OCH₃, —CH₂CH₂OCH₃. Values and specific values for the remainder of the variables are as described in the eighteenth specific embodiment.

In another more specific embodiment, for Structural Formulas (I)-(IV), R₄₃ and R₄₄ are, independently, selected from the group consisting of —H, methyl, ethyl, propyl, isopropyl, cyclopropyl, methoxy, ethoxy, propoxy, and cyclopropoxy. Values and specific values for the remainder of the variables are as described in the eighteenth specific embodiment.

In another more specific embodiment, for Structural Formulas (I)-(IV), X₄₂ is CR₄₄; Y₄₀ is CR₄₃; and R₄₃ and R₄₄ together with the carbon atoms to which they are attached form a cycloalkenyl, an aryl, heterocyclyl, or heteroaryl ring. In a more specific embodiment, X₄₁ is O. In a even more specific embodiment, R₄₃ and R₄₄ together with the carbon atoms to which they are attached form a C₅-C₈ cycloalkenyl or a C₅-C₈ aryl. In another even more specific embodiment, R₄₅ or CR₄₅ is selected from the group consisting of —H, —OR_{A}, —SR_{B}, —N(R_{C})₂, a lower alkoxy, and a lower dialkyl amino. Even more specifically, R₄₅ is selected from the group consisting of —H, —OR_{A}, methoxy and ethoxy. Values and specific values for the remainder of the variables are as described in the eighteenth specific embodiment.

In another more specific embodiment, for Structural Formulas (IA), (IA′) and (IIA), the variables can each be independently selected from the following lists of preferred values (values and specific values for the remainder of the substituents are as defined above in the eighteenth specific embodiment):

X₄₁ can be NR₄₂ and X₄₂ can be CR₄₄;

X₄₁ can be NR₄₂ and X₄₂ can be N;

R₄₁ can be selected from the group consisting of —H, lower alkyl, lower alkoxy, lower cycloalkyl, and lower cycloalkoxy;

R₄₁, can be selected from the group consisting of —H, methyl, ethyl, propyl, isopropyl, cyclopropyl, methoxy, ethoxy, propoxy, and cyclopropoxy;

X₄₁ can be NR₄₂, and R₄₂ can be selected from the group consisting of —H, a lower alkyl, a lower cycloalkyl, —C(O)N(R₂₇)₂, and —R_{C}, wherein R_{C} is a protected carboxyl group as defined above, and each R₂₇ is independently —H or a lower alkyl;

X₄₁ can be NR₄₂, and R₄₂ can be selected from the group consisting of —H, methyl, ethyl, n-propyl, isopropyl, cyclopropyl, n-butyl, sec-butyl, tert-butyl, n-pentyl, n-hexyl, —R_{C}, —(CH₂)ₘR_{C}, —CH₂OCH₃, —CH₂CH₂OCH₃, and —C(O)N(CH₃)₂, wherein R_{C} is a protected carboxyl group and m is 1 or 2;

R₄₃ and R₄₄ can be, independently, selected from the group consisting of —H, methyl, ethyl, propyl, isopropyl, cyclopropyl, methoxy, ethoxy, propoxy, and cyclopropoxy;

X₄₂ can be CR₄₄; Y₄₀ can be CR₄₃; and R₄₃ and R₄₄ together with the carbon atoms to which they are attached can form a cycloalkenyl, an aryl, heterocyclyl, or heteroaryl ring;

R₄₃ and R₄₄ together with the carbon atoms to which they are attached can form a C₅-C₈ cycloalkenyl or a C₅-C₈ aryl;

R₄₅ or CR₄₅ can be selected from the group consisting of —H, —ORₚ₁, —SRₚ₂, —NHRₚ₃, —N(Rₚ₃)₂, a lower alkoxy, —(CH₂)ₘ—NHRₚ₃, and —(CH₂)ₘ—N(Rₚ₃)₂, wherein m is an integer from 1 to 6;

R₄₅ can be selected from the group consisting of —H, —ORₚ₁, methoxy and ethoxy;

X₄₁ can be O.

In another more specific embodiment, for Structural Formulas (IB), (IIIB), (VIIB), (VIIIB), (IXB), and (XIB), R_{1b} is —SH or —OH; R_{3b} and R₂₅ are —OR₁₀₀; R₅₁ is ═O or ═S. More preferably, R_{1b} is —SH or —OH; R_{3b} and R₂₅ are —OR₁₀₀; R₅₁ is ═O or ═S; and R₄₅ is selected from the group consisting of —H, —OR₁₀₀, —SR₁₀₁, and —N(R₁₀₂)₂, a lower alkoxy and a protected lower alkyl amino. Values and specific values for the remainder of the variables are as described above in the eighteenth specific embodiment.

In another more specific embodiment, for Structural Formulas (IB), (IIIB), (VIIB), (VIIIB), (IXB), and (XIB), X₄₁ is NR₄₂ and X₄₂ is CR₄₄ or N; and values and specific values of the remaining variables are as described above in the eighteenth specific embodiment. More preferrably, X₄₁ is NR₄₂; and R₄₂ is selected from the group consisting of —H, a lower alkyl, a lower cycloalkyl, and an optionally substituted alkyl; and the values and specific values of the remaining variables are as described above in the eighteenth specific embodiment.

In another more specific embodiment, for Structural Formulas (IB), (IIIB), (VIIB), (VIIIB), (IXB), and (XIB), X₄₁ is NR₄₂; X₄₂ is CR₄₄; Y₄₀ is CR₄₃; and R₄₃ and R₄₄, together with the carbon atoms to which they are attached, form a cycloalkenyl, an aryl, heterocyclyl, heteroaryl ring. The values and specific values of the remaining variables are as described above in the eighteenth specific embodiment.

In another more specific embodiment, for Structural Formulas (IB), (IIIB), (VIIB), (VIIIB), (IXB), and (XIB), R_{1b} is —OH or —SH; and the values and specific values of the remaining variables are as described above in the eighteenth specific embodiment.

In a nineteenth specific embodiment, the compounds of Structural Formulas (I)-(IV), (IA), (IA′), (IIA), (IB), (IIIB), (VIIB), (VIIIB), (IXB) and (XIB) are defined as the following or a tautomer, a pharmaceutically acceptable salt, solvate, clathrate, or a prodrug thereof:

R₄ is selected from the group consisting of —H, methyl, ethyl, isopropyl, and cyclopropyl; R₄₂ is selected from the group consisting of —H, methyl, ethyl, n-propyl, isopropyl, n-butyl, n-pentyl, n-hexyl, —(CH₂)₂OCH₃;

R₄₃ and R₄₄ are each, independently, —H, methyl, ethyl, or isopropyl; or R₅₃ and R₅₄ taken together with the carbon atoms to which they are attached form a phenyl, cyclohexenyl, or cyclooctenyl ring; and

R₄₅ is selected from the group consisting of —H, —OCH₃, —OCH₂CH₃ and —OR₄₀₀. Values and specific values for the remainder of the variables are as described in the nineteenth specific embodiment.

In a more specific embodiment, for Structural Formula (IIA), R₂₁ is O. Values and specific values for the remainder of the variables are as described in the nineteenth specific embodiment.

In another more specific embodiment, for Structural Formulas (IA), (IA′) and (IIA), the variables can be each be independently selected from the following lists of preferred values:

X₄₂ can be CR₄₄, and R₄₃ and R₄₄ can be, independently, selected from the group consisting of —H, methyl, ethyl, propyl, isopropyl, cyclopropyl, methoxy, ethoxy, propoxy, and cyclopropoxy;

X₄₂ can be CR₄₄, and R₄₃ and R₄₄, taken together with the carbon atoms to which they are attached, can form a cycloalkenyl, aryl, heterocyclyl, or heteroaryl ring;

R₄₃ and R₄₄, taken together with the carbon atoms to which they are attached, can form a C₅-C₈ cycloalkenyl or a C₅-C₈ aryl;

X₄₂ can be CR₄₄; and

X₄₂ can be N.

In another more specific embodiment, for Structural Formulas (IB), (IIB), (IIIB), (VIIB), (VIIIB), (IXB) and (XIB), X₄₂ is CR₄₄, and R₄₃ and R₄₄ are, independently, —H, methyl, ethyl, propyl, isopropyl, cyclopropyl, methoxy, ethoxy, propoxy, cyclopropoxy, or, taken together with the carbon atoms to which they are attached, form a cycloalkenyl, aryl, heterocyclyl, or heteroaryl ring. Values and specific values for the

ring A is represented by the following Structural Formula:

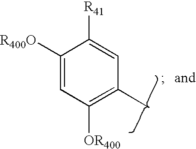

R₅ is represented by the following Structural Formula:

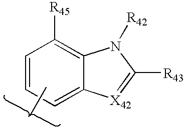

wherein values and specific values for the variables are as described in the eighteenth specific embodiment.

In a more specific embodiment, for Structural Formulas (I)-(IV), X₄₂ is CR₄₄. Even more specifically, R₄₃ and R₄₄ are, independently, selected from the group consisting of —H, methyl, ethyl, propyl, isopropyl, cyclopropyl, methooxy, ethoxy, propoxy, and cyclopropoxy. In another even more specific embodiment, R₄₃ and R₄₄ taken together with the carbon atoms to which they attached, form a cycloalkyenl, aryl, heterocyclyl, or heteroaryl ring. Even more specifically, R₄₃ and R₄₄, taken together with the carbon atoms to which they are attached, form a C₅—C₈ cycloalkyenyl or a C_{5—C}₈ aryl. Values and specific values for the remainder of the variables are as described in the nineteenth specific embodiment.

In another more specific embodiment, for Structural Formulas (I)-(IV), X₄₂ is N. Values and specific values for the remainder of the variables are as described in the nineteenth specific embodiment.

In another more specific embodiment, for Structural Formulas (I)-(IV), X₄₂ is CR₄₄ or N; remainder of the variables are as described above in the nineteenth specific embodiment.

In another more specific embodiment, for Structural Formulas (IB), (IIB), (IIIB), (VIIB), (VIIIB), (IXB) and (XIB), X₄₂ is CR₄₄; R₄₃ and R₄₄ are, independently, —H, methyl, ethyl, propyl, isopropyl, cyclopropyl, methoxy, ethoxy, propoxy, cyclopropoxy, or, taken together with the carbon atoms to which they are attached, form a cycloalkenyl, aryl, heterocyclyl, or heteroaryl ring; and R₆ are selected from the group consisting of —H, methyl, ethyl, isopropyl, and cyclopropyl. Values and specific values for the remainder of the variables are as described above in the nineteenth specific embodiment.

In a twentieth specific embodiment, the compounds of Structural Formulas (I)-(IV), (IA), (IA′), (IIA), (IB), (IIIB), (VIIB), (VIIIB), (IXB) and (XIB) are defined as the following or a tautomer, a pharmaceutically acceptable salt, solvate, clarthrate, or a prodrug thereof:

ring A is represented by the following Structural Formula:

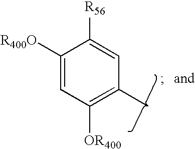

R₅ is represented by the following Structural Formula:

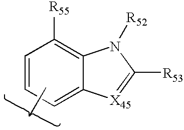

wherein:

X₄₅ is CR₅₄ or N;

R₅₂ for Structural Formulas (I)-(IV), (IB), (IIIB), (VIIB), (VIIIB), (IXB) and (XIB) is selected from the group consisting of —H, methyl, ethyl, n-propyl, isopropyl, n-butyl, n-pentyl, n-hexyl, —(CH₂)₂OCH₃, —CH₂C(O)OH, and —C(O)N(CH₃)₂;

R₅₂ for Structural Formulas (IA), (IA′) and (IIA) is selected from the group consisting of —H, methyl, ethyl, n-propyl, isopropyl, n-butyl, n-pentyl, n-hexyl, —(CH₂)₂OCH₃, —(CH₂)ₘR_{C}, wherein R_{C} is a protected carboxyl moiety and m is 1 or 2, and —C(O)N(CH₃)₂.

R₅₃ and R₅₄ are each, independently, —H, methyl, ethyl, or isopropyl; or R₅₃ and R₅₄ taken together with the carbon atoms to which they are attached form a phenyl, cyclohexenyl, or cyclooctenyl ring;

R₅₅ is selected from the group consisting of —H, —OH, —OCH₃, and —OCH₂CH₃; and

R₅₆ is selected from the group consisting of —H, methyl, ethyl, isopropyl, and cyclopropyl; and the remainder of the variables are as described in the nineteenth specific embodiment.

In a more specific embodiment, R₅₃ is H or a lower alkyl. Values and specific values for the remainder of the variables are as described in the twentieth specific embodiment.

In another more specific embodiment, X₄₅ is CR₅₄. Preferably, R₅₄ is H or a lower alkyl. Values and specific values for the remainder of the variables are as described in the twentieth specific embodiment.

In another specific embodiment, X₄₅ is N. Values and specific values for the remainder of the variables are as described in the twentieth specific embodiment.

In a twenty-first specific embodiment, the compounds of Structural Formulas (I)-(IV), (IA), (IA′), (IIA), (IB), (IIIB), (VIIB), (VIIIB), (IXB) and (XIB) are defined as the following:

ring A is represented by the following Structural Formula:

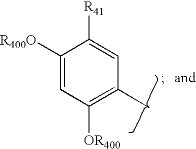

R₅ is represented by the following Structural Formula:

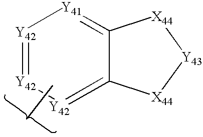

wherein:

X₄₄, for each occurrence, is independently, O, NR₄₂ or C(R₄₆)₂;

Y₄₃ is NR₄₂ or C(R₄₆)₂;

Y₄₁, Y₄₂, Z, R₄₁, R₄₂, and R₄₆ are as described in the eighteenth specific embodiment.

In a more specific embodiment, R₄₁ is selected from the group consisting of —H, lower alkyl, lower alkoxy, lower cycloalkyl, and lower cycloalkoxy. Values and specific values for the remainder of the variables are as described in the twenty-first specific embodiment.

In another more specific embodiment, R₄₁ is selected from the group consisting of —H, methyl, ethyl, propyl, isopropyl, cyclopropyl, methoxy, ethoxy, propoxy, and cyclopropoxy. Values and specific values for the remainder of the variables are as described in the twenty-first specific embodiment.

In another more specific embodiment, R₄₂ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂. Values and specific values for the remainder of the variables are as described in the twenty-first specific embodiment.

In another more specific embodiment, Y₄₁ is CR₄₅. Preferably, R₄₅ is H, a lower alkoxy, or —OH. Values and specific values for the remainder of the variables are as described in the twenty-first specific embodiment.

In another more specific embodiment, Y₄₂ is CH. Values and specific values for the remainder of the variables are as described in the twenty-first specific embodiment.

In another more embodiment, Y₄₃ is CH₂. Values and specific values for the remainder of the variables are as described in the twenty-first specific embodiment.

In another more specific embodiment, Y₄₃ is NR₄₂, wherein R₄₂ is H or a lower alkyl. Values and specific values for the remainder of the variables are as described in the twenty-first specific embodiment.

In another more specific embodiment, one of X₄₄ is NR₄₂ and the other is CH₂ or C(R₆)₂. Preferably, one of X₄₄ is NR₄₂ and the other is CH₂. Values and specific values for the remainder of the variables are as described in the twenty-first specific embodiment.

In a twenty-second specific embodiment, the compounds of Structural Formulas (I)-(IV), (IA), (IA′), (IIA), (IB), (IIIB), (VIIB), (VIIIB), (IXB) and (XIB) are defined as the following:

ring A is represented by the following Structural Formula:

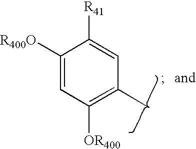

R₅ is represented by the following Structural Formula:

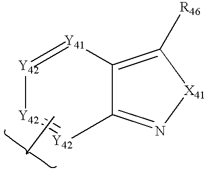

wherein the variables are as defined in the eighteenth specific embodiment.

In a more specific embodiment, R₄₁ is selected from the group consisting of —H, lower alkyl, lower alkoxy, lower cycloalkyl, and lower cycloalkoxy. Values and specific values for the remainder of the variables are as described in the twenty-second specific embodiment.

In another more specific embodiment, R₄₁ is selected from the group consisting of —H, methyl, ethyl, propyl, isopropyl, cyclopropyl, methoxy, ethoxy, propoxy, and cyclopropoxy. The remainder of the variables are as described in the twenty-second specific embodiment.

In another more specific embodiment, X₄₁ is NR₄₂. Preferably, R₄₂ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂. More preferably, R₄₂ is H or a lower alkyl. Values and specific values for the remainder of the variables are as described in the twenty-second specific embodiment.

In another more specific embodiment, X₄₁ is O. Values and specific values for the remainder of the variables are as described in the twenty-second specific embodiment.

In another more specific embodiment, X₄₁ is S. Values and specific values for the remainder of the variables are as described in the twenty-second specific embodiment.

In another more specific embodiment, Y₄₁ is CR₄₅. Preferably, R₄₅ is H, a lower alkoxy, or —OH. Values and specific values for the remainder of the variables are as described in the twenty-second specific embodiment.

In another more specific embodiment, Y₄₂ is CH. Values and specific values for the remainder of the variables are as described in the twenty-second specific embodiment.

In another more specific embodiment, R₄₆ is H or a lower alkyl. Values and specific values for the remainder of the variables are as described in the twenty-second specific embodiment.

In a twenty-third specific embodiment, the compounds of Structural Formulas (I)-(IV), (IA), (IA′), (IIA), (IB), (IIIB), (VIIB), (VIIIB), (IXB) and (XIB) are defined as the following:

ring A is represented by the following Structural Formula:

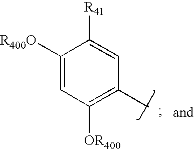

R₅ is represented by the following Structural Formula:

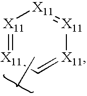

wherein X₁₁, for each occurrence, is independently CH, CR₉, N, N(O), or N⁺(R₁₇), provided that at least one X₁₁ is N, N(O), or N⁺(R₁₇) and at least two X₁₁ groups are independently selected from CH and CR₉; values and specific values for the remainder of the variables are as described above in the tenth specific embodiment.

In a more specific embodiment, one of the X₁₁ group is N, N(O), or N⁺(R₁₇) and the remaining X₁₁ groups are independently selected from CH and CR₉. More specifically, R₄₁ is a lower alkyl, C3-C6 cycloalkyl, lower alkoxy, a lower alkyl sulfanyl, or —NR₁₀R₁₁. Values and specific values for the remainder of the variables are as described above in the twenty-third specific embodiment.

In a twenty-fourth specific embodiment, the compound of formula (IIA) is represented by the following Structural Formula:

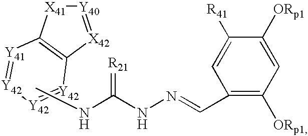

wherein the variables are as described above in the eighteenth specific embodiment.

In a more specific embodiment, the variables can each be independently selected from the following lists of specific values (values and specific values for the remainder of the substituents are as defined above in the twenty-third specific embodiment):

X₄₁ can be NR₄₂ and X₄₂ can be CR₄₄;

X₄₁ can be NR₄₂ and X₄₂ can be N;

R₄₁ can be selected from the group consisting of —H, lower alkyl, lower alkoxy, lower cycloalkyl, and lower cycloalkoxy;

R₄₁ can be selected from the group consisting of —H, methyl, ethyl, propyl, isopropyl, cyclopropyl, methoxy, ethoxy, propoxy, and cyclopropoxy;

X₄₁ can be NR₄₂, and R₄₂ can be selected from the group consisting of —H, a lower alkyl, a lower cycloalkyl, —C(O)N(R₂₇)₂, and —R_{C}, wherein R_{C} is a protected carboxyl group as defined above, and each R₂₇ is independently —H or a lower alkyl;

X₄₁ can be NR₄₂, and R₄₂ can be selected from the group consisting of —H, methyl, ethyl, n-propyl, isopropyl, cyclopropyl, n-butyl, sec-butyl, tert-butyl, n-pentyl, n-hexyl, —R_{C}, —(CH₂)ₘR_{C}, —CH₂OCH₃, —CH₂CH₂OCH₃, and —C(O)N(CH₃)₂, wherein R_{C} is a protected carboxyl group and m is 1 or 2;

R₄₃ and R₄₄ can be, independently, selected from the group consisting of —H, methyl, ethyl, propyl, isopropyl, cyclopropyl, methoxy, ethoxy, propoxy, and cyclopropoxy;

X₄₂ can be CR₄₄; Y₄₀ can be CR₄₃; and R₄₃ and R₄₄ together with the carbon atoms to which they are attached can form a cycloalkenyl, an aryl, heterocyclyl, or heteroaryl ring;

R₄₃ and R₄₄ together with the carbon atoms to which they are attached can form a C₅-C₈ cycloalkenyl or a C₅-C₈ aryl;

R₄₅ or CR₄₅ can be selected from the group consisting of —H, —ORₚ₁, —SRₚ₂, —NHRₚ₃, —N(Rₚ₃)₂, a lower alkoxy, —(CH₂)ₘ—NHRₚ₃, and —(CH₂)ₘ—N(Rₚ₃)₂, wherein m is an integer from 1 to 6;

R₄₅ can be selected from the group consisting of —H, —ORₚ₁, methoxy and ethoxy;

X₄₁ can be O.

In a twenty-fifth specific embodiment, the compound of formula (IIA) is represented by the following Structural Formula:

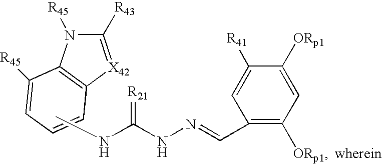

the variables are as described above in the twenty-third specific embodiment.

In a more specific embodiment, R₂₁ is O. Values and specific values for the remainder of the substituents are as defined above in the twenty-third specific embodiment.

In another more specific embodiment, the variables can each be independently selected from the following lists of specific values:

X₄₂ can be CR₄₄, and R₄₃ and R₄₄ can be, independently, selected from the group consisting of —H, methyl, ethyl, propyl, isopropyl, cyclopropyl, methoxy, ethoxy, propoxy, and cyclopropoxy;

X₄₂ can be CR₄₄, and R₄₃ and R₄₄, taken together with the carbon atoms to which they are attached, can form a cycloalkenyl, aryl, heterocyclyl, or heteroaryl ring;

R₄₃ and R₄₄, taken together with the carbon atoms to which they are attached, can form a C₅-C₈ cycloalkenyl or a C₅-C₈ aryl;

X₄₂ can be CR₄₄; and

X₄₂ can be N.

In a twenty-sixth specific embodiment, the compound of Structural Formula (IIA) is represented by the following Structural Formula:

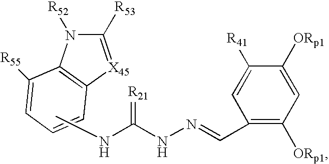

wherein:

X₄₅ is CR₅₄ or N;

R₂₁ is O;

R₅₆ is selected from the group consisting of —H, methyl, ethyl, isopropyl, and cyclopropyl;

R₅₂ is selected from the group consisting of —H, methyl, ethyl, n-propyl, isopropyl, n-butyl, n-pentyl, n-hexyl, —(CH₂)₂OCH₃, —(CH₂)ₘR_{C}, wherein R_{C} is a protected carboxyl moiety and m is 1 or 2, and —C(O)N(CH₃)₂;

R₅₃ and R₅₄ are each, independently, —H, methyl, ethyl, or isopropyl;

or R₅₃ and R₅₄ taken together with the carbon atoms to which they are attached form a phenyl, cyclohexenyl, or cyclooctenyl ring; and

R₅₅ is selected from the group consisting of —H, —OH, —OCH₃, and —OCH₂CH₃.

Values and specific values for the remainder of the substituents are as described in the twenty-fourth specific embodiment.

In one embodiment, the present invention is a method of preparing a compound of Structural Formula (XXXIA)

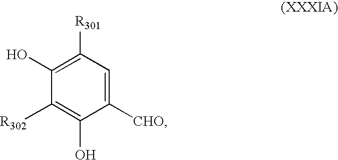

comprising the step of reacting the compound of for Structural Formula (XXXA)

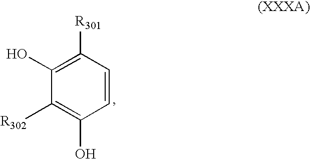

with POCl₃ in dimethyl formamide (DMF).

In one embodiment of the present invention, POCl₃ (typically in excess over the compound of Structural Formula (XXXA)) is added to cold DMF. Because the reaction is exothermic, the reagents are commonly added with cooling.

The molar ration of the POCl₃ to the compound of Structural Formula (XXXA) can be, for example, 10:1, 9:1, 8:1, 7:1, 6:1, 5:1, 4:1, 3:1, 2:1, 1.5:1, 1.2:1. ort 1.1:1.

Preferably, the molar ration is 5:1 to 1.5:1. More preferably, the moral ratio is 3:1 to 2:1.

Preferably, the product of the reaction between the compound of Structural Formula (XXXA) and POCl₃ in dimethyl formamide (DMF) is further reacted with a hydroxide base, such as NaOH. Typically, an excess of the base with respect to the starting reagent is used. In one embodiment, 12 equivalents of NaOH is used.

In Structural Formulas (XXXA) and (XXXIA), wherein R₃₀₁ and R₃₀₂ are each independently —H, an alkyl, an aryl, a heteroaryl, an aralkyl, a heteraralkyl, each optionally substituted by one or more of an alkyl, alkoxy, haloalkyl, halogen nitro, cyano or alkyl alkanoate groups.

Preferably, R₃₀₁ and R₃₀₂ are each independently —H, an optionally substituted C1-C6 alkyl, an optionally substituted phenyl, an optionally substituted benzyl, or an optionally substituted six-member heteroaryl. In one embodiment, R₃₀₁ and R₃₀₂ are not simultaneously hydrogens.

More preferably, R₃₀₁ and R₃₀₂ are each independently —H, an optionally substituted C₁-C₆ alkyl. Even more preferably, R₃₀₂ is H and R₃₀₁ is isopropyl, such that the compound of Structural Formula (XXXA) is compound 11A:

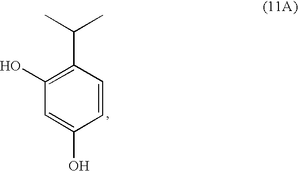

and the compound of formula (XXXIA) is compound 12A:

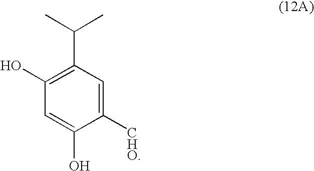

In another embodiment, the present invention is a method of synthesis of a compound of formula (XXA), comprising reacting a compound of formula (XXIA):

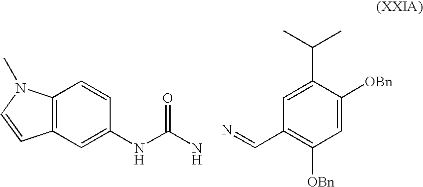

with an oxidizing agent, thereby producing a compound of formula (XXA):

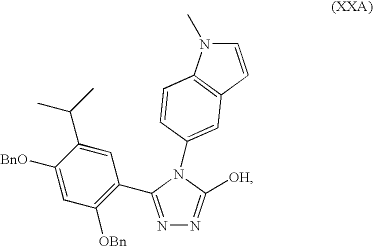

wherein Bn is a benzyl group.

The conditions for the reactions are described above with reference to Structural Formulas (IA), (IA′), (IIA), (IIIA) and (IVA). Preferably, the oxidizing agent is K₃Fe(CN)₆.

Preferably, the compound of formula (XXIA) is prepared by reacting a compound of formula (XXIIA)

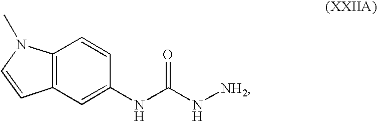

with a compound of formula (XXIIIA)

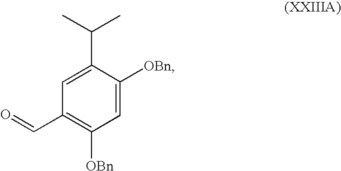

in the presence of an acid. Preferably, a catalytic amount of acid is used. The condition for this reaction are described above with reference to formulas (IA), (IA′), (IIA), (IIIA) and (IVA).

Preferably, the compound of formula (XXA) is further deprotected, thereby producing a compound of formula (XXIVA):

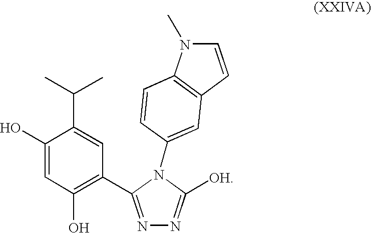

In one embodiment, the methods of present invention further comprises the step of deprotecting the compounds of Structural Formulas (I), (IA) and (IB). General conditions for deprotecting the compounds of Structural Formulas (I), (IA) and (IB) are known in the art and depend on the nature of the protecting group used. Examples are provided above with reference to Greene.

In another specific embodiment, the methods of the present invention comprise the step of deprotecting the compound of the following Structural Formula:

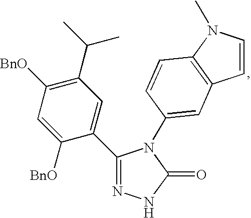

by reaction of hydrogen in the presence of ammonium formate in a polar solvent using Pd/C as catalyst, thereby forming a compound represented by the following Structural Formula:

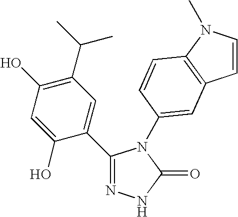

More specifically, the polar solvent is ethanol. More specifically, the reaction temperature is between 50° C.-60° C.

In one embodiment, for method III, the method further comprises the step of deprotecting the compound represented by the following Structural Formula:

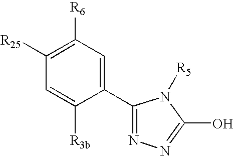

wherein R_{3b} and R₂₅ are —OR₁₀₀, thereby forming a triazole compound represented by the following Structural Formula:

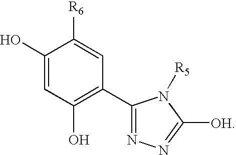

The remaining values and specific values are as described above in the fourteenth specific embodiment.

In another embodiment, for method III, the method further comprises the step of deprotecting the thioamide compounds represented by the following Structural Formula:

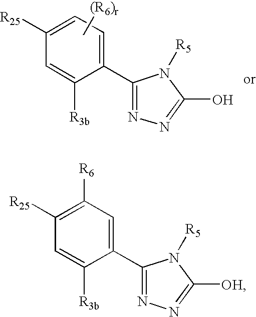

wherein:
R₅ is represented by the following Structural Formula:

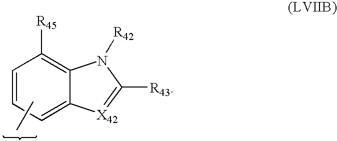

R_{3b} and R₂₅ are —OR₁₀₀, thereby forming a triazole compound represented by the following Structural Formula:

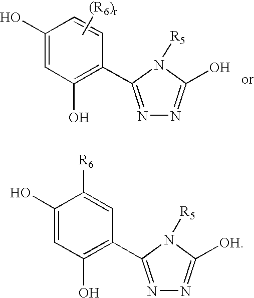

In another specific embodiment, the compounds represented by Structural Formula (IVB) is deprotected, thereby forming a triazole compound of the following Structural Formula:

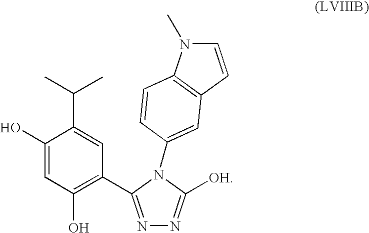

In a preferred embodiment, the second starting compound of Structural Formula (LVIIIB) used in the disclosed method III is prepared by reacting a thionation reagent with a compound represented by the following formula:

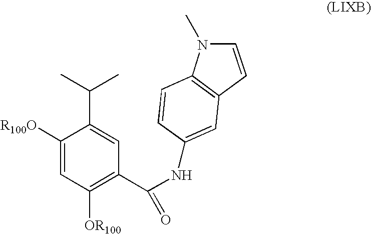

In one embodiment, the present invention comprises the step of deprotecting a compound of Structural Formulas (I), (IA), (IB), (IVB), (VIIB) and (XIB).

General conditions for deprotecting the compounds of Structural Formulas (I), (IA), (IB), (IVB), (VIIB) and (XIB) are known in the art and depend on the nature of protecting group used. Examples are provided above with reference to Greene.

In one embodiment, where a benzyl group is employed as a protecting group, the deprotection of compounds of Structural Formulas (I), (IA), (IB), (IVB), (VIIB) and (XIB) can be accomplished by catalytic hydrogenation. Any hydrogenation catalyst can be used, either soluble or insoluble in the reaction medium. Typical catalysts include palladium-on-charcoal, Raney nickel, NaBH₄-reduced nickel, platinum metal or its oxide, rhodium ruthenium or zinc oxide. Hydrogenation reactions are typically carried out at temperature from about 0° C. to about 50° C., preferably at 15-35° C. at atmospheric or slightly above atmospheric pressure.

The compounds of Structural Formulas (I), (IA), (IB), (IVB), (VIIB) and (XIB) are typically reacted with hydrogen at room temperature in a polar solvent. Preferably, palladium-on-charcoal is used as a catalyst.

The polar solvent can be one or more of a polar protic solvent, such as water or an alcohol; an ethereal solvent such as THF, dioxane and the like. For example, the solvent can be a mixture of THF and methanol. The mixture (by volume) can be 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10. Preferably, the THF/MeOH mixture is from about 4:1 to about 1:1 by volume.

In a specific embodiment, when R₃ of Structural Formula (I) is —OR_{A}; R₂₀ in Structural Formula (IA) is —ORₚ₁; or R_{3b} of Structural Formula (IB) is —OR₁₀₀, wherein R_{A}, Rₚ₁ and R₁₀₀ are benzyl groups, the deprotection step of compounds of Structural Formulas (I), (IA), (IB), (IVB), (VIIB) and (XIB) comprises reacting a compound of Structural Formulas (I), (IA), (IB), (IVB), (VIIB) and (XIB) with ammonium formate in the presence of a hydrogen catalyst. In one aspect, the hydrogen catalyst is palladium on activated carbon. In one aspect, the step of deprotecting is carried out at a temperature from 45 to 65° C. In one aspect, the step of deprotecting is carried out at about 55° C. In one aspect, the compound of Structural Formulas (I), (IA), (IB), (IVB), (VIIB) or (XIB) and the ammonium formate are reacted for about 1 to 5 hours in the presence of the palladium on activated carbon. In one aspect, the compound of Structural Formulas (I), (IA) or (IB) and the ammonium formate are reacted for about 1 hour in the presence of the palladium on activated carbon. In one aspect, the compound of Structural Formulas (I), (IA) or (IB) and the ammonium formate are reacted for about 12 hours in the presence of the palladium on activated carbon. In one aspect, the purity of the deprotected product of a compound of Structural Formulas (I), (IA), (IB), (IVB), (VIIB) or (XIB) is 99.0% or greater. In another aspect, the purity is 99.5% or greater. In a further aspect, the purity is 99.8% or greater.

Specific examples of compounds which can be prepared by the disclosed method III are provided below:
3-(2-Hydroxyphenyl)-4-(naphthalen-1-yl)-5-mercapto-triazole;
3-(2,4-Dihydroxyphenyl)-4-[4-(2-methoxyethoxy)-naphthalen-1-yl]-5-mercapto-triazole;
3-(2,4-Dihydroxyphenyl)-4-(2-methyl-4-bromophenyl)-5-mercapto-triazole;
3-(3,4-Dihydroxyphenyl)-4-(6-methoxy-naphthalen-1-yl)-5-mercapto-triazole;
3-(3,4-Dihydroxyphenyl)-4-(6-ethoxy-naphthalen-1-yl)-5-mercapto-triazole;
3-(3,4-Dihydroxyphenyl)-4-(6-propoxy-naphthalen-1-yl)-5-mercapto-triazole;
3-(2,4-Dihydroxy-5-ethyl-phenyl)-4-(5-methoxy-naphthalen-1-yl)-5-mercapto-triazole;
3-(3,4-Dihydroxyphenyl)-4-(6-isopropoxy-naphthalen-1-yl)-5-mercapto-triazole;
3-(2,4-Dihydroxyphenyl)-4-(2,6-diethylphenyl)-5-mercapto-triazole;
3-(2,4-Dihydroxyphenyl)-4-(2-methyl-6-ethylphenyl)-5-mercapto-triazole;
3-(2,4-Dihydroxyphenyl)-4-(2,6-diisopropylphenyl)-5-mercapto-triazole;
3-(2,4-Dihydroxyphenyl)-4-(1-ethyl-indol-4-yl)-5-mercapto-triazole;
3-(2,4-Dihydroxyphenyl)-4-(2,3-dihydro-benzo[1,4]dioxin-5-yl)-5-mercapto-triazole;
3-(2,4-Dihydroxyphenyl)-4-(3-methylphenyl)-5-mercapto-triazole;
3-(2,4-Dihydroxyphenyl)-4-(4-methylphenyl)-5-mercapto-triazole;
3-(2,4-Dihydroxyphenyl)-4-(2-chlorophenyl)-5-mercapto-triazole;
3-(2,4-Dihydroxyphenyl)-4-(3-chlorophenyl)-5-mercapto-triazole;
3-(2,4-Dihydroxyphenyl)-4-(4-chlorophenyl)-5-mercapto-triazole;
3-(2,4-Dihydroxyphenyl)-4-(2-methoxyphenyl)-5-mercapto-triazole;
3-(2,4-Dihydroxyphenyl)-4-(3-methoxyphenyl)-5-mercapto-triazole;
3-(2,4-Dihydroxyphenyl)-4-(3-fluorophenyl)-5-mercapto-triazole;
3-(2,4-Dihydroxyphenyl)-4-(2-ethylphenyl)-5-mercapto-triazole;
3-(2-Hydroxy-4-fluorophenyl)-4-(naphthalen-1-yl)-5-mercapto-triazole;
3-(2-Hydroxy-4-aminophenyl)-4-(naphthalen-1-yl)-5-mercapto-triazole;
3-(2,4-Dihydroxyphenyl)-4-(2-methyl-4-butyl-phenyl)-5-mercapto-triazole;
3-(2,4-Dihydroxyphenyl)-4-(2,4-dimethyl-phenyl)-5-mercapto-triazole;
3-(2,4-Dihydroxyphenyl)-4-(2,6-dimethyl-phenyl)-5-mercapto-triazole;
3-(2,4-Dihydroxyphenyl)-4-(2,6-dimethyl-phenyl)-5-mercapto-triazole;
3-(2,4-Dihydroxyphenyl)-4-(4-fluorophenyl)-5-mercapto-triazole;
3-(2,4-Dihydroxyphenyl)-4-(2-methylsulfanylphenyl)-5-mercapto-triazole;
3-(2,4-Dihydroxyphenyl)-4-(naphthalene-2-yl)-5-mercapto-triazole;
3-(2,4-Dihydroxyphenyl)-4-(2,3-dimethylphenyl)-5-mercapto-triazole;
3-(2,4-Dihydroxyphenyl)-4-(2-methyl-4-fluorophenyl)-5-mercapto-triazole;
3-(2,4-Dihydroxyphenyl)-4-(acenaphthalen-5-yl)-5-mercapto-triazole;
3-(2-Hydroxy-4-methoxy-phenyl)-4-(naphthalen-1-yl)-5-mercapto-triazole;
3-(2,4-Dihydroxyphenyl)-4-(2,3-dichlorophenyl)-5-mercapto-triazole;
3-(2,4-Dihydroxyphenyl)-4-(5-methoxynaphthalen-1-yl)-5-mercapto-triazole;
3-(2,4-Dihydroxyphenyl)-4-(pyren-1-yl)-5-mercapto-triazole;
3-(2,4-Dihydroxyphenyl)-4-(quinolin-5-yl)-5-mercapto-triazole;
3-(2,4-Dihydroxyphenyl)-4-(1,2,3,4-tetrahydronaphthalen-5-yl)-5-mercapto-triazole;
3-(2,4-Dihydroxyphenyl)-4-(anthracen-1-yl)-5-mercapto-triazole;
3-(2,4-Dihydroxyphenyl)-4-(biphenyl-2-yl)-5-mercapto-triazole;
3-(2,4-Dihydroxy-6-methyl-phenyl)-4-(naphthalene-1-yl)-5-mercapto-triazole;
3-(2,4-Dihydroxyphenyl)-4-(4-pentyloxyphenyl)-5-mercapto-triazole;
3-(2,4-Dihydroxyphenyl)-4-(4-octyloxyphenyl)-5-mercapto-triazole;
3-(2,4-Dihydroxyphenyl)-4-(4-chloronaphthalen-1-yl)-5-mercapto-triazole;
3-(2,4-Dihydroxy-5-ethyl-phenyl)-4-(naphthalen-1-yl)-5-mercapto-triazole;
3-(2,4-Dihydroxy-5-ethyl-phenyl)-4-(7-carboxymethoxy-naphthalen-1-yl)-5-mercapto-triazole;
3-(2,4-Dihydroxyphenyl)-4-(2-methyl-quinolin-4-yl)-5-mercapto-triazole;
3-(3-Hydroxypyridin-4-yl)-4-(naphthalen-1-yl)-5-mercapto-triazole;
3-(2-Hydroxy-4-acetylamino-phenyl)-4-(naphthalen-1-yl)-5-mercapto-triazole;
3-(2,4-Dihydroxy-phenyl)-4-(1,2,3,4-tetrahydronaphthalen-1-yl)-5-mercapto-triazole;
3-(2,4-Dihydroxy-phenyl)-4-(2,3-dihydro-benzo[1,4]dioxin-5-yl)-5-mercapto-triazole;
3-(2,4-Dihydroxy-phenyl)-4-(3,5-dimethoxyphenyl)-5-mercapto-triazole;
3-(2,4-Dihydroxy-phenyl)-4-(2,3-dimethyl-1H-indol-4-yl)-5-mercapto-triazole;
3-(2,4-Dihydroxy-3-propyl-phenyl)-4-(naphthalen-1-yl)-5-mercapto-triazole;
3-(4,6-Dihydroxy-1-ethyl-pyridin-3-yl)-4-(naphthalen-1-yl)-5-mercapto-triazole;
3-(4,6-Dihydroxy-1-methyl-pyridin-3-yl)-4-(naphthalen-1-yl)-5-mercapto-triazole;
3-(2,4-Dihydroxy-phenyl)-4-(3,5-di-tert-butylphenyl)-5-mercapto-triazole;
3-(2,6-Dihydroxy-5-fluoro-pyridin-3-yl)-4-(naphthalen-1-yl)-5-mercapto-triazole;
3-(2,4-Dihydroxy-5-methyl-phenyl)-4-(naphthalene-1-yl)-5-mercapto-triazole;
3-[2,4-Dihydroxy-phenyl]-4-(3-benzoylphenyl)-5-mercapto-triazole;
3-(2,4-Dihydroxy-phenyl)-4-(4-carboxy-naphthalen-1-yl)-5-mercapto-triazole;
3-(2,4-Dihydroxy-phenyl)-4-[4-(N,N-dimethylcarbamoyl)-naphthalen-1-yl]-5-mercapto-triazole;
3-(2,4-Dihydroxy-phenyl)-4-(4-propoxy-naphthalen-1-yl)-5-mercapto-triazole;
3-(2,4-Dihydroxy-phenyl)-4-(4-isopropoxy-naphthalen-1-yl)-5-mercapto-triazole;
3-(2,4-Dihydroxy-phenyl)-4-(5-isopropoxy-naphthalen-1-yl)-5-mercapto-triazole;
3-(2,4-Dihydroxy-phenyl)-4-(isoquinolin-5-yl)-5-mercapto-triazole;
3-(2,4-Dihydroxy-phenyl)-4-(5-propoxy-naphthalen-1-yl)-5-mercapto-triazole;
3-(2-Hydroxy-4-methanesulfonamino-phenyl)-4-(naphthalen-1-yl)-5-mercapto-triazole;
3-(2,4-Dihydroxy-3,6-dimethyl-phenyl)-4-(naphthalen-1-yl)-5-mercapto-triazole;
3-(2,4-Dihydroxy-phenyl)-4-[7-(2-methoxyethoxy)-naphthalen-1-yl]-5-mercapto-triazole;
3-(2,4-Dihydroxy-5-hexyl-phenyl)-4-(naphthalen-1-yl)-5-mercapto-triazole;
3-(2,4-Dihydroxy-5-ethyl-phenyl)-4-(4-methoxy-naphthalen-1-yl)-5-mercapto-triazole;
3-(2,4-Dihydroxy-5-ethyl-phenyl)-4-(6-methoxy-naphthalin-1-yl)-5-mercapto-triazole;
3-(2,4-Dihydroxy-3-chloro-5-ethyl-phenyl)-4-(naphthalen-1-yl)-5-mercapto-triazole;
3-(2,4-Dihydroxy-5-ethyl-phenyl)-4-(2,3-dimethyl-4-methoxy-phenyl)-5-mercapto-triazole;
3-(2,4-Dihydroxy-phenyl)-4-(7-isopropoxy-naphthalen-1-yl)-5-mercapto-triazole;
3-(2,4-Dihydroxy-phenyl)-4-(7-ethoxy-naphthalen-1-yl)-5-mercapto-triazole;
3-(2,4-Dihydroxy-phenyl)-4-(7-propoxy-naphthalen-1-yl)-5-mercapto-triazole;
3-(2-Hydroxy-4-methoxymethyoxy-phenyl)-4-(naphthalen-1-yl)-5-mercapto-triazole;
3-[2-Hydroxy-4-(2-hydroxy-ethoxy)-phenyl]-4-(naphthalen-1-yl)-5-mercapto-triazole;
3-(2,4-Dihydroxyphenyl)-4-(7-methoxy-naphthalen-1-yl)-5-mercapto-triazole;
3-(2,4-Dihydroxyphenyl)-4-(5-methoxy-naphthalen-1-yl)-5-mercapto-triazole;
3-(2,4-Dihydroxyphenyl)-4-(4-hydroxy-naphthalen-1-yl)-5-mercapto-triazole;
3-(2,4-Dihydroxyphenyl)-4-(1-isopropyl-indol-4-yl)-5-mercapto-triazole;
3-(2,4-Dihydroxy-5-tert-butyl-phenyl)-4-(naphthalen-1-yl)-5-mercapto-triazole;
3-(2,4-Dihydroxy-5-propyl-phenyl)-4-(naphthalen-1-yl)-5-mercapto-triazole;
3-(2,4-Dihydroxy-3-methyl-5-ethyl-phenyl)-4-(naphthalen-1-yl)-5-mercapto-triazole;
3-(2,4-Dihydroxy-5-isobutyl-phenyl)-4-(naphthalen-1-yl)-5-mercapto-triazole;
3-(2,4-Dihydroxy-phenyl)-4-(2,3-dimethoxy-phenyl)-5-mercapto-triazole;
3-(2,4-Dihydroxy-phenyl)-4-(2-methoxy-3-chloro-phenyl)-5-mercapto-triazole;
3-(2,4-Dihydroxy-phenyl)-4-(indol-4-yl)-5-mercapto-triazole;
3-(2,4-Dihydroxy-phenyl)-4-[1-(2-methoxyethoxy)-indol-4-yl]-5-mercapto-triazole;
3-(2,4-Dihydroxy-phenyl)-4-(naphthalen-1-yl)-5-hydroxy-triazole;
3-(1-Oxo-3-hydroxy-pyridin-4-yl)-4-(naphthalen-1-yl)-5-mercapto-triazole;
3-(2,5-Dihydroxy-4-carboxy)-4-(naphthalen-1-yl)-5-mercapto-triazole;
3-(2,4-Dihydroxy-5-ethyl-phenyl)-4-(1-isopropyl-indol-4-yl)-5-mercapto-triazole;
3-(2,4-Dihydroxy-5-ethyl-phenyl)-4-[1-(dimethyl-carbamoyl)-indol-4-yl]-5-mercapto-triazole;
3-(2,4-Dihydroxy-5-ethyl-phenyl)-4-(1-ethyl-benzoimidazol-4-yl)-5-mercapto-triazole;
3-(2,4-Dihydroxy-5-ethyl-phenyl)-4-(1,2,3-trimethyl-indol-5-yl)-5-mercapto-triazole;
3-(2,5-Dihydroxy-4-hydroxymethyl-phenyl)-4-(naphthalen-1-yl)-5-mercapto-triazole;
3-(2-Hydroxy-4-amino-phenyl)-4-(naphthalen-1-yl)-5-mercapto-triazole;
3-(2-Hydroxy-4-acetylamino-phenyl)-4-(naphthalen-1-yl)-5-mercapto-triazole;
3-(2,4-Dihydroxy-3-chloro-phenyl)-4-(naphthalen-1-yl)-5-mercapto-triazole;
3-(2,4-Dihydroxy-5-ethyl-phenyl)-4-(3-methox-phenyl)-5-hydroxy-triazole;
3-(2,4-Dihydroxy-5-ethyl-phenyl)-4-(naphthalen-1-yl)-5-hydroxy-triazole;
3-(2,4-Dihydroxy-5-ethyl-phenyl)-4-(1-isopropyl-indol-3-yl)-5-hydroxy-triazole;
3-(2,4-Dihydroxy-5-ethyl-phenyl)-4-(1-isopropyl-indol-4-yl)-5-amino-triazole;
3-(2,4-Dihydroxy-5-ethyl-phenyl)-4-(3-methoxy-phenyl)-5-amino-triazole;
3-(2,4-Dihydroxy-5-ethyl-phenyl)-4-(naphthalen-1-yl)-5-amino-triazole;
3-(2-Hydroxy-5-ethyloxy-phenyl)-4-(naphthalen-1-yl)-5-hydroxy-triazole;
3-(2-Hydroxy-5-isopropyl-phenyl)-4-(naphthalen-1-yl)-5-hydroxy-triazole;
3-(2-Dihydroxy-phenyl)-4-(7-fluoro-naphthalen-1-yl)-5-hydroxy-triazole;
3-(2,4-Dihydroxy-phenyl)-4-(2,3-difluorophenyl)-5-hydroxy-triazole;
3-(2,4-Dihydroxy-phenyl)-4-[2-(1H-tetrazol-5-yl)-phenyl]-5-hydroxy-triazole;
3-(2,4-Dihydroxy-phenyl)-4-(benzothiazol-4-yl)-5-hydroxy-triazole;
3-(2,4-Dihydroxy-phenyl)-4-(9H-purin-6-yl)-5-hydroxy-triazole;
3-(2,4-Dihydroxy-phenyl)-4-{4-[2-(moropholin-1-yl)-ethoxy]-phenyl}-5-hydroxy-triazole;
3-(2,4-Dihydroxy-phenyl)-4-cyclopentyl-5-hydroxy-triazole;
3-(2,4-Dihydroxy-5-methoxy-phenyl)-4-(naphthalen-1-yl)-5-mercapto-triazole;
3-(2,4-Dihydroxy-5-ethyl-phenyl)-4-(5-hydroxy-naphthalen-1-yl)-5-mercapto-triazole;
3-(2,4-Dihydroxy-phenyl)-4-(naphthalen-1-ylmethyl)-5-mercapto-triazole;
3-(2-Hydroxy-4-methoxyphenyl)-4-(naphthalen-1-yl)-5-mercapto-triazole;
3-(2,4-Dihydroxy-phenyl)-4-(biphenyl-3-yl)-5-mercapto-triazole;
3-(2,4-Dihydroxy-phenyl)-4-(2-methyl-5-hydroxymethyl-phenyl)-5-mercapto-triazole;
3-(2,4-Dihydroxy-phenyl)-4-(1-dimethylcarbamoyl-indol-4-yl)-5-mercapto-triazole;
3-(2,4,5-Trihydroxy-phenyl)-4-(naphthalene-1-yl)-5-mercapto-triazole;
3-(2,4-Dihydroxy-5-ethyl-phenyl)-4-(2,3-dimethyl-indol-5-yl)-5-mercapto-triazole;
3-(2,4-Dihydroxy-5-ethyl-phenyl)-4-(3-t-butyl-4-methoxy-phenyl)-5-mercapto-triazole;
3-(2,4-Dihydroxy-5-ethyl-phenyl)-4-(1-ethyl-1H-benzoimidazol-4-yl)-5-mercapto-triazole, HCl salt;
3-(2,4-Dihydroxy-5-ethyl-phenyl)-4-(1-isopropyl-7-methoxy-indol-4-yl)-5-mercapto-triazole; and
3-(2,4-Dihydroxy-5-cyclopropyl-phenyl)-4-(naphthalene-1-yl)-5-mercapto-triazole, or a tautomer, pharmaceutically acceptable salt, solvate, clathrate or prodrug thereof.

Exemplary compounds that can be prepared by the disclosed method I and method III are depicted in Tables 1 and 2 below, including tautomers, pharmaceutically acceptable salts, solvates, clathrates, hydrates, polymorphs or prodrugs and synthetic intermediates thereof represented by Structural Formula (II), (III), or (IV). Exemplary compounds that can be prepared by the disclosed method II include compounds 97, 137-173, 176, 220, and 232 depicted in Table 1 below.

TABLE 1

| No. | Structure | Tautomeric Structure | Name |
|---|---|---|---|
| 1 | 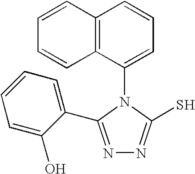 | 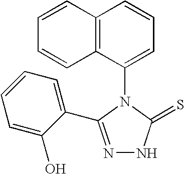 | 3-(2-Hydroxyphenyl)-4-(naphthalen-1-yl)-5-mercapto-[1,2,4]triazole |
| 2 | 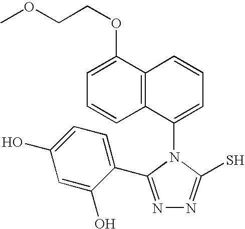 | 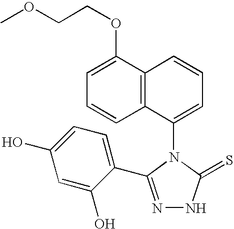 | 3-(2,4-Dihydroxyphenyl)-4-[4-(2-methoxyethoxy)-naphthalen-1-yl]-5-mercapto-[1,2,4]triazole |
| 3 | 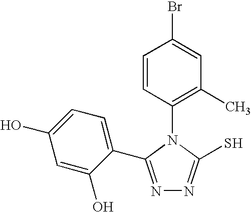 | 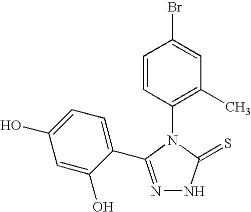 | 3-(2,4-Dihydroxyphenyl)-4-(2-methyl-4-bromophenyl)-5-mercapto-[1,2,4]triazole |
| 4 | 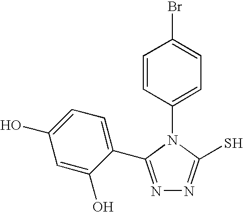 | 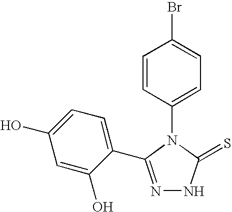 | 3-(2,4-Dihydroxyphenyl)-4-(4-bromophenyl)-5-mercapto-[1,2,4]triazole |
| 5 | 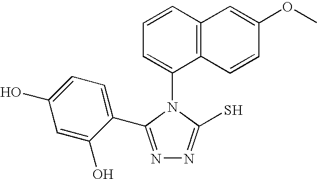 | 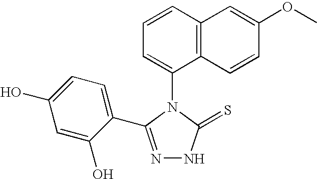 | 3-(3,4-Dihydroxyphenyl)-4-(6-methoxy-naphthalen-1-yl)-5-mercapto-[1,2,4]triazole |
| 6 | 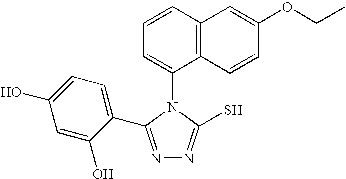 | 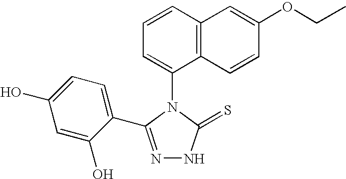 | 3-(3,4-Dihydroxyphenyl)-4-(6-ethoxy-naphthalen-1-yl)-5-mercapto-[1,2,4]triazole |
| 7 | 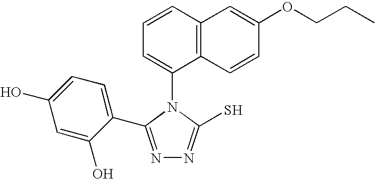 | 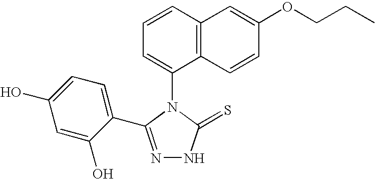 | 3-(3,4-Dihydroxyphenyl)-4-(6-propoxy-naphthalen-1-yl)-5-mercapto-[1,2,4]triazole |
| 8 | 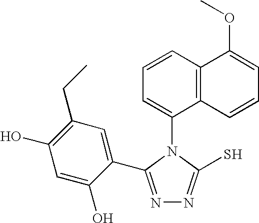 | 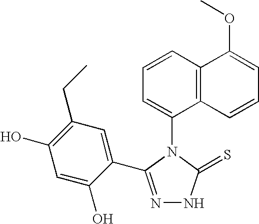 | 3-(2,4-Dihydroxy-5-ethyl-phenyl)-4-(5-methoxy-naphthalen-1-yl)-5-mercapto-[1,2,4]triazole |
| 9 | 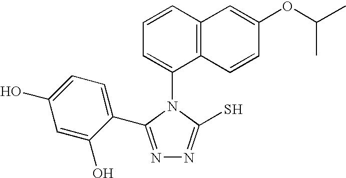 | 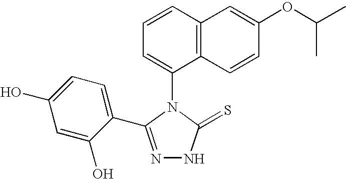 | 3-(3,4-Dihydroxyphenyl)-4-(6-isopropoxy-naphthalen-1-yl)-5-mercapto-[1,2,4]triazole |
| 10 | 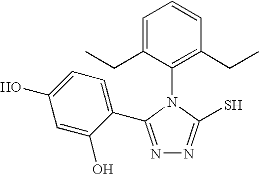 | 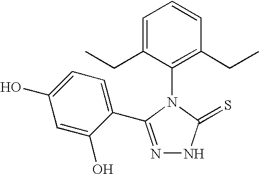 | 3-(2,4-Dihydroxyphenyl)-4-(2,6-diethylphenyl)-5-mercapto-[1,2,4]triazole |
| 11 | 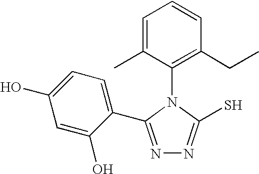 | 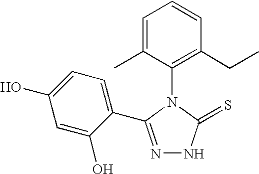 | 3-(2,4-Dihydroxyphenyl)-4-(2-methy-6-ethylphenyl)-5-mercapto-[1,2,4]triazole |
| 12 | 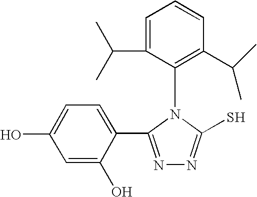 | 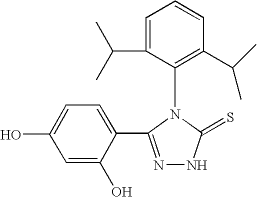 | 3-(2,4-Dihydroxyphenyl)-4-(2,6-diisopropylphenyl)-5-mercapto-[1,2,4]triazole |
| 13 | 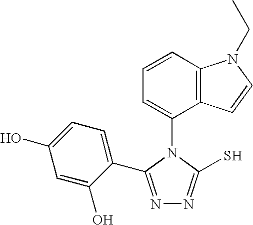 | 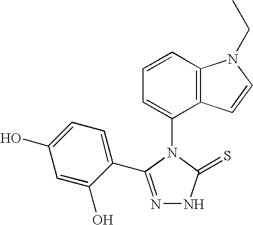 | 3-(2,4-Dihydroxyphenyl)-4-(1-ethyl-indol-4-yl)-5-mercapto-[1,2,4]triazole |
| 14 | 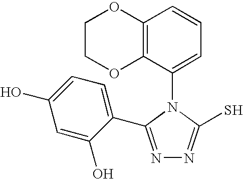 | 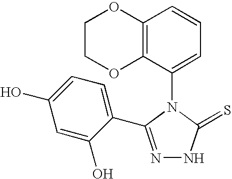 | 3-(2,4-Dihydroxyphenyl)-4-(2,3-dihydro-benzo[1,4]dioxin-5-yl)-5-mercapto-[1,2,4]triazole |
| 15 | 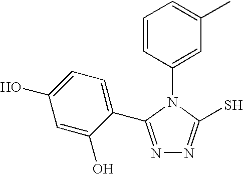 | 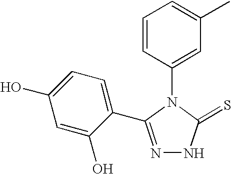 | 3-(2,4-Dihydroxyphenyl)-4-(3-methylphenyl)-5-mercapto-[1,2,4]triazole |
| 16 | 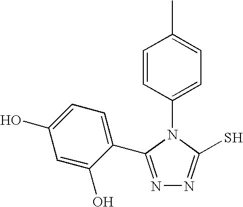 | 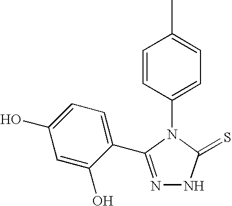 | 3-(2,4-Dihydroxyphenyl)-4-(4-methylphenyl)-5-mercapto-[1,2,4]triazole |
| 17 | 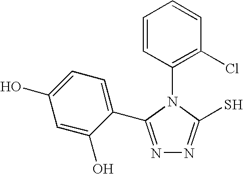 | 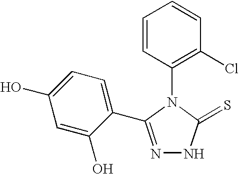 | 3-(2,4-Dihydroxyphenyl)-4-(2-chlorophenyl)-5-mercapto-[1,2,4]triazole |
| 18 | 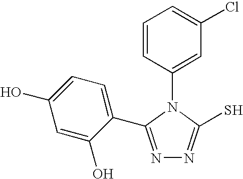 | 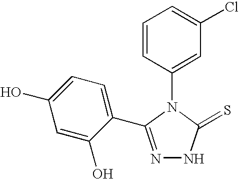 | 3-(2,4-Dihydroxyphenyl)-4-(3-chlorophenyl)-5-mercapto-[1,2,4]triazole |
| 19 | 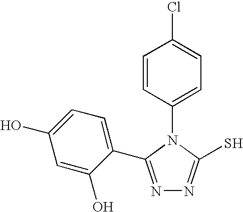 | 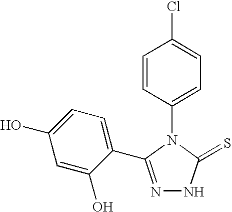 | 3-(2,4-Dihydroxyphenyl)-4-(4-chlorophenyl)-5-mercapto-[1,2,4]triazole |
| 20 | 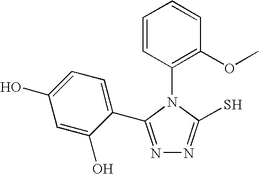 | 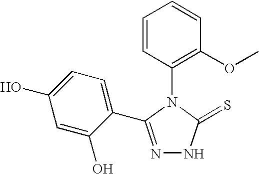 | 3-(2,4-Dihydroxyphenyl)-4-(2-methoxyphenyl)-5-mercapto-[1,2,4]triazole |
| 21 | 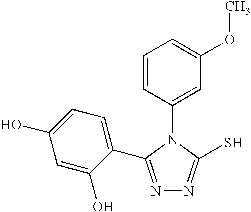 | 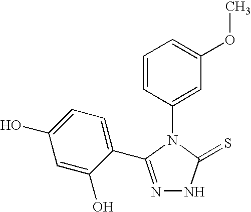 | 3-(2,4-Dihydroxyphenyl)-4-(3-methoxyphenyl)-5-mercapto-[1,2,4]triazole |
| 22 | 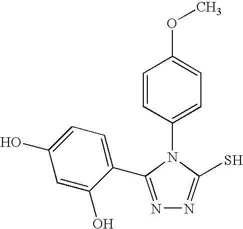 | 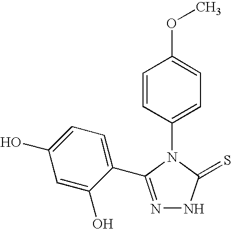 | 3-(2,4-Dihydroxyphenyl)-4-(4-methoxyphenyl)-5-mercapto-[1,2,4]triazole |
| 23 | 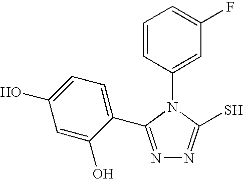 | 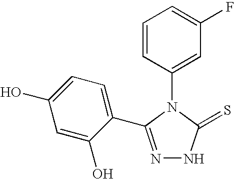 | 3-(2,4-Dihydroxyphenyl)-4-(3-fluorophenyl)-5-mercapto-[1,2,4]triazole |
| 24 | 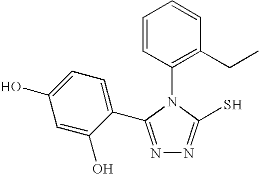 | 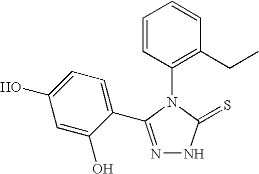 | 3-(2,4-Dihydroxyphenyl)-4-(2-ethylphenyl)-5-mercapto-[1,2,4]triazole |
| 25 | 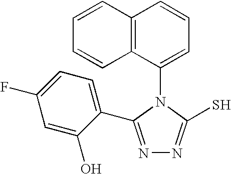 | 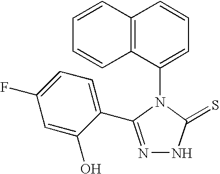 | 3-(2-Hydroxy-4-fluorophenyl)-4-(naphthalen-1-yl)-5-mercapto-[1,2,4]triazole |
| 26 | 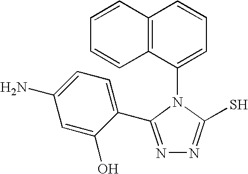 | 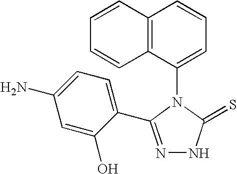 | 3-(2-Hydroxy-4-aminophenyl)-4-(naphthalen-1-yl)-5-mercapto-[1,2,4]triazole |
| 27 | 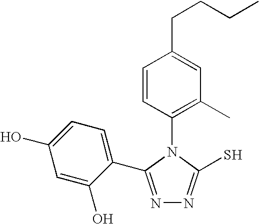 | 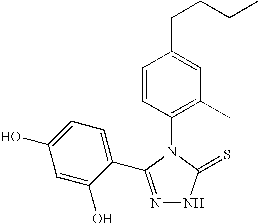 | 3-(2,4-Dihydroxyphenyl)-4-(2-methyl-4-butyl-phenyl)-5-mercapto-[1,2,4]triazole |
| 28 | 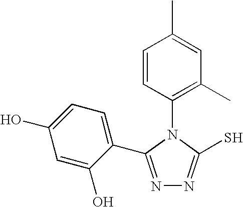 | 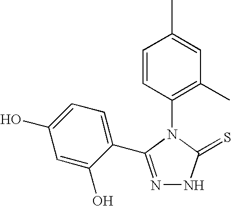 | 3-(2,4-Dihydroxyphenyl)-4-(2,4-dimethyl-phenyl)-5-mercapto-[1,2,4]triazole |
| 29 | 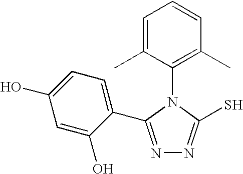 | 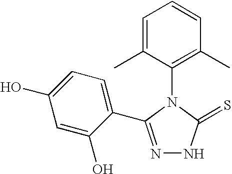 | 3-(2,4-Dihydroxyphenyl)-4-(2,6-dimethyl-phenyl)-5-mercapto-[1,2,4]triazole |
| 30 | 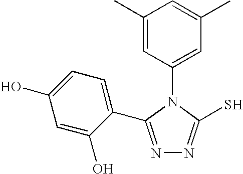 | 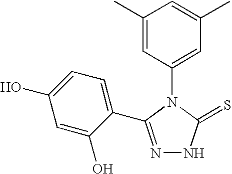 | 3-(2,4-Dihydroxyphenyl)-4-(2,6-dimethyl-phenyl)-5-mercapto-[1,2,4]triazole |
| 31 | 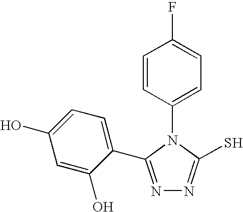 | 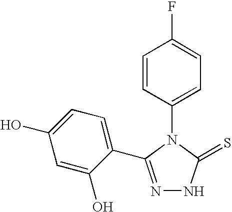 | 3-(2,4-Dihydroxyphenyl)-4-(4-fluorophenyl)-5-mercapto-[1,2,4]triazole |
| 32 | 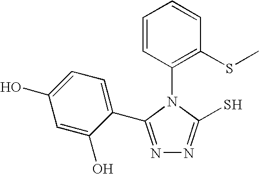 | 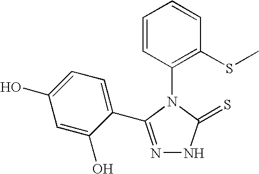 | 3-(2,4-Dihydroxyphenyl)-4-(2-methylsulfanylphenyl)-5-mercapto-[1,2,4]triazole |
| 33 | 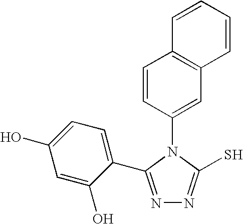 | 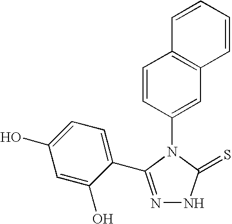 | 3-(2,4-Dihydroxyphenyl)-4-(naphthalene-2-yl)-5-mercapto-[1,2,4]triazole |
| 34 | 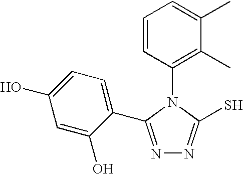 | 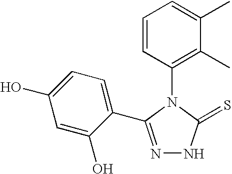 | 3-(2,4-Dihydroxyphenyl)-4-(2,3-dimethylphenyl)-5-mercapto-[1,2,4]triazole |
| 35 | 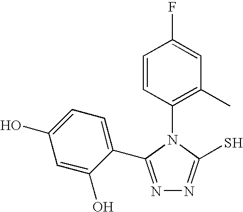 | 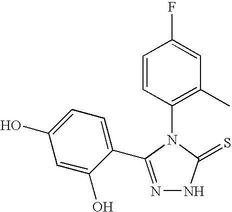 | 3-(2,4-Dihydroxyphenyl)-4-(2-methyl-4-fluorophenyl)-5-mercapto-[1,2,4]triazole |
| 36 | 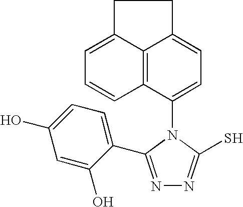 | 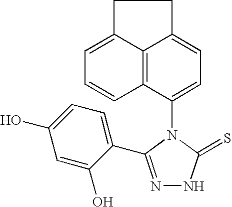 | 3-(2,4-Dihydroxyphenyl)-4-(acenaphthalen-5-yl)-5-mercapto-[1,2,4]triazole |
| 37 | 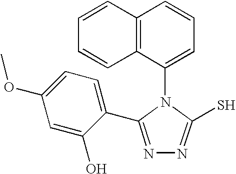 | 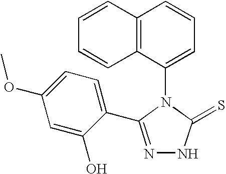 | 3-(2-Hydroxy-4-methoxy-phenyl)-4-(naphthalen-1-yl)-5-mercapto-[1,2,4]triazole |
| 38 | 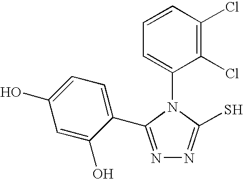 | 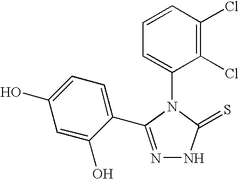 | 3-(2,4-Dihydroxyphenyl)-4-(2,3-dichlorophenyl)-5-mercapto-[1,2,4]triazole |
| 39 | 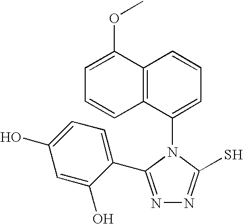 | 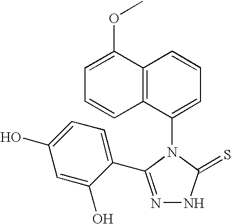 | 3-(2,4-Dihydroxyphenyl)-4-(5-methoxynaphthalen-1-yl)-5-mercapto-[1,2,4]triazole |
| 40 | 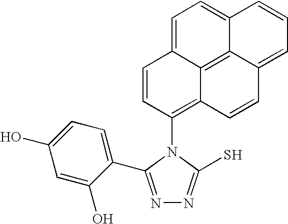 | 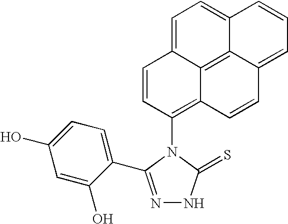 | 3-(2,4-Dihydroxyphenyl)-4-(pyren-1-yl)-5-mercapto-[1,2,4]triazole |
| 41 | 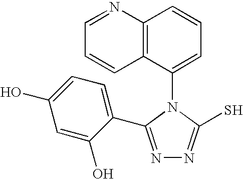 | 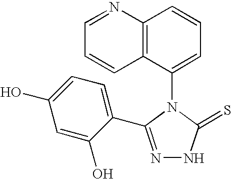 | 3-(2,4-Dihydroxyphenyl)-4-(quinolin-5-yl)-5-mercapto-[1,2,4]triazole |
| 42 | 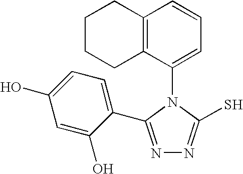 | 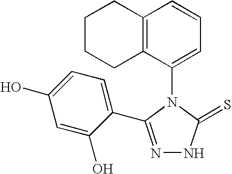 | 3-(2,4-Dihydroxyphenyl)-4-(1,2,3,4-tetrahydronaphthalen-5-yl)-5-mercapto-[1,2,4]triazole |
| 43 | 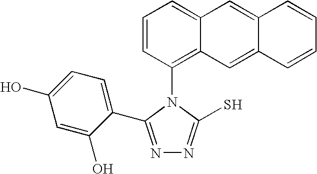 | 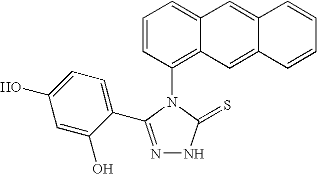 | 3-(2,4-Dihydroxyphenyl)-4-(anthracen-1-yl)-5-mercapto-[1,2,4]triazole |
| 44 | 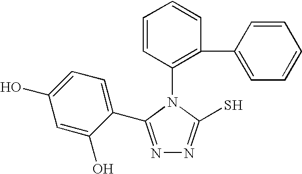 | 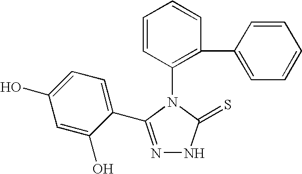 | 3-(2,4-Dihydroxyphenyl)-4-(biphenyl-2-yl)-5-mercapto-[1,2,4]triazole |
| 45 | 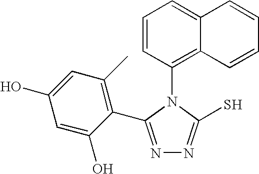 | 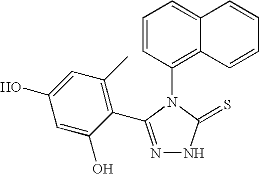 | 3-(2,4-Dihydroxy-6-methyl-phenyl)-4-(naphthalene-1-yl)-5-mercapto-[1,2,4]triazole |
| 46 | 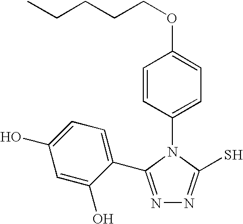 | 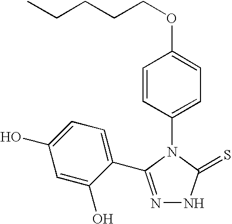 | 3-(2,4-Dihydroxyphenyl)-4-(4-pentyloxyphenyl)-5-mercapto-[1,2,4]triazole |
| 47 | 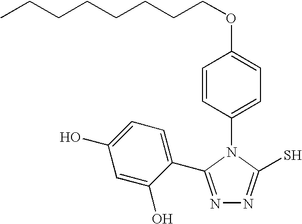 | 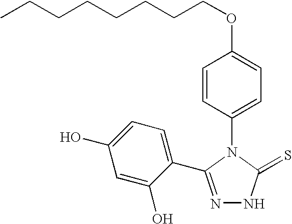 | 3-(2,4-Dihydroxyphenyl)-4-(4-octyloxyphenyl)-5-mercapto-[1,2,4]triazole |
| 48 | 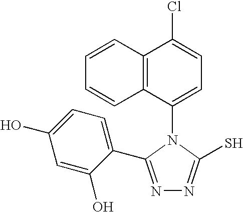 | 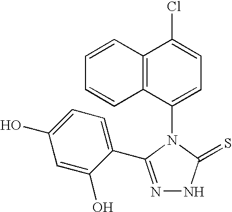 | 3-(2,4-Dihydroxyphenyl)-4-(4-chloronaphthalen-1-yl)-5-mercapto-[1,2,4]triazole |
| 49 | 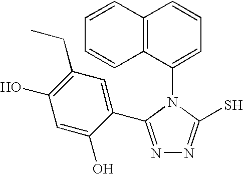 | 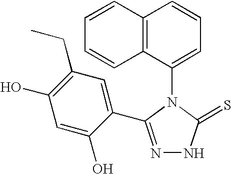 | 3-(2,4-Dihydroxy-5-ethyl-phenyl)-4-(naphthalen-1-yl)-5-mercapto-[1,2,4]triazole |
| 50 | 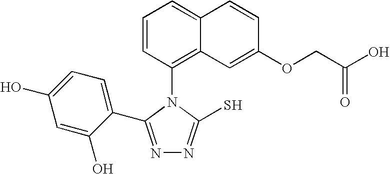 | 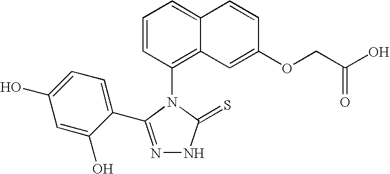 | 3-(2,4-Dihydroxy-5-ethyl-phenyl)-4-(7-carboxymethoxy-naphthalen-1-yl)-5-mercapto-[1,2,4]triazole |
| 51 | 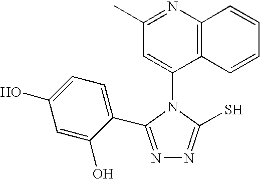 | 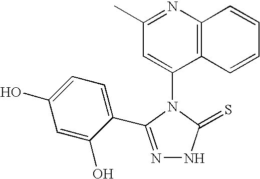 | 3-(2,4-Dihydroxyphenyl)-4-(2-methyl-quinolin-4-yl)-5-mercapto-[1,2,4]triazole |
| 52 | 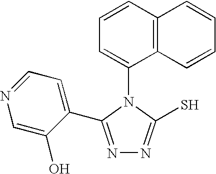 | 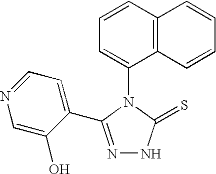 | 3-(3-Hydroxypyridin-4-yl)-4-(naphthalen-1-yl)-5-mercapto-[1,2,4]triazole |
| 53 | 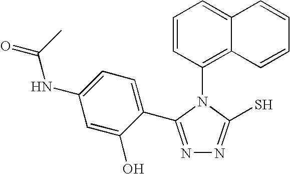 | 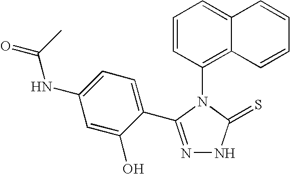 | 3-(2-Hydroxy-4-acetylamino-phenyl)-4-(naphthalen-1-yl)-5-mercapto-[1,2,4]triazole |
| 54 | 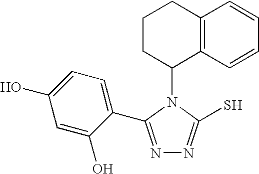 | 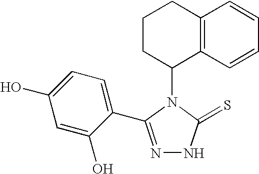 | 3-(2,4-Dihydroxy-phenyl)-4-(1,2,3,4-tetrahydronaphthalen-1-yl)-5-mercapto-[1,2,4]triazole |
| 55 | 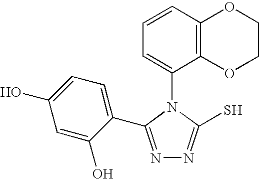 | 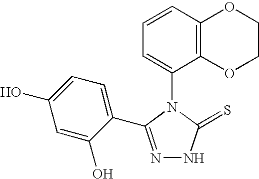 | 3-(2,4-Dihydroxy-phenyl)-4-(2,3-dihydro-benzo[1,4]dioxin-5-yl)-5-mercapto-[1,2,4]triazole |
| 56 | 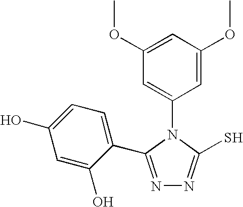 | 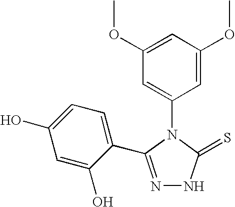 | 3-(2,4-Dihydroxy-phenyl)-4-(3,5-dimethoxyphenyl)-5-mercapto-[1,2,4]triazole |
| 57 | 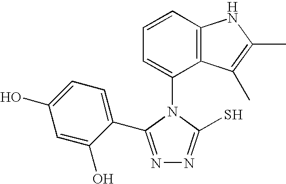 | 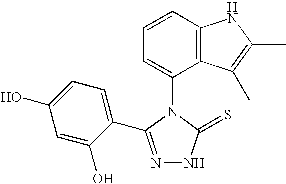 | 3-(2,4-Dihydroxy-phenyl)-4-(2,3-dimethyl-1H-indol-4-yl)-5-mercapto-[1,2,4]triazole |
| 58 | 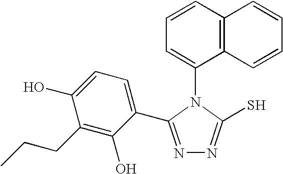 | 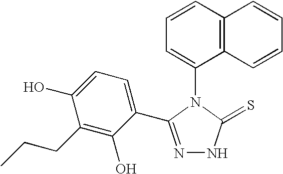 | 3-(2,4-Dihydroxy-3-propyl-phenyl)-4-(naphthalen-1-yl)-5-mercapto-[1,2,4]triazole |
| 59 | 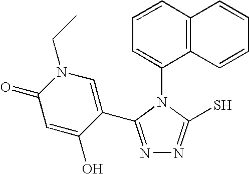 | 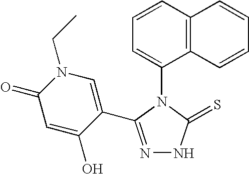 | 3-(1-ethyl-4-hydroxy-6-oxo-1,6-dihydro-pyridin-3-yl)-4-(naphthalen-1-yl)-5-mercapto-[1,2,4]triazole |
| 60 | 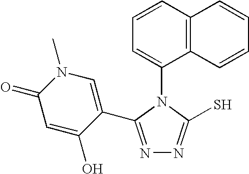 | 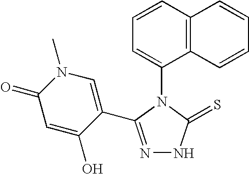 | 3-(4-hydroxy-6-oxo-pyridin-3-yl)-4-(naphthalen-1-yl)-5-mercapto-[1,2,4]triazole |
| 61 | 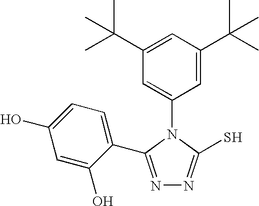 | 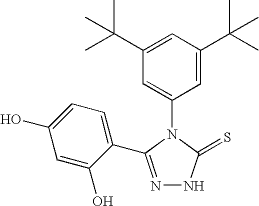 | 3-(2,4-Dihydroxy-phenyl)-4-(3,5-di-tert-butylphenyl)-5-mercapto-[1,2,4]triazole |
| 62 | 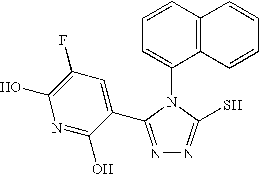 | 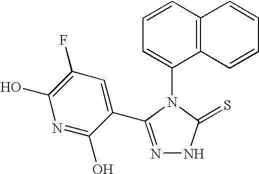 | 3-(2,6-Dihydroxy5-fluoro-pyridin-3-yl) 4-(naphthalen-1-yl)-5-mercapto-[1,2,4]triazole |
| 63 | 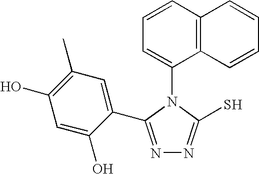 | 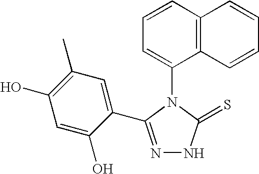 | 3-(2,4-Dihydroxy-5-methyl-phenyl)-4-(naphthalene-1-yl)-5-mercapto-[1,2,4]triazole |
| 64 | 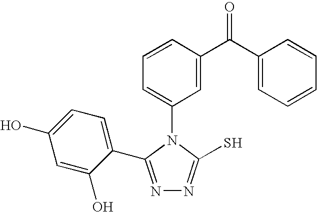 | 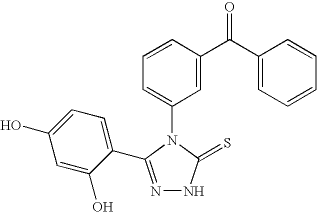 | 3-[2,4-Dihydroxy-phenyl]-4-(3-benzoylphenyl)-5-mercapto-[1,2,4]triazole |
| 65 | 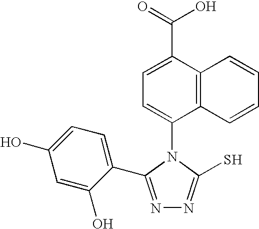 | 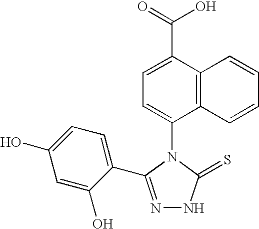 | 3-(2,4-Dihydroxy-phenyl)-4-(4-carboxy-naphthalen-1-yl)-5-mercapto-[1,2,4]triazole |
| 66 | 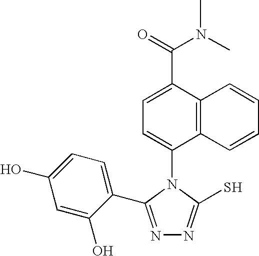 | 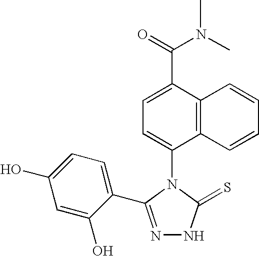 | 3-(2,4-Dihydroxy-phenyl)-4-[4-(N,N-dimethylcarbamoyl)-naphthalen-1-yl]-5-mercapto-[1,2,4]triazole |
| 67 | 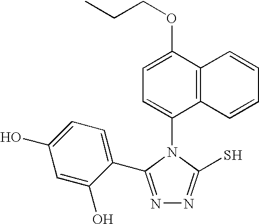 | 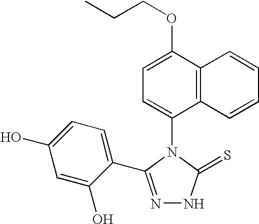 | 3-(2,4-Dihydroxy-phenyl)-4-(4-propoxy-naphthalen-1-yl)-5-mercapto-[1,2,4]triazole |
| 68 | 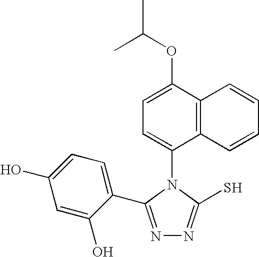 | 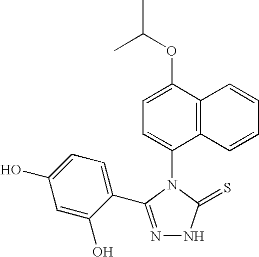 | 3-(2,4-Dihydroxy-phenyl)-4-(4-isopropoxy-naphthalen-1-yl)-5-mercapto-[1,2,4]triazole |
| 69 | 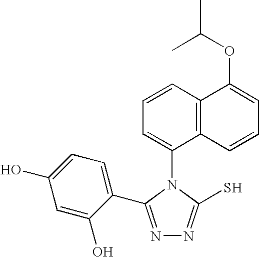 | 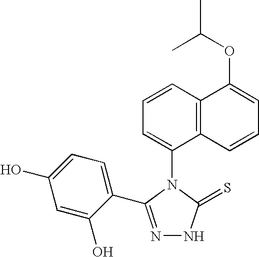 | 3-(2,4-Dihydroxy-phenyl)-4-(5-isopropoxy-naphthalen-1-yl)-5-mercapto-[1,2,4]triazole |
| 70 | 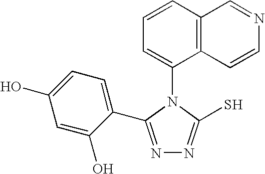 | 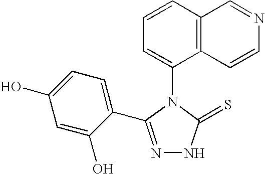 | 3-(2,4-Dihydroxy-phenyl)-4-(isoquinolin-5-yl)-5-mercapto-[1,2,4]triazole |
| 71 | 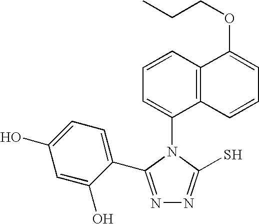 | 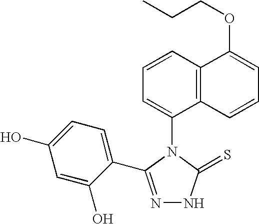 | 3-(2,4-Dihydroxy-phenyl)-4-(5-propoxy-naphthalen-1-yl)-5-mercapto-[1,2,4]triazole |
| 72 | 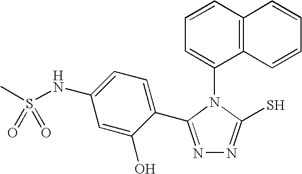 | 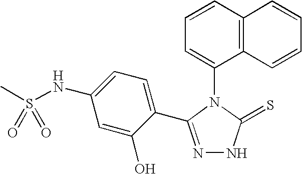 | 3-(2-Hydroxy-4-methanesulfonamino-phenyl)-4-(naphthalen-1-yl)-5-mercapto-[1,2,4]triazole |
| 73 | 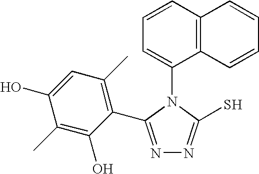 | 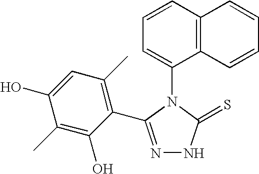 | 3-(2,4-Dihydroxy-3,6-dimethyl-phenyl)-4-(naphthalen-1-yl)-5-mercapto-[1,2,4]triazole |
| 74 | 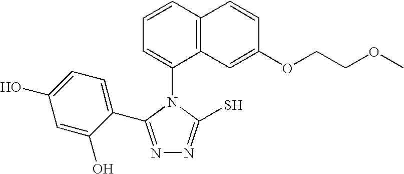 | 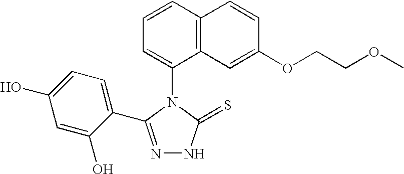 | 3-(2,4-Dihydroxy-phenyl)-4-[7-(2-methoxyethoxy)-naphthalen-1-yl]-5-mercapto-[1,2,4]triazole |
| 75 | 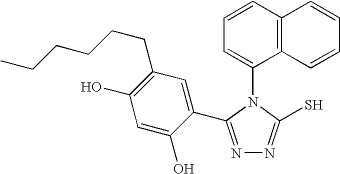 | 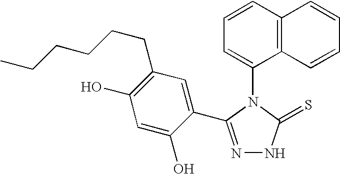 | 3-(2,4-Dihydroxy-5-hexyl-phenyl)-4-(naphthalen-1-yl)-5-mercapto-[1,2,4]triazole |
| 76 | 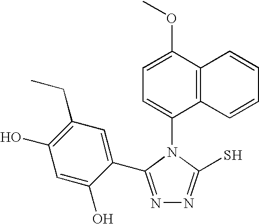 | 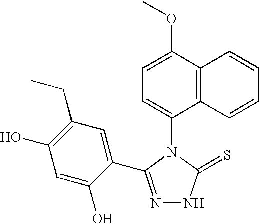 | 3-(2,4-Dihydroxy-5-ethyl-phenyl)-4-(4-methoxy-naphthalen-1-yl)-5-mercapto-[1,2,4]triazole |
| 77 | 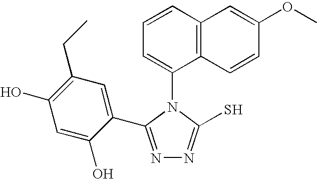 | 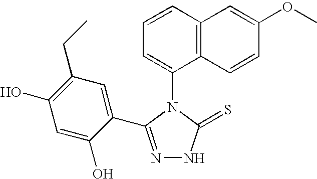 | 3-(2,4-Dihydroxy-5-ethyl-phenyl)-4-(6-methoxy-naphthalin-1-yl)-5-mercapto-[1,2,4]triazole |
| 78 | 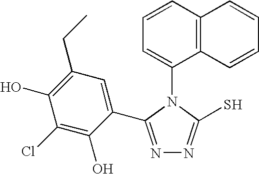 | 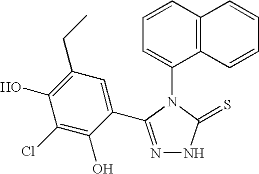 | 3-(2,4-Dihydroxy-3-chloro-5-ethyl-phenyl)-4-(naphthalen-1-yl)-5-mercapto-[1,2,4]triazole |
| 79 | 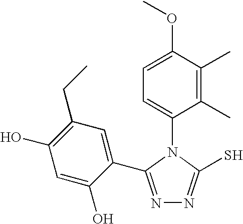 | 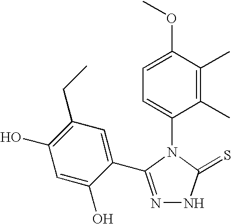 | 3-(2,4-Dihydroxy-5-ethyl-phenyl)-4-(2,3-dimethy-4-methoxy-phenyl)-5-mercapto-[1,2,4) triazole |
| 80 | 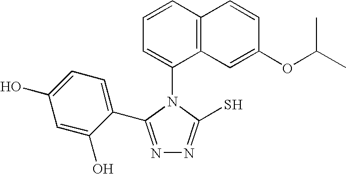 | 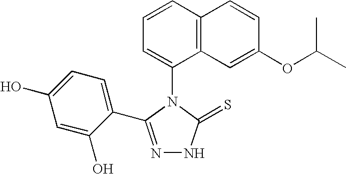 | 3-(2,4-Dihydroxy-phenyl)-4-(7-isopropoxy-naphthalen-1-yl)-5-mercapto-[1,2,4]triazole |
| 81 | 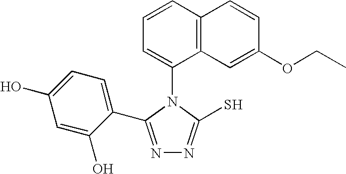 | 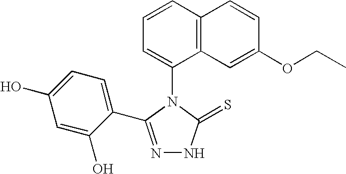 | 3-(2,4-Dihydroxy-phenyl)-4-(7-ethoxy-naphthalen-1-yl)-5-mercapto-[1,2,4]triazole |
| 82 | 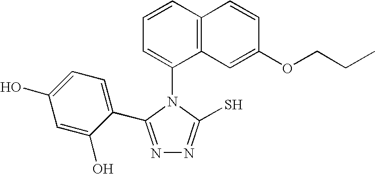 | 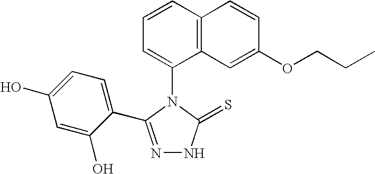 | 3-(2,4-Dihydroxy-phenyl)-4-(7-propoxy-naphthalen-1-yl)-5-mercapto-[1,2,4]triazole |
| 83 | 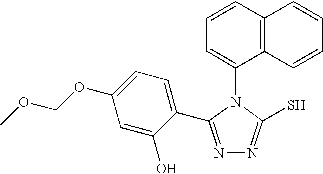 | 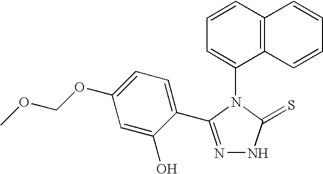 | 3-(2-Hydroxy-4-methoxymethyoxy-phenyl)-4-(naphthalen-1-yl)-5-mercapto-[1,2,4]triazole |
| 84 | 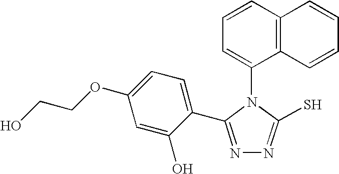 | 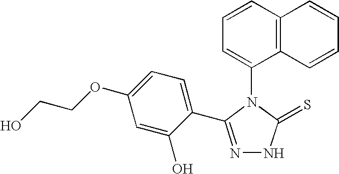 | 3-[2-Hydroxy-4-(2-hydroxy-ethoxy)-phenyl]-4-(naphthalen-1-yl)-5-mercapto-[1,2,4]triazole |
| 85 | 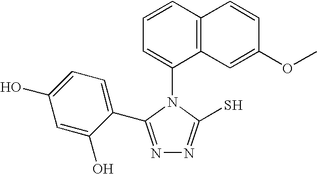 | 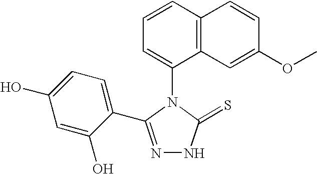 | 3-(2,4-Dihydroxyphenyl)-4-(7-methoxy-naphthalen-1-yl)-5-mercapto-[1,2,4]triazole |
| 86 | 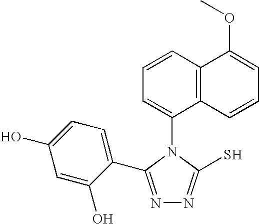 | 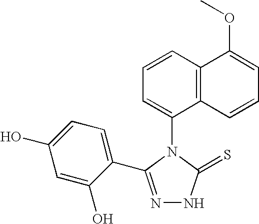 | 3-(2,4-Dihydroxyphenyl)-4-(5-methoxy-naphthalen-1-yl)-5-mercapto-[1,2,4]triazole |
| 87 | 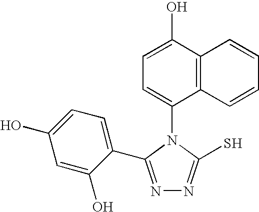 | 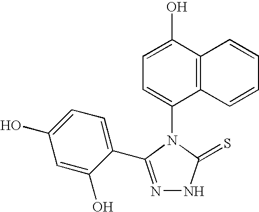 | 3-(2,4-Dihydroxyphenyl)-4-(4-hydroxy-naphthalen-1-yl)-5-mercapto-[1,2,4]triazole |
| 88 | 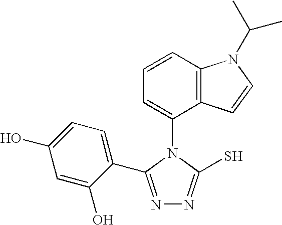 | 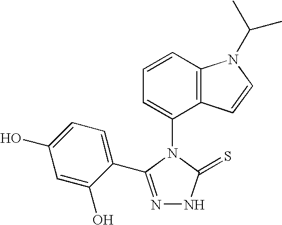 | 3-(2,4-Dihydroxyphenyl)-4-(1-isopropyl-indol-4-yl)-5-mercapto-[1,2,4]triazole |
| 89 | 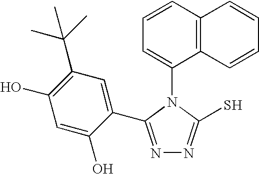 | 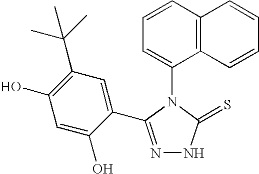 | 3-(2,4-Dihydroxy-5-tert-butyl-phenyl)-4-(naphthalen-1-yl)-5-mercapto-[1,2,4]triazole |
| 90 | 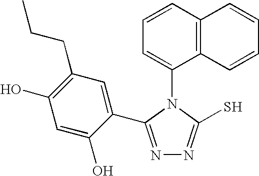 | 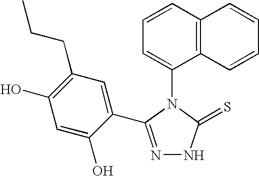 | 3-(2,4-Dihydroxy-5-propyl-phenyl)-4-(naphthalen-1-yl)-5-mercapto-[1,2,4]triazole |
| 91 | 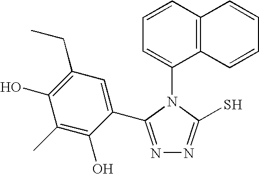 | 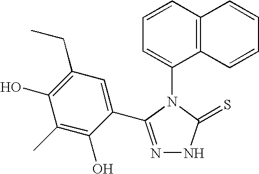 | 3-(2,4-Dihydroxy-3-methyl-5-ethyl-phenyl)-4-(naphthalen-1-yl)-5-mercapto-[1,2,4]triazole |
| 92 | 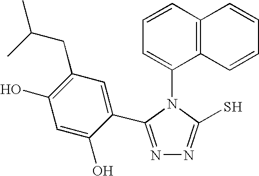 | 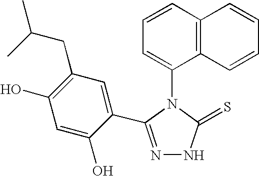 | 3-(2,4-Dihydroxy-5-isobutyl-phenyl)-4-(naphthalen-1-yl)-5-mercapto-[1,2,4]triazole |
| 93 | 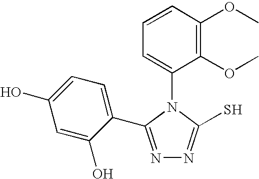 | 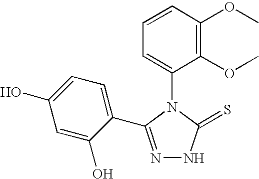 | 3-(2,4-Dihydroxy-phenyl)-4-(2,3-dimethoxy-phenyl)-5-mercapto-[1,2,4]triazole |
| 94 | 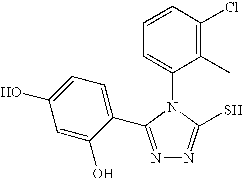 | 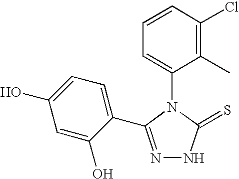 | 3-(2,4-Dihydroxy-phenyl)-4-(2-methoxy-3-chloro-phenyl)-5-mercapto-[1,2,4]triazole |
| 95 | 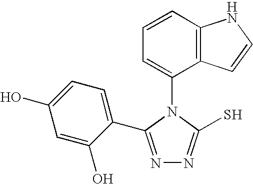 | 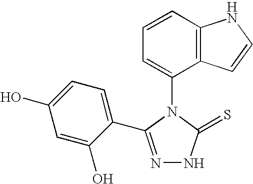 | 3-(2,4-Dihydroxy-phenyl)-4-(indol-4-yl)-5 -mercapto-[1,2,4]triazole |
| 96 | 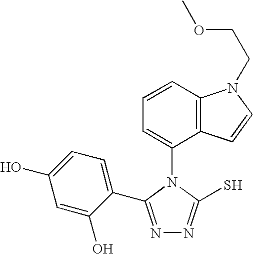 | 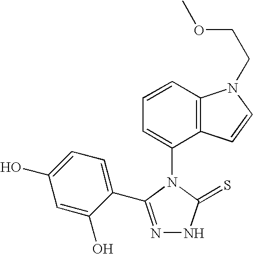 | 3-(2,4-Dihydroxy-phenyl)-4-[1-(2-methoxyethoxy)-indol-4-yl]-5 -mercapto-[1,2,4]triazole |
| 97 | 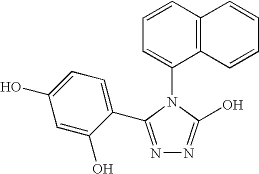 | 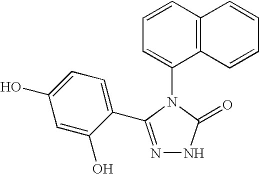 | 3-(2,4-Dihydroxy-phenyl)-4-(naphthalen-1-yl)-5-hydroxy-[1,2,4]triazole |
| 98 | 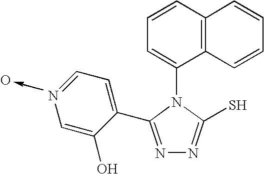 | 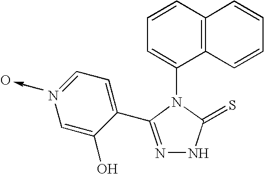 | 3-(1-Oxo-3-hydroxy-pyridin-4-yl)-4-(naphthalen-1-yl)-5-mercapto-[1,2,4]triazole |
| 99 | 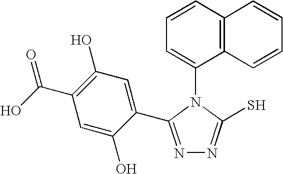 | 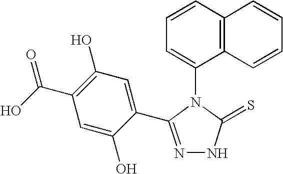 | 3-(2,5-Dihydroxy-4-carboxy)-4-(naphthalen-1-yl)-5-mercapto-[1,2,4]triazole |
| 100 | 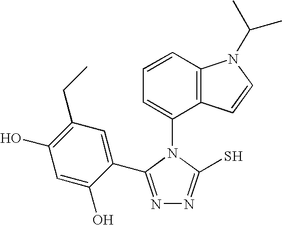 | 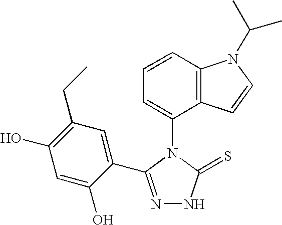 | 3-(2,4-Dihydroxy-5-ethyl-phenyl)-4-(1-isopropyl-indol-4-yl)-5-mercapto-[1,2,4]triazole |
| 101 | 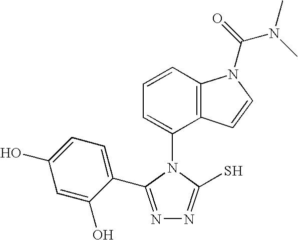 | 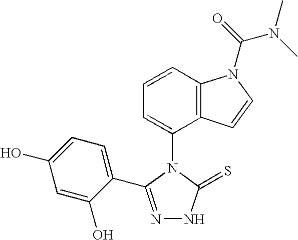 | 3-(2,4-Dihydroxy-5-ethyl-phenyl)-4-[1-(dimethyl-carbamoyl)-indol-4-yl]-5-mercapto-[1,2,4]triazole |
| 102 | 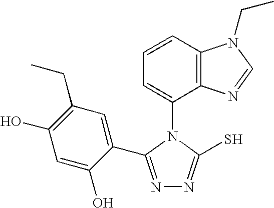 | 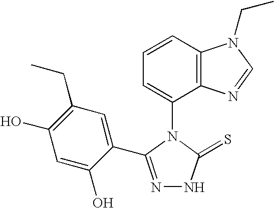 | 3-(2,4-Dihydroxy-5-ethyl-phenyl)-4-(1-ethyl-benzoimidazol-4-yl)-5-mercapto-[1,2,4]triazole |
| 103 | 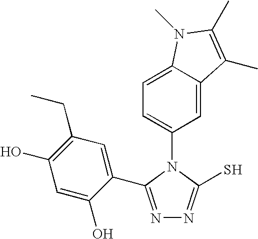 | 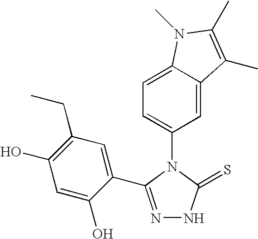 | 3-(2,4-Dihydroxy-5-ethyl-phenyl)-4-(1,2,3-trimethyl-indol-5-yl)-5-mercapto-[1,2,4]triazole |
| 104 | 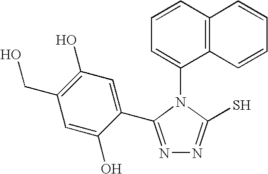 | 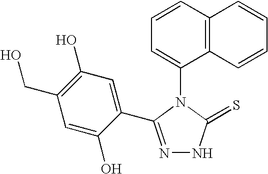 | 3-(2,5-Dihydroxy-4-hydroxymethyl-phenyl)-4-(naphthalen-1-yl)-5-mercapto-[1,2,4]triazole |
| 105 | 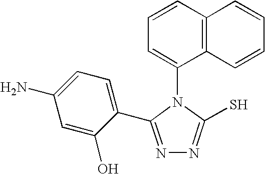 | 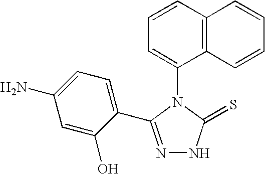 | 3-(2-Hydroxy-4-amino-phenyl)-4-(naphthalen-1-yl)-5-mercapto-[1,2,4]triazole |
| 106 | 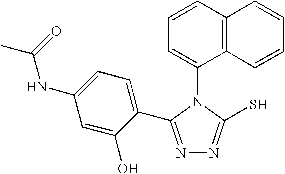 | 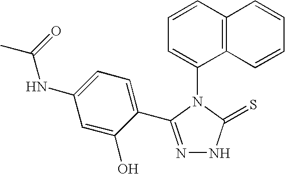 | 3-(2-Hydroxy-4-acetylamino-phenyl)-4-(naphthalen-1-yl)-5-mercapto-[1,2,4]triazole |
| 107 | 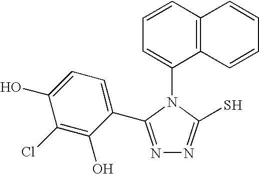 | 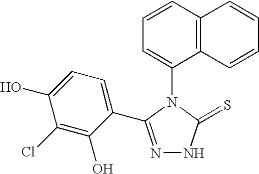 | 3-(2,4-Dihydroxy-3-chloro-phenyl)-4-(naphthalen-1-yl)-5-mercapto-[1,2,4]triazole |
| 108 | 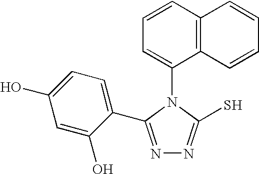 | 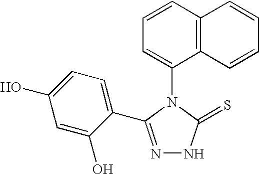 | 3-(2,4-Dihydroxy-phenyl)-4-(naphthalen-1-yl)-5-mercapto-[1,2,4]triazole |
| 109 | 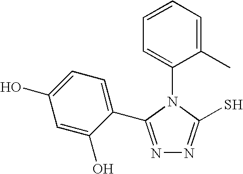 | 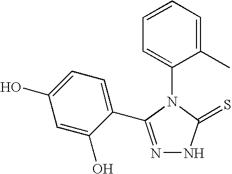 | 3-(2,4-Dihydroxy-phenyl)-4-(2-methyl-phenyl)-5-mercapto-[1,2,4]triazole |
| 110 | 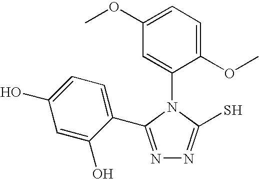 | 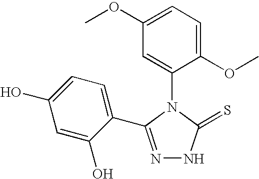 | 3-(2,4-Dihydroxy-phenyl)-4-(2,5-dimethoxy-phenyl)-5-mercapto-[1,2,4]triazole |
| 111 | 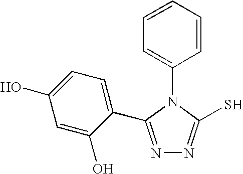 | 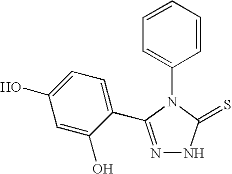 | 3-(2,4-Dihydroxy-phenyl)-4-phenyl-5-mercapto-[1,2,4]triazole |
| 112 | 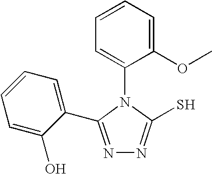 | 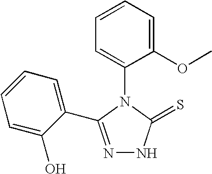 | 3-(2-Hydroxy-phenyl)-4-(2-methoxy-phenyl)-5-mercapto-[1,2,4]triazole |
| 113 | 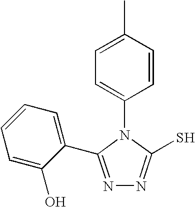 | 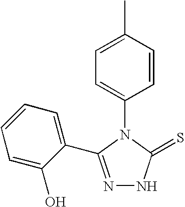 | 3-(2-Hydroxy-phenyl)-4-(4-methyl-phenyl)-5-mercapto-[1,2,4]triazole |
| 114 | 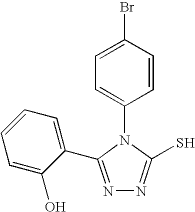 | 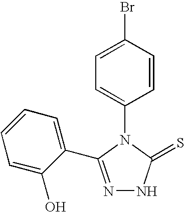 | 3-(2-Hydroxy-phenyl)-4-(4-bromo-phenyl)-5-mercapto-[1,2,4]triazole |
| 115 | 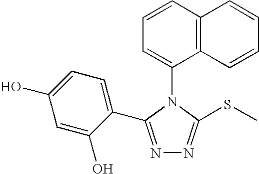 | | 3-(2,4-Dihydroxy-phenyl)-4-(naphthalen-1-yl)-5-(methyl sulfanyl)-[1,2,4]triazole |
| 116 | 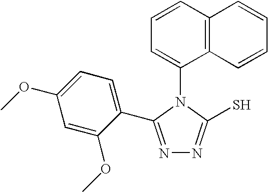 | 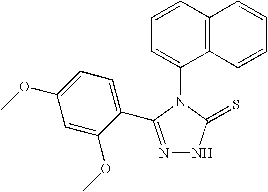 | 3-(2,4-Dimethoxy-phenyl)-4-(naphthalen-1-yl)-5-mercapto-[1,2,4]triazole |
| 117 | 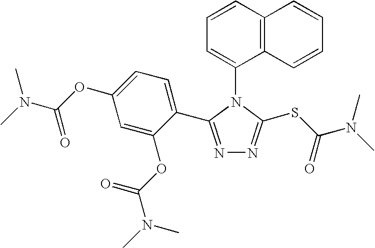 | | 3-[2,4-Di-(dimethyl-carbamoyloxy)-phenyl]-4-(naphthalen-1-yl)-5-(dimethyl-carbamoylsulfanyl)-[1,2,4]triazole |
| 118 | 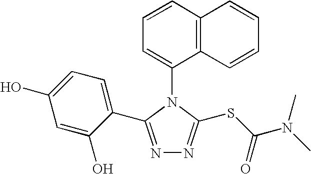 | | 3-(2,4-Dihydroxy-phenyl)-4-(naphthalen-1-yl)-5-(dimethylcarbamoylsulfanyl)-[1,2,4]triazole |
| 119 | 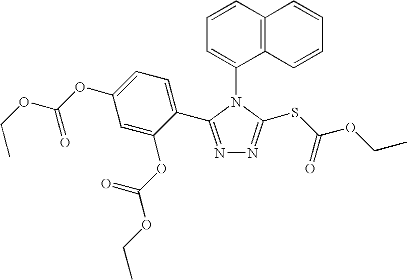 | | 3-(2,4-Diethoxycarbonyloxy-phenyl)-4-(naphthalen-1-yl)-5-(ethoxycarbonylsulfanyl)-[1,2,4]triazole |
| 120 | 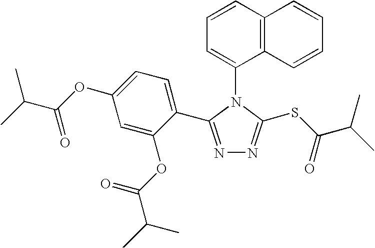 | | 3-(2,4-Di-isobutyryloxy-phenyl)-4-(naphthalen-1-yl)-5-(isobutyrylsulfanyl)-[1,2,4]triazole |
| 121 | 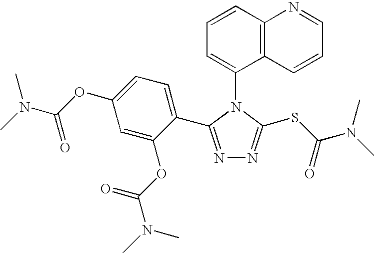 | | 3-[2,4-Di-(dimethyl-carbamoyloxy)-phenyl]-4-(quinolin-5-yl)-5-(dimethyl-carbamoylsulfanyl)-[1,2,4]triazole |
| 122 | 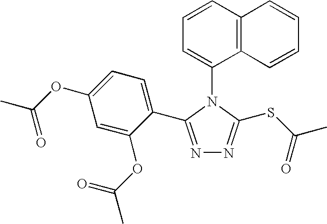 | | 3-(2,4-Diacetoxy-phenyl)-4-(naphthalen-1-yl)-5-(acetylsulfanyl)-[1,2,4]triazole |
| 123 | 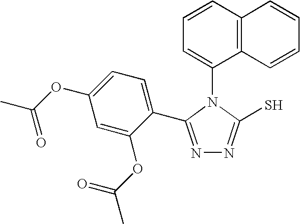 | 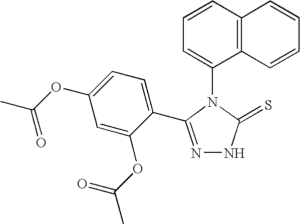 | 3-(2,4-Diacetoxy-phenyl)-4-(naphthalen-1-yl)-5-mercapto-[1,2,4]triazole |
| 124 | 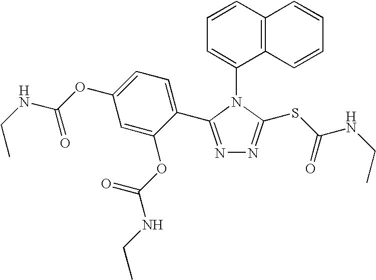 | | 3-(2,4-Diethylcarbamoyloxy-phenyl)-4-(naphthalen-1-yl)-5-(ethylcarbamoylsulfanyl)-[1,2,4]triazole |
| 125 | 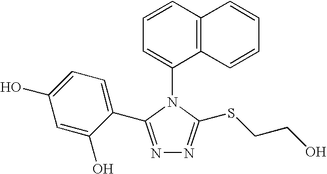 | | 3-(2,4-Dihydroxy-phenyl)-4-(naphthalen-1-yl)-5-(2-hydroxyethylsulfanyl)-[1,2,4]triazole |
| 126 | 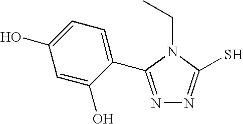 | 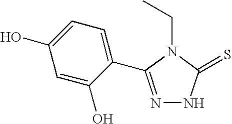 | 3-(2,4-Dihydroxy-phenyl)-4-ethyl-5-mercapto-[1,2,4]triazole |
| 127 | 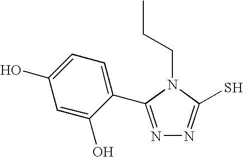 | 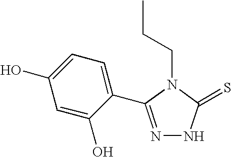 | 3-(2,4-Dihydroxy-phenyl)-4-propyl-5-mercapto-[1,2,4]triazole |
| 128 | 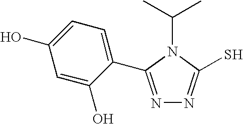 | 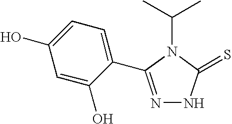 | 3-(2,4-Dihydroxy-phenyl)-4-isopropyl-5-mercapto-[1,2,4]triazole |
| 129 | 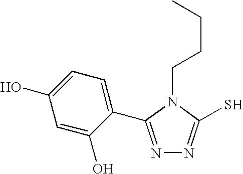 | 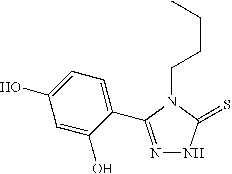 | 3-(2,4-Dihydroxy-phenyl)-4-butyl-5-mercapto-[1,2,4]triazole |
| 130 | 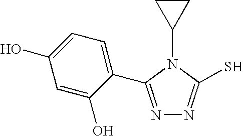 | 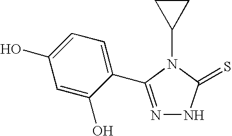 | 3-(2,4-Dihydroxy-phenyl)-4-cyclopropyl-5-mercapto-[1,2,4]triazole |
| 131 | 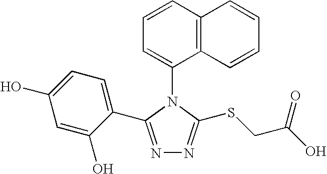 | | 3-(2,4-Dihydroxy-phenyl)-4-(naphthalen-1-yl)-5-(carboxyethysulfanyl)-[1,2,4]triazole |
| 132 | 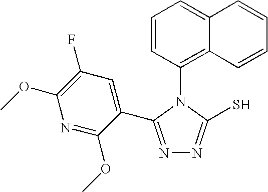 | 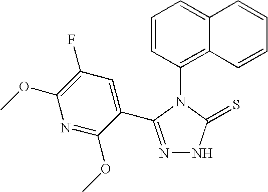 | 3-(2,6-Dimethoxy-5-fluoro-pyridin-3-yl)-4-(naphthalen-1-yl)-5-mercapto-[1,2,4]triazole |
| 133 | 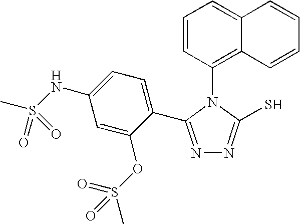 | 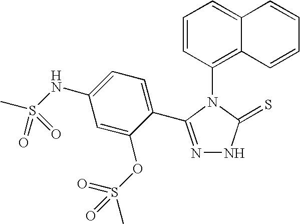 | 3-(2-Methanesulfonyloxy-4-methanesulfonylamino-phenyl)-4-(naphthalen-1-yl)-5-mercapto-[1,2,4]triazole |
| 134 | 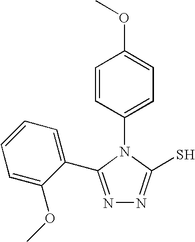 | 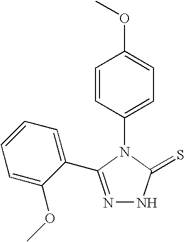 | 3-(2-Methoxy-phenyl)-4-(4-methoxy-phenyl)-5-mercapto-[1,2,4]triazole |
| 135 | 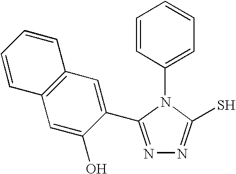 | 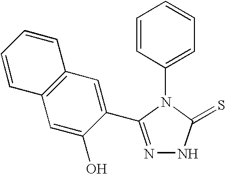 | 3-(3-Hydroxy-naphthalen-2-yl)-4-phenyl-5-mercapto-[1,2,4]triazole |
| 136 | 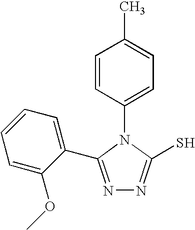 | 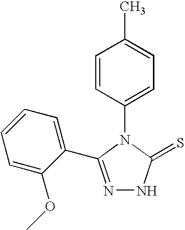 | 3-(2-Methoxy-phenyl)-4-(4-methyl-phenyl)-5-mercapto-[1,2,4]triazole |
| 137 | 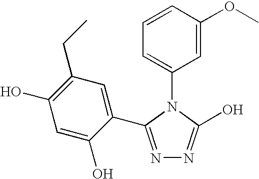 | 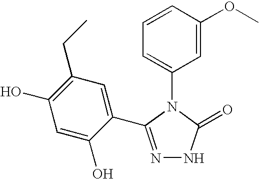 | 3-(2,4-Dihydroxy-5-ethyl-phenyl)-4-(3-methox-phenyl)-5-hydroxy-[1,2,4]triazole |
| 138 | 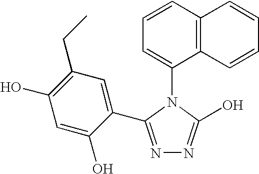 | 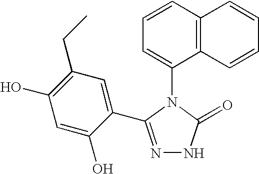 | 3-(2,4-Dihydroxy-5-ethyl-phenyl)-4-(naphthalen-1-yl)-5-hydroxy-[1,2,4]triazole |
| 139 | 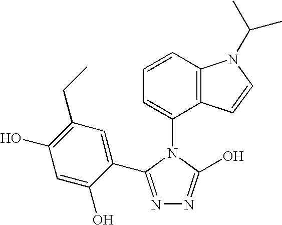 | 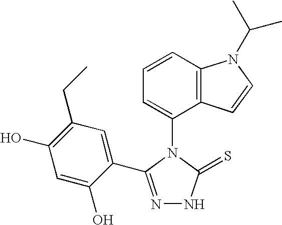 | 3-(2,4-Dihydroxy-5-ethyl-phenyl)-4-(1-isopropyl-indol-3-yl)-5-hydroxy-[1,2,4]triazole |
| 140 | 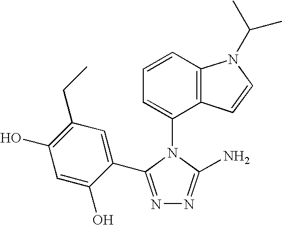 | 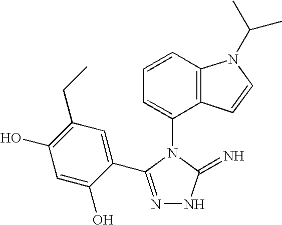 | 2,4-Dihydroxy-5-ethyl-phenyl)-4-(1-isopropyl-indol-4-yl)-5-amino-[1,2,4]triazole |
| 141 | 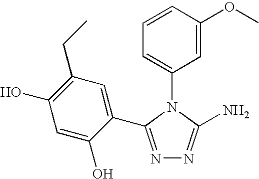 | 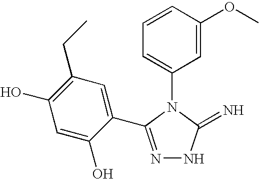 | 3-(2,4-Dihydroxy-5-ethyl-phenyl)-4-(3-methoxy-phenyl)-5-amino-[1,2,4]triazole |
| 142 | 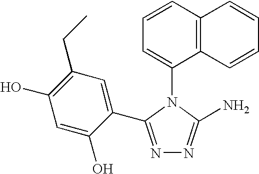 | 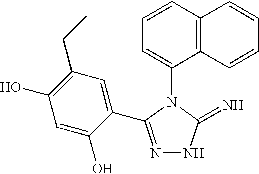 | 3-(2,4-Dihydroxy-5-ethyl-phenyl)-4-(naphthalen-1-yl)-5-amino-[1,2,4]triazole |
| 143 | 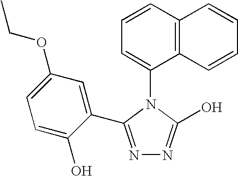 | 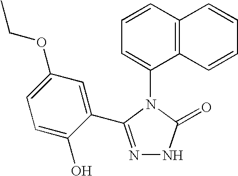 | 3-(2-Hydroxy-5-ethyloxy-phenyl)-4-(naphthalen-1-yl)-5-hydroxy-[1,2,4]triazole |
| 144 | 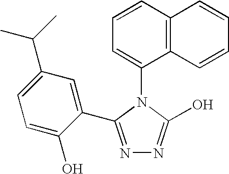 | 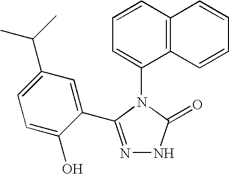 | 3-(2-Hydroxy-5-isopropyl-phenyl)-4-(naphthalen-1-yl)-5-hydroxy-[1,2,4]triazole |
| 145 | 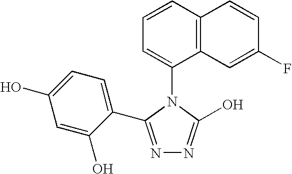 | 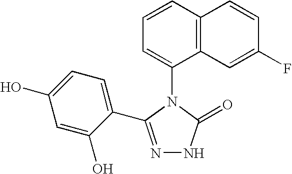 | 3-(2-Dihydroxy-phenyl)-4-(7-fluoro-naphthalen-1-yl)-5-hydroxy-[1,2,4]triazole |
| 146 | 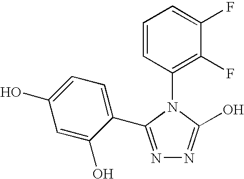 | 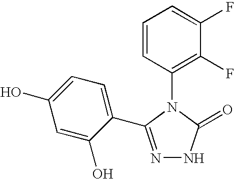 | 3-(2,4-Dihydroxy-phenyl)-4-(2,3-difluorophenyl)-5-hydroxy-[1,2,4]triazole |
| 147 | 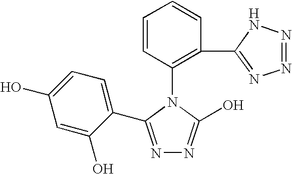 | 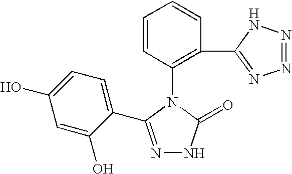 | 3-(2,4-Dihydroxy-phenyl)-4-[2-(1H-tetrazol-5-yl)-phenyl]-5-hydroxy-[1,2,4]triazole |
| 148 | 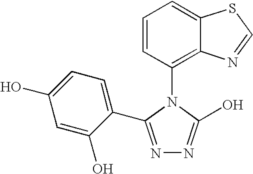 | 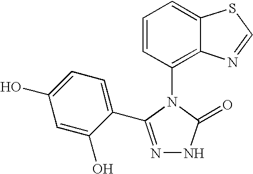 | 3-(2,4-Dihydroxy-phenyl)-4-(benzothiazol-4-yl)-5-hydroxy-[1,2,4]triazole |
| 149 | 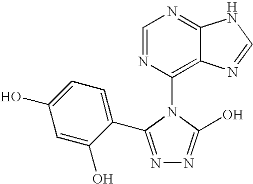 | 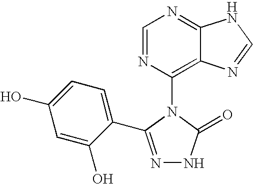 | 3-(2,4-Dihydroxy-phenyl)-4-(9H-purin-6-yl)-5-hydroxy-[1,2,4]triazole |
| 150 | 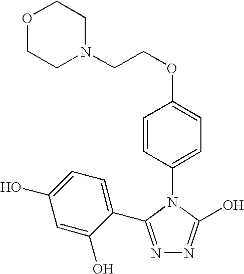 | 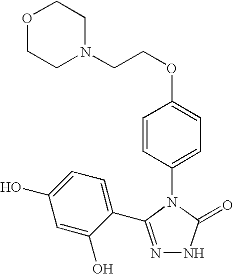 | 3-(2,4-Dihydroxy-phenyl)-4-{4-[2-(moropholin-1-yl)-ethoxyl]-phenyl}-5-hydroxy-[1,2,4]triazole |
| 151 | 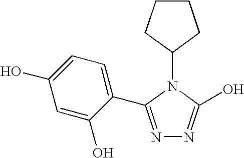 | 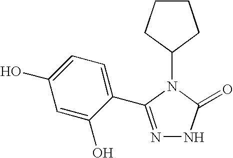 | 3-(2,4-Dihydroxy-phenyl)-4-cyclopentyl-5-hydroxy-[1,2,4]triazole |
| 152 | 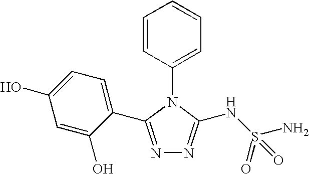 | | 3-(2,4-Dihydroxy-phenyl)-4-phenyl-5-(sulfamoylamino)-[1,2,4]triazole |
| 153 | 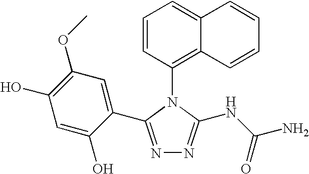 | | 3-(2,4-Dihydroxy-5-methoxy-phenyl)-4-(naphthalene-1-yl)-5-ureido-[1,2,4]triazole |
| 154 | 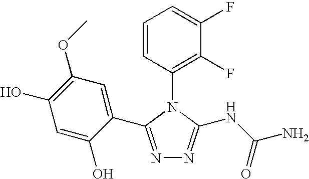 | | 3-(2,4-Dihydroxy-5-methoxy-phenyl)-4-(2,3-difluorophenyl)-5-ureido-[1,2,4]triazole |
| 155 | 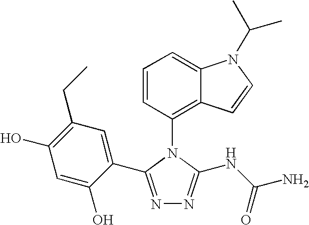 | | 3-(2,4-Dihydroxy-5-ethyl-phenyl)-4-(1-isopropyl-indol-4-yl)-5-ureido-[1,2,4]triazole |
| 156 | 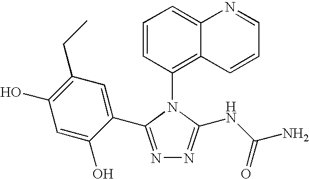 | | 3-(2,4-Dihydroxy-5-ethyl-phenyl)-4-(quinolin-5-yl)-5-ureido-[1,2,4]triazole |
| 157 | 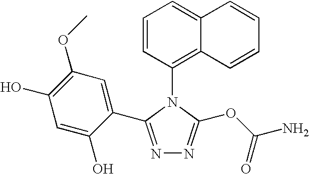 | | 3-(2,4-Dihydroxy-5-methoxy-phenyl)-4-(naphthalene-1-yl)-5-carbamoyloxy-[1,2,4]triazole |
| 158 | 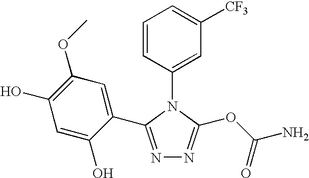 | | 3-(2,4-Dihydroxy-5-ethyl-phenyl)-4-(3-trifluoromethyl-phenyl)-5-carbamoyloxy-[1,2,4]triazole |
| 159 | 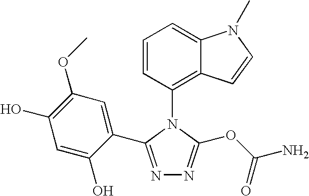 | | 3-(2,4-Dihydroxy-5-ethyl-phenyl)-4-(1-methyl-indol-4-yl)-5-carbamoyloxy-[1,2,4]triazole |
| 160 | 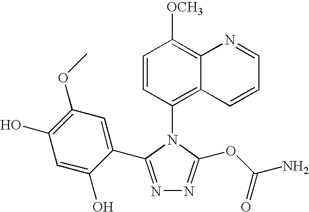 | | 3-(2,4-Dihydroxy-5-methoxy-phenyl)-4-(8-methoxy-quinolin-5 -yl)-5-carbamoyloxy-[1,2,4]triazole |
| 161 | 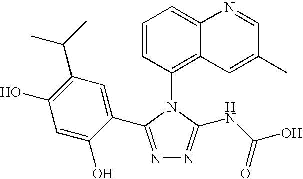 | | 3-(2,4-Dihydroxy-5-isopropyl-phenyl)-4-(3-methyl-quinolin-5-yl)-5-carboxyamino-[1,2,4]triazole |
| 162 | 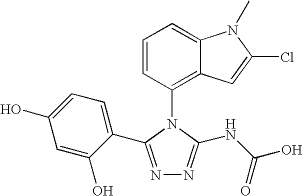 | | 3-(2,4-Dihydroxy-phenyl)-4-(1-methyl-2-chloro-indol-4-yl)-5-carbamoyloxy-[1,2,4]triazole |
| 163 | 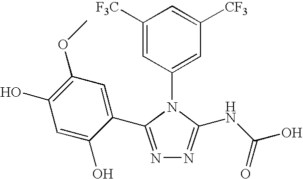 | | 3-(2,4-Dihydroxy-5-methoxy-phenyl)-4-[3,5-di-(trifluoromethyl)-phenyl]-5-carbamoyloxy-[1,2,4]triazole |
| 164 | 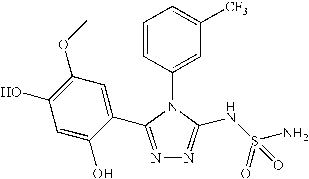 | | 3-(2,4-Dihydroxy-5-methoxy-phenyl)-4-(3-trifluoromethyl-phenyl)-5-(sulfamoylamino)-[1,2,4]triazole |
| 165 | 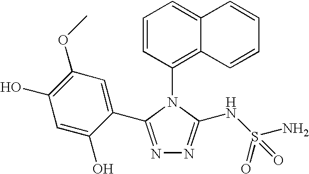 | | 3-(2,4-Dihydroxy-5-methoxy-phenyl)-4-(naphthalene-1-yl)-5-(sulfamoylamino)-[1,2,4]triazole |
| 166 | 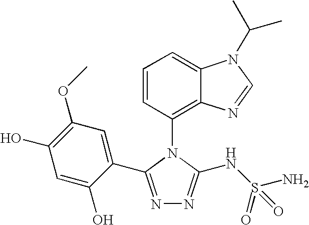 | | 3-(2,4-Dihydroxy-5-methoxy-phenyl)-4-(1-isopropyl-benzoimidazol-4-yl)-5-(sulfamoylamino)-[1,2,4]triazole |
| 167 | 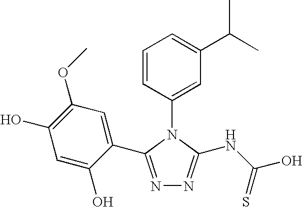 | | 3-(2,4-Dihydroxy-5-methoxy-phenyl)-4-(3-isopropylphenyl)-5-(thiocarboxyamino)-[1,2,4]triazole |
| 168 | 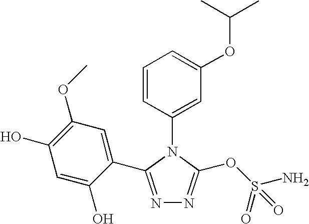 | | 3-(2,4-Dihydroxy-5-methoxy-phenyl)-4-(3-isopropyloxy-phenyl)-5-(sulfamoyloxy)-[1,2,4]triazole |
| 169 | 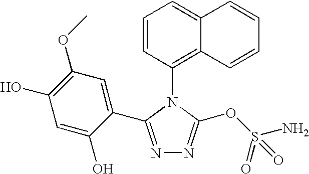 | | 3-(2,4-Dihydroxy-5-methoxy-phenyl)-4-(naphthalene-1-yl)-5-(sulfamoyloxy)-[1,2,4]triazole |
| 170 | 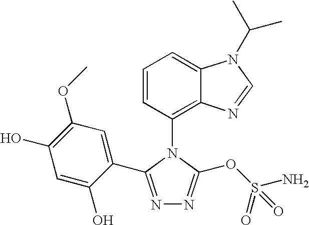 | | 3-(2,4-Dihydroxy-5-methoxy-phenyl)-4-(1-isopropyl-benzoimidazol-4-yl)-5-(sulfamoyloxy)-[1,2,4]triazole |
| 171 | 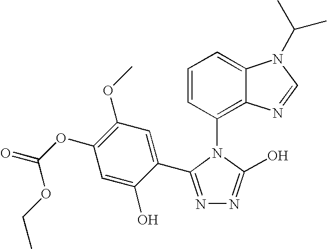 | 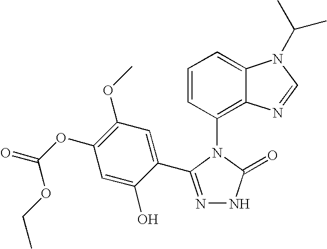 | 3-(2-Hydroxy-4-ethoxycarbonyoxy-5-methoxy-phenyl)-4-(1-isopropyl-benzoimidazol-4-yl)-5-hydroxy-[1,2,4]triazole |
| 172 | 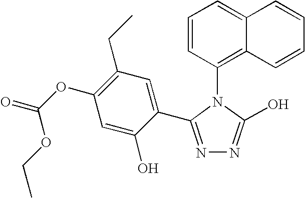 | 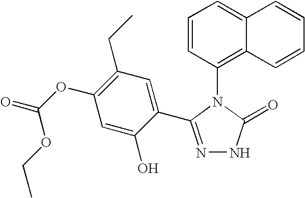 | 3-(2-Hydroxy-4-ethoxycarbonyoxy-5-ethyl-phenyl)-4-(naphthalin-2-yl)-5-hydroxy-[1,2,4]triazole |
| 173 | 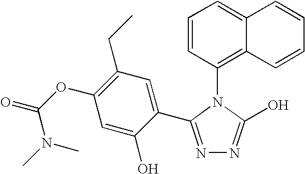 | 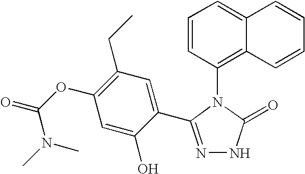 | 3-[2-Hydroxy-4-(dimethyl-carbamoyoxy)-5-ethyl-phenyl]-4-(naphthalin-2-yl)-5-hydroxy-[1,2,4]triazole |
| 174 | 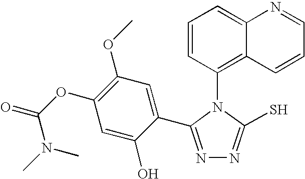 | 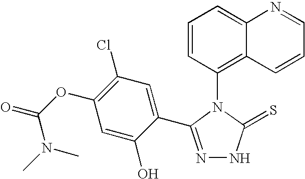 | 3-[2-Hydroxy-4-(dimethyl-carbamoyoxy)-5-chloro-phenyl]-4-(quinolin-5-yl)-5-mercapto-[1,2,4]triazole |
| 175 | 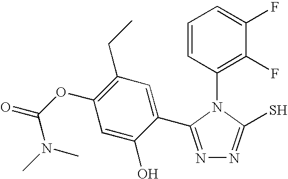 | 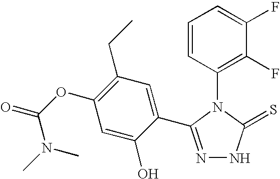 | 3-[2-Hydroxy-4-(dimethyl-carbamoyoxy)-5-ethyl-phenyl]-4-(2,3-difluoro-phenyl)-5-mercapto-[1,2,4]triazole |
| 176 | 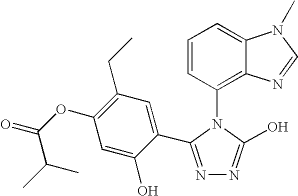 | 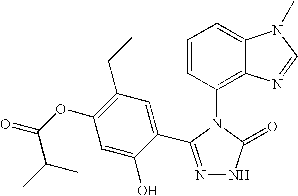 | 3-[2-Hydroxy-4-isobutyryloxy-5-ethyl-phenyl]-4-(1-methyl-benzo-imidazol-4-yl)-5-hydroxy-[1,2,4]triazole |
| 177 | 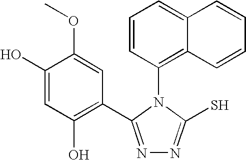 | 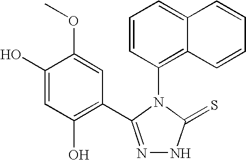 | 3-(2,4-Dihydroxy-5-methoxy-phenyl)-4-(naphthalen-1-yl)-5-mercapto-[1,2,4]triazole |
| 178 | 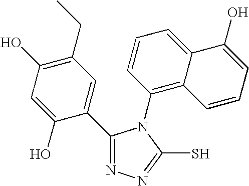 | 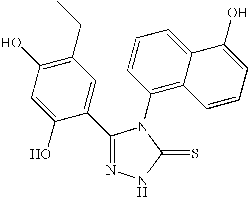 | 3-(2,4-Dihydroxy-5-ethyl-phenyl)-4-(5-hydroxy-naphthalen-1-yl)-5-mercapto-[1,2,4]triazole |
| 179 | 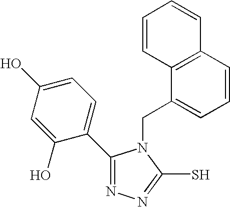 | 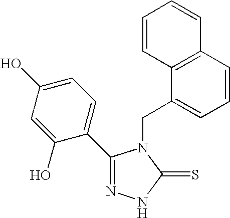 | 3-(2,4-Dihydroxy-phenyl)-4-(naphthalen-1-ylmethyl)-5-mercapto-[1,2,4]triazole |
| 180 | 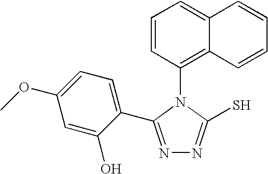 | 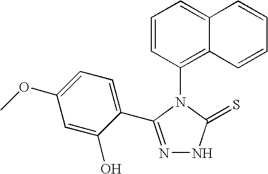 | 3-(2-Hydroxy-4-methoxyphenyl)-4-(naphthalen-1-yl)-5-mercapto-[1,2,4]triazole |
| 181 | 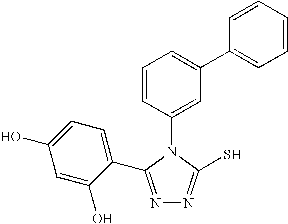 | 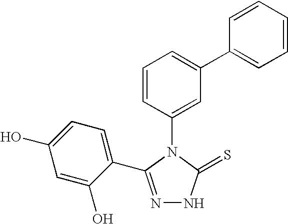 | 3-(2,4-Dihydroxy-phenyl)-4-(biphenyl-3-yl)-5-mercapto-[1,2,4]triazole |
| 182 | 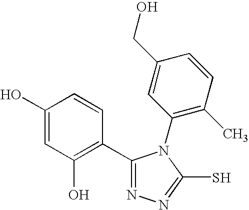 | 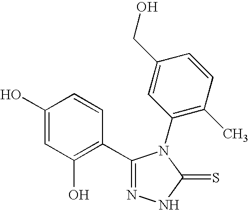 | 3-(2,4-Dihydroxy-phenyl)-4-(2-methyl-5-hydroxymethyl-phenyl)-5-mercapto-[1,2,4]triazole |
| 183 | 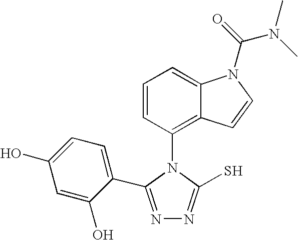 | 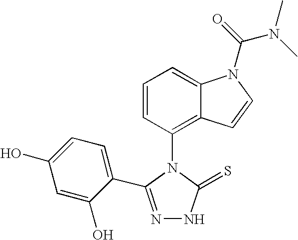 | 3-(2,4-Dihydroxy-phenyl)-4-(1-dimethylcarbamoyl-indol-4-yl)-5-mercapto-[1,2,4]triazole |
| 184 | 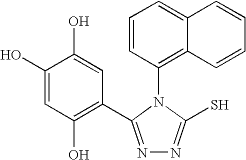 | 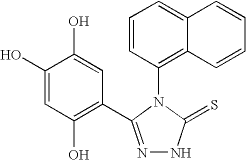 | 3-(2,4,5-Trihydroxy-phenyl)-4-(naphthalene-1-yl)-5-mercapto-[1,2,4]triazole |
| 185 | 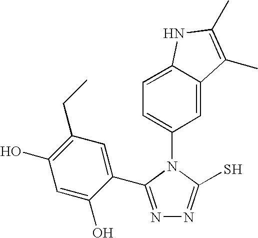 | 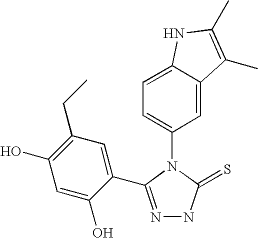 | 3-(2,4-Dihydroxy-5-ethyl-phenyl)-4-(2,3-dimethyl-indol-5-yl)-5-mercapto-[1,2,4]triazole |
| 186 | 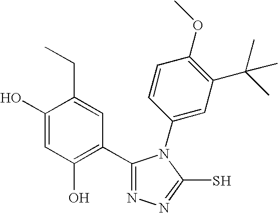 | 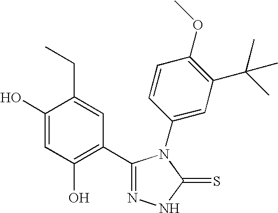 | 3-(2,4-Dihydroxy-5-ethyl-phenyl)-4-(3-t-butyl-4-methoxy-phenyl)-5-mercapto-[1,2,4]triazole |
| 187 | 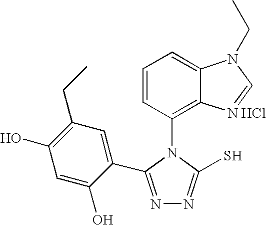 | 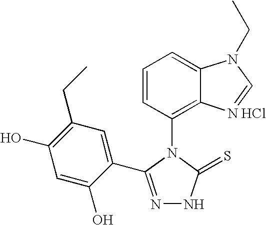 | 3-(2,4-Dihydroxy-5-ethyl-phenyl)-4-(1-ethyl-1H-benzoimidazol-4-yl)-5-mercapto-[1,2,4]triazole,HCl salt |
| 188 | 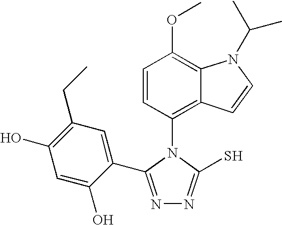 | 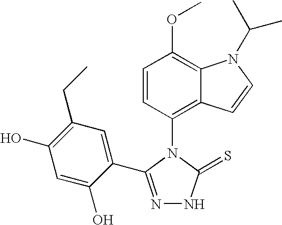 | 3-(2,4-Dihydroxy-5-ethyl-phenyl)-4-(1-isopropyl-7-methoxy-indol-4-yl)-5-mercapto-[1,2,4]triazole |
| 189 | 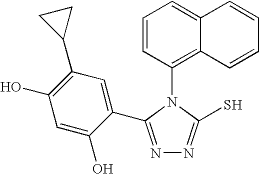 | 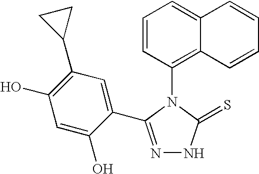 | 3-(2,4-Dihydroxy-5-cyclopropyl-phenyl)-4-(naphthalene-1-yl)-5-mercapto-[1,2,4]triazole |
| 190 | 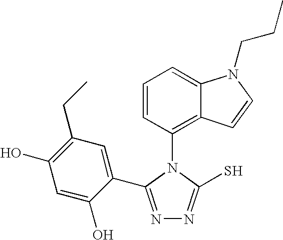 | 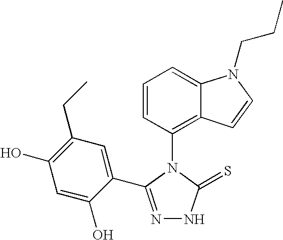 | 3-(2,4-dihydroxy-5-ethyl-phenyl)-4-(1-propyl-indol-4-yl)-5-mercapto-[1,2,4]triazole |
| 191 | 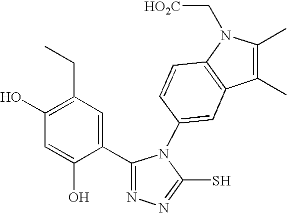 | 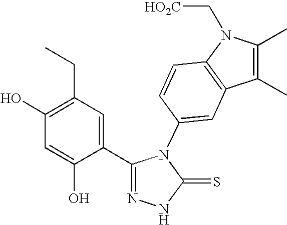 | 3-(2,4-dihydroxy-5-ethyl-phenyl)-4-(1-acetyl-2,3-dimethyl-indol-5-yl)-5-mercapto-[1,2,4]triazole |
| 192 | 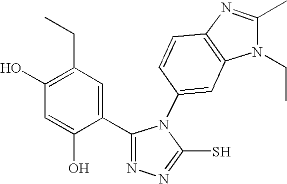 | 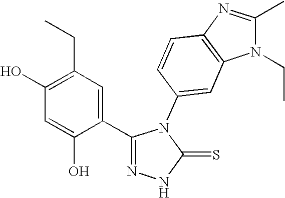 | 3-(2,4-dihydroxy-5-ethyl-phenyl)-4-(2-methyl-3-ethyl-benzimidazol-5-yl)-5-mercapto-[1,2,4]triazole |
| 193 | 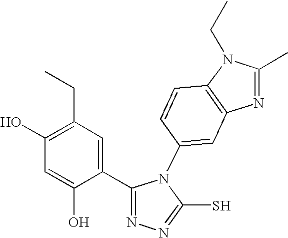 | 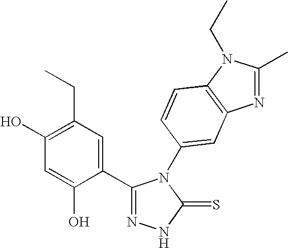 | 3-(2,4-dihydroxy-5-ethyl-phenyl)-4-(1-ethyl-2-methyl-benzimidazol-5-yl)-5-mercapto-[1,2,4]triazole |
| 194 | 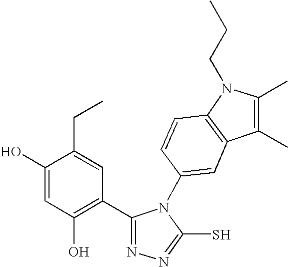 | 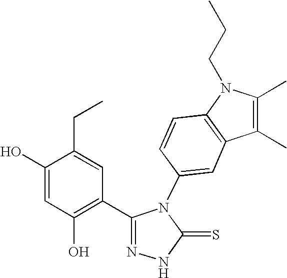 | 3-(2,4-dihydroxy-5-ethyl-phenyl)-4-(1-propyl-2,3-dimethyl-indol-5-yl)-5-mercapto-[1,2,4]triazole |
| 195 | 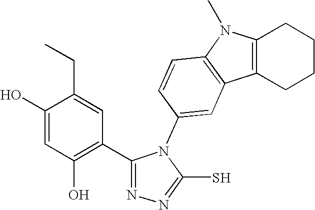 | 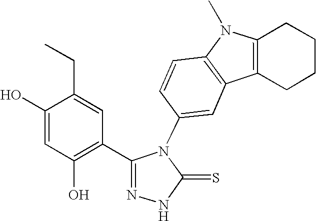 | 3-(2,4-dihydroxy-5-ethyl-phenyl)-4-(N-methyl-tetrahydrocarbozol-7-yl)-5-mercapto-[1,2,4]triazole |
| 196 | 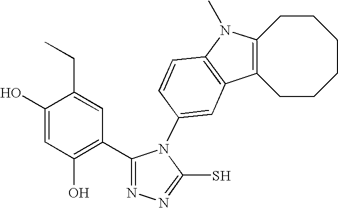 | 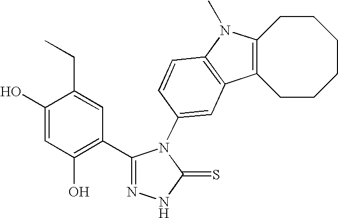 | 3-(2,4-dihydroxy-5-ethyl-phenyl)-4-(N-methyl-cyclononan[a]indol-5-yl)-5-mercapto-[1,2,4]triazole |
| 197 | 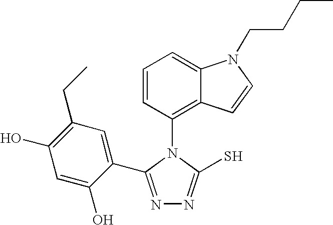 | 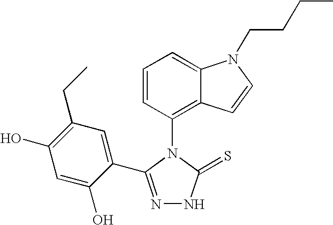 | 3-(2,4-dihydroxy-5-ethyl-phenyl)-4-(1-n-butyl-indol-4-yl)-5-mercapto-[1,2,4]triazole |
| 198 | 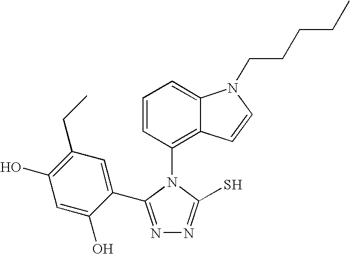 | 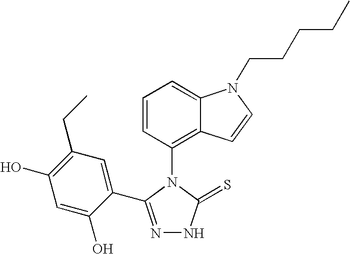 | 3-(2,4-dihydroxy-5-ethyl-phenyl)-4-(1-n-pentyl-indol-4-yl)-5-mercapto-[1,2,4]triazole |
| 199 | 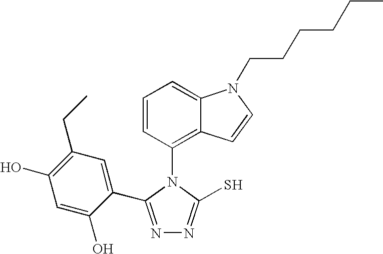 | 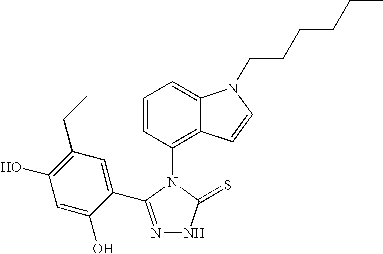 | 3-(2,4-dihydroxy-5-ethyl-phenyl)-4-(1-n-hexyl-indol-4-yl)-5-mercapto-[1,2,4]triazole |
| 200 | 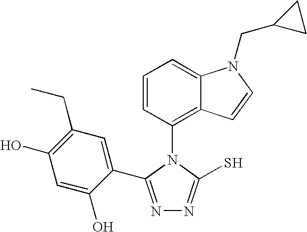 | 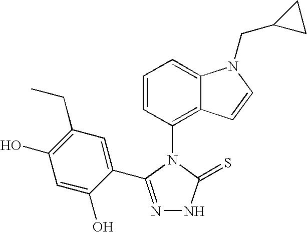 | 3-(2,4-dihydroxy-5-cyclopropyl-phenyl)-4-(1-(1-methylcyclopropyl)-indol-4-yl)-5-mercapto-[1,2,4]triazole |
| 201 | 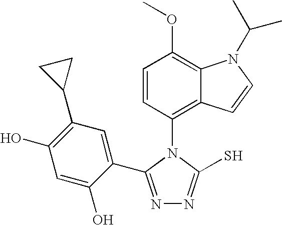 | 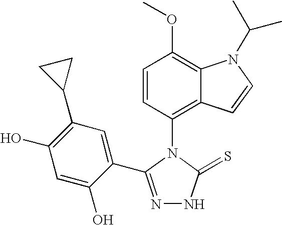 | 3-(2,4-dihydroxy-5-cyclopropyl-phenyl)-4-(1-isopropyl-7-methoxy-indol-4-yl)-5-mercapto-[1,2,4]triazole |
| 202 | 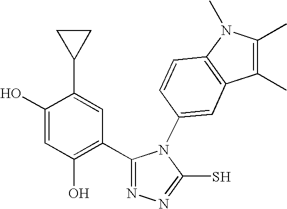 | 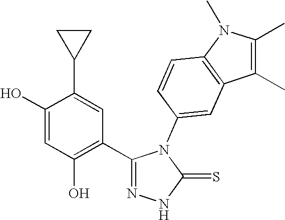 | 3-(2,4-dihydroxy-5-cyclopropyl-phenyl)-4-(1,2,3-trimethyl-indol-5-yl)-5-mercapto-[1,2,4]triazole |
| 203 | 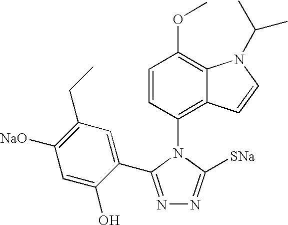 | | 3-(2,4-dihydroxy-5-ethyl-phenyl)-4-(1-isopropyl-7-methoxy-indol-4-yl)-5-mercapto-[1,2,4]triazoledisodium salt |
| 204 | 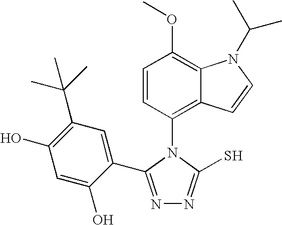 | 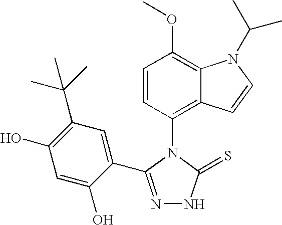 | 3-(2,4-dihydroxy-5-tert-butyl-phenyl)-4-(1-isopropyl-7-methoxy-indol-4-yl)-5-mercapto-[1,2,4]triazole |
| 205 | 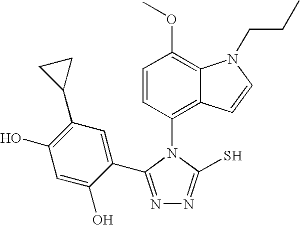 | 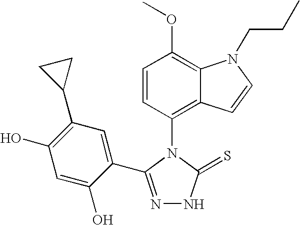 | 3-(2,4-dihydroxy-5-cyclopropyl-phenyl)-4-(1-propyl-7-methoxy-indol-4-yl)-5-mercapto-[1,2,4]triazole |
| 206 | 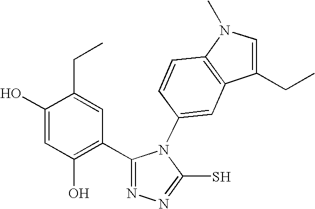 | 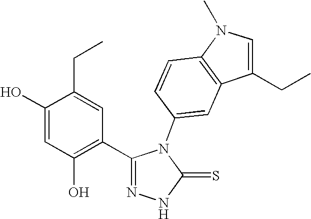 | 3-(2,4-dihydroxy-5-ethyl-phenyl)-4-(1-methyl-3-ethyl-indol-5-yl)-5-mercapto-[1,2,4]triazole |
| 207 | 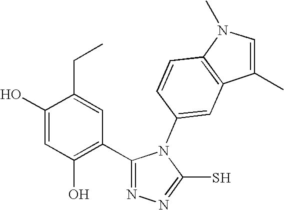 | 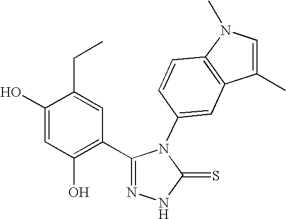 | 3-(2,4-dihydroxy-5-ethyl-phenyl)-4-(1,3-dimethyl-indol-5-yl)-5-mercapto-[1,2,4]triazole |
| 208 | 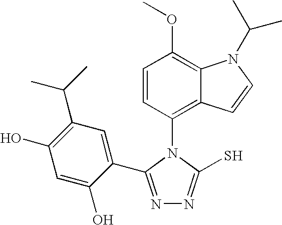 | 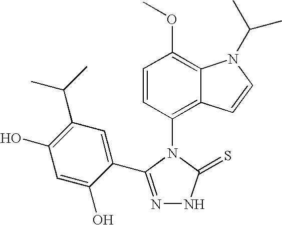 | 3-(2,4-dihydroxy-5-isopropyl-phenyl)-4-(1-isopropyl-7-methoxy-indol-4-yl)-5-mercapto-[1,2,4]triazole |
| 209 | 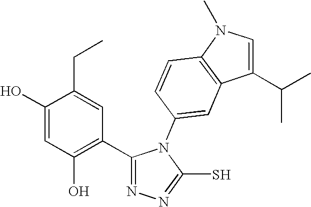 | 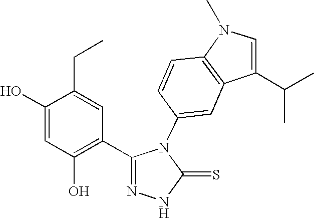 | 3-(2,4-dihydroxy-5-ethyl-phenyl)-4-(1-methyl-3-isopropyl-indol-5-yl)-5-mercapto-[1,2,4]triazole |
| 210 | 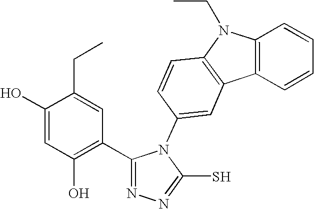 | 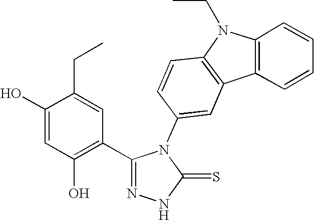 | 3-(2,4-dihydroxy-5-ethyl-phenyl)-4-(N-ethyl-carbozol-7-yl)-5-mercapto-[1,2,4]triazole |
| 211 | 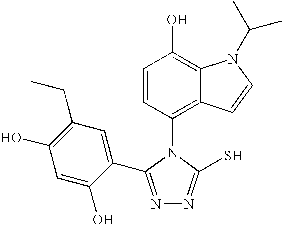 | 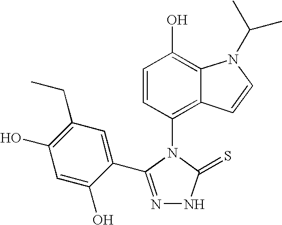 | 3-(2,4-dihydroxy-5-ethyl-phenyl)-4-(1-isopropyl-7-hydroxy-indol-4-yl)-5-mercapto-[1,2,4]triazole |
| 212 | 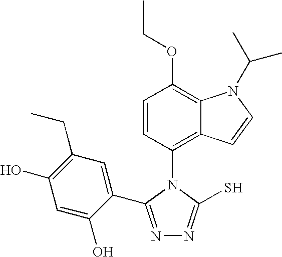 | 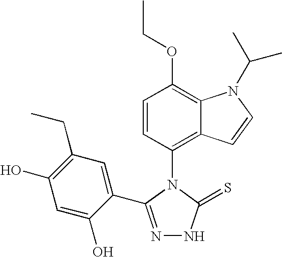 | 3-(2,4-dihydroxy.5-ethyl-phenyl)-4-(1-isopropyl-7-ethoxy-indol-4-yl)-5-mercapto-[1,2,4]triazole |
| 213 | 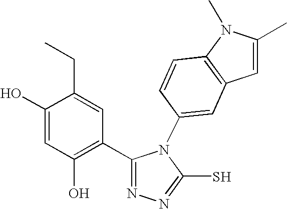 | 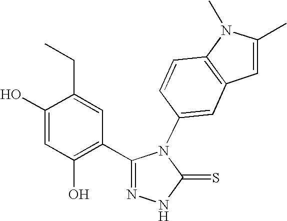 | 3-(2,4-dihydroxy-5-ethyl-phenyl)-4-(1,2-dimethyl-indol-5-yl)-5-mercapto-[1,2,4]triazole |
| 214 | 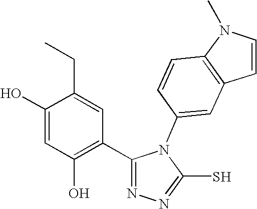 | 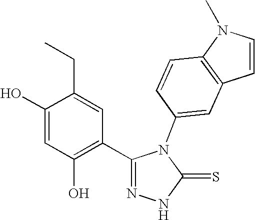 | 3-(2,4-dihydroxy-5-ethyl-phenyl)-4-(N-methyl-indol-5-yl)-5-mercapto-[1,2,4]triazole |
| 215 | 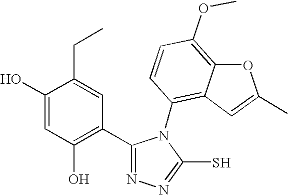 | 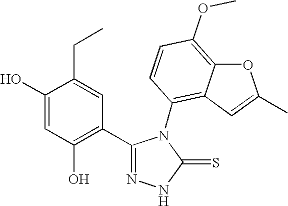 | 3-(2,4-dihydroxy-5-ethyl-phenyl)-4-(2-methyl-7-methoxy-benzofuran-4-yl)-5-mercapto-[1,2,4]triazole |
| 216 | 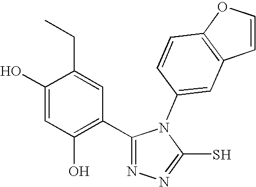 | 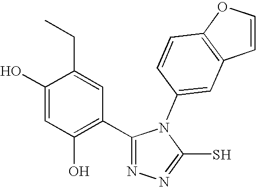 | 3-(2,4-dihydroxy-5-ethyl-phenyl)-4-(benzofuran-5-yl)-5-mercapto-[1,2,4]triazole |
| 217 | 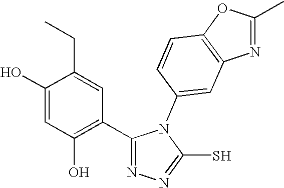 | 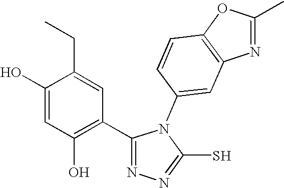 | 3-(2,4-dihydroxy-5-ethyl-phenyl)-4-(2-methyl-1,3-benzoxaz-5-yl)-5-mercapto-[1,2,4]triazole |
| 218 | 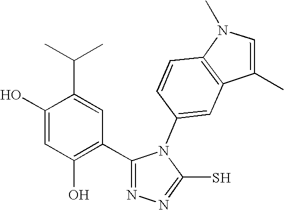 | 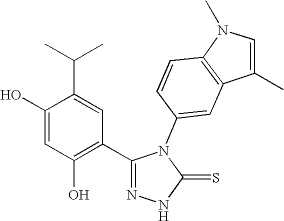 | 3-(2,4-dihydroxy-5-isopropyl-phenyl)-4-(1,3-dimethyl-indol-5-yl)-5-mercapto-[1,2,4]triazole |
| 219 | 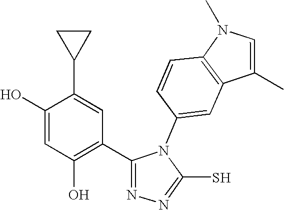 | 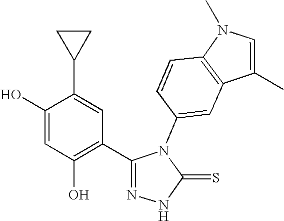 | 3-(2,4-dihydroxy-5-cyclopropyl-phenyl)-4-(1,3-dimethyl-indol-5-yl)-5-mercapto-[1,2,4]triazole |
| 220 | 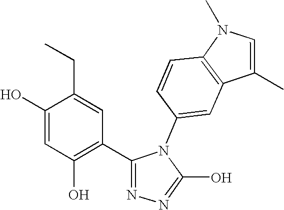 | 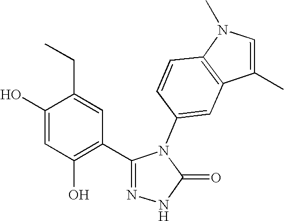 | 3-(2,4-dihydroxy-5-ethyl-phenyl)-4-(1,3-dimethyl-indol-5-yl)-5-hydroxy-[1,2,4]triazole |
| 221 | 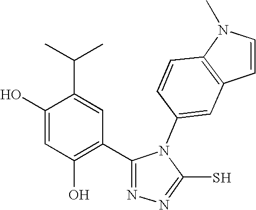 | 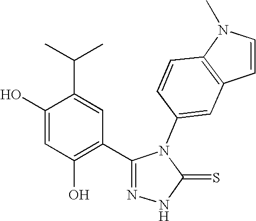 | 3-(2,4-dihydroxy-5-isopropyl-phenyl)-4-(N-methyl-indol-5-yl)-5-mercapto-[1,2,4]triazole |
| 222 | 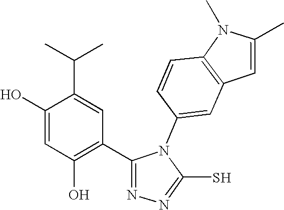 | 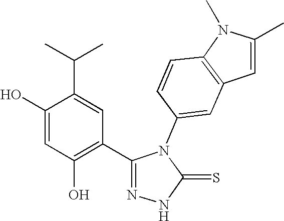 | 3-(2,4-dihydroxy-5-isopropyl-phenyl)-4-(1,2-dimethyl-indol-5-yl)-5-mercapto-[1,2,4]triazole |
| 223 | 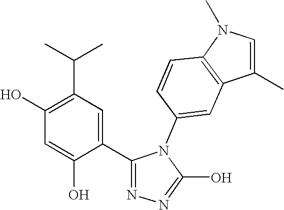 | 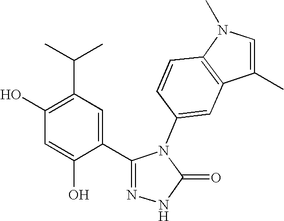 | 3-(2,4-dihydroxy-5-isopropyl-phenyl)-4-(1,3-dimethyl-indol-5-yl)-5-hydroxy-[1,2,4]triazole |
| 224 | 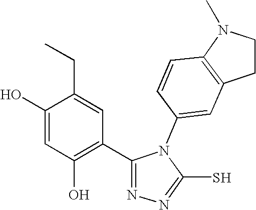 | 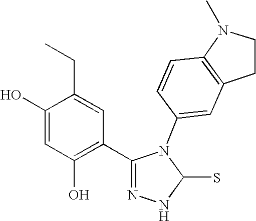 | 3-(2,4-dihydroxy-5-cyclopropyl-phenyl)-4-(1-methyl-indol-5-yl)-5-mercapto-[1,2,4]triazole |
| 225 | 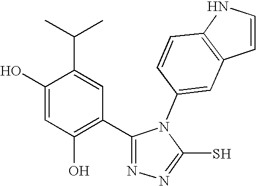 | 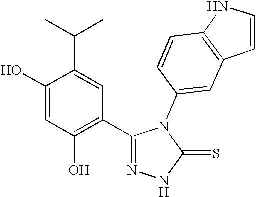 | 3-(2,4-dihydroxy-5-isopropyl-phenyl)-4-(1H-indol-5-yl)-5-mercapto-[1,2,4]triazole |
| 226 | 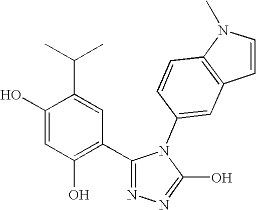 | 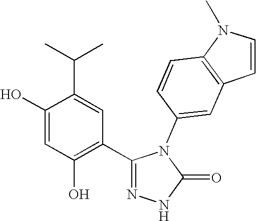 | 3-(2,4-dihydroxy-5-isopropyl-phenyl)-4-(1-methyl-indol-5-yl)-5-hydroxy-[1,2,4]triazole |
| 227 | 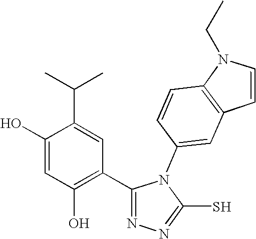 | 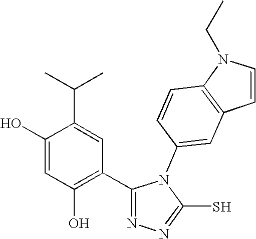 | 3-(2,4-dihydroxy-5-isopropyl-phenyl)-4-(1-ethyl-indol-5-yl)-5-mercapto-[1,2,4]triazole |
| 228 | 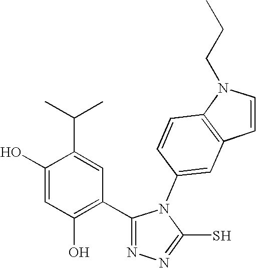 | 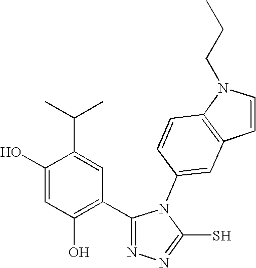 | 3-(2,4-dihydroxy-5-isopropyl-phenyl)-4-(1-propyl-indol-5-yl)-5-mercapto-[1,2,4]triazole |
| 229 | 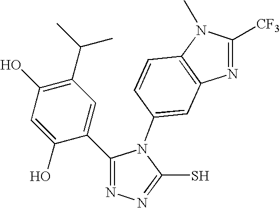 | 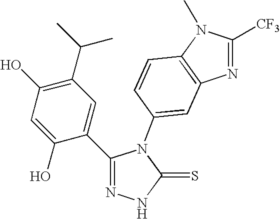 | 3-(2,4-dihydroxy-5-isopropyl-phenyl)-4-(1-methyl-2-trifluoromethyl-benzimidazol-5-yl)-5-mercapto-[1,2,4]triazole |
| 230 | 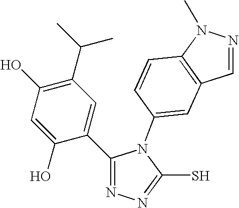 | 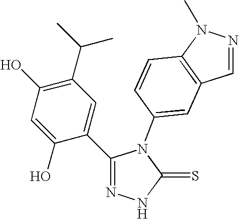 | 3-(2,4-dihydroxy-5-isopropyl-phenyl)-4-(1-methyl-indazol-5 -yl)-5-mercapto-[1,2,4]triazole |
| 231 | 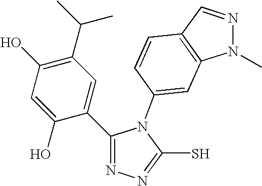 | 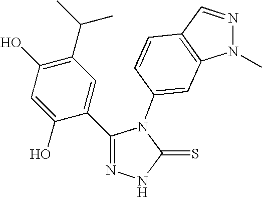 | 3-(2,4-dihydroxy-5-isopropyl-phenyl)-4-(1-methyl-indazol-6-yl)-5-mercapto-[1,2,4]triazole |
| 232 | 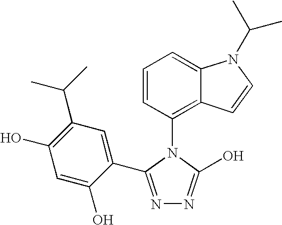 | 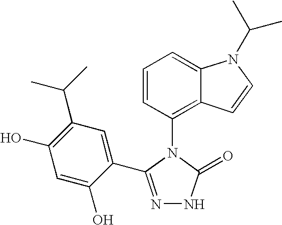 | 3-(2,4-dihydroxy-5-isopropyl-phenyl)-4-(1-isopropyl-indol-4-yl)-5-hydroxy-[1,2,4]triazole |
| 233 | 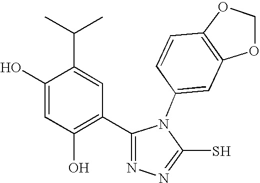 | 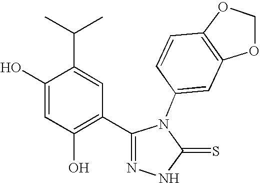 | 3-(2,4-dihydroxy-5-isopropyl-phenyl)-4-(1,3-benzodiaxol-5-yl)-5-mercapto-[1,2,4]triazole |
| 234 | 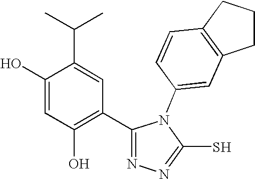 | 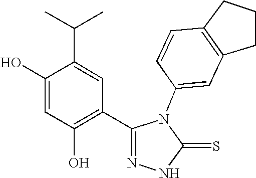 | 3-(2,4-dihydroxy-5-isopropyl-phenyl)-4-(indan-5-yl)-5-mercapto-[1,2,4]triazole |
| 235 | 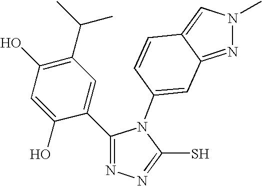 | 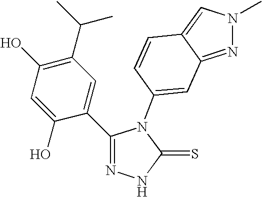 | 3-(2,4-dihydroxy-5-isopropyl-phenyl)-4-(2-methyl-indazol-6-yl)-5-mercapto-[1,2,4]triazole |
| 236 | 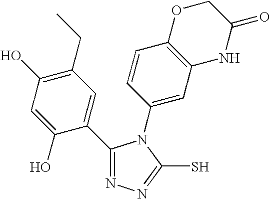 | 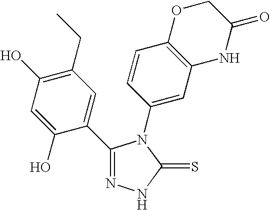 | 3-(2,4-dihydroxy-5-ethyl-phenyl)-4-(3-oxo-benzo[1,4]oxazin-6-yl)-5-mercapto-[1,2,4]triazole |
| 237 | 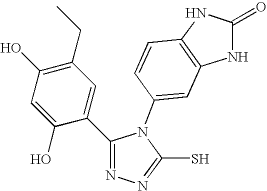 | 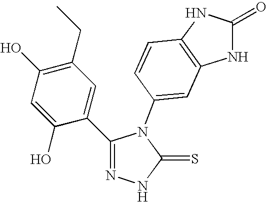 | 3-(2,4-dihydroxy-5-ethyl-phenyl)-4-(2-oxo-1,3-dihydro-benzoimidazol-5-yl)-5-mercapto-[1,2,4]triazole |
| 238 | 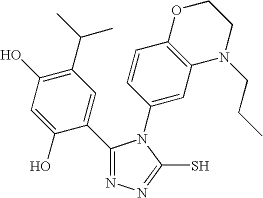 | 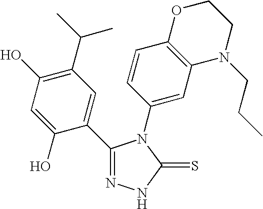 | 3-(2,4-dihydroxy-5-isopropyl-phenyl)-4-(2H-benzo[1,4]oxazin-6-yl)-5-mercapto-[1,2,4]triazole |
| 239 | 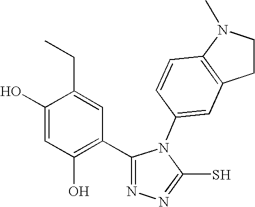 | 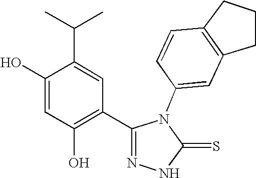 | 4-Ethyl-6-[5-mercapto-4-(1-methyl-2,3-dihydro-1H-indol-5-yl)-4H-[1,2,4]triazol-3-yl]-benzene-1,3-diol |
| 240 | 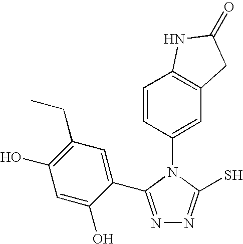 | 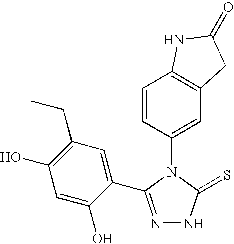 | 5-(3-(5-ethyl-2,4-dihydroxyphenyl)-5-mercapto-4H-1,2,4-triazol-4-yl)indolin-2-one |
| 241 | 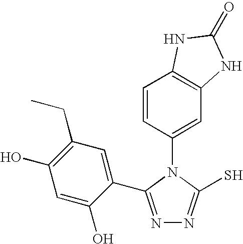 | 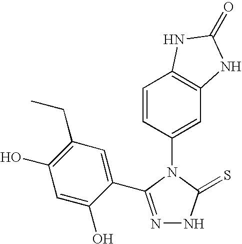 | 5-(3-(5-ethyl-2,4-dihydroxyphenyl)-5-mercapto-4H-1,2,4-triazol-4-yl)-1H-benzo[d]imidazol-2(3H)-one |
| 242 | 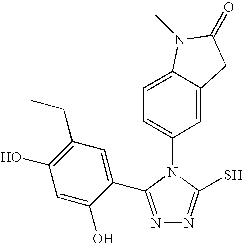 | 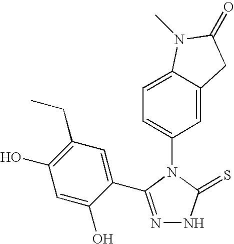 | 5-(3-(5-ethyl-2,4-dihydroxyphenyl)-5-mercapto-4H-1,2,4-triazol-4-yl)-1-methylindolin-2-one |
| 243 | 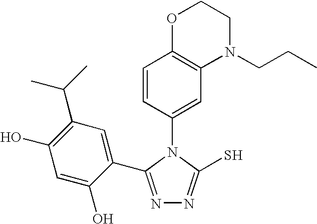 | 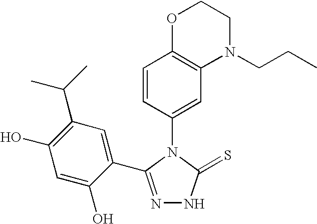 | 4-isopropyl-6-(5-mercapto-4-(4-propyl-3,4-dihydro-2H-benzo[b][1,4]oxazin-6-yl)-4H-1,2,4-triazol-3-yl)benzene-1,3-diol |
| 244 | 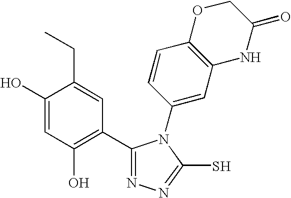 | 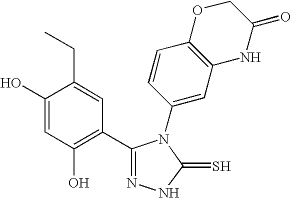 | 6-(3-(5-ethyl-2,4-dihydroxyphenyl)-5-mercapto-4H-1,2,4-triazol-4-yl)-2H-benzo[b][1,4]oxazin-3(4H)-one |
| 245 | 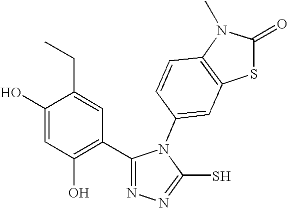 | 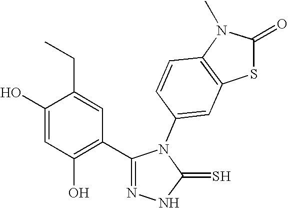 | 6-(3-(5-ethyl-2,4-dihydroxyphenyl)-5-mercapto-4H-1,2,4-triazol-4-yl)-3-methylbenzo[d]thiazol-2(3H)-one |
| 246 | 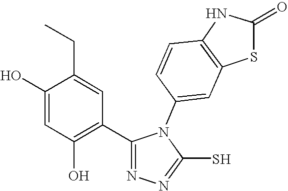 | 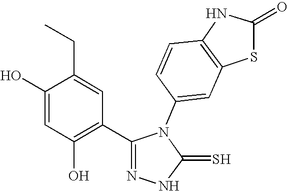 | 6-(3-(5-ethyl-2,4-dihydroxyphenyl)-5-mercapto-4H-1,2,4-triazol-4-yl)benzo[d]thiazol-2(3H)-one |

TABLE 2

| NO. | Structure | Tautomeric structure | Name |
|---|---|---|---|
| 247 | 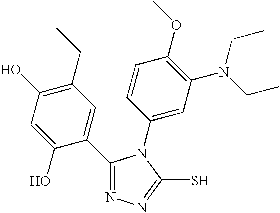 | 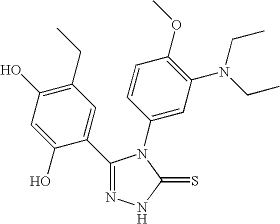 | 4-(4-(3-(diethylamino)-4-methoxyphenyl)-5-mercapto-4H-1,2,4-triazol-3-yl)-6-ethylbenzene-1,3-diol |
| 248 | 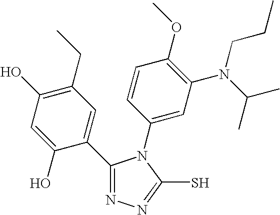 | 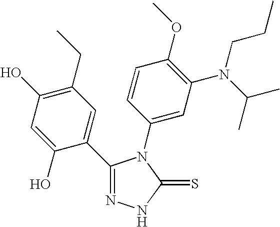 | 4-(4-(3-(N-isopropyl-N-propylamino)-4-methoxyphenyl)-5-mercapto-4H-1,2,4-triazol-3-yl)-6-ethylbenzene-1,3-diol |
| 249 | 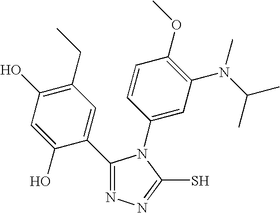 | 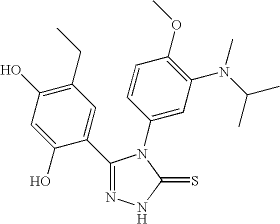 | 4-(4-(3-(N-isopropyl-N-methylamino)-4-methoxyphenyl)-5-mercapto-4H-1,2,4-triazol-3-yl)-6-ethylbenzene-1,3-diol |
| 250 | 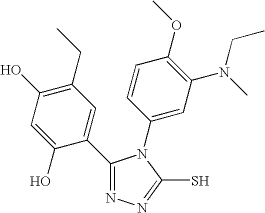 | 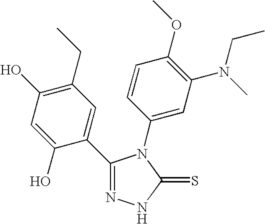 | 4-(4-(3-(N-ethyl-N-methylamino)-4-methoxyphenyl)-5-mercapto-4H-1,2,4-triazol-3-yl)-6-ethylbenzene-1,3-diol |
| 251 | 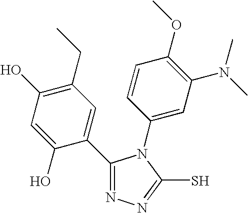 | 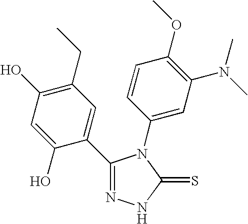 | 4-(4-(3-(dimethylamino)-4-methoxyphenyl)-5-mercapto-4H-1,2,4-triazol-3-yl)-6-ethylbenzene-1,3-diol |
| 252 | 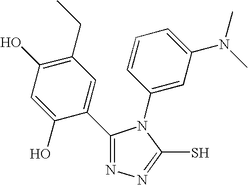 | 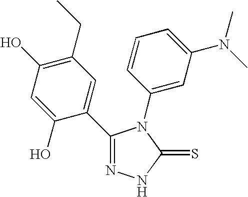 | 4-(4-(3-(dimethylamino)phenyl)-5-mercapto-4H-1,2,4-triazol-3-yl)-6-ethylbenzene-1,3-diol |
| 253 | 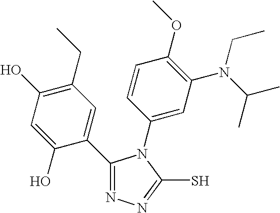 | 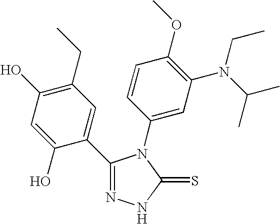 | 4-(4-(3-(N-ethyl-N-isopropylamino)-4-methoxyphenyl)-5-mercapto-4H-1,2,4-triazol-3-yl)-6-ethylbenzene-1,3-diol |
| 254 | 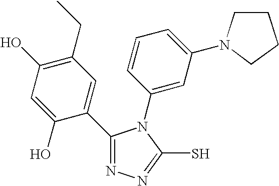 | 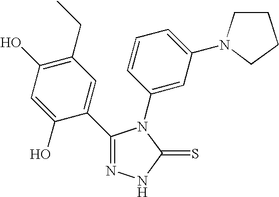 | 4-ethyl-6-(5-mercapto-4-(3-(pyrrolidin-1-yl)phenyl)-4H-1,2,4-triazol-3-yl)benzene-1,3-diol |
| 255 | 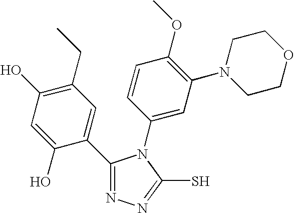 | 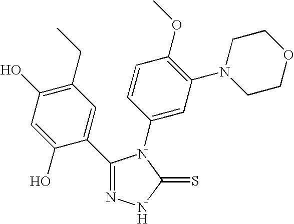 | 4-ethyl-6-(5-mercapto-4-(4-methoxy-3-morpholinophenyl)-4H-1,2,4-triazol-3-yl)benzene-1,3-diol |
| 256 | 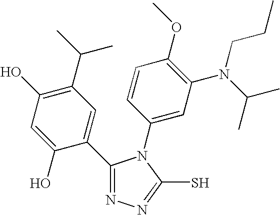 | 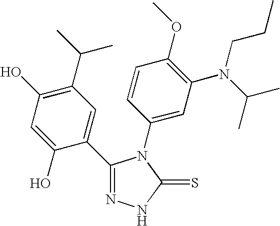 | 4-(4-(3-(N-isopropyl-N-propylamino)-4-methoxyphenyl)-5-mercapto-4H-1,2,4-triazol-3-yl)-6-isopropylbenzene-1,3-diol |
| 257 | 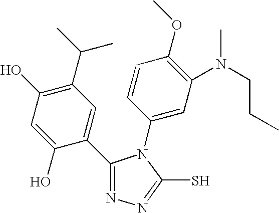 | 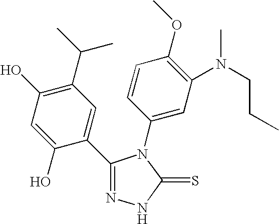 | 4-(4-(3-(N-methyl-N-propylamino)-4-methoxyphenyl)-5-mercapto-4H-1,2,4-triazol-3-yl)-6-isopropylbenzene-1,3-diol |
| 258 | 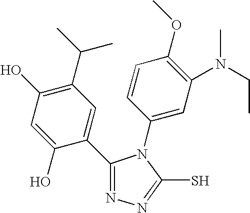 | 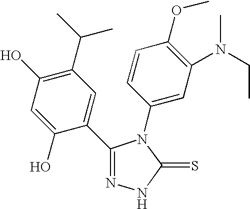 | 4-(4-(3-(N-methyl-N-ethylamino)-4-methoxy-phenyl)-5-mercapto-4H-1,2,4-triazol-3-yl)-6-isopropylbenzene-1,3-diol |
| 259 | 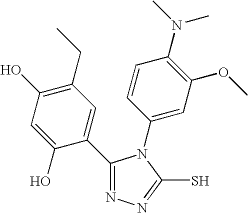 | 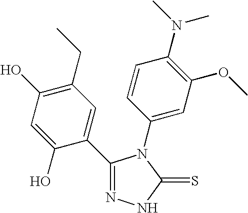 | 4-(4-(4-(dimethylamino)-3-methoxyphenyl)-5-mercapto-4H-1,2,4-triazol-3-yl)-6-ethylbenzene-1,3-diol |
| 260 | 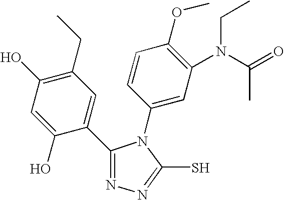 | 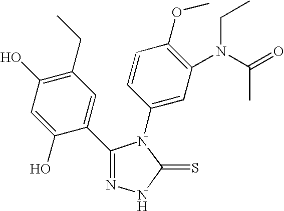 | |
| 261 | 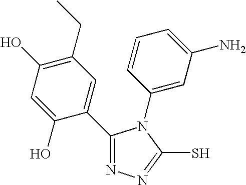 | 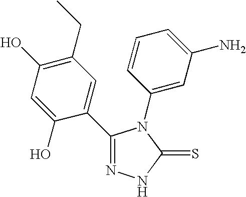 | 4-(4-(3-aminophenyl)-5-mercapto-4H-1,2,4-triazol-3-yl)-6-ethylbenzene-1,3-diol |
| 262 | 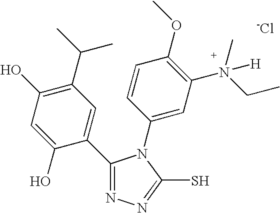 | 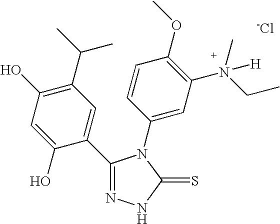 | |
| 263 | 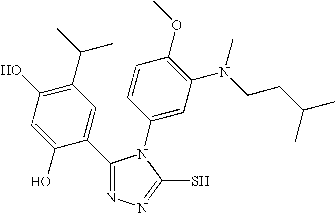 | 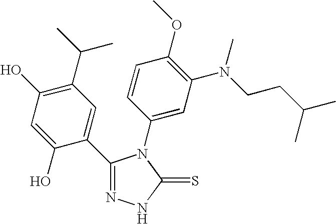 | 4-(4-(3-(N-isopentyl-N-methylamino)-4-methoxyphenyl)-5-mercapto-4H-1,2,4-triazol-3-yl)-6-isopropylbenzene-1,3-diol |
| 264 | 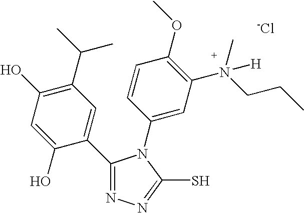 | 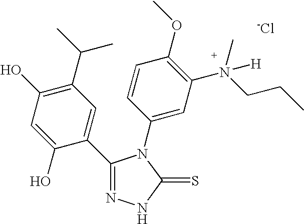 | |
| 265 | 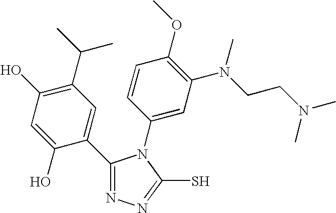 | 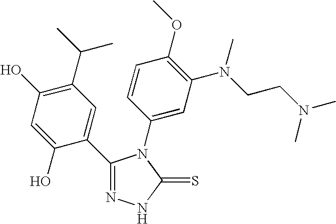 | 4-(4-(3-(N-(2-(dimethylamino)ethyl)-N-methylamino)-4-methoxyphenyl)-5-mercapto-4H-1,2,4-triazol-3-yl)-6-isopropylbenzene-1,3-diol |
| 266 | 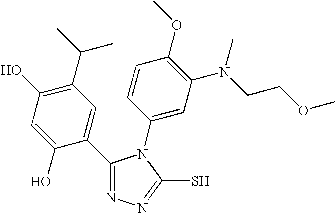 | 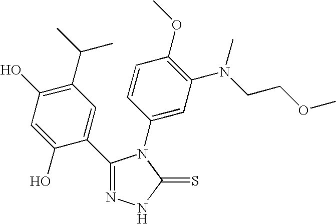 | 4-(4-(3-(N-(2-methoxyethyl)-N-methylamino)-4-methoxyphenyl)-5-mercapto-4H-1,2,4-triazol-3-yl)-6-isopropylbenzene-1,3-diol |
| 267 | 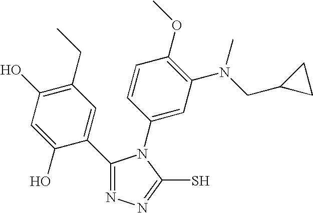 | 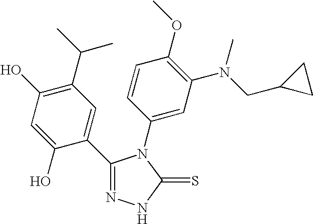 | 4-(4-(3-(N-(cyclopropylmethyl)-N-methylamino)-4-methoxyphenyl)-5-mercapto-4H-1,2,4-triazol-3-yl)-6-isopropylbenzene-1,3-diol |
| 268 | 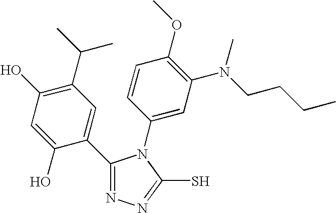 | 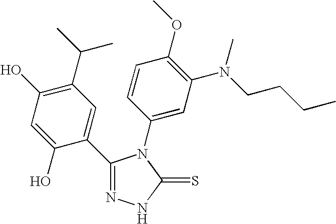 | 4-(4-(3-(N-butyl-N-methylamino)-4-methoxyphenyl)-5-mercapto-4H-1,2,4-triazol-3-yl)-6-isopropylbenzene-1,3-diol |
| 269 | 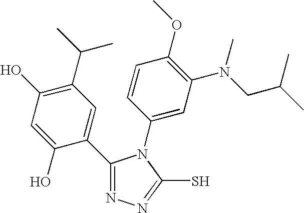 | 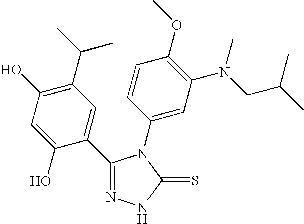 | 4-(4-(3-(N-isobutyl-N-methylamino)-4-methoxyphenyl)-5-mercapto-4H-1,2,4-triazol-3-yl)-6-isopropylbenzene-1,3-diol |
| 270 | 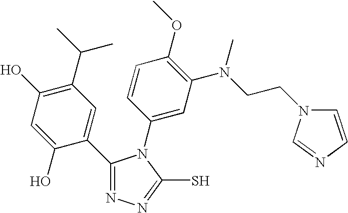 | 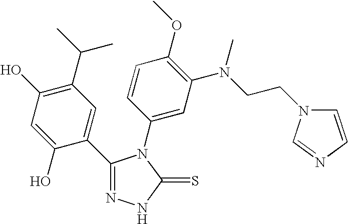 | 4-(4-(3-(N-(2-(1H-imidazol-1-yl)ethyl)-N-methylamino)-4-methoxyphenyl)-5-mercapto-4H-1,2,4-triazol-3-yl)-6-isopropylbenzene-1,3-diol |
| 271 | 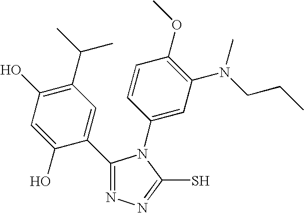 | 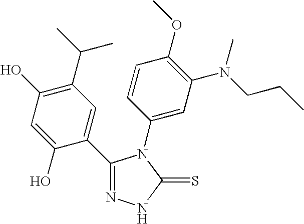 | 4-(4-(3-(N-methyl-N-propylamino)-4-methoxyphenyl)-5-mercapto-4H-1,2,4-triazol-3-yl)-6-isopropylbenzene-1,3-diol |
| 272 | 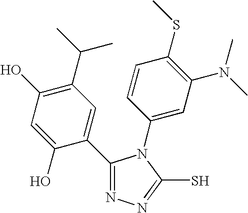 | 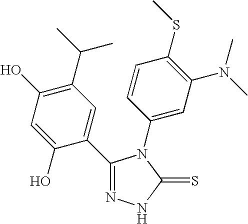 | 4-(4-(3-(dimethylamino)-4-(methylthio)phenyl)-5-mercapto-4H-1,2,4-triazal-3-yl)-6-isopropylbenzene-1,3-diol |
| 273 | 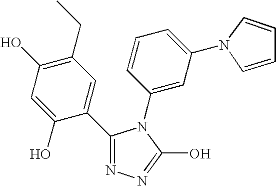 | 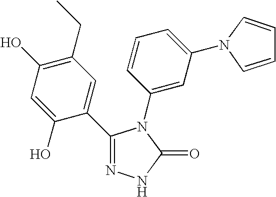 | 4-(4-(3-(1H-pyrrol-1-yl)phenyl)-5-hydroxy-4H-1,2,4-triazol-3-yl)-6-ethylbenzene-1,3-diol |
| 274 | 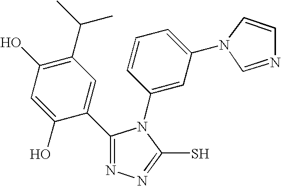 | 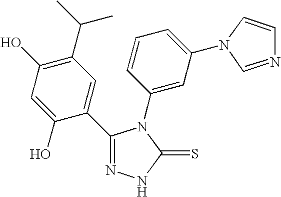 | 4-(4-(3-(1H-imidazol-1-yl)phenyl)-5-mercapto-4H-1,2,4-triazol-3-yl)-6-isopropylbenzene-1,3-diol |
| 275 | 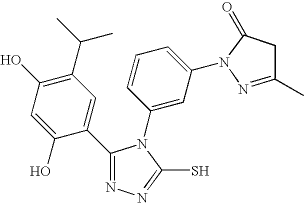 | 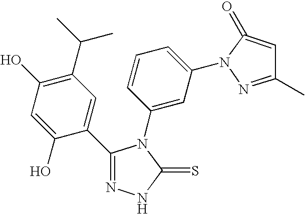 | |
| 276 | 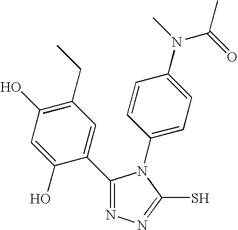 | 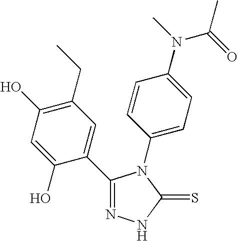 | |
| 277 | 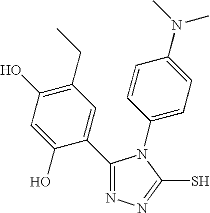 | 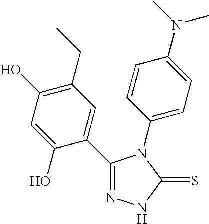 | 4-(4-(4-(dimethylamino)phenyl)-5-mercapto-4H-1,2,4-triazol-3-yl)-6-ethylbenzene-1,3-diol |
| 278 | 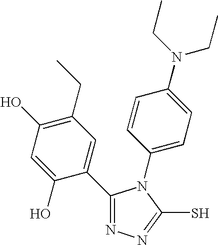 | 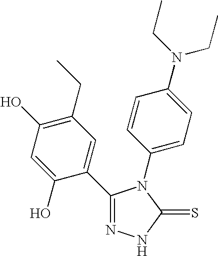 | 4-(4-(4-(diethylamino)phenyl)-5-mercapto-4H-1,2,4-triazol-3-yl)-6-ethylbenzene-1,3-diol |
| 279 | 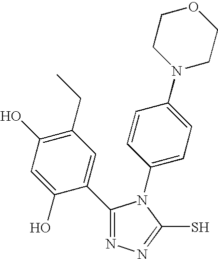 | 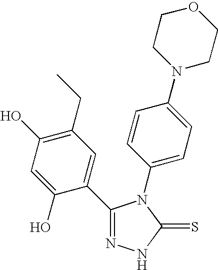 | 4-ethyl-6-(5-mercapto-4-(4-morpholinophenyl)-4H-1,2,4-triazol-3-yl)benzene-1,3-diol |
| 280 | 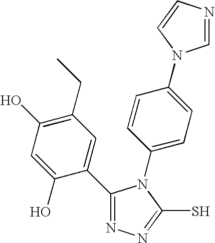 | 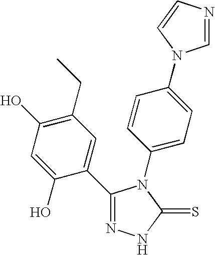 | 4-(4-(4-(1H-imidazol-1-yl)phenyl)-5-mercapto-4H-1,2,4-triazol-3-yl)-6-ethylbenzene-1,3-diol |
| 281 | 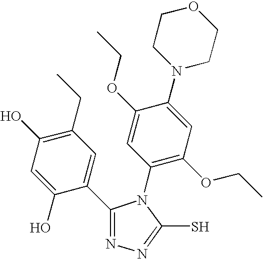 | 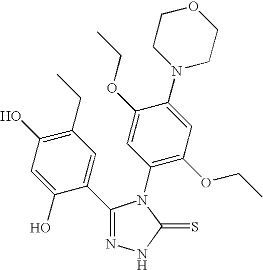 | 4-(4-(2,5-diethoxy-4-morpholinophenyl)-5-mercapto-4H-1,2,4-triazol-3-yl)-6-ethylbenzene-1,3-diol |
| 282 | 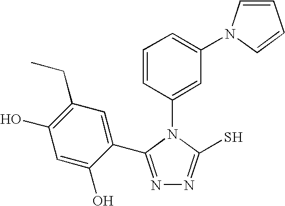 | 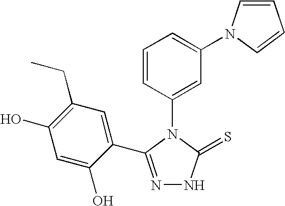 | 4-(4-(3-(1H-pyrrol-1-yl)phenyl)-5-mercapto-4H-1,2,4-triazol-3-yl)-6-ethylbenzene-1,3-diol |
| 283 | 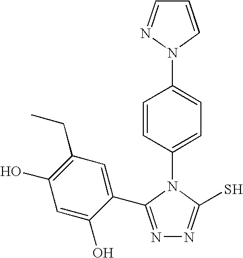 | 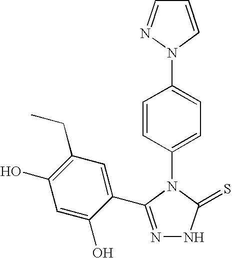 | 4-(4-(4-(1H-pyrazol-1-yl)phenyl)-5-mercapto-4H-1,2,4-triazol-3-yl)-6-ethylbenzene-1,3-diol |
| 284 | 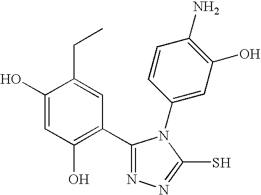 | 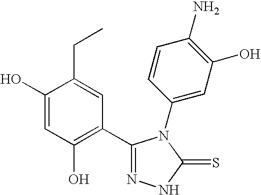 | 4-(4-(4-(amino)-3-hydroxyphenyl)-5-mercapto-4H-1,2,4-triazol-3-yl)-6-ethylbenzene-1,3-diol |
| 285 | 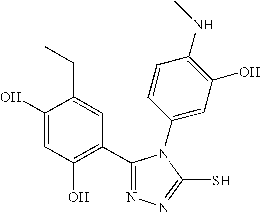 | 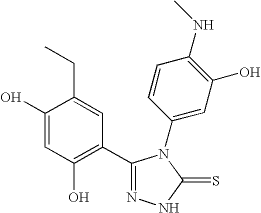 | 4-(4-(4-(methylamino)-3-hydroxyphenyl)-5-mercapto-4H-1,2,4-triazol-3-yl)-6-ethylbenzene-1,3-diol |
| 286 | 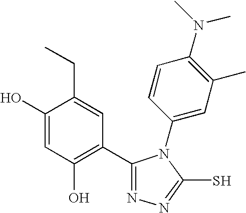 | 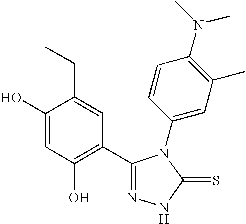 | 4-(4-(4-(dimethylamino)-3-methylphenyl)-5-mercapto-4H-1,2,4-triazol-3-yl)-6-ethylbenzene-1,3-diol |

The invention is illustrated with the following examples which are not intended to be limiting in any way.

### EXEMPLIFICATION

### Example 1

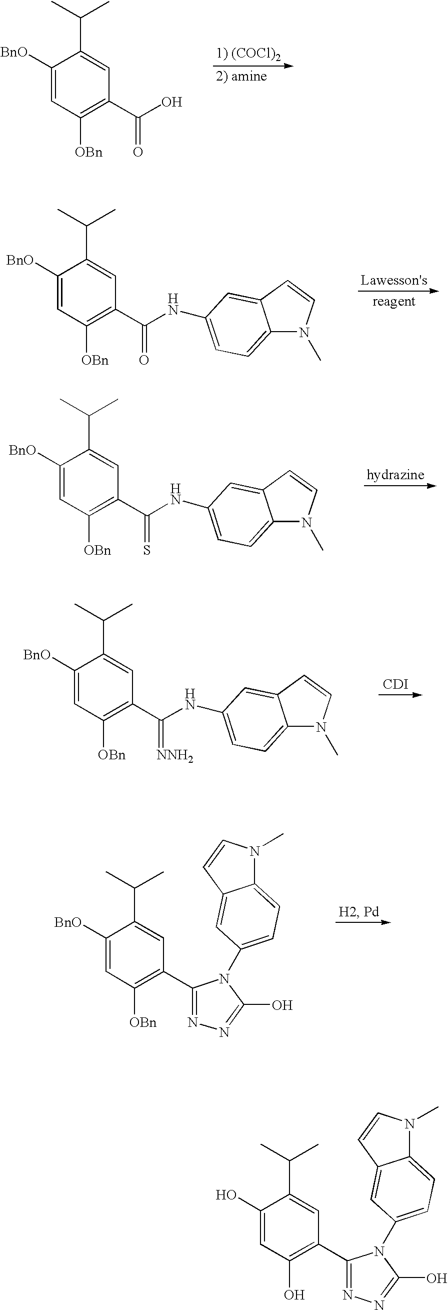

### Preparation of 3-(2,4-dihydroxy-5-isopropyl-phenyl)-4-(N-methyl-indol-5-yl)-5-hydroxy-[1,2,4]triazole

2,4-dibenzyloxy-5-isopropylbenzoic acid (43.0 mmol, 1.00 equiv.) in 300 mL dichloromethane at room temperature was treated with oxalyl chloride (47.3 mmol, 1.10 equiv.) and catalytic amount of DMF (0.5 mL) for 1 hour. Solvent and excess oxalyl chloride were removed on rotary evaporator. The residue was dissolved in 300 mL dichloromethane, and treated with 1,3-dimethyl-5-aminoindole (43.0 mmol, 1.00 equiv.) and triethylamine (64.5 mmol, 1.50 equiv.) at 0° C. for 1 hour. Normal aqueous workup and removal of solvent gave a light brown solid which was washed with ether to yield off-white solid (39.95 mmol, 93%).

Procedure 1. The off-white solid (4 mmol) of the amide obtained above was treated with Lawesson's reagent (970 mg, 0.6 equiv.) in 40 mL toluene at 110° C. for 1.5 hour. Water was added and extracted with ethyl acetate, washed with water 2 times. Dried, concentrated and crystallized by the combination of sonication and addition of hexanes to give an orange solid (80% yield)

Procedure 2. The off-white solid (4 mmol) of the amide obtained above was treated with Lawesson's reagent (970 mg, 0.6 equiv.) in 40 mL toluene at 110° C. for 1.5 hour. The reaction was allowed to cool. Aqueous ammonium hydroxide solution was added (2 mol equiv.) and stirred vigorously at room temperature for 10 min. Water (200 mL) and ethyl acetate (100 mL) were added. The organic layer was washed with water (2×200 mL). The organic layer was then treated with activated carbon (10 g) and stirred at room temperature for 1 hour. Filtration and removal of solvent under reduced pressure gave a bright yellow solid.

The yellow solid of the thioamide obtained from either Procedure 1 or Procedure 2 was treated with hydrazine (anhydrous, 50.0 equiv.) in ethanol at 80° C. for 1.5 hour. The reaction mixture was subjected to EtOAc/aqueous workup to remove excess hydrazine. The organic layer was dried and filtered to remove drying agent.

Carbonyldiimidazole (1.1 equiv.) was added to the solution, and the solution was stirred at 35° C. for 2 hours. Solvent was pumped off, and the residue was treated with 20 mL THF and 10 mL NaOH (2M) to destroy excess carbonyldiimidazole. Normal workup (EtOAc/aqueous) and filtration gave the desired product 5-(2,4-bis(benzyloxy)-5-isopropylphenyl)-4-(1-methyl-1H-indol-5-yl)-4H-1,2,4-triazole-3-ol as light brown solid.

The solid was redissolved in THF/MeOH (1:1 100 mL) and palladium (10 wt % on activated carbon) was added. The reaction was stirred under a hydrogen atmosphere (50 psi) for 18 h. Filtration through celite and removal of solvent under reduced pressure produced the desired product (80% yield).

### Example 2

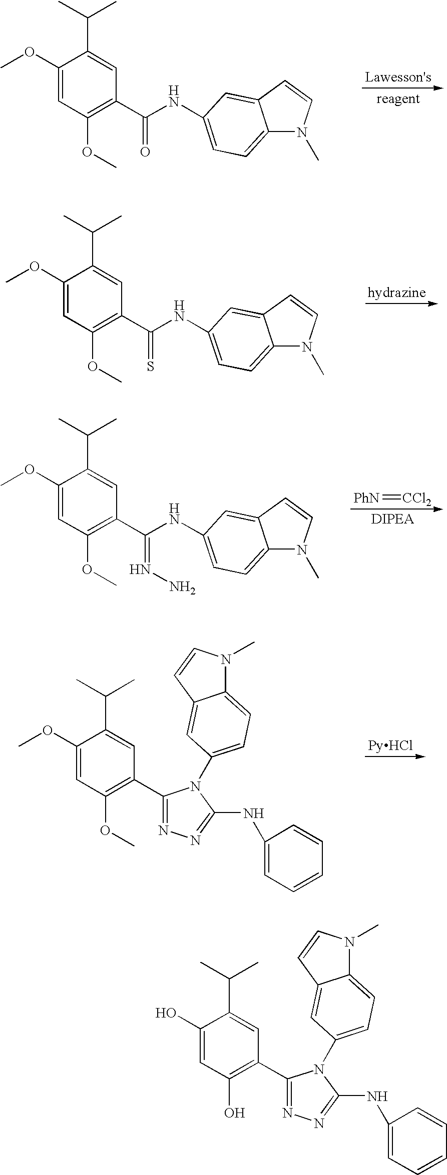

### Preparation of 4-isopropyl-6-[4-(1-methyl-1H-indol-5-yl)-5-phenylamino-4H-[1,2,4-triazol-3-yl]-benzene-1,3-diol

5-isopropyl-2,4-dimethoxy-N-1-methyl-1H-indol-5-yl)-benzamide was prepared reacting 2,4-dimethoxy-5-isopropylbenzoic acid with 1,3-dimethyl-5-aminoindole by a procedure similar that described in Example 1. The corresponding thioamide was prepared by reacting the amide with Lawesson's reagent by a similar procedure as described in Procedure 1 of Example 1. A flask was charged with the thiobenzamide (123 mg, 0.33 mmol), dioxane (2 mL), and hydrazine (0.5 mL). The reaction was heated to 100° C. for one hour, and the solvent was removed by evaporation to give a solid cake. To the solid cake was added ethyl acetate (10 mL) and 10% aqueous potassium carbonate (1 mL), and the mixture was shaken until the solid was completely dissolved. The organic layer was isolated, and dried with sodium sulfate. To the crude intermediate in the organic layer was added diisopropylethylamine (86 mg, 0.66 mmol) and phenylisocyanide dichloride (88 mg, 1.5 equivalent). The reaction was stirred overnight, and washed with saturated aqueous ammonium chloride, dried with sodium sulfate, and the product was purified by column chromatography to give [5-(5-isopropyl-2,4-dimethoxy-phenyl)-4-(1-methyl-1H-indol-5-yl)-4H-[1,2,4]triazol-3-yl]-phenylamine (64 mg).

A flask was charged with [5-(5-isopropyl-2,4-dimethoxy-phenyl)-4-(1-methyl-1H-indol-5-yl)-4H-[1,2,4]triazol-3-yl]-phenylamine (27 mg, 0.06 mmol) and pyridium chloride (2 g). The reactants were placed under a nitrogen atmosphere, and the reaction was heated to 210° C. for 25 minutes. To the cooled reaction mixture was added dichloromethane and saturated ammonium chloride solution. The organic fraction was isolated, and the product was purified by column chromatography to give 4-isopropyl-6-[4-(1-methyl-1H-indol-5-yl)-5-phenylamino-4H-[1,2,4-triazol-3-yl]-benzene-1,3-diol (18 mg, 0.04 mmol). ¹H-NMR (CDCl₃): 7.70 (d, 1H); 7.59 (d, 1H); 7.50 (m, 3H); 7.29 (m, 2H); 7.22 (dd, 1H); 6.98 (m, 1H); 6.62 (d, 1H); 6.40 (s, 1H); 6.28 (s, 1H); 3.95 (s, 2H); 2.83 (q, 1H); 0.57 (d, 3H); 0.44 (d, 3H). ESMS calcd (C₂₆H₂₅N₅O₂): 347.13; Found: 348.1 (M+H)⁺.

### Example 3

The compounds shown below were prepared by similar procedures as described in Procedure 1 of Example 1. Analytical data is provided for these compounds.

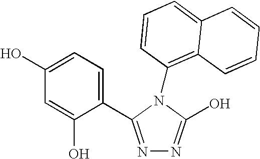

ESMS calcd (C₁₈H₁₃N₃O₃): 319.1; Found: 320 (M+H)⁺.

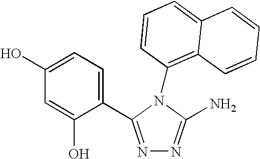

ESMS calcd (C₁₈H₁₄N₄O₃): 318.11; Found: 319.2 (M+H)⁺.

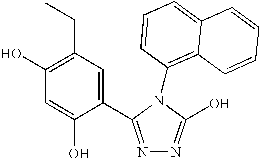

ESMS calcd (C₂₀H₁₇N₃O₃): 347.13; Found: 348.1 (M+H)⁺.

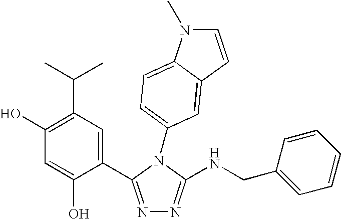

ESMS calcd (C₂₇H₂₇N₅O₂): 453.22; Found: 454.4 (M+H)⁺.

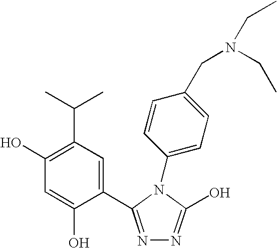

¹H-NMR (DMSO-d₆): 11.85 (s, 1H); 9.61 (s, 1H); 9.43 (s, 1H); 7.30 (d, J=7.5 Hz, 2H); 7.11 (d, J=7.5 Hz, 2H); 6.76 (s, 1H); 6.26 (s, 1H); 3.50 (s, 2H); 3.00-2.90 (m, 1H); 2.47-2.42 (m, 4H); 0.98-0.93 (m, 12H).

ESMS calcd (C₂₂H₂₈N₄O₃): 453.22; Found: 454.4 (M+H)⁺.

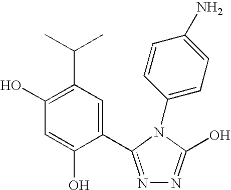

ESMS calcd (C₁₇H₁₈N₄O₃): 326.14; Found: 327.1 (M+H)⁺.

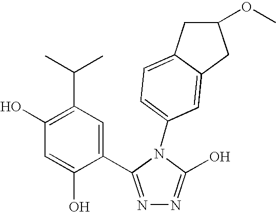

¹H-NMR (DMSO-d₆): 11.90 (s, 1H); 9.59 (s, 1H); 9.44 (s, 1H); 7.18 (d, J=8.1 Hz, 1H); 7.11 (s, 1H); 6.88 (dd, J=8.1, 1.5 Hz, 1H); 6.82 (s, 1H); 6.25 (s, 1H); 4.21-4.15 (m, 1H); 3.23 (s, 3H); 3.10-2.93 (m, 3H); 2.88-2.79 (m, 2H); 0.97 (d, J=6.9 Hz, 6H).

ESMS calcd (C₂₁H₂₃N₃O₄): 381.4; Found: 382.4 (M+H)⁺.

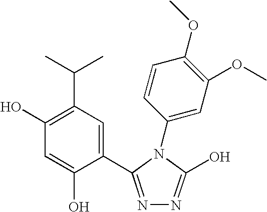

ESMS calcd (C₁₉H₂₁N₃O₅): 371.15; Found: 372.2 (M+H)⁺.

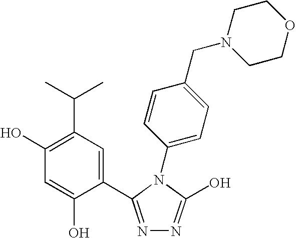

ESMS calcd (C₂₂H₂₆N₄O₄): 410.20; Found: 411.1 (M+H)⁺.

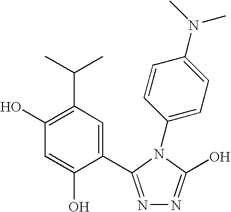

ESMS calcd (C₁₉H₂₂N₄O₃): 354.17; Found: 355.2 (M+H)⁺.

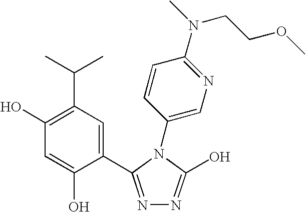

ESMS calcd (C₂₀H₂₅N₅O₄): 399.19; Found: 400.1 (M+H)⁺.

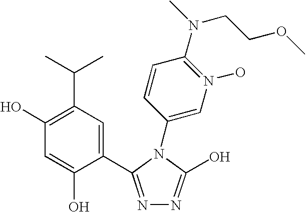

ESMS calcd (C₂₀H₂₅N₅O₅): 415.19; Found: 416.1 (M+H)⁺.

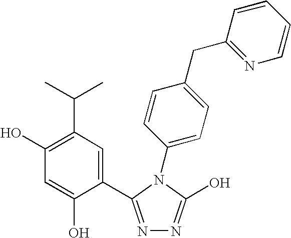

ESMS calcd (C₂₃H₂₂N₄O₃): 402.17; Found: 403.2 (M+H)⁺.

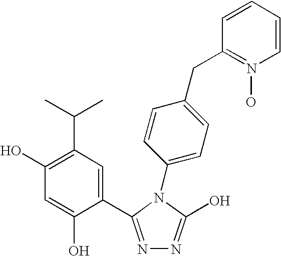

ESMS calcd (C₂₃H₂₂N₄O₄): 418.16; Found: 419.2 (M+H)⁺.

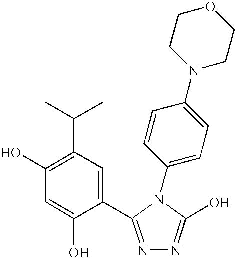

ESMS calcd (C₂₁H₂₄N₄O₄): 396.18; Found: 397.2 (M+H)⁺.

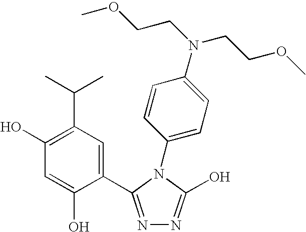

ESMS calcd (C₂₃H₃₀N₄O₅): 442.22; Found: 443.2 (M+H)⁺.

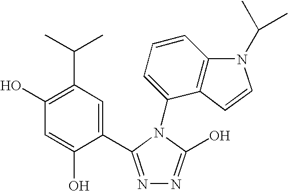

¹H-NMR (DMSO): 12.01 (s, 1H); 9.64 (s, 1H); 9.58 (s, 1H); 7.61 (d, J=8.4 Hz, 1H); 7.52 (d, J=3.3 Hz, 1H); 7.17 (m, 1H); 6.92 (d, J=6.9, 1H); 6.23 (s, 1H); 6.19 (d, J=3.3 Hz, 1H); 4.79 (m, 1H); 2.76 (m, 1H); 1.44 (bs, 6H); 0.57 (d, J=6.9 Hz, 6H).

ESMS calcd (C₂₃H₃₀N₄O₅): 442.22; Found: 443.2 (M+H)⁺.

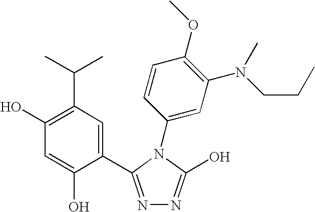

¹H-NMR (DMSO): 11.89 (s, 1H); 9.55 (s, 1H); 9.39 (s, 1H); 6.88 (d, J=8.7 Hz, 1H); 6.77-6.79 (m, 2H); 6.50 (d, J=2.1 Hz, 1H); 6.24 (s, 1H); 3.26 (s, 3H); 2.97 (m, 1H); 2.79 (t, J=7.5 Hz, 2H); 2.48 (s, 3H); 1.30 (m, 2H); 0.96 (d, J=6.9 Hz, 6H); 0.73 (t, J=7.5 Hz, 3H).

ESMS calcd (C₂₂H₂₈N₄O₄): 412.21; Found: 413.1 (M+H)⁺.

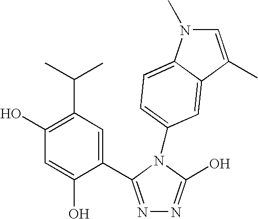

¹H-NMR (DMSO-d₆): 11.86 (s, 1H); 9.51 (s, 1H); 9.43 (s, 1H); 7.34 (d, J=6.6 Hz, 1H); 7.33 (s, 1H); 7.13 (d, J=1.8 Hz, 1H); 6.92 (dd, J=6.6 Hz, 1.8 Hz, 1H); 6.81 (s, 1H); 6.20 (s, 1H); 3.70 (s, 3H); 2.93 (hept, J=6.9 Hz, 1H); 2.15 (s, 3H); 0.88 (d, J=6.9 Hz, 6H).

ESMS calcd (C₂₁H₂₃N₄O₃): 378.17; Found: 379.1 (M+H)⁺.

### Example 4

### Synthesis of the Compound of Formula (XXIVA)

### Step 1: Synthesis of phenyl 1-methyl-1H-indol-5-ylcarbamate 5A

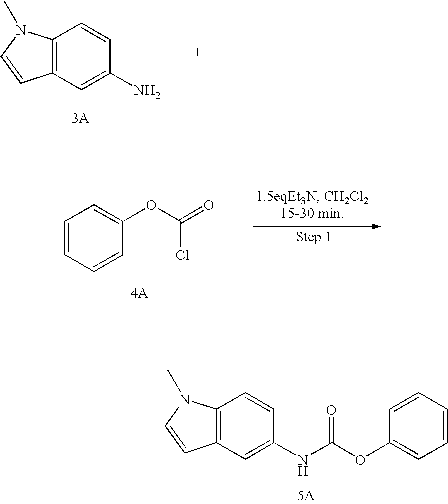

To a solution of 5.62 g (35.91 mmols) of phenylchloroformate 4A in 25 mL of dichloromethane at 0° C. was added, a solution of 5.0 g (34.20 mmols) of indoleamine 3A in 25 mL of dichloromethane drop wise (20 min) at 0° C. The resultant mixture was then stirred for 10 min at 0° C. and a solution of 6 mL (42.75 mmols) of triethylamine in mL of dichloromethane was added drop wise (15 min) at 0° C. and stirred for 5 min. To the mixture was then added 50 mL of water and organic layer separated. The aqueous layer was then extracted with 20 mL of dichloromethane and organic layers combined and dried over Na₂SO₄. The solution was then passed through a pad of silica gel, eluted with additional 50 mL of 3:1 hexane:ethylacetate and concentrated. The crude product was then crystallized with 4:1 hexane:ethylacetate to obtain 7.8 g (85.7%, 99.5% pure, I crop) and 0.78 g (8.5%, 98% pure, II crop) with a combined yield of 94% product.

### Step 2: Synthesis of N-(1-methyl-1H-indol-5-yl)hydrazinecarboxamide 6A

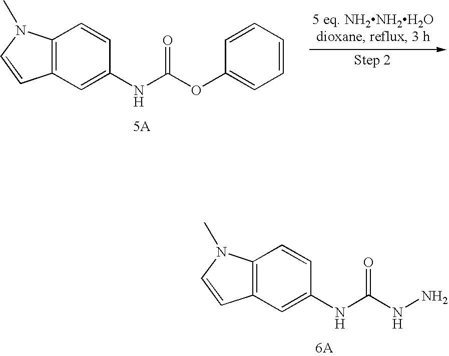

To a stirred suspension of 35.0 g (0.131 mols) of the carbamate 5A in 120 mL of 1,4-dioxane was added 32 mL (0.657 mols) of hydrazine hydrate and the resultant mixture was refluxed for 3 h and concentrated. To the crude mixture was added approx. 250 mL of cold water and the resultant light brown precipitate was filtered and vacuum dried. The crude solid was again treated with 150 mL of ether and stirred for 1 h and filtered. Drying in vacuum afforded 21.6 g (80%) of 6A as grey solid.

### Step 3: Synthesis of 3-(2,4-Bis-benzyloxy-5-isopropyl)benzylideneamino-1-(1-Methyl-1H-indol-′-yl)-urea 8A

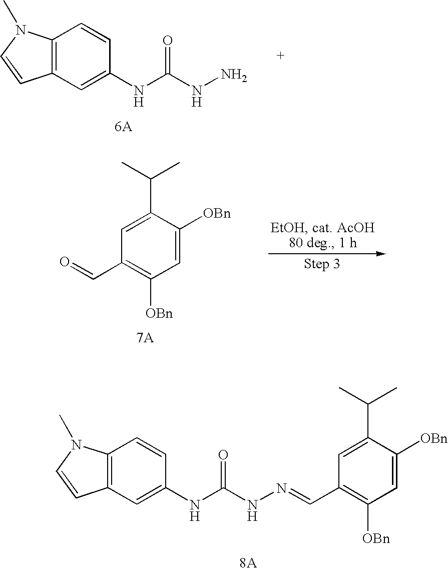

To a suspension of 23.0 g (63.8 mmols) of the aldehyde 7A in 150 mL of ethanol was added 2 mL of AcOH and stirred. To the resultant mixture was added 13.0 g (63.8 mmols) of 6A portion wise (solid, 10 min) at room temperature and the resultant mixture was heated at 80° C. for 1 h. During this time, stirring was difficult due to precipitate formation, therefore an additional 50 mL of ethanol was added. The mixture was cooled to RT and filtered the precipitate, washed with 50 mL of cold ethanol and 100 mL of ether and dried. Vacuum drying afforded 33.7 g (97%) of the product 8A as off-white solid.

ESMS calcd. for C₃₄H₃₄N₄O₃ (M+H)⁺: 546.26; Found: 547.3

### Step 4: Synthesis of 5-(2,4-Bis-benzyloxy-5-isopropylphenyl)-4-(1-methyl-1H-indol-5-yl)-4H-[1,2,4]triazol-3-ol 9A

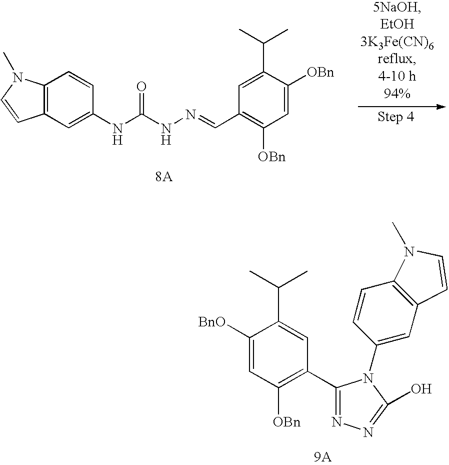

To a stirred suspension of 32.5 g (59.49 mmols) of 8A in 200 mL of ethanol was added 7.14 g (0.178 mmols) of NaOH and stirred. To the resultant mixture, was added 39.17 g (0.118 mmols) of K₃Fe(CN)₆ at once and the resultant mixture was stirred at reflux temperature (100° C. oil bath external temperature) for 8 h (till the reaction is complete, checked by TLC). The mixture was cooled and the inorganics were filtered off. The residues were thoroughly washed with EtOH (50 mL) and a 1:1 mixture of EtOAc:MeOH (150 mL) and filtrates were collected. The combined filtrates were concentrated and crude mixture was dissolved in approx 200 mL of water (still a suspension). The mixture was then acidified with cHCl till pH 2-3 was reached. The resultant precipitate was filtered, washed thoroughly with water and dried. The crude product was then taken up in 90 mL of MeOH and stirred at 50° C. for 30 min and the solid obtained was filtered washed with cold MeOH and dried to obtain 27 g of the off white solid. From the mother liquor another 3.8 g of the grey solid 9A was isolated. Total yield=30.8 g (95%).

ESMS calcd. for C₃₄H₃₂N₄O₃ (M+H)⁺: 544.25; Found: 545.3.

### Step 5: Synthesis of 4-(5-hydroxy-4-(1-methyl-1H-indol-5-yl)-4H-1,2,4-triazol-3-yl)-6-isopropylbenzene-1,3-diol (XXIVA)

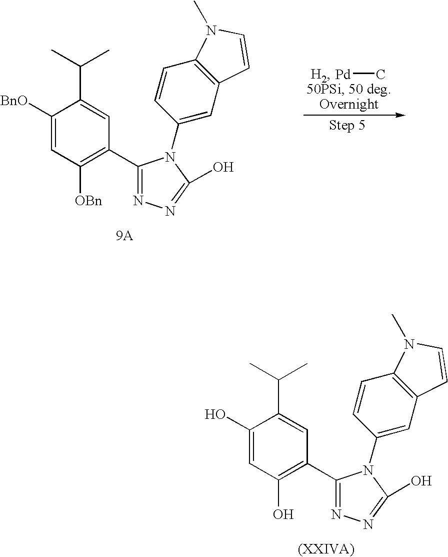

Compound 9A (1 g, 1.84 mmol, 1.0 eq) was hydrogenated by balloon pressure of hydrogen at room temperature in 8 mL of THF and 4 mL of methanol for 6 h. The reaction mixture was filtered through Celite, and washed with THF and EtOAc. After removal solvents, the reaction mixture was dissolved in 20 mL of 1 N NaOH solution, and acidified with 1N HCl until pH is 3˜4. The white precipitate thus obtained was filtered, washed with water and dried using the vacuum oven to produce off-white solid of 4-(5-hydroxy-4-(1-methyl-1H-indol-5-yl)-4H-1,2,4-triazol-3-yl)-6-isopropylbenzene-1,3-diol 10A (0.638 g, 1.75 mmol, 95%).

¹H-NMR (DMSO, 300 MHz) of (XXIVA). δ 11.86 (s, 1H), 9.53 (s, 1H), 9.41 (s, 1H), 9.40-9.36 (m, 3H), 6.91 (dd, J=2.1, 9 Hz, 1H), 6.77 (s, 1H), 6.40 (d, J=3 Hz, 1H), 6.20 (s, 1H), 3.77 (s, 3H), 2.90 (hept., J=6.9 Hz, 1H), 0.87 (d, J=6.9 Hz, 6H).

ESMS calcd. for C₂₀H₂₀N₄O₃ (M+H)⁺: 364.15; Found: 365.2

### Step 5b: Synthesis of 4-(5-hydroxy-4-(1-methyl-1H-indol-5-yl)-4H-1,2,4-triazol-3-yl)-6-isopropylbenzene-1,3-diol (XXIVA)

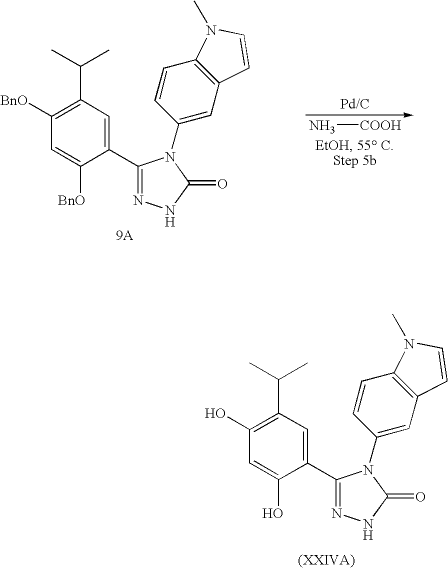

5-(2,4-bis(benzyloxy)-5-isopropylphenyl)-4-(1-methyl-1H-indol-5-yl)-4H-1,2,4-triazol-3-ol, (1.03 g, 1.89 mmol) was stirred with ammonium formate (0.6 g, 9.5 mmol, 5 equiv.) in the presence of palladium on activated carbon (100 mg, 0.1 equiv., 10 wt. %) at 55° C. in reagent grade ethanol (25 ml) and water (0.5 ml) for 1 hour. Completion was judged by TLC. The ‘hot’ reaction mixture was filtered through Celite and washed with hot ethanol (25 ml×3), and concentrated to around ¼ volume. To this mixture was added 100 mL of water. The white precipitate was filtered, washed with water and dried with vacuum oven overnight to give 672 mg of 4-(5-hydroxy-4-(1-methyl-1H-indol-5-yl)-4H-1,2,4-triazol-3-yl)-6-isopropylbenzene-1,3-diol (97.7% yield, 99.8% HPLC purity at 232 mu).

The following table shows the results for various reaction conditions in Step 5b.

| | | | | Ammonium | | |
|---|---|---|---|---|---|---|
| Run | Compd 9A | Catalyst | Time | Formate | Yield | Purity |
| 1 | 0.5 g | 100 wt % | 3.5 h | 5 eq | 96.4% | 98.9% |
| | | Pd/C | (in 30 min, complete | | | |
| | | | reaction, judged by | | | |
| | | | TLC and LC-MS) | | | |
| 2 | 1 g | 10 wt % | 1 h | 5 eq | 97.7% | 99.8% |
| | | Pd/C | (complete reaction, | | | |
| | | | judged by TLC and | | | |
| | | | LC-MS) | | | |
| 3 | 1 g | 24 wt % | overnight | 5 eq | 89% | 99.9% |
| | | EnCat | (in 1 h, complete | | | |
| | | Pd/C | reaction, judged by | | | |
| | | | TLC and LC-MS) | | | |
| 4 | 10 g | 10 wt % | 2 h | 5 eq | 95.7% | 99.8% |
| | | Pd/C | (complete reaction, | | | |
| | | | judged by TLC and | | | |
| | | | LC-MS) | | | |

### Synthesis of Aldehyde 12A and the Compound of Formula (XXIIIA)

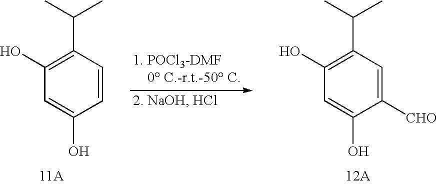

To 70 mL of cold and stirred DMF (ice-bath) was added 31 mL (0.328 mols, 2.5 eq. of reagent) of POCl₃ drop wise over 15 min. The resultant mixture was stirred at ice-bath temperature (0-5° C.) for 30 min. To the mixture was then added 20 g (0.13 mols) of 11A in 40 mL of anhydrous DMF drop wise at ice-bath temperature (0-5° C.) over 25 min. The resultant viscous mixture was stirred at room temperature for 1 h and at 50° C. for 1 h.

The mixture was then poured cautiously to a cold solution of 63 g (12 eq.) of NaOH in 400 mL of water (over 10 min) with vigorous stirring. A red colored solution was then obtained. The mixture was then heated at 70° C. for 15 min and then cooled. It was then acidified with ice-bath cooling with cHCl till pH2-3. The solution turned yellow-orange with same colored precipitate formed. The mixture was stirred further (over weekend; alternatively, anywhere between 15 min. to 1 h stirring should be fine) and filtered. The orange colored precipitate was washed successively with water and vacuum dried at 50° C. to obtain 17.25 g (73%) of orange-light brown powder.

The compound of formula (XXIIIA) is synthesized from compound 12A according to the following scheme:

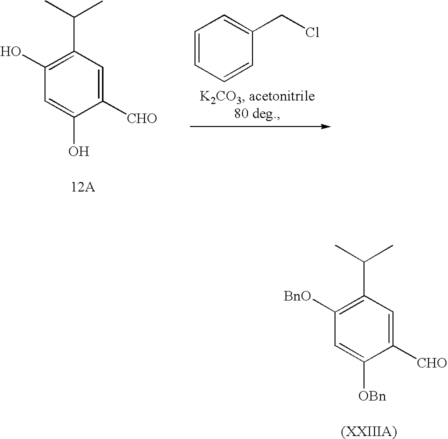

### Exemplary Compounds Synthesized by the Methods of the Invention

Exemplary compounds of formula (IA′) that can be synthesized by the method II of the present invention are compounds 97, 137-173, 176, 220, and 232 depicted in Table 1 above, including tautomers, pharmaceutically acceptable salts, solvates, clathrates, hydrates, polymorphs or prodrugs thereof.

### Example 5

### Preparation of 3-(2,4-dihydroxy-5-isopropyl-phenyl)-4-(N-methyl-indol-5-yl)-5-hydroxy-[1,2,4]triazole

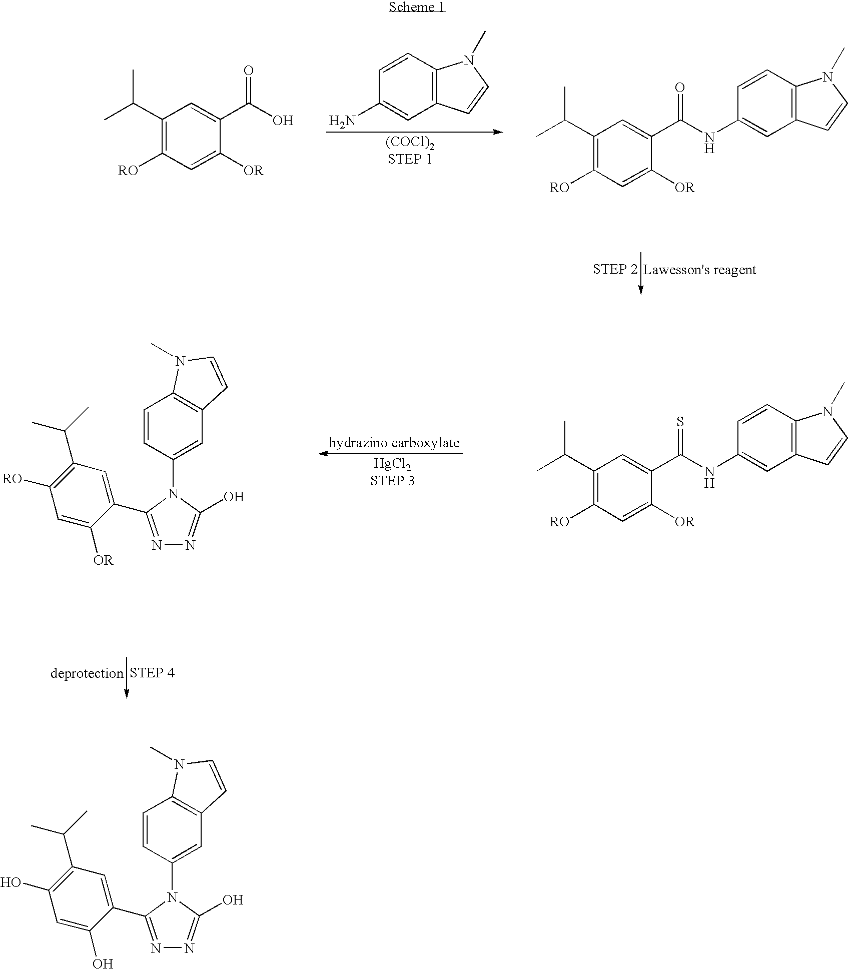

2,4-dibenzyloxy-5-isopropylbenzoic acid (43.0 mmol, 1.00 equiv.) in 300 mL dichloromethane at room temperature was treated with oxalyl chloride (47.3 mmol, 1.10 equiv.) and catalytic amount of DMF (0.5 mL) for 1 hour. Solvent and excess oxalyl chloride were removed on rotary evaporator. The residue was dissolved in 300 mL dichloromethane, and treated with 1,3-dimethyl-5-aminoindole (43.0 mmol, 1.00 equiv.) and triethylamine (64.5 mmol, 1.50 equiv.) at 0° C. for 1 hour. Normal aqueous workup and removal of solvent gave a light brown solid which was washed with ether to yield off-white solid (39.95 mmol, 93%).

Step 1. The off-white solid (4 mmol) of the amide obtained above was treated with Lawesson's reagent (970 mg, 0.6 equiv.) in 40 mL toluene at 110° C. for 1.5 hour. Water was added and extracted with ethyl acetate, washed with water 2 times. Dried, concentrated and crystallized by the combination of sonication and addition of hexanes to give an orange solid (80% yield).

Step 2. The off-white solid (4 mmol) of the amide obtained above was treated with Lawesson's reagent (970 mg, 0.6 equiv.) in 40 mL toluene at 110° C. for 1.5 hour. The reaction was allowed to cool. Aqueous ammonium hydroxide solution was added (2 mol equiv.) and stirred vigorously at room temperature for 10 min. Water (200 mL) and ethyl acetate (100 mL) were added. The organic layer was washed with water (2×200 mL). The organic layer was then treated with activated carbon (10 g) and stirred at room temperature for 1 hour. Filtration and removal of solvent under reduced pressure gave a bright yellow solid.

Step 3

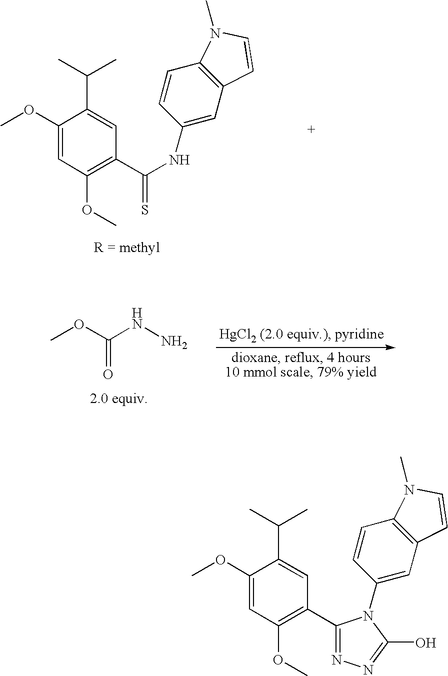

Thioamide (3.682 g, 10.00 mmol, 1.0 equiv), methyl hydrazino carboxylate (1.80 g, 20.0 mmol, 2.0 equiv), pyridine (2.37 mL, around 30.0 mmol, 3.0 equiv) and 40 mL dioxane were mixed in a 100 mL round bottom flask. Mercury (II) chloride (5.43 g, 20.0 mmol, 2.0 equiv) was added to the flask, and stirred at room temperature for half an hour. The mixture was refluxed for 4 hours. Enough Na₂S was added to the mixture after it was cooled to room temperature and stirred for 30 minutes to quench excess mercury chloride. Solid was removed by filtration through celite, and the solution was subjected to EtOAc/aqueous workup. Flash chromatography purification gave an off-white solid (3.10 g, 79%).

¹H NMR (300 MHz, CDCl₃), δ (ppm): 8.96 (br s, 1H); 7.40 (dd, J=2.1 Hz, 0.6 Hz, 1H); 7.24-7.26 (m, 1H); 7.20 (s, 1H); 7.00-7.05 (m, 2H); 6.42 (dd, J=3.0 Hz, 0.6 Hz, 1H); 6.19 (s, 1H); 3.77 (s, 3H); 3.76 (s, 3H); 3.38 (s, 3H); 3.15 (hept, J=7.2 Hz, 1H); 1.10 (d, J=7.2 Hz, 6H). ESMS calcd. for C₂₂H₂₅N₄O₃ (M+H)⁺: 392.2; Found: 392.2.

All publications, patent applications, patents, and other documents cited herein are incorporated by reference in their entirety. In case of conflict, the present specification, including definitions, will control. In addition, the materials, methods, and examples are illustrative only and not intended to be limiting.

While this invention has been particularly shown and described with references to example embodiments thereof, it will be understood by those skilled in the art that various changes in form and details may be made therein without departing from the scope of the invention encompassed by the appended claims.

1. A method of preparing a triazole compound represented by the following Structural Formula: 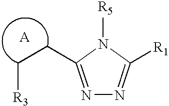
or a tautomer, a pharmaceutically acceptable salt, a solvate, a clathrate, or a prodrug thereof, comprising the steps of:
a) reacting an amide represented by the following Structural Formula: 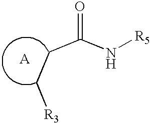
with a thionation reagent to form a thioamide represented by the following Structural Formula: 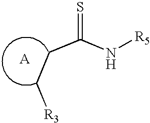
b) reacting the thioamide of step a) with hydrazine to form a hydrazonamide represented by the following Structural Formula: 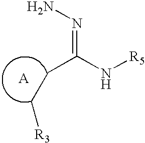
c) reacting the hydrazonamide of step b) with a carbonylation, a thiocarbonylation reagent; or a compound of structural formula R₇N═C(X)₂
wherein:
R₁ is —OH, —SH or —NHR₇;
ring A is an aryl or a heteroaryl optionally further substituted with one or more substituents in addition to R₃;
R₃ is —OR₂₆, —SR₂₆, —O(CH₂)ₘOR_{A}, —O(CH₂)ₘSR_{B}, —O(CH₂)ₘNR₇R_{C}, —S(CH₂)ₘOR_{A}, —S(CH₂)ₘSR_{B}, —S(CH₂)ₘNR₇R_{C}, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —SS(O)ₚOR₇, —NR₇S(O)ₚOR₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂, —OR_{A}, —SR_{B}, —NR₇R_{C}, —NR₂₆R_{C}, or —N(R_{C})₂, wherein R_{A} is a hydroxyl protecting group; R_{B} is a thiol protecting group, R_{C}, for each occurrence, is H or an amine protecting group, provided at least one R_{C} is an amine protecting group;
R₅ is an optionally substituted cycloalkyl, an optionally substituted cycloalkenyl, a substituted alkyl, a substituted phenyl, an optionally substituted heteroaryl, or an optionally substituted 8 to 14 membered aryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₂₆ is a C₁-C₆ alkyl;
p, for each occurrence, is, independently, 0, 1 or 2;
m, for each occurrence, is independently, 1, 2, 3, or 4; and X is a leaving group.

2.-3. (canceled)

4. A method of preparing a thioamide represented by the following Structural Formula: 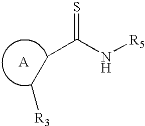
comprising the step of reacting in a reaction mixture an amide represented by the following Structural Formula: 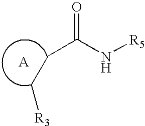
with a thionation reagent, wherein:
ring A is an aryl or a heteroaryl optionally substituted with one or more substituents in addition to R₃;
R₃ is —OR₂₆, —SR₂₆, —O(CH₂)ₘOR_{A}, —O(CH₂)ₘSR_{B}, —O(CH₂)ₘNR₇R_{C}, S(CH₂)ₘOR_{A}, —S(CH₂)ₘSR_{B}, S(CH₂)ₘNR₇R_{C}, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —SS(O)ₚOR₇, —NR₇S(O)ₚOR₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂, —OR_{A}, —SR_{B}, —NR₇R_{C}, —NR₂₆R_{C}, or —N(R_{C})₂, wherein R_{A} is a hydroxyl protecting group; R_{B} is a thiol protecting group, R_{C}, for each occurrence, is H or an amine protecting group, provided at least one R_{C} is an amine protecting group;
R₅ is an optionally substituted cycloalkyl, an optionally substituted cycloalkenyl, a substituted alkyl, a substituted phenyl, an optionally substituted heteroaryl, or an optionally substituted 8 to 14 membered aryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl; R₂₆ is a C₁-C₆ alkyl;
p, for each occurrence, is, independently, 0, 1 or 2; and
m, for each occurrence, is independently, 1, 2, 3, or 4.

5.-7. (canceled)

8. A method of preparing a hydrazonamide represented by the following Structural Formula: 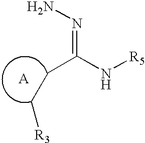
comprising the step of reacting a thioamide represented by the following Structural Formula: 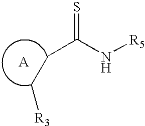
with hydrazine, wherein
ring A is an aryl or a heteroaryl optionally substituted with one or more substituents in addition to R₃;
R₃ is —OR₂₆, —SR₂₆, —O(CH₂)ₘOR_{A}, —O(CH₂)ₘSR_{B}, —O(CH₂)ₘNR₇R_{C}, —S(CH₂)ₘOR_{A}, —S(CH₂)ₘSR_{B}, —S(CH₂)ₘNR₇R_{C}, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —SS(O)ₚOR₇, —NR₇S(O)ₚOR₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂, —OR_{A}, —SR_{B}, —NR₇R_{C}, —NR₂₆R_{C}, or —N(R_{C})₂, wherein R_{A} is a hydroxyl protecting group; R_{B} is a thiol protecting group, R_{C}, for each occurrence, is H or an amine protecting group, provided at least one R_{C} is an amine protecting group;
R₅ is an optionally substituted cycloalkyl, an optionally substituted cycloalkenyl, a substituted alkyl, a substituted phenyl, an optionally substituted heteroaryl, or an optionally substituted 8 to 14 membered aryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁ taken together with the nitrogen to which they are attached, form an optionally substituted heterocyclyl or an optionally substituted heteroaryl;
R₂₆ is a C₁-C₆ alkyl;
p, for each occurrence, is, independently, 0, 1 or 2; and
m, for each occurrence, is independently, 1, 2, 3, or 4.

9. A method of preparing a triazole compound represented by the following Structural Formula: 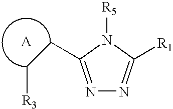
or a tautomer, a pharmaceutically acceptable salt, a solvate, a clathrate, or a prodrug thereof, comprising the step of reacting a hydrazonamide represented by the following Structural Formula: 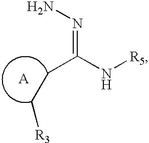
with a carbonylation or a thiocarbonylation reagent, wherein:
ring A is an aryl or a heteroaryl optionally substituted with one or more substituents in addition to R₃;
R₃ is —OR₂₆, —SR₂₆, —O(CH₂)ₘOR_{A}, —O(CH₂)ₘSR_{B}, —O(CH₂)ₘNR₇R_{C}, —S(CH₂)ₘOR_{A}, —S(CH₂)ₘSR_{B}, —S(CH₂)ₘNR₇R_{C}, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —SS(O)ₚOR₇, —NR₇S(O)ₚOR₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂, —OR_{A}, —SR_{B}, —NR₇R_{C}, —NR₂₆R_{C} or N(R_{C})₂, wherein R_{A} is a hydroxyl protecting group; R_{B} is a thiol protecting group, R_{C}, for each occurrence, is H or an amine protecting group, provided at least one R_{C} is an amine protecting group;
R₅ is an optionally substituted cycloalkyl, an optionally substituted cycloalkenyl, a substituted alkyl, a substituted phenyl, an optionally substituted heteroaryl, or an optionally substituted 8 to 14 membered aryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl; R₂₆ is a C1-C6 alkyl;
p, for each occurrence, is, independently, 0, 1 or 2; and
m, for each occurrence, is independently, 1, 2, 3, or 4.

10.-12. (canceled)

13. A method of preparing of a triazole compound of Structural Formula (IA), comprising reacting a compound of Structural Formula (IIA): 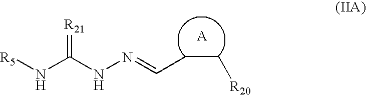
with an oxidizing agent, thereby producing a compound of Structural Formula (IA): 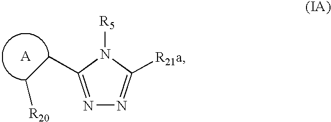
or a tautomer, a pharmaceutically acceptable salt, a solvate, a clathrate, or a prodrug thereof, wherein:
ring A is an aryl or a heteroaryl, wherein the aryl or the heteroaryl are optionally further substituted with one or more substituents in addition to R₂₀;
R₅ is an optionally substituted cycloalkyl, an optionally substituted cycloalkenyl, a substituted alkyl, a substituted phenyl, an optionally substituted heteroaryl or an optionally substituted 8 to 14 membered aryl;
R₂₀ is —ORₚ₁, —NHRₚ₃ or —N(Rₚ₃)₂, wherein Rₚ₁, for each occurrence, is independently selected from groups suitable for protecting hydroxyl, and Rₚ₃, for each occurrence, is independently selected from groups suitable for protecting an amino group;
R₂₁ is O, NH, or NR₂₆, and R₂₁ₐ is OH, NH₂ or NHR₂₆; and
R₂₆ is a C1-C6 alkyl.

14. The method of claim 13, wherein the oxidizing agent is K₃Fe(CN)₆, MnO₂, Br₂, N-bromosuccinimide or N-chlorosuccinimide.

15. (canceled)

16. The method of claim 13, further comprising the step of deprotecting the compound of Structural Formula (IA) 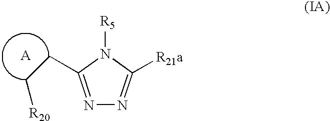
thereby producing a compound of Structural Formula (IA′) 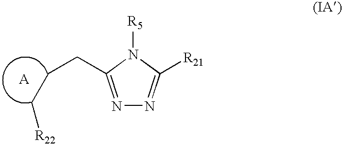
wherein R₂₂ is —OH, or —NH₂.

17. The method of claim 16, wherein R₂₀ is —ORₚ₁, Rₚ₁ is a benzyl group and the step of deprotecting comprises reacting a compound of formula (IA) with hydrogen in the presence of hydrogenation catalyst.

18. A method of preparing a compound of Structural Formula (IIA), 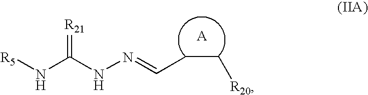
comprising:
reacting a compound of Structural Formula (IIIA) 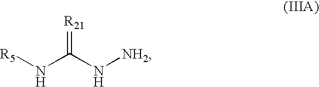
with a compound of Structural Formula (IVA) 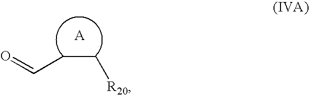
in the presence of an acid, thereby producing a compound of formula (IIA), wherein:
ring A is an aryl or a heteroaryl, wherein the aryl or the heteroaryl are optionally further substituted with one or more substituents in addition to R₂₀;
R₅ is an optionally substituted cycloalkyl, an optionally substituted cycloalkenyl, a substituted alkyl, a substituted phenyl, an optionally substituted heteroaryl or an optionally substituted 8 to 14 membered aryl;
R₂₀ is —ORₚ₁, —NHRₚ₃ or —N(Rₚ₃)₂, wherein Rₚ₁, for each occurrence, is independently selected from groups suitable for protecting hydroxyl, and Rₚ₃, for each occurrence, is independently selected from groups suitable for protecting an amino group;
R₂₁ is O, NH, or NR₂₆, and R₂₁ₐ is OH, NH₂ or NHR₂₆; and
R₂₆ is a C1-C6 alkyl.

19. A method of preparing a triazole compound represented by the following Structural Formula: 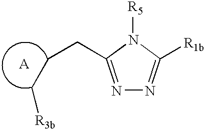
or a tautomer, a pharmaceutically acceptable salt, a solvate, a clathrate, or a prodrug thereof, wherein the method comprises the step of reacting a first starting compound represented by the following Structural Formula: 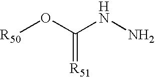
in the presence of a mercuric salt, with a second starting compound represented by the following Structural Formula: 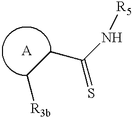
R_{1b} is —OH, —SH or —NHR₆₀, wherein R₆₀ is H, an optionally substituted alkyl group, or an optionally substituted cycloalkyl group;
ring A is an aryl or a heteroaryl, wherein the aryl group and the heteroaryl group represented by ring A is optionally further substituted with one or more substituents in addition to R₃;
R_{3b} is —OR₁₀₀, —SR₁₀₁, —N(R₁₀₂)₂, —NR₇R₁₀₂, —OR₂₆, —SR₂₆, —NR₂₆R₁₀₂, —O(CH₂)ₘOR₁₀₀, —O(CH₂)ₘSR₁₀₁, —O(CH₂)ₘNR₇R₁₀₂, —S(CH₂)ₘOR₁₀₀, S(CH₂)ₘSR₁₀₁, S(CH₂)ₘNR₇R₁₀₂, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(R₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂;
each R₁₀₀, independently, is a hydroxyl protecting group;
each R₁₀₁, independently, is a thiol protecting group;
each R₁₀₂, independently, is —H or an amino protecting group, provided that at least one group represented by R₁₀₂ is a protecting group;
R₅ is an optionally substituted aryl group, an optionally substituted heteroaryl group, an optionally substituted cycloalkyl group, an optionally substituted cycloakenyl group, or a substituted alkyl group, wherein each of the aryl group, heteroaryl group, cycloaryl group, cycloalkyl group, cycloalkenyl group, and alkyl group is substituted with one or more substituents independently selected from the group consisting of an optionally substituted alkyl group, an optionally substituted alkynyl, an optionally substituted cycloalkyl group, an optionally substituted cycloalkenyl group, an optionally substituted heteroaryl group, an optionally substituted aralyalkyl group, or an optionally substituted heteraralkyl group;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, or R₇, taken together with the oxygen atom to which it is bonded, forms an optionally substituted heterocyclyl or an optionally substituted heteroaryl;
R₁₀ and R₁₁, for each occurrence, are, independently, amine protecting group, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₂₆ is a C1-C6 alkyl group;
R₅₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₅₁ is ═O, ═S or ═NR₆₀;
p, for each occurrence, is, independently, 0, 1 or 2; and
m, for each occurrence, is, independently, 1, 2, 3, or 4.

20.-27. (canceled)

28. The method of claim 13, wherein R₅ is represented by the following Structural Formula: 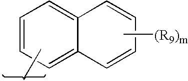
wherein:
R₉, for each occurrence, is independently a substituent selected from: —ORₚ₁, —NHRₚ₃, —N(Rₚ₃)₂, —O(CH₂)ₘORₚ₁, or —(CH₂)ₘORₚ₁; an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl; halo, cyano, nitro, —NR₁₀R₁₁, —OR₇, —O(CH₂)ₘNR₇Rₚ₃, —C(O)R₇, —C(O)OR₇; —C(O)NR₁₀R₁₁; —OC(O)R₇, —OC(O)OR₇, —OC(O)NR₁₀R₁₁; —NR₈C(O)R₇, —NR₇C(O)NR₁₀R₁₁, —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁, —NR₈S(O)ₚR₇, —NR₇S(O)ₚNR₁₀R₁₁, or —NR₇S(O)ₚOR₇; or two R₉ groups taken together with the carbon atoms to which they are attached form a fused ring;
R₇ and R₉, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
or R₁₀ and R₁₁, taken together with the nitrogen to which they are attached, form an optionally substituted heterocyclyl or an optionally substituted heteroaryl;
p, for each occurrence, is, independently, 0, 1 or 2; and
m, for each occurrence, is independently, 1, 2, 3, or 4.

29.-35. (canceled)

36. The method of claim 13, wherein R₅ is represented by the following Structural Formula: 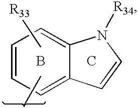
wherein:
R₃₃ is H, —ORₚ₁, —NHRₚ₃, —N(Rₚ₃)₂, a halo, a lower alkyl, a lower alkoxy, a lower haloalkyl, or a lower haloalkoxy;
R₃₄ is H, —ORₚ₁, —NHRₚ₃, —N(Rₚ₃)₂, a C1-C6 alkyl, or a lower alkylcarbonyl; and
Ring B and Ring C are optionally substituted with one or more substituents in addition to R₃₃ and R₃₄.

37.-38. (canceled)

39. The method of claim 13, wherein R₅ is selected from the group consisting of: 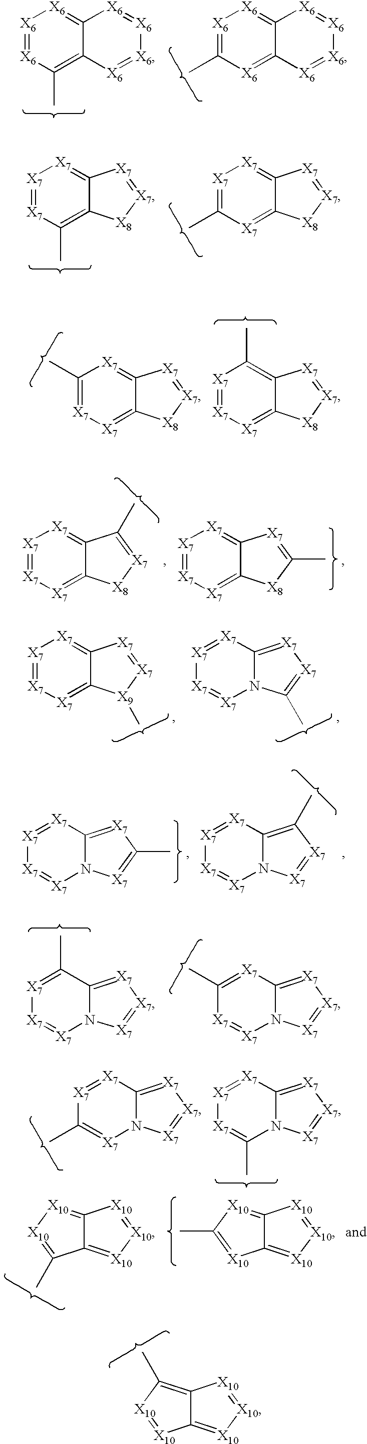
wherein:
X₆, for each occurrence, is independently CH, CR₉, N, N(O), N⁺(R₁₇), provided that at least three X₆ groups are independently selected from CH and CR₉;
X₇, for each occurrence, is independently CH, CR₉, N, N(O), N⁺(R₁₇), provided that at least three X₇ groups are independently selected from CH and CR₉;
X₈, for each occurrence, is independently CH₂, CHR₉, C(R₉)₂, S(O)ₚ, NR₇, or NR₁₇;
X₉, for each occurrence, is independently N or CH;
X₁₀, for each occurrence, is independently CH, CR₉, N, N(O), N⁺(R₁₇), provided that at least one X₁₀ is selected from CH and CR₉;
R₉, for each occurrence, is independently a substituent selected from:
—ORₚ₁, —NHRₚ₃, —N(Rₚ₃)₂, —O(CH₂)ₘORₚ₁, or —(CH₂)ₘORₚ₁;
an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl;
halo, cyano, nitro, —NR₁₀R₁₁, —OR₇, —O(CH₂)ₘNR₇Rₚ₃, —C(O)R₇, —C(O)OR₇; —C(O)NR₁₀R₁₁; —OC(O)R₇, —OC(O)OR₇, —OC(O)NR₁₀R₁₁; —NR₈C(O)R₇, —NR₇C(O)NR₁₀R₁₁, —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁, —NR₈S(O)ₚR₇, —NR₇S(O)ₚNR₁₀R₁₁, or —NR₇S(O)ₚOR₇;
or two R₉ groups taken together with the carbon atoms to which they are attached form a fused ring; and
R₁₇, for each occurrence, is independently —H, an alkyl, an aralkyl, —C(O)R₇, —C(O)OR₇, or —C(O)NR₁₀R₁₁;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
or R₁₀ and R₁₁, taken together with the nitrogen to which they are attached, form an optionally substituted heterocyclyl or an optionally substituted heteroaryl;
p, for each occurrence, is, independently, 0, 1 or 2; and
m, for each occurrence, is independently, 1, 2, 3, or 4.

40.-42. (canceled)

43. The method of claim 13, wherein R₅ is selected from the group consisting of: 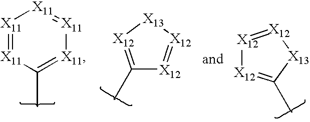
wherein:
X₁₁, for each occurrence, is independently CH, CR₉, N, N(O), or N⁺(R₁₇),
provided that at least one X₁₁ is N, N(O), or N⁺(R₁₇) and at least two X₁₁ groups are independently selected from CH and CR₉;
X₁₂, for each occurrence, is independently CH, CR₉, N, N(O), N⁺(R₁₇), provided that at least one X₁₂ group is independently selected from CH and CR₉;
X₁₃, for each occurrence, is independently O, S, S(O)ₚ, NR₇, or NR₁₇;
R₉, for each occurrence, is independently a substituent selected from:
—ORₚ₁, —NHRₚ₃, —N(Rₚ₃)₂, —O(CH₂)ₘORₚ₁, or —(CH₂)ₘORₚ₁;
an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl;
halo, cyano, nitro, —NR₁₀R₁₁, —OR₇, —O(CH₂)ₘNR₇Rₚ₃, —C(O)R₇, —C(O)OR₇; —C(O)NR₁₀R₁₁; —OC(O)R₇, —OC(O)OR₇, —OC(O)NR₁₀R₁₁; —NR₈C(O)R₇, —NR₇C(O)NR₁₀R₁₁, —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁, —NR₈S(O)ₚR₇, —NR₇S(O)ₚNR₁₀R₁₁, or —NR₇S(O)ₚOR₇;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
or R₁₀ and R₁₁, taken together with the nitrogen to which they are attached, form an optionally substituted heterocyclyl or an optionally substituted heteroaryl;
R₁₇, for each occurrence, is independently —H, an alkyl, an aralkyl, —C(O)R₇, —C(O)OR₇, or —C(O)NR₁₀R₁₁; and
p, for each occurrence, is, independently, 0, 1 or 2; and
m, for each occurrence, is independently, 1, 2, 3, or 4.

44.-45. (canceled)

46. The method of claim 13, wherein R₅ is an optionally substituted cycloalkyl, optionally substituted cycloalkenyl, or a substituted alkyl, wherein the alkyl group or the cycloalkyl group is substituted with one or more substituents independently selected from the group consisting of:
—ORₚ₁, —NHRₚ₃, —N(Rₚ₃)₂, —O(CH₂)ₘORₚ₁, or —(CH₂)ₘORₚ₁;
an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl;
halo, cyano, nitro, —NR₁₀R₁₁, —OR₇, —O(CH₂)ₘNR₇Rₚ₃, —C(O)R₇, —C(O)OR₇; —C(O)NR₁₀R₁₁; —OC(O)R₇, —OC(O)OR₇, —OC(O)NR₁₀R₁₁; —NR₈C(O)R₇, —NR₇C(O)NR OR₁₁, —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁, —NR₈S(O)ₚR₇, —NR₇S(O)ₚNR₁₀R₁₁, or —NR₇S(O)ₚOR₇;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
or R₁₀ and R₁₁, taken together with the nitrogen to which they are attached, form an optionally substituted heterocyclyl or an optionally substituted heteroaryl;
R₁₇, for each occurrence, is independently —H, an alkyl, an aralkyl, —C(O)R₇, —C(O)OR₇, or —C(O)NR₁₀R₁₁; and
p, for each occurrence, is, independently, 0, 1 or 2; and
m, for each occurrence, is independently, 1, 2, 3, or 4.

47.-50. (canceled)

51. The method of claim 13, wherein R₅ is a phenyl group substituted with one to five substituents selected from:
—ORₚ₁, —NHRₚ₃, —N(Rₚ₃)₂, —O(CH₂)ₘORₚ₁, or —(CH₂)ₘORₚ₁;
an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl;
halo, cyano, nitro, —NR₁₀R₁₁, —OR₇, —O(CH₂)ₘNR₇Rₚ₃, —C(O)R₇, —C(O)OR₇; —C(O)NR₁₀R₁₁; —OC(O)R₇, —OC(O)OR₇, —OC(O)NR₁₀R₁₁; —NR₈C(O)R₇, —NR₇C(O)NR₁₀R₁₁, —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁, —NR₈S(O)ₚR₇, —NR₇S(O)ₚNR₁₀R₁₁, or —NR₇S(O)ₚOR₇;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
or R₁₀ and R₁₁, taken together with the nitrogen to which they are attached, form an optionally substituted heterocyclyl or an optionally substituted heteroaryl;
R₁₇, for each occurrence, is independently —H, an alkyl, an aralkyl, —C(O)R₇, —C(O)OR₇, or —C(O)NR₁₀R₁₁; and
p, for each occurrence, is, independently, 0, 1 or 2; and
m, for each occurrence, is independently, 1, 2, 3, or 4.

52.-53. (canceled)

54. The method of claim 13, wherein ring A is represented by the following Structural Formula: 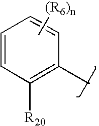
wherein:
R₆, for each occurrence, is independently a substituent selected from:
—ORₚ₁, —NHRₚ₃, —N(Rₚ₃)₂, —O(CH₂)ₘORₚ₁, or —(CH₂)ₘORₚ₁;
an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl;
halo, cyano, nitro, —NR₁₀R₁₁, —OR₇, O(CH₂)ₘNR₇Rₚ₃, —C(O)R₇, —C(O)OR₇; —C(O)NR₁₀R₁₁; —OC(O)R₇, —OC(O)OR₇, —OC(O)NR₁₀R₁₁; —NR₈C(O)R₇, —NR₇C(O)NR₁₀R₁₁, —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁, —NR₈S(O)ₚR₇, —NR₇S(O)ₚNR₁₀R₁₁, or —NR₇S(O)ₚOR₇; and
n is zero of an integer from 1 to 4;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
or R₁₀ and R₁₁, taken together with the nitrogen to which they are attached, form an optionally substituted heterocyclyl or an optionally substituted heteroaryl;
R₁₇, for each occurrence, is independently —H, an alkyl, an aralkyl, —C(O)R₇, —C(O)OR₇, or —C(O)NR₁₀R₁₁; and
p, for each occurrence, is, independently, 0, 1 or 2; and
m, for each occurrence, is independently, 1, 2, 3, or 4.

55.-60. (canceled)

61. The method of claim 54, wherein ring A is represented by the following Structural Formula: 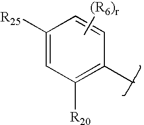
wherein:
R₂₅ is:
—ORₚ₁, —NHRₚ₃, —N(Rₚ₃)₂, —O(CH₂)ₘORₚ₁, or —(CH₂)ₘORₚ₁;
an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl;
halo, cyano, nitro, —NR₁₀R₁₁, —OR₇, —O(CH₂)ₘNR₇Rₚ₃, —C(O)R₇, —C(O)OR₇; —C(O)NR₁₀R₁₁; —OC(O)R₇, —OC(O)OR₇, —OC(O)NR₁₀R₁₁; —NR₈C(O)R₇, —NR₇C(O)NR₁₀R₁₁, —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁, —NR₈S(O)ₚR₇, —NR₇S(O)ₚNR₁₀R₁₁, or —NR₇S(O)ₚOR₇; and
r is zero or an integer from 1 to 3,

62.-83. (canceled)

84. The method of claim 13, wherein ring A is represented by the following Structural Formula: 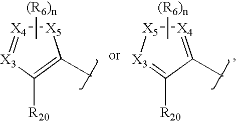
wherein:
X₃ and X₄ are each, independently, N, N(O), N⁺(R₁₇), CH or CR₆;
X₅ is O, S, NR₁₇, CH═CH, CH═CR₆, CR₆═CH, CR₆═CR₆, CH═N, CR₆═N, CH═N(O), CR₆═N(O), N═CH, N═CR₆, N(O)═CH, N(O)═CR₆, N⁺(R₁₇)═CH, N⁺(R₁₇)═CR₆, CH═N⁺(R₁₇), CR₆═N⁺(R₁₇), or N═N;
R₆, for each occurrence, is independently:
—ORₚ₁, —NHRₚ₃, —N(Rₚ₃)₂, —O(CH₂)ₘORₚ₁, or —(CH₂)ₘORₚ₁;
an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl;
halo, cyano, nitro, —NR₁₀R₁₁, —OR₇, —O(CH₂)ₘNR₇Rₚ₃, —C(O)R₇, —C(O)OR₇; —C(O)NR₁₀R₁₁; —OC(O)R₇, —OC(O)OR₇, —OC(O)NR₁₀R₁₁; —NR₈C(O)R₇, —NR₇C(O)NR₁₀R₁₁, —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁, —NR₈(O)ₚR₇, —NR₇S(O)ₚNR₁₀R₁₁, or —NR₇S(O)ₚOR₇; and
n is zero or an integer from 1 to 4;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
or R₁₀ and R₁₁, taken together with the nitrogen to which they are attached, form an optionally substituted heterocyclyl or an optionally substituted heteroaryl;
R₁₇, for each occurrence, is independently —H, an alkyl, an aralkyl, —C(O)R₇, —C(O)OR₇, or —C(O)NR₁₀R₁₁; and
p, for each occurrence, is, independently, 0, 1 or 2; and
m, for each occurrence, is independently, 1, 2, 3, or 4.

85.-101. (canceled)

102. The method of claim 13, wherein ring A is represented by the following Structural Formula: 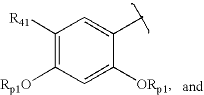
R₅ is represented by the following Structural Formula: 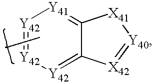
wherein:
X₄₁ is O, S, or NR₄₂;
X₄₂ is CR₄₄ or N;
Y₄₀ is N or CR₄₃;
Y₄₁ is N or CR₄₅;
Y₄₂, for each occurrence, is independently N, C or CR₄₆;
R₄₁ is —H, —ORₚ₁, —NHRₚ₃, —N(Rₚ₃)₂, —O(CH₂)ₘORₚ₁, or —(CH₂)ₘORₚ₁; an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl; halo, cyano, nitro, —NR₁₀R₁₁, —OR₇, —O(CH₂)ₘNR₇Rₚ₃, —C(O)R₇, —C(O)OR₇; —C(O)NR₁₀R₁₁; —OC(O)R₇, —OC(O)OR₇, —OC(O)NR₁₀R₁₁; —NR₈C(O)R₇, —NR₇C(O)NR₁₀R₁₁, —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁, —NR₈S(O)ₚR₇, —NR₇S(O)ₚNR₁₀R₁₁, or —NR₇S(O)ₚOR₇;
R₄₂ is —H, —ORₚ₁, —NHRₚ₃, —N(Rₚ₃)₂, —O(CH₂)ₘORₚ₁, or —(CH₂)ₘORₚ₁;
an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl;
halo, cyano, nitro, —NR₁₀R₁₁, —OR₇, —O(CH₂)ₘNR₇Rₚ₃, —C(O)R₇, —C(O)OR₇; —C(O)NR₁₀R₁₁; —OC(O)R₇, —OC(O)OR₇, —OC(O)NR₁₀R₁₁; —NR₈C(O)R₇, —NR₇C(O)NR₁₀R₁₁, —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁, —NR₈S(O)ₚR₇, —NR₇S(O)ₚNR₁₀R₁₁, or —NR₇S(O)ₚOR₇;
R₄₃ and R₄₄ are, independently, —H, —ORₚ₁, —NHRₚ₃, —N(Rₚ₃)₂, —O(CH₂)ₘORₚ₁, or —(CH₂)ₘORₚ₁; an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl;
halo, cyano, nitro, —NR₁₀R₁₁, —OR₇, —O(CH₂)ₘNR₇Rₚ₃, —C(O)R₇, —C(O)OR₇; —C(O)NR₁₀R₁₁; —OC(O)R₇, —OC(O)OR₇, —OC(O)NR₁₀R₁₁; —NR₈C(O)R₇, —NR₇C(O)NR₁₀R₁₁, —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁, —NR₈S(O)ₚR₇, —NR₇S(O)ₚNR₁₀R₁₁, or —NR₇S(O)ₚOR₇;
or R₄₃ and R₄₄ taken together with the carbon atoms to which they are attached form an optionally substituted cycloalkenyl, an optionally substituted aryl, an optionally substituted heterocyclyl, or an optionally substituted heteroaryl;
R₄₅ is —H, —ORₚ₁, —NHRₚ₃, —N(Rₚ₃)₂, —O(CH₂)ₘORₚ₁, or —(CH₂)ₘORₚ₁;
an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, alkoxyalkyl, haloalkoxyalkyl, a heteroalkyl, or a haloalkyl;
halo, cyano, nitro, —NR₁₀R₁₁, —OR₇, —O(CH₂)ₘNR₇Rₚ₃, —C(O)R₇, —C(O)OR₇; —C(O)NR₁₀R₁₁; —OC(O)R₇, —OC(O)OR₇, —OC(O)NR₁₀R₁₁; —NR₈C(O)R₇, —NR₇C(O)NR₁₀R₁₁, —NR₇C(O)OR₇; —S(O)ₚR₇, —OS(O)ₚR₇, —OS(O)ₚOR₇, —OS(O)ₚNR₁₀R₁₁, —S(O)ₚOR₇, —S(O)ₚNR₁₀R₁₁, —NR₈S(O)ₚR₇, —NR₇S(O)ₚNR₁₀R₁₁, or —NR₇S(O)ₚOR₇;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
or R₁₀ and R₁₁, taken together with the nitrogen to which they are attached, form an optionally substituted heterocyclyl or an optionally substituted heteroaryl;
R₁₇, for each occurrence, is independently —H, an alkyl, an aralkyl, —C(O)R₇, —C(O)OR₇, or —C(O)NR₁₀R₁₁; and
p, for each occurrence, is, independently, 0, 1 or 2; and
m, for each occurrence, is independently, 1, 2, 3, or 4.

103.-126. (canceled)

127. The method of claim 102, wherein R₅ is represented by the following Structural Formula:

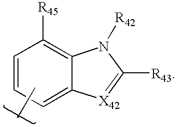

128. The method of claim 127, wherein X₄₂ is CR₄₄, and R₄₃ and R₄₄ are, independently, selected from the group consisting of —H, methyl, ethyl, propyl, isopropyl, cyclopropyl, methoxy, ethoxy, propoxy, and cyclopropoxy.

129. The method of claim 128, wherein X₄₂ is CR₄₄, and R₄₃ and R₄₄, taken together with the carbon atoms to which they are attached, form a cycloalkenyl, aryl, heterocyclyl, or heteroaryl ring.

130.-131. (canceled)

132. The method of claim 127, wherein X₄₂ is N.

133.-140. (canceled)

141. The method of claim 13, wherein the triazole compound is represented by Structural Formula (XXA), or a tautomer, a pharmaceutically acceptable salt, solvate, or clathrate or a prodrug thereof, and the method comprising reacting a compound of Structural Formula (XXIA): 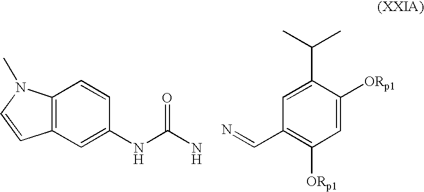
with an oxidizing agent, thereby producing the compound of Structural Formula (XXA): 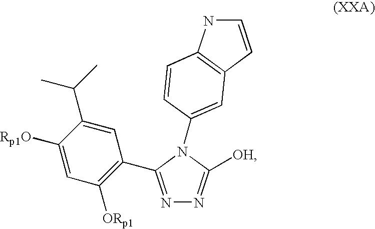
wherein Rₚ₁, for each occurrence, is independently selected from groups suitable for protecting hydroxyl.

142.-150. (canceled)

151. A compound represented by the following Structural Formula: 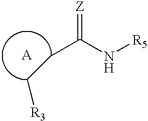
wherein:
Z is S or N—NH₂;
ring A is an aryl or a heteroaryl optionally substituted with one or more substituents in addition to R₃;
R₃ is —OR₂₆, —SR₂₆, —O(CH₂)ₘOR_{A}, —O(CH₂)ₘSR_{B}, —O(CH₂)ₘNR₇R_{C}, S(CH₂)ₘOR_{A}, —S(CH₂)ₘSR_{B}, —S(CH₂)ₘNR₇R_{C}, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —SS(O)ₚOR₇, —NR₇S(O)ₚOR₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂, —OR_{A}, —SR_{B}, NR₇R_{C}, NR₂₆R_{C} or N(R_{C})₂, wherein R_{A} is a hydroxyl protecting group; R_{B} is a thiol protecting group, R_{C}, for each occurrence, is H or an amine protecting group, provided at least one R_{C} is an amine protecting group;
R₅ is an optionally substituted heteroaryl, an optionally substituted aryl, an optionally substituted cycloaliphatic, or an optionally substituted alkyl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl; R₂₆ is a C1-C6 alkyl;
p, for each occurrence, is, independently, 0, 1 or 2; and
m, for each occurrence, is independently, 1, 2, 3, or 4.

152. A compound represented by the following Structural Formula (IIA): 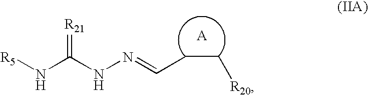
wherein:
ring A is an aryl or a heteroaryl, wherein the aryl or the heteroaryl are optionally further substituted with one or more substituents in addition to R₂₀;
R₅ is an optionally substituted cycloalkyl, an optionally substituted cycloalkenyl, a substituted alkyl, a substituted phenyl, an optionally substituted heteroaryl or an optionally substituted 8 to 14 membered aryl;
R₂₀ is —ORₚ₁, —NHRₚ₃ or —N(Rₚ₃)₂, wherein Rₚ₁, for each occurrence, is independently selected from groups suitable for protecting hydroxyl, and Rₚ₃, for each occurrence, is independently selected from groups suitable for protecting an amino group;
R₂₁ is O, NH, or NR₂₆, and R₂₁ₐ is OH, NH₂ or NHR₂₆; and
R₂₆ is a C1-C6 alkyl.

153.-164. (canceled)
